SEQUENCE LISTING (132 sequences)

SEQ ID NO: 1 - DNA, 2736 bp, Solanum lycopersicoides
  source 1..2736: mol_type = unassigned DNA
atggaggctacaatcgttgcagcgttgagtccggcggcgacaaaagcggtaagctttctggtggacagtctatcgcagttactatcggaaaatgtggaactgataagaggtgcagatagagatttccagcaattactggatgaaattacacccataaatgagttactatctggagattatgcacaattgaaaagcaacaacaacaacgatttggataaattgttccagaatattcaacgaacagtatataaagctgaagatgcgatcgataaattcctaattcaggcgaaaattaacgaagctaacgtgttcaataagttcggtccgtttgtcaagtggaacaacaataggaaaattacaccggaattcagaaaaattctcgaacaagtgactgaaattcgccaacagactcaacaggttttggagaaaaccggtatacagagttctgctttccagcctggagaaactacccggccacagggtcctgctgaggaggatagcgaagtggttggttttaataagcctgcagaagatgtgaaaaagcgactttgtgaaggatcaaaagatcttgatgttatacctattgtgggtatgccaggacttggaaagaccacactttcaagaaaagtttacaatgattcttccattgatttttatttttaccataaaatgtggatttatgttgggacatcaaagaaaccaaaggatattcttgttgagattgtgaaagatgtcgcgcaaagcaatagtaaggaactaattaaagacaaggatgaggaccaattagctcatatcatacgtgattttcttgttaaaagaggtaaacatctcattgtcttggatgatgtgtgggacacacaagttgtagattttgtcaagaaagctttcccaaacaacaaatcctggccccgaggggacagaatcatgttgacaactcgccaacgacgtgtggctgaagctgtcagcgctcgtcctcactatctggaaaacttgtcaaaagaggatagtataaagttgttggaacagagagtttttgccaataaaaggacatgtcctattgagttagaaggatatcgagatgggattgtagataaatgttgtggtgtaccacttgccatagtggtgatttcaggagctttgagaggtgttatggacgaaagtgaatggagagtagtcgaggaaaatgtggggaagcaccttataaacaaggacgaccataaaagctgcttgaaatttgttgaaacgagttacaatcatttgctgcaagagaaaaaggcagccttcttgtattttggagtatttcctcaaggttttgatattcctgcttggaaccttattcgcttatgggttgctgaggggctaataaagtccgatcataaagacagtgaaatcgagaaggttgcggagacttacttgagcgactttgctagtaggaatttagtgatggtgatgcaaaagagatctaacggtcaaatcaaaacgtgtcgtcttcatgacatgttgcatgagttctgcattattgaggctcaaaggataagtctctttcaacaagtatatctccaacctggtgttcgagtttttccttctatagaagatccaaataattctcgtcgattatgtattcaatcctctattccgtataattttatccctaaagatagaattgtacagcacgttaggtctctcttatgtttttcctcagaccaaaagcaaattgacttgtctaatctagatgtccaactcatccccaatgcctttccactcatcagggtgttggacattcaatccctcatatttgaattctccaagatgttttatggtctatttcacttgaggtatattgccatcaagggcgacttcactgtcattccttcactctttcgtaatttttggtatttacaaacccttatacttcgtaatgatgatacacatacttcaagatccaccctcgagataaaagaggacatatggaaactgttacaattgagacatctgcactccgaccttcatgtgaaattgcctcctcctcctaccccaacaagcaatagtcgtacttcttgtctacaaactctttctaaggttacaccagatagttgcaaaaaaactgtgtttgcaaaggcttgtcatctcaaaaaattgggtattgaagggcaattggcacttcttcttggaaaatctaccaagggacgtggattcgacagtttccaagagctaaggtgcgtcgaaaaattgaaattgttgaacaatgattttagtgaagagcttcaccttcctccacactttttcagcttacaaaaaacgctgaacaagttaactttgtcaagtacaaggtttgagtggagtgaggcagatatgttggggaagttggaatgccttaaggtactaaaattgaaatataatgcattcatagggaagaattggaagcccaagaaaggaagttttagcaagctccaagtcctgcacattgtctgggcaggagactggcaaacttgggatgcatcaaatcgtccgttccaaagtcttacacaccttgttcttatttcctgtaatcatctcaaggctgtgccacatgagctggctgatttaccttatcttcaagagatgaagctgacgcgcacatacgacgcagtcagttctgccatagaaatcaaaagaaagatactacaaaggcaagatcccgaaagcaacatcaaattcaatctcattacatttccccctacctcgtccacaaattaa

SEQ ID NO: 2 - DNA, 2613 bp, Solanum lycopersicoides
  source 1..2613: mol_type = unassigned DNA
atggcggctacttacggtgcactaacttctgtgttgggaaccatagagaaggttttacggtccaaccatagagaatatggttggaccgtaaaattgaaatcactgtatgggttgtttgacactctaattgtgaaatgtgatgtcggagaagcaattattagcaaggatttgctaagaagaatcaaagatgttgcagtttatgcagaagatgaagttgaatcactaatgaaacaacttattattattgggatgaatgatgaatgttgtggtgcgaagcttgataaagtctctcaacaggtaatacaagtaactcattcagtcaacaatattaattgtccaaaaaaagttgtttctccacgattagatgatgattcaatccatgagaactttatggaagggtacaatgaagaaagagtaaatatggttcaaagacttaccaaaggatcaaatagactgcaagtggtttctgttgtggggatggcgggcataggtaagacaacttttgccaaaactacattattacatattaacaaggaggactttcctattcgtggttggattacggtgtctaaaaactatgatttaagaaagttgctcctactcctccttcatgatgttattgaaatgaaaggtaagaattttgatggaatggataatggacaactagctgctcatctaaagcaagttttgcagggaaaaaggtatttgattgttgtggatgacatatggagcaaagaagattgggatgaaattaaacattggtttccagattgtggtgacaaaagtcgaattttgttgacttctcgagacaaagaggttggtgagtatgctgctaaagaaggtttggtactgatgcgtcctctgacgcaagatgaaagtcggaatctgttttaccatagggcatttgggaaaaattacactattcgagggtcagatattgatgaattcgaaaaggttggagaaaaagttataacaaattgcaaaggattaccactaatgattactgcagttgctggtatactccgtagtaagagtaaactggatgagtggatggaagtagctcaaagtgtaagctcattagtaaatgatgatgattacaaacaatgcttacaagttgttgctttgagctacaataatcttccttctctaatgaaagcttgctttttgcatttcggagtttttccaaaagcccatgtcatttctgtgaagaagttgatcagattatggattgcagaaggactcgtaaatctaaagggagttgaggaattcgaacaagcagctgatcgtgttttacatgatcttattggtaaaagtgtagttattgttgacaagcgaagtttggatgggaaaattaagacatgtaggattcatgatctttttcatgatttctgctccaaggaagctgaaagcgagaatcttctgtatgttgttggttcagattctcaagtacacacaaatttccgtcaaggttgtaggtgggtgtcagttcaatcaaaacttgattatcgtcaccgcagacggtatactcctattgaaatatcctctttttacatgctatataatattgttatttttgacaaaaaagtttttcatttcaaactactaagggtattggacttggaggtacatgatatcataggactcagtgaaattactagagaccttgtttgtttaaggtatttggctgtcgggattaagtatgtaagttttgtggatctcccgattaccaatctttggaatctacacactctcattttaggtaaatctttcactaatagtcttatacataaagtcttcagttttccaaaagatatttggcaaatgtcacaattaaggcatctttatgcaaaaggcatttatctatcttcgcctggagaaaaggttctcgaaaacttacagagtgtttctggtttgagtccttcttgttgtacaaaggaaatatttgaagggattaagaatgtgaaaaaattgatcattcgtggaaagagggaggattatcctacagatattaaatggatagataatcttaaatatttgcaacatctcgagtcactgagtattgaaactaccgattttctatttcataagaataaaaccaggttttttagtcttacaagtccagattcttttccacaaaaactcaagaaattgaaacttagctacacatatctaccgtgggaatacatgtccattatcagcaagttgcccgaacttgaggtactcaaactgaagtgtggttccttaattggcgacgagtggaaagcaacagaccagattgggttcccgaagttgaggttcttgctccttgagaatctcttccttggaaaatgggaaaccaccacgggctctcatgatcatttccccagccttgagcgcttaattatcacaaatttcagtttcttaaaagagattcctcaaggatttgctgatagcaagaaactggagctgattgagttacacaattgtgacccttccttggtggcttttgctaagcagatacaggttgaacacgaggatttggggaggaacaaacttaaagttactgccttcgatacaggtcgaggatggaacagaggaatcttagcttcaagaaataacaggatttccagctaa

SEQ ID NO: 3 - DNA, 2727 bp, Solanum lycopersicoides
  source 1..2727: mol_type = unassigned DNA
atggaggctacaatcgttgcagcggtgagtccggcggcgacaaaagcggtaagctttctggtggacagtctatcgcagctactatcggaaaatgtggaactgataagaggtgcagatagagatttccagcgattactggatgaaattaaacccataaatgagttactagctggagattatgcacaattgaaaagcaacaacaacaacgatttggataaattgttccagaatattcaacgaacagtatataaagctgaagatgcgatcgataaattcctaattcaggctaaaattgacgaagctaacctgttcaataagttcggtccgttcgtcaagtggaacaacaatcggaaaattgcaccggaattcgagaaaattttaaaacaagtggccggaattcgccaacagactcaacaggttttggacaaaaccggtatacagagttctgctttccagcctggagaaactacggggacacagggtcctgctgaggaggatatcgaagtggttggttttaataagcctgcagaagatgtgaaaaagcgactctgtgaaggatcaaaggatgttgatgttatacctattgtgggtatgccaggacttggaaagaccactctgtcaagaaaagtttacaatgattgttcccttgatttttatttttaccataaaatgtggatttatgttgggacatcaaagaaaccaaaggatattcttattgagattgtgaaagaagtcgcgcaaagcaatagtaaggaactaattatagacaaggatgaggaccaattagctcatatcatacgtggttttcttgttgaaagaggtaaacatctcattgtcttggatgatgtgtgggacacacaagttgtagattttgtcaagaaagctttcccaaacaacaaatcccggccccgaggggacagaatcatgttgacaactcgccaacgacgtgtggctgaagctgtcagcgctcgtcctcactatctagaaaacttgtcaaaaaaggatagtataaagttgttggaacagagagtttttgccgataaaaggacatgtcctattgagttagaagtatatcgagatgggattgtagataaatgttgtggtgtaccacttgccatagtggtgatttcaggagctttgagaggttgtatgggtgtgggagtagtgcaagaaaatatggggaagcaccttataaacaaggacgaccataaaagctgcttgaaatttgttgaaacgagttacaatcatttgccgcaagagaaaaaggcagccttcttgtattttggagtatttcctcaaggttttgatattcctgcttggaaccttattcgcttatgggttgctgaggggctaataaagtccagtcataaagacagtgaaatcgagaaggttgcggagacttacttgagcgactttgctagtaggaatttagtgatggtgatgcaaaagagatctaacggtcaaatcaaaacgtgtcgtctccatgacatgttgcatgagttctgcattattgaggctcaaaggataagtctctttcaacaagtatatctccaacctggtgttcgagtttttccttctatagaagatccaaatacttctcgccgattatgtattcaatcctctattccgtataattttatcactaaagatagaattgtacagcatgttaggtctctcttatgtttttcctcagaccaaaagcaaattgacttgtctaatctagatgtccaacttatccccaatgcctttccactcatcagggtgttggacattcaatccctcatatttgaattctccaagatgttttatggtctatttcacttgaggtatattgccatcaaaggcgacttcactgtcattccttcactctttcgtaatttttggtatttacaaacccttatacttcgtaatgatgatacacatacttcaagctccacccttgagataaaagaggacatatggaaactgttacaattgagacatctgcactccgaccttcctgtgaaattgcctccccctcctaccccaacaagcgagagtcgtacttcttgtctacaaactctttctaaggttacgccagatagttgcaaaaaaactgtgtttgcaaaggcttgtcatctcaaaaaattgggtattgaagggcaattggcacttcttcttggaaaatctaccaagggaagtggattcgacagtttccaagagctaaggtgcgtcgaaaaattgaaattgttgaacaatgattttagtgaagagcttcaccttcctccacactttttcagcttacaaaaaacactgaacaagttaactttgtcaagtacaaggtttgagtggagtgaggcagatatgttggggaagttggaatgccttaaggtactaaaattgaaagataatgcattcatagggaagaattggaagcccaagaaaggaagttttagcaagctccaagtcctgcacattgtctgggcaggagactggcaaacttgggatgcatcaaatcgtccgttcctaagtcttacacaccttgttcttatttcctgttatgatctcaaggctgtgccacacgagctggctgatttaccttatcttcaagagatgaagctgacgcgcacattccaggcagtcagttctgccatagaaatcaaaagaaagatactacaaaggcaagatcccgaaagcagcatcaaattcaatctcattacatttccccctaactcgtccacaaattaa

SEQ ID NO: 4 - DNA, 1359 bp, Solanum lycopersicoides
  source 1..1359: mol_type = unassigned DNA
atggcggctacttacggtgcactaacttctgtgttgggaaccatagagaaggttttacggtccaaccatagagaatgtggttggaccgtaaaattgaaatcactgtatgagttatttgacactctacttggcaaatgtgatgtcggagaagcaattattagcaaggatttgctaagaagaatcaaagatgttgcagtttatgcagaagatgaagttgaatcactaatgaaacaacttattgttattgggctgaatgatgaatgttgtggtgcgaagcttgataaagtctctcaacaggtaatacaagtaactcattatgtcaacaatattaattgtccaaaaaaaattgtttctccacgattagatgatgattcaatctatgagaactttatggaagggtacaatgaagaaagagtaaatatggttcaaagacttaccaaaggatcaaatagactgcaagtggtttctgttgtggggatggcgggcataggtaagacaacttttgccaaaactatattatttcatgaccatcttaagaaggaggactttcctattcgtggttggattactgtgtctaacaactatgatttaagaaagttgctcctactcctccttcacgatgttattgaaatgaaaggcaagaattttgatgaaatggataatggagaactatctggtcacgtaaagcaaggtttgcagggaaaaaggtatttgattgttgtggatgacatatggagcaataaagattgggatagtattaaacatttgtttccagattttggtgacagaagtcgaattttgttgacttctcgagacagggaggttggtgagtatgctgctaccaatcctaaagatggtttggtactgatgcgtcctctgacgcaagatgaaagtcggaatctgttttaccatagggtatttgggaaaaattacactattcgagggtcagatattgatgaattcaaaaaggttggagaaaaggttataacaaattgcaaaggattaccactaatgattactgcagttgctggtatactccgtagtaagagtaaactggatgagtggatggaagtagctcaaagtgtaagctcattagtaaatgatgatgattacaaacaatgcttacaagttgttgctttgagctacaataatgttccttctcttatgaaagcttgctttttgcatttcggagtttttccaaaagcccatgtcatttctgtgaagaagttgatcagattatggattgcagaaggactcgtaaatctaaagggagttgaggaattcgaacaagcagctgatcgtgttttacatgatcttattggtaaaagtgtagttattgttgacagctgtcatgactga

SEQ ID NO: 5 - DNA, 2157 bp, Solanum lycopersicoides
  source 1..2157: mol_type = unassigned DNA
atggaagggtacaatgaagaaagagtaaatatggttcaaagacttaccaaaggatcaaataaactgcaagtggtttctgttgtggggatggcgggcatagttaagacaacttttgtcaaaactatattattacatattaacaaggaggactttcctattcgtggtaggattactgtgtctaaaaactatgatttaagaaagttgctccaactcctcctccatgatgttattgaaatgaaaggtaagaattttgatggaatggatactggaaaaaggtatttgattgttgtggatgacatatggagcaaaaaagattgggatgaaattaaacattggtttccagattgtggtgacagaagtcgaattttgttgacttctcgagacagggaggttggtgagtatgctgctaaagatggtttggtaccgatgcgtcctctgacgcaagatgaaagtcggaatctgttttaccatagggcatttgggaaaaattacactattcgagggtcagatattgatgaattcgaaaaggttggagaaaaagttataacaaattgcaaaggattaccactaatgattactgcagttgctggtatactccgtagtaagagtaaactggatgagtggatggaagtagctcaaagtgtaagctcattagtaaatgatgatgattacaaacaatgcttacaagttgttgctttgagctacaataatcttccttctctaatgaaagcttgctttttgcatttcggagtttttccaaaagcccatgtcatttctgtgaagaagttgatcagattatggattgcagaaggactcgtaaatctaaagggagttgaggaattcgaacaagcagctgatcgtgttttacatgatcttattggtaaaagtgtagttattgttgacaagcgaagtttggatgggaaaattaagacatgtaggattcatgatctttttcatgatttctgctccaaggaagctgaaagcgagaatcttctgtatgttgttggttcagattctcaagtacacacaaatttccgtcaaggttgtaggtgggtgtcagttcaatcaaaacttgattatcgtcaccgcagacggtatactcctattgaaatatcctctttttacatgctatataatattgttatttttgacaaaaaagtttttcatttcaaactactaagggtattggacttggaggtacatgatatcataggaatcagtgaaattactagagaccttgtttgtttaaggtatttggctgtcgggattaagtatgtagattttgtggatctcccaattaccaatctttggaatctacacactctcattttaggtaaatattttattcataatagtcttatgcataaagtcttcacttttccaaaagatatttggcaaatgtcacaattaaggcatctttatgcaaaaggcattcatctatgttcgcctggagataataaggttctcgaaaacttactgagtgtttctggtttgagtccttcttgttgtacaaaggaaatatttgaagggattaagaaagtgaaaaaattggtcattcgtggaacgagggaggaatgtcctacagatgttaaatggatagataatcttaaatatttgcaacatctcgagtcactaagtattgaaaatatcaaatttatatttcatgagaatgaaaccaggttttttagtcttacaagtccagattcttttccacaaaaactcaagaagttgaaacttagctacacatatctaccgtgggaatacatgtccattatcagcaagttgcccgaacttgaggtactcaaactgaagcgtagttccttaattggcaacgagtggaaagcaacagaacagattgggttcccgaagttgaggttcttgctccttgagaatctcttccttagaaaatgggaaagcaccacgggctatcatgatcatttccccagccttgagcgcttaattatcacaaatttcaaattcttaaaagagattcctcaaggatttgctgatagcaagaaactggagctgattgagttacacaattgtgacccttccttggtggattttgctaagaaggtgcagcttgaacacgaggaggttttggggaggaacaaacttaaagttactgccttcaatacaggtaaatatatctattag

SEQ ID NO: 6 - DNA, 1176 bp, Solanum lycopersicoides
  source 1..1176: mol_type = unassigned DNA
atggccgctacttacgctgcactaacttctgtgttgggaaccatagacaagcttttacggtccaacttattagttggcgtagaagaggttcataagcaacaattagaatcactcgacaagatgtttggcactctgcaagtgtctctaattggcaaatgtgatggcgaagaagcaattattagcaaggatttgcaaagaagaatcaaagatgttgcagttgatgcagaagatgaagttgaatcactaatgagacaacttattattattgagctgaatgatgaatgttgtggtgcgaagcttgataaagtctctcaacaggttatacaagtaactcattctgtcaatgaagagctgatcatcatcaacaatattaattgtccagaaaaagctgatgaaaatagtgcttcttctccacgattaggtgattcaatccgtgagaacgttatggaagggtacaatgaagaaagagaaaggatggtgcaaagacttaccaggggctcaggatcaaatagacgggaagtggtctctgttgtggggatgccgggcataggtaagacaacttttgccaaaactatattattcgataactctatcaagagggtctttcgtattcgtggttggattactgtgtctaacaagtatgatttaaaaaagttgcttctactgctccttcatgatgttattgaaatgaaaggcagcaataattatgatgaaatggatattggacaactatctggtcacgtaaagcaaggtttgcagggacaaaggtatttgattgttgtggatgacatatggagcaataaagattgggatacaatttcacattggtttccagattgtggtgacagaagtcgaattttgttgacttctcgagactacaaggttggtgagtatgctgcaaccaatcctaaagatggtttggtactgatgcgtcctctgacgcaagatgaaagtcggaatctgttttaccatagggcatttgggaaaaattacagtattcgagggtcagatattgatgaattcgagaaagttggagaaaaagttataacaaattgcaaaggattaccactaatgattactgcggttgctggtatactctgtagcaagagtaaactggatgagtggatggaaatagctcaaagtgtaagctcattagtaaatgatgatgcttactaa

SEQ ID NO: 7 - DNA, 1968 bp, Solanum lycopersicoides
  source 1..1968: mol_type = unassigned DNA
atgctgtatgcgacaacagatgaacggaaaatgaagcatttcaagattcaagtacttgagggtgttagatttacgccattcaagattctattagcatctaagataggatgttatggaggcacggattatgatagagggttagtcgaaagatatggtacattgagtcatgaggcagctgagatgcttaaacttgcttgttatgatgctgaacaagtcattgacaaggacgttgatagagataccgttatcgaagcacttttaaaatcaggaaatgaagcaaccctttttgtatatcccattgttggagttgaaggcattggaaaaactactcttgccaaattggtgtataatgatccaaggatagttagtcagtttcagctgcgtatatgggttcgtgtgtctcgtgtgtttaaagtagaggaagtattagaacaaattgtgaattcagttagagaagatttatgtgagaaacttgatatgaatgagctgaagaatctagttcatgagactttgtatggaaagaattatttgattgtgttagatgatgtgtggaatgaggacccagtgaagtgggatgaacttaagaagttgttgatggtaggtggttgtggaagtaagattcttgtaactactcggaaaaaggaagtagcttcgataatggggacggttcctgcatactgtttggagggtttgttccatgaagatagtctgactttgttcttgagtaaggcatctgaacaaggatcttcattgcgcaagaaaactgagagacgggagtgggagatagttaacaatcacagcgggtggaactcaactcagaatgacgaaatttcatctgcactaagagtcgcagaaggtctcattagtaagtctaatgaatcagaagatcttgaggacgttgccattcaacatttccgagagttattgtcgagatccttctttcaagacgttgaagaatatcgttccgtttatacttcaatctgtacaatgcatgaccttgtacatgatcttgcactgtcagcagcaggggttgaattctgtacagtaaattctcacatacaaaacatttctgatgaggtcagacatgtggtgttttctgactatgatttatcaggcaaggaactgccagcatcccttctcggtaaccaggcattaaggaccatatccttctccgttgatggagtagggccgatgagtacaatgttcgttgagaattgcttagcaagattcatgcaacttaaggtgctagatatcagtgattcatgtttcgatgagctgcctagctctgttggcgaattgaagtatttaagatatcttgatgtaagttccaatggaagcattaaagaattacctgattcgattaacaagttgctgagcttacagacacttcgagtttctcattgtccacaacttgaaggtctgcctaaagatattggaaatttgatcagcctaagacacctgtatataaccaccaagcaagcatgttttcctgaaaagcaattggctgcttatcatctcttcgttctttgtacattcatagctgcaacaatctcgtatctttgtctgaagcaccttactgctttaaagaatctgttgattgttgactgtaaagagcttacattgttggagtggcaagatattgaaggacttaggatgcttcggtcattggttatcggaggcttacctgaattggagtcaaaagatgttcactgcctcgggagccttcagatgttggtacttgctggtttaccagagttagttactttgccgcgatggcttgaaggtgctagtgctactctacaatacctgagggtggaaaggtgcctgaattttgcagcattgccaaactggctggcaaatcttactgcacttgaaaaacttgaaatttccaagtgccgtaaatcattttcattgcccgagggtatgagttgcctcacgaatctgaaggtacttaagaacgacaattga

SEQ ID NO: 8 - DNA, 2202 bp, Solanum lycopersicoides
  source 1..2202: mol_type = unassigned DNA
atgcatttccagaaaagctttaggaagaaggtaagaagattcttttccagttcaaatccaattatatatcgattcaagattggcagaaaggtaaaagaaatcagggagctgctgaatgagattgcagatgataggagaaatttccacttcacggaacatacttatgtaattccagctgagaatacgagtagagaacaaacacactcctttgtgagggcctcagatatcattggtagagatgatgatcaagaaaacattgtaaaacagctgatagattctcatgatgaggaaaatatttctgtgattcctattgttggacttggagggcttggaaaaaccacacttgttaagttggtttataacaataatagggttgttcagaattttgaccttagaatgtgggttagtatttcagaagatttcagtctgagcaaggtaattgagaaaattctgaggtccgcaacaggagagagttttgaccacctagatatggaccaattacaatgttgtttgggagaggttttgcaacagaaaaggtatttacttgtgctggatgatgtttggaacgaagatcaacacaagtggacggatctgagggagttgctgatgaattgttccagaggtagtaaaattgttgtcactacacgtagtaagatggttgctttgattactggaacagttccgccttattatttgggaggccttgctaatgatgactgcttatctttatttttgaaatgtgcatttggaggacaggacaatttgtttcctaatctagtagaaataggaaaagaaattgtgaaaaagtgtggaggagtgcctttggctgtgaaaaccttgggaaggttgttgtacatgaaaacagacgagaatgaatggttgcagataagagataatgagatatgggaaatcgaacagaataaatctgacattttaccaatattgagattgagctatgaacagatgccatcacatctaagacagtgctttgcctattgctccatgttacccaaaggtcaagaaattccgagggaggattttatcaatcgctggattgctcaaggatttatacagagttccaacagaaacaggaagctggaagatatcggtaatcagtactttgatgagttgctatcaaggttttgcttcctagatgtggtacaagcttttgatggagaaatattggcttgtaagatacacaatcttgtgcatgatcttgcacagtcagtatcaggtgcagagtgcttaaatgtgaaacccaatgctttcgtggtctctgaaagagttcgccacttatttttccatgcagaagatatgtctaggaaacacttccccagatttttgcttcctttgcaaaagttgaggtctttctcttattcttttaacattggacctgtaaacaagttctttgtcaagacaatgttgtcaaatttcaaatgccttcggatgctagtcttgaacaatctagatcttgaggagttgccaacttcgataggtcacttgaaggaattaagatacctcaaccttagtgacagtggtaagatcaagtttcttccaaggtctatgagcaaattagtaaatctgcacaccctaaacctcattaactgtgaacagcttaaggagttgccaagagattttagaaagttaatcagcctgaagaccttgtatttgactacacatcagatgtcagcagggatcaagaatcaacattctttcacttctcttcaatttttacttcttttcaaatgttgtttcccaaaattgcagccagaactggtgcagcattttactgcacttcgggttttgcgtatctatgaatgcccaagtttatgttctcttccaagcagtattagatatctgacttcacttgaaaagctatggatctggaactgtgaagaacttgatttgattgatggagaagggatgtcaggcctaacaagtcttcaatccttgcttctaatggggcttcctaagttggtgactctaccattggaacttaaagatactgctcctacaacattaaagtacttcagaatcgccgattgtcccaacctggtggagcttccagagtggctgcctaattgctcctcacttcagagactctatatagaggattgtcctgttttggcatcgatacctcaaggaatctacagccacaatgccaacgtccatataatcgactgtccattgctaggtggatga

SEQ ID NO: 9 - DNA, 2553 bp, Solanum lycopersicoides
  source 1..2553: mol_type = unassigned DNA
atggctgatgcctttgtgtcatttgcagttaaaaaattgggtgatttcctaatacaacaagtttccctgagaaaaaatatgagagatgaagttacatggctgagaaatgagctactcttcatgcagtctttcctcagagatgccgaacaaaagcaatatggagatcaaagagttcaacaatgggtgtttgagatcaactctcttgctaatgatgctgtcgctatactcgagacttacagcttcgaggcttgtaaaggtgatgaggatggatttgctagtcgtctcaaggcttgcgcttacatctgtaggaaggagaagaaattctacaatgtcgccaaggagattcaatcactcaagcaacgaatcatggatatctctcgcaaacgagatacttatggtattacaaatatcaatagtactaattcaggagaagggccaagtaatcagtctgccatggttagaacattgaggagaactacctcctatgtggatgaccaggactacatatttgttggacttcaagatgttgtacgaacattgctagatgaacttctcaaagcagagcctcgtcgaagtgtcctctccatttatggcatgggcggattaggcaagaccactcttgcaagaaacctctacaacagtcctaacatagtcaatagcttccctacacgcgcttggatatgtgtttctcaagagtacaacacaatggatctccttaggaatatcataaaatccatccaaggttgcaccaaggaaactttagatatgttggaaaggatgacagaaagagatctagaaatataccttcgtgatctattaaaagaacgcaaataccttgtggtggttgatgatgtatggaggagagaagcatgggagagtttgaaaagagcatttccagatagcaagaatggaagcagagttattattaccacgcgcaaagaggatgtcgctgaaagagcagatgacagaggttttgtccataaacttcgcttcctaagccaagaagaaagttgggatctcttttgtaggaaactacttgatgttcaagcaatggcctccacaatggagaggctagctaaagatatggtggacaagtgtggaggcttacctcttgcaattgttgtactgagcggactactttcgcataaaagggggcaagacgaatggaaaaaggtgaaagatcacctttggcagaacattgaaaacaactctattgaaatctcctacatactatcattgagctataatgatttgtcaattgcactcaagcagtgttttctgtactttggcctttttccagaagatgaagaggtcgatgctgaatacataatatggttgtggatggccgagggtttcatacctaatggagaagaaagaatggaagatgtcgctgaaggcttcttgaatgagctgataagacgaagcttagttcaagtggctggaacactttgggagaaagttattctatgtagggttcatgatgcagttcgtgatctttccatacaaaaggcaatggaggtaaacttctatgacatttataatccaagaaaccattccatatccttcttacctattagacatgccattcatagtcaaggagaaaggtacctctcacttgatctttctaatttaaagttgaggtcaattatgttcttcggtccagattttcgtaacatgaatcttataaactttagtagtgcgttccaacatatatatgtgttgtacttggataatcgtggtggttctatatctatagtacctgatgcgataggaagtttgtaccacctcaagttcttaagcttgagaggcatccatgatcttccctcctccattggcaacctcaagaatttacagacactttctgtcgtgaatgaaattggacacccattcaaactaccctgtgagtcagttgatctaataaatctaagacatttagttgctccgtatacaaaacctttgaaacgtataagcaaactcaaaaatcttcaagttcttaaaggcactgcttgtgatcagtggaaagatgttgaccctgttgatttagtcaatcttcgagaattaagcatgcatgatattaccaaaatttacgccctgaacaacattagcaacttgaaaaaccttagcacactcagattgttttgtagaggtggtgaatcattcgctgcccttgaatttcttagttcttgtcaaaagctcgagaaattgttgttaaaagggagaatagagaaacttcctttgtttgtaaattccatcacaatgatggttttttggaactcaaaactcatggaagatcccatgcctatcttgggaatgttgccaaacctaagggatctctatttagtagcaacttatgaaggaaaagaaataatgtgcggtgacaacagcttcaggcaactcgagttccttcgtcttcatcgtcttgagaacctagaaacatggcatttagccacaggttccttgcctctgattaaaggtcttcgtatctatgattgtccaaagctgaaggagattccagatagattgaaacatgtgaagcttttcaaccatatatga

SEQ ID NO: 10 - DNA, 1275 bp, Solanum lycopersicoides
  source 1..1275: mol_type = unassigned DNA
atgtcttttgcacttggaaaattgggtgatttactcttcataaaatctttcctcaaagatgcagaactaaagcaatgtggagatcaaagagttggagaatgggtatttgagatcaactctatagctaatgatgttgttgctacactcgagccttgcatctttgaggttggatgttgtacaaacattgctagctcaccttctcaaagcagagcctcgtcgaagcgtcctctccatttatggtatggggggttaggcaagaccactcttgccagaaaactttacattagtcctaatatagcctctagtttccctacacgtgcttggatatgtgtttcgcaagagtacaacaccattgatcttcttaggaatatcatgaaatccctccaaggtcgcaccaaggaaactctagatttgttggaaaggatgatcgaaggagatctagaaatttatcttcgtgatcgattaaaaaaacacaaatgccttgtggtggttgatgatgtgtggcagaaagaagcatgggagagtttgaaaagagaattcccggatagcaagaatggaagcggagtcattattaccacgcccaaaagggaagtcgctgaaagagcagatgacagagaagatcaagtgctcaaggctgataacataatacggttgtggacggccgagggtttcatacccagaggagaagaaagaatggaggataccgctgaaggcttcttcaatgagcttataatacgaagcttggttcaagtggctaaaacattttggggaagagttactgaatgtagggttcatgatttactccatgatcttgcgatacaaaaggcattggaggtaaacatcttttacatttatgatccaaaaagccactccatatcctctttatgtatcagacatgtcattcatagtcaaggagaaaggtacccctcacttgatctttctaacttaaagttgaggtcacttatgttcttcgatccagattttcgtaagatgagtcttataaacttcaggagtgtgttccaacatctatatgtgttgtacttggagatgcgtgttgacaatatgtctattgtattagtatctgatgccataggaagtttgtaccacctcaagttcttaagcttgagagctccagaaattgaggttagaaggggtgatagagaaactgcctcatctgtttccaaattccatcacaatgatggttctgagggactcaagactgacagaagatctgatgcctattttggaaacgttgccaaacttaaggaatcatga

SEQ ID NO: 11 - DNA, 2388 bp, Solanum lycopersicoides
  source 1..2388: mol_type = unassigned DNA
atgagagatgaagttacatggctgagaaatgagctactctttatacaatctttcctcaaagatgcagaacaaaagcaaagtagagatcaaagagttcaacaatgggtatttgagatcaactctattgctaatgatgttgttgctatactcgagacttatagcattgaggctggtaaacgtgctagtcgtctcaaagcttgcgcttgcatatgtagcaaggagaagaaattctacagtgttgccgaggagattcaatcactcaagcaacgaatcatggatatctctcgcaaaccagagacttatggtattacaaatatcaattataattcaggagaagggccaagtaatcatgattacatttttgttggctttaacgatgttgtacaaacattgctagctcaacttctcaaagcagagcctcgtcgaagtgtcctctccatttatggcatgggcggattagggaagaccactcttgccagaaacctctacaacagtcatgatatattcaatagcttccatacacgcgcttggatatgtgtctctcaagagtacaacacaatggatcttcttaggaataccataaatccatccaaggaaactctagatttgttggaatgggtgacagaaggagatctagaaatttatcttcgtgatctattaaaagaacgcaaataccttgtggtggttgatgatgtatggaatagagaagcatgggagcgtttgaagagagcattcccggatagcaagaatggcagcagagtcattattaccacgcgcaaagaggatgtcgctgaaaagcaaacgatagaggtttttcaagaagaaagttgggatctcttttgtaggaaactacttgatgttcgagcaatggtttcagaaatgggaagtctagctaaggatatggtggaaaagtgtagaggcttacctcttgcaattgttgtattgagcggactactttcgcataaaaaagggctaaacgaatggcaaaaggtgaaagaccacctttggaagaatattaaagaaaataaatctattgaaatctctaacatactatctttaagctacaatgatttgtcaactgctctcaagcagttttttctctactttggtatttttccagaagatcaagtggtaaaggctgataacataatacggttgtggatggtcgagggtttcatacccagaggagaagaaagaatggaggatgtcgctgaaggcttgttgaatgaactgataagacgaagcttggttcaagtggctaatacattttggggaagagttactgaatgtagtgttcatgatttactcgatcttgcgatacaaaaggcatcggaggtaaacttctttgacatttatgatccaagaagccactccatatcctctttatgtatcagacacttcattcatagtcaaggagaaaggtacctctcacttgatctttctaacttaaagttgaggtcaattatgttcttcgatcgagatttttgtaagacgagtcttataaacttcaggagtgtgttcctacatctatatgtgtttcacttggatatgaatgttgggaatatgtctatagatgtacctgatgccattggaagtttgtaccacctcaagttcttaagcttgagaggtatccgtgatcttccctcttccattggcaacctcaagaatctacagacacttgttgtcgatgtgggaggatacactttccaactaccccgcgagacagtttacctgataaatttaagacatttagttgctcggtattcaaaacctctggtacatataagcaaactaactagtcttcaagttcttgaaggtgttggttgtgatcaatggaaagatgttgaccctgttgatttagtcaatcttggagaattaagtatgtttgatattagcaaatcttactccctaaacaacattaggagcttgaaaaaccttagcactctcacattgtcgggggggtatcggaactcatctccatttccagaccttgaatttgttaattgttgtgaaaagctccagaaattgaggttagaaggggtgatagagaaactgcctcatctctttccaaattccatcacaatgatgcttctttggaagtcaagactgacagaagatccgatgcttattttgggaatgttgccaaacctaaggaatctcgatttggttagtgcttatgaaggaaaagaaataatttgcagtgataacagtttcaatcaactagagttccttcaacttgaggatcttcgaaatctagaaagatggtatttaggtacaagtgccatgcctctaattaaaggtcttggtttccatgactgtccaaatttaaaggagattcctatgagaatgaaagacgtggagctgctgaagaggaattatatgtggtga

SEQ ID NO: 12 - DNA, 1782 bp, Solanum lycopersicoides
  source 1..1782: mol_type = unassigned DNA
atggaaaatgaatatcagaagctggaggatatcagaattgggttgcatcaaagagcggaggctgatcggagaaacttactagtcatttcacctaatgttgaggcttggtttactcgtgttgatactactactgcagatgtggaagctgtaatgcgacgagaggttgaaagatatggctggtgcccaaaattgaagtcacgttactcgctgagcaggaaagctaaaaaatttgcactggaattgattgaacttcgaaatcaaggcaatgattatgctgttttctcctatcctgcagtcaaaattgaagttctacctagtaacagtggtgaggagtttgactccagaaaattgcaagaggaagaggttatggcagctttgagagatgatggggtcactatgattgggatatgtggtatgggtggttttggtaagacaacactggctgagaaaatcaggcaaaaggcgaaacaaggaagtttgtttgatgaggttgtcatggtaactatcagtcaacaaccagacttgaaaagaattcaaggtgaggtagcagggggtgttggtctgacgttgcaaggggacaatttctggaatcgtggagatcagctgcgctcaaggttaatggttcagaacagctgtgtcctagtaatcttggatgatgtttgggaggctcttcatgaactagagaaacttggaattcccagatgtagcaaccacaaccaacgttgtaaagtgacattgacaacgcgattccgagatgtttgtgaagctatggaagctcaaaagatcatggaagttggaactttacctgaagaggaagcatggatccttttcagggagaaagttggtaatataatcgacgacgatccttctgtacttgacatagcaaaagttgttgccaaagaatgcaaggggttgccgcttgcaattattacagttgcaagagcacttaagcgtaaaaccaagccttcgtgggaggatgcccttgtacaattacaaagatcaacaccaataaatattccaggagtaattaaaaatgtgtatcaatctctcaaacttagctatgattacttggaaagtaatgaagtcaggtccctgtttttgctttgttccttgttcgaggaagatagtaatatctggactgaacaattacttagatatggaatggggcttgacatcttttcagaaattaaaaacttagaagaagcaaggaagcgggcgtgtcatctcttagaaacattgaaagatcgtttcttcttgtccttaggttcaacagaaaattatgtcaaaatgcatgatatggtccgtgatgtggctataagtattgcctctatgggagagtatagctttatggtaagtcaccatgtgaactcgcaagagctcctaagaagaacttcttacaagcaatacagtcacatttcaattgttgcaaataaatttgatgagcttcctaacccaatattttgcaaaaaactgaagcttctaatgttgactctcgattttaaaaatccatttaaattacaggaagatttttttgatgaaatggctgaactcaatgtcttaagtttgagtggatatggatccattctgccttttccaacatccattgagaggttgtcaagtttgaagacattatgtctgagtcatttaaggttagatgacatatccattattgggatacttgtcactttagaaattctcagcataagacattctgacatagaggagctgccagttgagataggaaatttgaccaatctaattatgttaagagtttcggaatag

SEQ ID NO: 13 - DNA, 3219 bp, Solanum lycopersicoides
  source 1..3219: mol_type = unassigned DNA
atggatcatcttatgccttacctgctgaaagaaatcaaagattactattacaaggagtatgagatctctcaatgctcttccgagatctcttctctacttatcgacataatgcctcttgagctggaggttctatacatttttacttctaagctcatgaaagaatcaaggtcaacagaactagaagtgtttgttaagcaaatcctaaaagcatctccgaagattcttcaaaattatctgattcttatgaaaggatgcatggaaggttcagtagctgtcagttacgctccaattcaatgcattaatgtcatgatggagttcctattgatctttctcacttatataccaaagcgctatatccgtcgtgacaaactgaatgatatgttggcacatgttggagtacttacaaggaagatatctattctggtgagcaagctgatggaggagagctctgagaataatatcaacgaagcggacttttcagctgcatacttgttgcaagaaattgaacaaatgaagggagatatcagacagctttttttgaaagccccagagtcatctcaaattcgcttttctatggatgatggtttactcttcatgaatcttctactcggacatttaaatgatttgctcatttccaatgcttattcagttgctctgataaaaaaagaaattgggatagtgaaagaaagccttgaattcctaagatcatctttcggggaagtcaggcaaacattggatgacactagcggattagttaaagattgttgggtgcgtgctttagatgtggcatatgaagcagaacatatcattaattccattcttgtcagagataatgctctcacacatctcatattctcacttccgaatgtcacagataagatcaagcttatcgtggcagaagtcacctgcagtgtacatctggaggataaaaatggggatgacccccttgatgcaaagtctttcgacgagtcaattgagtcaacctcatcatcttttgttgaggtaacagttggtcatgaggaagacgaagcctggatcgttgaccagctccttgataagcatgaatccaagcttgatgtcatttcgattgtcggaatgccaggagtcggtaaaactactctagccaacaaagttcatttcaatgttcatgtttggtgcactgtttcccaaaagtttaacaagtcaaaggtgttgcgggtgattcttcagcaagttacagggtcggaagacaagaaacaaagtaatgagggtgataaagttgttgatcttgctgaaaagctacgagaagaactatacgataaaaggtacctcatcgtcttggatgatgtgtgggatattgcaacagtggagatgttaatagcatgctttccgagggttgagagaggaaatagaattatcttaactagccgaagtagtgaggtaggtttgcaagttaaatgtcgtaatgatcttctctaccttcaacttctaacacatgaaaaaagttggaatttatttgaaaaaaggatcaaggaagctgccctgctgaactgtcagaagttggacaccaaatatatagttgagaaatgtcaagggcttcctctggctgttgtgttgattgctggagtaattgttagaggaaagaaaaatgaaaaagatttgtggcttaagattcaacataatatggattccgttatttctgccaacaacaatttgcagatgatgaaggttatgcaattaagttatgaccacttaccataccacctaaagccgttgttgctttactttggaagatctcaaaagaacaaactaactccagtctctaagttgatgcaattgtggatggccgaagggtttgtggatcattgtatcccgtctaagagtagtttagaggaaataacccaaagttacttggaagctttaatttccagtagcctggtaatggtggatcgtagcgtgtccaagagtagtcaccctttttctgttactatcaaggtttgctatgtgcatgatgttgtgcatgatttttgttcagtaaaagcaaagaaggaaatgtttttcaagttaatcaattcaggtgctccatttcatgcttcggatttcatacatcgtcgtctaaccatttatactgacaaatcccaactccacaaaagatgtgttttgtttaattctaataagtgttcagctggtagtaagcatctcatatctttgaaagtgaaaaattggcttgatcccttcagtcacattagacactttggacttgttagagtgttgcaacttggtaacattattctggaagagtcatcaatggaagaaataggctccctatttcatttgaggtttttgaggtttcagacatatgaaaaatctctcccgctttcgtggttgaacctccagaatctggaaactctgtggatcctaaacaggcattccaccatggtactactcgacgaactgtcaaagctgaaacatgtgagcatcgacgctagatctttctttgaagaggacaaggacaacattatgcatcagccaagtagaatattggaagctgagtgttcaaagttagaatacttgacaactttatcccgagttgatatctcatattctcaaggcacaactgatgctctggggaagttcccaaatcttcaggactttgattgcaacattctggtaccgaatgatcctcctgcaaacggcgattggtttcccaagtttgatgtccttaataaacttgaatcactcattctaagttacagttatgtctggagcagtactgataaatgtctggattattccaagtttgaaaaaatccgaaatcccaatgaatatcacttccctaccagtttgcaagagttacggttgcataggtttcccctgagacctgctttgttgttagcaatcgcgacattgtctgagcttgaaattctggagattatagaatctaatttcctcgaggatgagtgggacgcaagtaaggacatctatcaaagtctaaagactttgcatttagcagatgtctttcttacagaatggcaagttgataagggaacttttcccaagcttgtggaattaaaactagaacattatcacgggcttatggacatcccttatgcatttggggatatagacactttaaagtccattcgagtggttcaaacaagccgtcaccttggaaattcagccacaaaaattaaggaagatgtagaagcttacacgggaaaggaaagacttaatgtccac

SEQ ID NO: 14 - DNA, 3480 bp, Solanum lycopersicoides
  source 1..3480: mol_type = unassigned DNA
atggatggattggctgaaactggatcttcttcttcttcttcttcttcttctttgtggccatgcacttatgatgttttcttaagttttagaggagacgatgtacggaagaatttcgtcgatcatctatatacagctttgcagcaaagaggaattcacactttcaaagatgatgaaaaacttgaaagagggaaatctatttcaccttcacttttcaaagctatcgaagaatcgatgatttccatcatcatattctctcaaaactatgcttcttcttcgtggtgtctagatgagcttgttaagatcactcaatgcatgaaactcaggggacagattgttcttcccgtcttctatgacgtggatccatctgtcgtaagaaaacaaaaggcaaatgttggtgagctcttcgctaaacatgagttagatttcaaagatgatgaagaaagggtgaagagatggcgtactgctatgacagaagcagcaaatgtatctggttgggatttgccaaatatagctaacgggcacgaatcaaagtgtatcgatcaagttgtagaatgtgtcatggagatattaggtcatactgcttctgatgctactgaaagtcttattgggatacgctcaagaatggggacggtgtattccttgttgaatctggagtctgataaagttcaattcgttggaatatggggaatgagcggaataggaaaaacaactatagcaagagccatctatgacaagattttccgttactttcaaggtgctactttccttcatgaagttggagaaacttcagccaaacatggtatccaacatttgcagaagatacttctttctgaactacttctgttaaaagatctaagaataaacaacgtatttgaaggaaccagcttgataagaagacgactaaaagggaaacgagtcctaattgttcttgatgatgtcaatcatggaaaccagttagatgccctagctaaaagccatgactggtttggtgcaggcagtgtaatcatcataacaacaaaggataagcagttgcttcgtcaatataacgtggacaaaatgtataaagtgagtctgttaaacactgatgaaagtattgaactccttagttcgtatgcattccagaatcgtcatcccaaaagtggatatggagagattatagctgaagttgttcggtatgctggtggtcttcctttagctcttaaagttttgggttgctctctgtatggcggaggcatgattgaatggagagaaacagtggagagactaaaacaaattccagaaggcgaaattgtagaaaagctcaaagtaagtttcaatggactaagtgagactgaccaaaagatcttcttagatattgcatgtttctttaaagggaagaagaaaggttctgtcattagaattcttcgtagtttcagttttactcctgtcgttggcataaggaatctcatcgaaaaatctcttgtaactgtttcaaaaggtaggattgtgatgcatcagttgatccaagagatgggttggcatattgttcgcaaagaagcttcaaacaatcttggcaagtatactaggctctggtctaccgatgatattcttcaggtactatctgaaaatacggccacagaagctgtggaaggcatatggttgcacttgcctataccgaaagacataaatgttggtgcagaagccttcaaacaaacggacaacctgaggctgctcaagatacacaatgcaagtgtctctgtagctccagattgtcttcctaataaattgatatggcttcattggcatggctacccaatgaagtcacttccagcaagttttcgagcagaaaggcttgtttgtctgaaaatgcagtatagccgcgttgtacacttgtggaagggagtaaaattcctacacaaactgaagtttctcaaccttagtcactcccaaaagctagtcagctgtccagatttcacaggggtgcccaatctcgaaaagttggttcttgaagattgttcgagtataactgagatccatccttctgtgggatatctcaaaaatcttgttctactaaacctgaagaactgcaagaatcttaagacccttccaaacattattcgattggataatcttgagactttaattctttctggctgcttgaaactcgcgaatttcccagaaatcatgagtgacatgaattgcttatctgaggtctacttggaagctacagatgtaaaagagttgccttcatcaattgaacgcctccctggccttcgattgatgaatctaggctactgcaggaatcttacaaatttaccaaaaaccataggtagattaaaatctcttaggattcttattctttctggatgttcaaagctagaaaagttgccagaggaactgggacatatagaaatcttggaggaactctattgcgacgaaacttccattcaaagcccaccatcatccattacactgttgaagaaccttaagaccttatcttttcatggatgtaaaggcatggtatctcaatcatggagttcacttttctatgcatggcttcagccaagaaaacataatcacaagccaacaagtctgatgtttacttccttttctggtttattttctttgaggaaattggatcttagtgactgttgtatgttggatgaaggaattcctagtgatcttggatgcttgtcttctttggttgagctaaatcttagtgggaataattttgtggatctctctcaagcaagtctcaacatgcttccgcggctcagaatccttgagctagttggttgtgagaggcttgaaaggttgccagaacttccaacaacaatagaggaagtttttgcagataattgtacatacctgatgactgataacatgggaatattgaccaactacaagatgttacagcgaatatcgttcactaattgtgttggactactgcagaatcagcagacgcgtgacatggctacctcattgtggcttcacctattcaagaagtgcattgttaaaagtggtcactttagcatttacctacctggagaacaagttccagaatggtttggctacaaactcaacggaacttcagtttcaatgcagctgccaaacgattggtacaatgataaattcatgggatttgccatctgtgttgtgtctgaacttgaaacaacatggttatcagttcatgaaggttacctacaggaaatgccaggcatttcgattgagtttacaatcaaaagccacctccggaggagcacgagttgcctaatgaacattggttttgtagggacaaacaagaatgttgcttcagatcacacatgccttgcctatgtgccatttgaagaatattggtcaatgtacaaaaaccacttagacagcccaaacaattggtatcagatcgatttttctgcaaactctttgaggaaacatatattcctaaaaagttggggaattcgtctcgtgtacactgatgacttacagattttcacatcttga

SEQ ID NO: 15 - DNA, 2463 bp, Solanum lycopersicoides
  source 1..2463: mol_type = unassigned DNA
atggcggtgacggactttttcgccggcgaaattaccaccgaactcataaaatacctgctgttaatagttaaaaaatccactttatgccgttcaagcgcagagaatctcattgacaatatcaatggtctccttccaatcatccaagaaatcaaacaaaccggtgttgaacttccacagatacggcaaactcagctcgacgatttctccaaacttctccgagatggctacgaactcgccggaaaagttctgcactccggccgttggaacatgtacaggaacctacagttggctaggaaaatggagaggttagagaaaagagtagcgaggttcatgcaagttacaatgcaagctcatgtactagctgatgttcatcatgttaggtttaatatggagcagagatttgatgtgcttgagcataggcttaaagctataaaaatcggagttgacgatagaagtggtggaggaggagggtgtttaggagaagctgtgaaaagaatggaagaagatgagaaatggtttgaggatagttttgtaaatttaggtgctgggattgaattggggaagaggaaggtgaaggagatgctgatgggtgaacaagataggggtgtgtttgagatttgtggaattgggggtagtggcaaaactaccttggctaaggagatttgtaaagatgatcaagttaaaagttatttcaaggacaagattttctttttcactgtttctcaatctccaaatgtggagcaattaaggaaaatgatttgggaaaagatatcagggtgcaatctccatggttatggatacggggagatgtttccccagtggaacctacagtaccaatggaatacgaaatgtgcatccccggtactcttgattcttgatgatgtgtggtctgcatctgttctagagccactagttttcaagatccccggatgcaagatcctagttgtatcgcgcatcaagtttcctctatcgatcattgactgtatttatgatttagagttgttaaggaagatgaagctatgtccttattttgccattttgcttttggacacaattcctttccgcgtgattgtggatgaatgtgaagggcttcctttggctcttaaggtcattggatcttcattgaagggaaaacctgagatgttatggacaagtgcaaaaaacagattatcacgatgccaacctgtctgcgagtctcatgaactgcagttgcttgagcgaatgaaattgagtattgactgtttgcctgagaaggtgcgagagtgtttcctggacttgggtgctttcccggaggacaaaaggattcctgttgatgttctaattaacatgtgggtggagctacatgatattgatgaggaggaggcttttcacattcttgttgaactttcagacaaaaatctcctaaatctagtcaaagatgcacgagccggagacatgtatacaagttactatgagatatcggtgtttcagcatgatgtattacgagacctagcaattcatatgagcaactgtgatgatataaatcagagaaagcgattggttatgccgcggagagacacaagcttcccaagagaatgggaaagaaatgtggacgaacctttccatgcacgagttatctctgtgcatacagatgaaatgagagaaatggactggttcagaatggattgcccgaaagctgaagtactgattctcaactttgcctcatctgagtacttcttgcctccttttctggagaacatgccaaagctaagggcattgataatcataaactatagtgctggcaattcagttcttcataacatgtctgtattcagtcatttaaccaacttgagaagcctttggttcgagaagatatccatcactcacttatctgactccacaaatcctctcaataacctgcgaaagatatctctagtgctttgtgacatgaaaaacagcttcgatgagtcagatgtagacctccctggtttgttcccacagctctcggagttcacaatggatcattgcatcaacttcaacaagctaccatcgagcatttgccggttgcataagctcaacagccttagtatcactaattgtgatagtctttgtgaacttccatctgatttaggtgaattacaaactctacaagttttaaggatatatgcctgtccacatctgaaaaggcttcccccgggaattggtcatctggtaaagctaaagtaccttgacatttcacaatgtgttggtttgagatgtctccctgaagcaattggttgctgtagaaacttagagaagattgatatgagggagtgccctcaaatcgacagtttgcctagcgctctatcctttcttgaatcattacgttgtgttatctgtgacgatgaagttttttgtcaatggaaagacgttgagaaggctgtaccaggtctctgtgtacaggttgccgaggagtgccatactcttgactggctatctcaataa

SEQ ID NO: 16 - protein, 255 aa, Solanum lycopersicoides
MKIAPVAINHSPLSREVPSHAAPTQAKQTNLQSEAGDLDARKSSASSPETRALLATKTVLGRHKIEVPAFGGWFKKKSSKHETGGSSANADSSSVASDSTEKPLFRLTHVPYVSQGNERMGCWYACARMVGHSVEAGPRLGLPELYEGREGPAGLQDFSDVERFIHNEGLTRVDLPDNERFTHEELGALLYKHGPIIFGWKTPNDSWHMSVLTGVDKETSSITFHDPRQGPDLAMPLDYFNQRLAWQVPHAMLYR

SEQ ID NO: 17 - protein, 321 aa, Solanum lycopersicoides
MGSKYSKATNSINDALSSSYLVPFESYRVPLVDLEEATNNFDHKFLIGHGVFGKVYKGVLRDGAKVALKRRTPESSQGIEEFETEIETLSFCRHPHLVSLIGFCDERNEMILIYKYMENGNLKRHLYGSDLPTMSMSWEQRLEICIGAARGLHYLHTRAIIHRDVKSINILLDENFVPKITDFGISKKGTELDQTHLSTVVKGTLGYIDPEYFIKGRLTEKSDVYSFGVVLFEVLCARSAIVQSLPREMVNLAEWAVESHNNGQLEQIVDPNLADKIRPESLRKFGDTAVKCLALSSEDRPSMGDVLWKLEYALRLQESVI

SEQ ID NO: 18 - DNA, 2472 bp, Solanum lycopersicoides
  source 1..2472: mol_type = unassigned DNA
atggcggaatctttcttgttcaatatcattgaacgggttttggctaaagtttcttcaattgctgtatatgagatcagtctagcttggaatgttaagacagagctaaggaaactccaaagtactctatccaccatcaaagctgtacttctagatgcaaacgagcaaaaggcaaagaaccatgaagtgagagattggctggaaaagctcagagatgttgtttatgatgtcgatgatttgatggatgatttatcaacacaactgttgctgcaaatgcatttccagaaaagctttaggaagaaggtaagaagattcttttccagttcaaatccaattatatatcgattcaagattggcagaaaggtaaaagaaatcagggagctgctgaatgagattgcagatgataggagaaatttccacttcacggaacatacttatgtaattccagctgagaatacgagtagagaacaaacacactcctttgtgagggcctcagatatcattggtagagatgatgatcaagaaaacattgtaaaacagctgatagattctcatgatgaggaaaatatttctgtgattcctattgttggacttggagggcttggaaaaaccacacttgttaagttggtttataacaataatagggttgttcagaattttgaccttagaatgtgggttagtatttcagaagatttcagtctgagcaaggtaattgagaaaattctgaggtccgcaacaggagagagttttgaccacctagatatggaccaattacaatgttgtttgggagaggttttgcaacagaaaaggtatttacttgtgctggatgatgtttggaacgaagatcaacacaagtggacggatctgagggagttgctgatgaattgttccagaggtagtaaaattgttgtcactacacgtagtaagatggttgctttgattactggaacagttccgccttattatttgggaggccttgctaatgatgactgcttatctttatttttgaaatgtgcatttggaggacaggacaatttgtttcctaatctagtagaaataggaaaagaaattgtgaaaaagtgtggaggagtgcctttggctgtgaaaaccttgggaaggttgttgtacatgaaaacagacgagaatgaatggttgcagataagagataatgagatatgggaaatcgaacagaataaatctgacattttaccaatattgagattgagctatgaacagatgccatcacatctaagacagtgctttgcctattgctccatgttacccaaaggtcaagaaattccgagggaggattttatcaatcgctggattgctcaaggatttatacagagttccaacagaaacaggaagctggaagatatcggtaatcagtactttgatgagttgctatcaaggttttgcttcctagatgtggtacaagcttttgatggagaaatattggcttgtaagatacacaatcttgtgcatgatcttgcacagtcagtatcaggtgcagagtgcttaaatgtgaaacccaatgctttcgtggtctctgaaagagttcgccacttatttttccatgcagaagatatgtctaggaaacacttccccagatttttgcttcctttgcaaaagttgaggtctttctcttattcttttaacattggacctgtaaacaagttctttgtcaagacaatgttgtcaaatttcaaatgccttcggatgctagtcttgaacaatctagatcttgaggagttgccaacttcgataggtcacttgaaggaattaagatacctcaaccttagtgacagtggtaagatcaagtttcttccaaggtctatgagcaaattagtaaatctgcacaccctaaacctcattaactgtgaacagcttaaggagttgccaagagattttagaaagttaatcagcctgaagaccttgtatttgactacacatcagatgtcagcagggatcaagaatcaacattctttcacttctcttcaatttttacttcttttcaaatgttgtttcccaaaattgcagccagaactggtgcagcattttacygcacttcgggttttgcgtatctatgaatgcccaagtttatgttctcttccaagcagtattagatatctgacttcacttgaaaagctatggatctggaactgtgaagaacttgatttgattgatggagaagggatgtcaggcctaacaagtcttcaatccttgcttctaatggggcttcctaagttggtgactctaccattggaacttaaagatactgctcctacaacattaaagtacttcagaatcgccgattgtcccaacctggtggagcttccagagtggctgcctaattgctcctcacttcagagactctatatagaggattgtcctgttttggcatcgatacctcaaggaatctacagccacaatgccaacgtccatataatcgactgtccattgctaggtggatga

SEQ ID NO: 19 - protein, 823 aa, Solanum lycopersicoides
MAESFLFNIIERVLAKVSSIAVYEISLAWNVKTELRKLQSTLSTIKAVLLDANEQKAKNHEVRDWLEKLRDVVYDVDDLMDDLSTQLLLQMHFQKSFRKKVRRFFSSSNPIIYRFKIGRKVKEIRELLNEIADDRRNFHFTEHTYVIPAENTSREQTHSFVRASDIIGRDDDQENIVKQLIDSHDEENISVIPIVGLGGLGKTTLVKLVYNNNRVVQNFDLRMWVSISEDFSLSKVIEKILRSATGESFDHLDMDQLQCCLGEVLQQKRYLLVLDDVWNEDQHKWTDLRELLMNCSRGSKIVVTTRSKMVALITGTVPPYYLGGLANDDCLSLFLKCAFGGQDNLFPNLVEIGKEIVKKCGGVPLAVKTLGRLLYMKTDENEWLQIRDNEIWEIEQNKSDILPILRLSYEQMPSHLRQCFAYCSMLPKGQEIPREDFINRWIAQGFIQSSNRNRKLEDIGNQYFDELLSRFCFLDVVQAFDGEILACKIHNLVHDLAQSVSGAECLNVKPNAFVVSERVRHLFFHAEDMSRKHFPRFLLPLQKLRSFSYSFNIGPVNKFFVKTMLSNFKCLRMLVLNNLDLEELPTSIGHLKELRYLNLSDSGKIKFLPRSMSKLVNLHTLNLINCEQLKELPRDFRKLISLKTLYLTTHQMSAGIKNQHSFTSLQFLLLFKCCFPKLQPELVQHFTALRVLRIYECPSLCSLPSSIRYLTSLEKLWIWNCEELDLIDGEGMSGLTSLQSLLLMGLPKLVTLPLELKDTAPTTLKYFRIADCPNLVELPEWLPNCSSLQRLYIEDCPVLASIPQGIYSHNANVHIIDCPLLGG

SEQ ID NO: 20 - DNA, 20 bp, synthetic construct
  misc_feature 1..20: note = primer Spenn-ch04_541669
  source 1..20: mol_type = other DNA
taatgaggcagagcaagttt

SEQ ID NO: 21 - DNA, 20 bp, synthetic construct
  misc_feature 1..20: note = primer Spenn-ch04_5416620
  source 1..20: mol_type = other DNA
ccctcaagaaccatgaatca

SEQ ID NO: 22 - DNA, 36 bp, synthetic construct
  misc_feature 1..36: note = primer oCM26
  source 1..36: mol_type = other DNA
caccatgaaaattgctccagttgccataaatcacag

SEQ ID NO: 23 - DNA, 20 bp, synthetic construct
  misc_feature 1..20: note = primer oCM27
  source 1..20: mol_type = other DNA
cacacgcaatgctctaccgc

SEQ ID NO: 24 - DNA, 31 bp, synthetic construct
  misc_feature 1..31: note = primer oCM20
  source 1..31: mol_type = other DNA
atcgggaattcgtgctgatggatgctgcagg

SEQ ID NO: 25 - DNA, 41 bp, synthetic construct
  misc_feature 1..41: note = primer oCM21
  source 1..41: mol_type = other DNA
ccacgtgaagataccttgctgcttgttaagtcgtccgttgg

SEQ ID NO: 26 - DNA, 23 bp, synthetic construct
  misc_feature 1..23: note = primer oCM22
  source 1..23: mol_type = other DNA
agcaaggtatcttcacgtggcgg

SEQ ID NO: 27 - DNA, 31 bp, synthetic construct
  misc_feature 1..31: note = primer oCM23
  source 1..31: mol_type = other DNA
atcggcccgggttctgcgagcgatttgcggg

SEQ ID NO: 28 - DNA, 23 bp, synthetic construct
  misc_feature 1..23: note = primer oCM38
  source 1..23: mol_type = other DNA
ctgatcatgtgtgccttgacccc

SEQ ID NO: 29 - DNA, 22 bp, synthetic construct
  misc_feature 1..22: note = primer oCM39
  source 1..22: mol_type = other DNA
ggactgcagggtgtttatcggg

SEQ ID NO: 30 - DNA, 21 bp, synthetic construct
  misc_feature 1..21: note = primer 3719
  source 1..21: mol_type = other DNA
ttagctcactcattaggcacc

SEQ ID NO: 31 - DNA, 19 bp, synthetic construct
  misc_feature 1..19: note = primer 3720
  source 1..19: mol_type = other DNA
cctcttcgctattacgcca

SEQ ID NO: 32 - DNA, 30 bp, synthetic construct
  misc_feature 1..30: note = primer oCM28
  source 1..30: mol_type = other DNA
atagaggttccagcccgtggagggtggttc

SEQ ID NO: 33 - DNA, 28 bp, synthetic construct
  misc_feature 1..28: note = primer oCM29
  source 1..28: mol_type = other DNA
ccctccacgggctggaacctctatcttg

SEQ ID NO: 34 - DNA, 26 bp, synthetic construct
  misc_feature 1..26: note = primer oCM30
  source 1..26: mol_type = other DNA
gcgaatgggagcttggtatgcctgcg

SEQ ID NO: 35 - DNA, 27 bp, synthetic construct
  misc_feature 1..27: note = primer oCM31
  source 1..27: mol_type = other DNA
cataccaagctcccattcgctcattac

SEQ ID NO: 36 - DNA, 25 bp, synthetic construct
  misc_feature 1..25: note = primer oCM74
  source 1..25: mol_type = other DNA
gcgaatgggatattggtatgcctgc

SEQ ID NO: 37 - DNA, 29 bp, synthetic construct
  misc_feature 1..29: note = primer oCM75
  source 1..29: mol_type = other DNA
gcataccaatatcccattcgctcattacc

SEQ ID NO: 38 - DNA, 26 bp, synthetic construct
  misc_feature 1..26: note = primer oCM76
  source 1..26: mol_type = other DNA
agaatggttgaccattctgtcgaagc

SEQ ID NO: 39 - DNA, 26 bp, synthetic construct
  misc_feature 1..26: note = primer oCM77
  source 1..26: mol_type = other DNA
agcttcgacagaatggtcaaccattc

SEQ ID NO: 40 - DNA, 30 bp, synthetic construct
  misc_feature 1..30: note = primer oCM104
  source 1..30: mol_type = other DNA
cctaagactgccggagctctatgagggaag

SEQ ID NO: 41 - DNA, 34 bp, synthetic construct
  misc_feature 1..34: note = primer oCM105
  source 1..34: mol_type = other DNA
agagctccggcagtcttaggcgaggcccagcttc

SEQ ID NO: 42 - DNA, 32 bp, synthetic construct
  misc_feature 1..32: note = primer oCM86
  source 1..32: mol_type = other DNA
ctatgagggaaggtcaggcccagctgggctac

SEQ ID NO: 43 - DNA, 32 bp, synthetic construct
  misc_feature 1..32: note = primer oCM87
  source 1..32: mol_type = other DNA
gtagcccagctgggcctgaccttccctcatag

SEQ ID NO: 44 - DNA, 26 bp, synthetic construct
  misc_feature 1..26: note = primer oCM32
  source 1..26: mol_type = other DNA
gtgcactgttgtgtaagcacgggccg

SEQ ID NO: 45 - DNA, 29 bp, synthetic construct
  misc_feature 1..29: note = primer oCM33
  source 1..29: mol_type = other DNA
ccgtgcttacacaacagtgcacccaactc

SEQ ID NO: 46 - DNA, 26 bp, synthetic construct
  misc_feature 1..26: note = primer oCM109
  source 1..26: mol_type = other DNA
gttgggtgcactgttgtctaagcacg

SEQ ID NO: 47 - DNA, 24 bp, synthetic construct
  misc_feature 1..24: note = primer oCM110
  source 1..24: mol_type = other DNA
ccgtgcttagacaacagtgcaccc

SEQ ID NO: 48 - DNA, 31 bp, synthetic construct
  misc_feature 1..31: note = primer oCM80
  source 1..31: mol_type = other DNA
gttgtataagcacaggccgattatatttggg

SEQ ID NO: 49 - DNA, 31 bp, synthetic construct
  misc_feature 1..31: note = primer oCM81
  source 1..31: mol_type = other DNA
cccaaatataatcggcctgtgcttatacaac

SEQ ID NO: 50 - DNA, 30 bp, synthetic construct
  misc_feature 1..30: note = primer oCM106
  source 1..30: mol_type = other DNA
gctgggctatgtcggtcctcactggtgtcg

SEQ ID NO: 51 - DNA, 37 bp, synthetic construct
  misc_feature 1..37: note = primer oCM107
  source 1..37: mol_type = other DNA
gaggaccgacatagcccagctgtcattcggagttttc

SEQ ID NO: 52 - DNA, 30 bp, synthetic construct
  misc_feature 1..30: note = primer oCM34
  source 1..30: mol_type = other DNA
ctcactggtgtcgaaaaagagacgtcgtcc

SEQ ID NO: 53 - DNA, 30 bp, synthetic construct
  misc_feature 1..30: note = primer oCM35
  source 1..30: mol_type = other DNA
gacgtctctttttcgacaccagtgaggacc

SEQ ID NO: 54 - DNA, 20 bp, synthetic construct
  misc_feature 1..20: note = primer Chr4_4976565F
  source 1..20: mol_type = other DNA
gaagccaatgtctgcagctg

SEQ ID NO: 55 - DNA, 20 bp, synthetic construct
  misc_feature 1..20: note = primer Chr4_4976565R
  source 1..20: mol_type = other DNA
gccggtcatcagctaggtac

SEQ ID NO: 56 - DNA, 20 bp, synthetic construct
  misc_feature 1..20: note = primer Spennch04_6606838F
  source 1..20: mol_type = other DNA
ctggcctcattcttatggtt

SEQ ID NO: 57 - DNA, 20 bp, synthetic construct
  misc_feature 1..20: note = primer Spennch04_6606838R
  source 1..20: mol_type = other DNA
taaagggagctttcgaacaa

SEQ ID NO: 58 - DNA, 20 bp, synthetic construct
  misc_feature 1..20: note = primer Spennch04_7503010F
  source 1..20: mol_type = other DNA
ctaagcatggattttagcgc

SEQ ID NO: 59 - DNA, 20 bp, synthetic construct
  misc_feature 1..20: note = primer Spennch04_7503010R
  source 1..20: mol_type = other DNA
caacccttgaatcatggaga

SEQ ID NO: 60 - DNA, 20 bp, synthetic construct
  misc_feature 1..20: note = primer Spenn-ch04_5416619
  source 1..20: mol_type = other DNA
taatgaggcagagcaagttt

SEQ ID NO: 61 - DNA, 20 bp, synthetic construct
  misc_feature 1..20: note = primer Spenn-ch04_5416620
  source 1..20: mol_type = other DNA
ccctcaagaaccatgaatca

SEQ ID NO: 62 - DNA, 36 bp, synthetic construct; identical to SEQ ID NO: 22
  misc_feature 1..36: note = primer oCM26
  source 1..36: mol_type = other DNA

SEQ ID NO: 63 - DNA, 20 bp, synthetic construct
  misc_feature 1..20: note = primer oCM27
  source 1..20: mol_type = other DNA
cacacgcaatgctctaccgc

SEQ ID NO: 64 - DNA, 20 bp, synthetic construct
  misc_feature 1..20: note = primer KS12
  source 1..20: mol_type = other DNA
atggctcgtccaaatgtccc

SEQ ID NO: 65 - DNA, 20 bp, synthetic construct
  misc_feature 1..20: note = primer KS13
  source 1..20: mol_type = other DNA
gccagggacaacaacaccac

SEQ ID NO: 66 - DNA, 22 bp, synthetic construct
  misc_feature 1..22: note = primer KS14
  source 1..22: mol_type = other DNA
ttaccagggacaacaacaccac

SEQ ID NO: 67 - DNA, 19 bp, synthetic construct
  misc_feature 1..19: note = primer KS15
  source 1..19: mol_type = other DNA
atggctcgtgcaaatgtgc

SEQ ID NO: 68 - DNA, 20 bp, synthetic construct
  misc_feature 1..20: note = primer KS16
  source 1..20: mol_type = other DNA
gccacaaaggaaagcagcag

SEQ ID NO: 69 - DNA, 22 bp, synthetic construct
  misc_feature 1..22: note = primer KS17
  source 1..22: mol_type = other DNA
tcaccacaaaggaaagcagcag

SEQ ID NO: 70 - DNA, 23 bp, synthetic construct
  misc_feature 1..23: note = primer KS18
  source 1..23: mol_type = other DNA
atggcaaaacactcacaagtacc

SEQ ID NO: 71 - DNA, 20 bp, synthetic construct
  misc_feature 1..20: note = primer KS19
  source 1..20: mol_type = other DNA
gactccgaccccatggaaag

SEQ ID NO: 72 - DNA, 22 bp, synthetic construct
  misc_feature 1..22: note = primer KS20
  source 1..22: mol_type = other DNA
tcaactccgaccccatggaaag

SEQ ID NO: 73 - DNA, 22 bp, synthetic construct
  misc_feature 1..22: note = primer KS24
  source 1..22: mol_type = other DNA
atggcacgttcgaatgtaccaa

SEQ ID NO: 74 - DNA, 23 bp, synthetic construct
  misc_feature 1..23: note = primer KS25
  source 1..23: mol_type = other DNA
tcattttcctccaaagccaaagc

SEQ ID NO: 75 - DNA, 47 bp, synthetic construct
  misc_feature 1..47: note = primer oCM206
  source 1..47: mol_type = other DNA
tttggagaggacagggtaccatggcggaatctttcttgttcaatatc

SEQ ID NO: 76 - DNA, 36 bp, synthetic construct
  misc_feature 1..36: note = primer oCM207
  source 1..36: mol_type = other DNA
gtcgtatgggtaaggtccacctagcaatggacagtc

SEQ ID NO: 77 - DNA, 40 bp, synthetic construct
  misc_feature 1..40: note = primer oCM208
  source 1..40: mol_type = other DNA
tttggagaggacagggtaccatggctgatgcctttgtgtc

SEQ ID NO: 78 - DNA, 47 bp, synthetic construct
  misc_feature 1..47: note = primer oCM209
  source 1..47: mol_type = other DNA
gtcgtatgggtaagggattgaaacatgtgaagcttttcaaccatata

SEQ ID NO: 79 - DNA, 57 bp, synthetic construct
  misc_feature 1..57: note = primer oSM03
  source 1..57: mol_type = other DNA
ggggacaagtttgtacaaaaaagcaggctttgagctatgaacagatgccatcacatc

SEQ ID NO: 80 - DNA, 60 bp, synthetic construct
  misc_feature 1..60: note = primer oSM04
  source 1..60: mol_type = other DNA
ggggaccactttgtacaagaaagctgggtaaagcattgggtttcacatttaagcactctg

SEQ ID NO: 81 - DNA, 57 bp, synthetic construct; identical to SEQ ID NO: 79
  misc_feature 1..57: note = primer oSM15
  source 1..57: mol_type = other DNA

SEQ ID NO: 82 - DNA, 60 bp, synthetic construct; identical to SEQ ID NO: 80
  misc_feature 1..60: note = primer oSM16
  source 1..60: mol_type = other DNA

SEQ ID NO: 83 - DNA, 47 bp, synthetic construct; identical to SEQ ID NO: 75
  misc_feature 1..47: note = primer oCM206
  source 1..47: mol_type = other DNA

SEQ ID NO: 84 - DNA, 36 bp, synthetic construct; identical to SEQ ID NO: 76
  misc_feature 1..36: note = primer oCM207
  source 1..36: mol_type = other DNA

SEQ ID NO: 85 - protein, 254 aa, Solanum lycopersicum
MARPNVPKFGNWENDDNTPYTVYFEKARQTRGTGKMMNPNDPEENPDMFRNLAPPPEVAPQSKPKRQTEEPPIGRGGPARQTRDHRLSKEDGEFRQYANSPARKESVGRKGANEPSHQRGRGSNSGRTGRQSIGSEHSFDKSPLHPHYQAKVSNAGRGVASPAWEGKNNSYDSSHGTPGRSKVKQDKSDRGAAVPRFGEWDENDPQSADNYTHIFNKFREEKQGNPSGTPSRTSNNTQKHNSEEKQRKWCCCPW

SEQ ID NO: 86 - protein, 243 aa, Solanum lycopersicum
MARANVPKFGNWGNEDNTPYTVVFENARKNRGGKMINPNDPQENPDMFPNVAPSSRPKTPPTEEPMGMETARQTNKRRVSKEDGDFRASSPARNEPTTHQRHGGGRGSNSGRPSRQSGGSDHSIAKSPLHPNSQAKISGRVAASPVWEGKNLYDSSHGTPGRSFESSHATPGRHQMKQESPDRGTVVPKFGGWDDNDPQDAENYTEVFNKVREQRHVDTGNMPAAGVRTSYSTQRQQRNEKQK

SEQ ID NO: 87 - protein, 224 aa, Solanum lycopersicum
MAGSHVPKFGNWDGENVPYTAYFENARKSNSKGGKMINPNDPEENPEAFAYCGDEDANINISPLVEKHQYHYDHRRNPSVESGQNKSIGPTNSNSESFGDSQRKSVSGFSVNQPTRRRRTSDVKKNKNDRGNGFVPPSPNRPMKNSRNPSDDLSCSSAASVPKFGAWDEKDPKSGEGFTVIFNKVKEEKHIAAAKFPVVQPQSNMSSSNNHKKNAKSKVFCCLF

SEQ ID NO: 88 - protein, 211 aa, Arabidopsis thaliana
MARSNVPKFGNWEAEENVPYTAYFDKARKTRAPGSKIMNPNDPEYNSDSQSQAPPHPPSSRTKPEQVDTVRRSREHMRSREESELKQFGDAGGSSNEAANKRQGRASQNNSYDNKSPLHKNSYDGTGKSRPKPTNLRADESPEKVTVVPKFGDWDENNPSSADGYTHIFNKVREERSSGANVSGSSRTPTHQSSRNPNNTSSCCCFGFGGK

SEQ ID NO: 89 - protein, 239 aa, Malus domestica
MAQRSHVPKFGNWEDQESVPYTAYFDKARKGRTGVGGKMINPNDPEENPDILSDTSASSPPKVRPEPGKPVHERRRSREDNDLRFANSPAQRRSSGEHQPNRGRGVSSGETHRRAARPSAGSENSVERSPLHRNARVSGRDSPSWEGKASYESSHGTPARSRLKPRDESPEKGAAVPKFGEWDENDPASADGFTHIFNKVREEKAGKAPGTPSHPSYQDARKQGSNDSAKCCCFPWGRK

SEQ ID NO: 90 - protein, 241 aa, Malus domestica
MAQRSHVPKFGNWEGEESVPYTAYFDKARKDRTGVGGKMINPNDPQENPDILSDISASSPPKVRPEPEKPVHEQRRSREDNDLRFANSPAQRRNSGESAHQPSRGRGVSSGETRRRPARPSAGSENSVERSPLHRNARVTGRDSPSWEGKASYETSHGTPGRSRLKPRDESPEKGAAVPKFGEWDENDPASADGFTHIFNKVREERAGKVPGTPSQPSYQDARRQGSNDSAKSCCFPWSRK

SEQ ID NO: 91 - protein, 14 aa, Solanum lycopersicoides
VGLGGLGKTTLVKL

SEQ ID NO: 92 - protein, 14 aa, synthetic construct
  REGION 1..14: note = Multi Species NB ARC P LOOP Concensus
VGMGGIGKTTLAKK

SEQ ID NO: 93 - protein, 17 aa, Solanum lycopersicoides
QQKRYLLVLDDVWNEDQ

SEQ ID NO: 94 - protein, 17 aa, synthetic construct
  REGION 1..17: note = Multi Species NB ARC Kinase 2 Concensus
KGKRYLIVLDDVWDTDQ

SEQ ID NO: 95 - protein, 12 aa, Solanum lycopersicoides
SKIVVTTRSKMV

SEQ ID NO: 96 - protein, 12 aa, synthetic construct
  REGION 1..12: note = Multi Species NB ARC Kinase 3a Concensus
  SITE 5: note = misc_feature - Xaa can be any naturally occurring amino acid
  REGION 10..11: note = misc_feature - Xaa can be any naturally occurring amino acid
SRIIXTTRDXXV

SEQ ID NO: 97 - protein, 13 aa, Solanum lycopersicoides
VKKCGGVPLAVKT

SEQ ID NO: 98 - protein, 13 aa, synthetic construct
  REGION 1..13: note = Multi Species NB ARC GLPL Concensus
VKKCKGLPLALKV

SEQ ID NO: 99 - protein, 8 aa, Solanum lycopersicoides
NLVHDLAQ

SEQ ID NO: 100 - protein, 8 aa, synthetic construct
  REGION 1..8: note = Multi Species NB ARC MHD Concensus
CRMHDLIR

SEQ ID NO: 101 - protein, 14 aa, Solanum lycopersicoides
LRMLVLNNLDLEEL

SEQ ID NO: 102 - protein, 14 aa, Solanum lycopersicoides
LRYLNLSDSGKIKL

SEQ ID NO: 103 - protein, 10 aa, Solanum lycopersicoides
LVNLHTLNLI

SEQ ID NO: 104 - protein, 9 aa, Solanum lycopersicoides
LKELPRDFR

SEQ ID NO: 105 - protein, 9 aa, Solanum lycopersicoides
LISLKTLYL

SEQ ID NO: 106 - protein, 8 aa, Solanum lycopersicoides
LQFLLLFK

SEQ ID NO: 107 - protein, 15 aa, Solanum lycopersicoides
LQPELVQHFTALRVL

SEQ ID NO: 108 - protein, 9 aa, Solanum lycopersicoides
LCSLPSSIR

SEQ ID NO: 109 - protein, 17 aa, Solanum lycopersicoides
LTSLEKLWIWNCEELDL

SEQ ID NO: 110 - protein, 14 aa, Solanum lycopersicoides
LTSLQSLLLMGLPK

SEQ ID NO: 111 - protein, 16 aa, Solanum lycopersicoides
LVTLPLELKDTAPTTL

SEQ ID NO: 112 - protein, 10 aa, Solanum lycopersicoides
LVELPEWLPN

SEQ ID NO: 113 - protein, 12 aa, Solanum lycopersicoides
LQRLYIEDCPVL

SEQ ID NO: 114 - DNA, 2492 bp, Solanum lycopersicum
  source 1..2492: mol_type = unassigned DNA
atgtagatccttgaacgtgttttggcgaaatctttcttgttcaatatcattgaacgtgttttggctaaagtttcttcaattgctatatatgagatcagtctagcttggaatgttaagacagagctaaggaaactccaaagtactctatccaccatcaaagttgtacttctagatgcaaacgagcagcaggcgaagaaccatgaagtgagagattggctggaaaagctcagagatgttgtttatgatgtcgatgatttgatggatgatttatcaacacaactgttgctgcgaatgcatttcgagtaaagctttaggaagaaggtaaggaagttcttttcaggttcaaatccaattatatatcgattcaagattggcagaaaagtaaaagaaatcaaggagctgctgaatgagattgcagatgattggagaaatttccacttcacggaacatacttatgtaactccagctgagaatacgagtagagaacaaacacactcctttgtgagggcatcagatatcattggtagagatgatgatcaagaaaacattgtataacagctgatagattctcatgatgaggaaaatatttttgtgattcctattgttggacttagagggcttggaaaaaccacacttgttaagttggtttataacaataatagggttgttcagaatttcaaccttagattgtgggttagtatttctgaagattttagtctgagcaaggtaattgagaaaattgtgaggtccggaacaggagagagttttgaccacctagatatggaccaattacaaggttgtttgggagaggttttgcaacagaaaaggtatttacttgtgctggatgatgtgtggaatgaagatcaacacaagtggatagatatgagggagttgccgatgaattgttccagaggtagtaaaattgttgtcactacacgtagtaagatggttgctttgattactggaacagttccgccttattatttgagaggccttggtaatgatgactgcttatcttatttttgaaatgtgcatttggagggcaggacaatttgtttcctaatctagtagaaataggaaaagaaattgtgaaaaagtgtggaggagtgcctttggctgtgaaaaccttgggaaggttgttgtacatgaaaacagacaagaacgaatggttgcagataagagacgatgagatatgggaaatcgaacagaataaatctgacatcttaccaatattgaggttgagctatgaacagatgccatcacatctaagacagtgctttgcctattgccccatgttacccaaaggtcaagaaattccgtgggaggattttatcaatcgctgggctcaaggatttatccagagttcaaacagaaacaggaagttggaagatatcggtaatcagtactttgatgagttgctatcaaggttttgcttcctagatgtgatacaagcagcttttgatggagaaatattggcttgtaagatacacaatcttgtgcatgatcttgcacagtcagtatcaggtgcagagtgcttaaatgtgaaacccaatgctttcgtggtttctgagagagttcgccacttattcttccatgcagaagatatgtctaggaaatacttccccagatttttgcttcctttgcataaggtgaggtctttttttttattcgtttaatattggacctgtaaacaagttttttgtcaagacaatgttgtcaaatttcaaatgccttcggatgttagtcttgaacaatctagatcttgaggagttgccaacttcgataggtaacttgaatgaattaagataccttaaccttagtgacagtggtaatatcaagtttattccaaggtctatgagcaaattagtaaatttgcacacccttaacctcattaactgtgaacagcttaaggagttgccaagagactttagaaagttaatcagcctgaagaccttgtatttgaccacacatcagatattagcagggatcaagaatcaacattctttcacttctcttcaatttttgcttcttttcgaatgttgtttcccaaaattgcagccagaactggtgcagcattttactgaacttcggttgttgcgtatctacgaatgccaagtttatgttctcttccaagaagtattagatatcttacttcacttgaaaagctatggatctggaactgtgaagaacttgatttgattgatagagaaggaatgtcaggcctaacaagtcttcaatccttgcttctaatggggcttcctaagttggtgactctaccattggaactcaaagataccgctcctacaacattgaagtactttagaatcgccgattgtccctacctggtggagcttccagagtggctgccgaattgctcctcacttcagagactgtatatagaggattgtcctgttttggcatcgatacctcaaggaatctacaaccacaatgccaacgtccatataatcgactgtccattgctaggtggatga

SEQ ID NO: 115 - DNA, 2195 bp, Solanum pennellii
  source 1..2195: mol_type = unassigned DNA
atgtagatccttgaacgtgttttggcgaaatctttcttgttcaatatcattgaacgtgttttggctaaagtttcttcaattgctatatatgagatcagtctagcttggaatgttaagacagagctaaggaaactccaaagtactctatccaccatcaaagttgtacttctagatgcaaacgagcagcaggcgaagaaccatgaagtgagagattggctggaaaagctcagagatgttgtttatgatgtcgatgatttgatgggtgatttatcaacacaactgttgctgcgaatgcatttccagaaaagctttaggaagaaggcaaggaagttcttttcaagttcaaatccaattatatatcgattcaagattggcagaaaagtaaaagaaatcaaggagctgctgaatgagattgcagatgataggagaaatttccacttcacggaacatacttatgtaattccagctgaaaatacgagtagagaacaaacacactcctttgtgagggcatcagatatcattggtagagatgatgatcaagaaaacattgtataacagctgatagattctcatgatgaggaaaatatttttgtgattcctattgttggacttagagggcttggaaaaaccacacttgttaagttggtttataacaataatagggttgttcagaatttcaaccttagattgtgggttagtatttctgaagattttagtctgagcaaggtaattgagaaaattgttaggtccggaacaggagagagttttgaccacctagatatggaccaattacaaggttgtttgggagaggttttgcaacagaaaaggtatttacttgtgctggatgatgtgtggaatgaagatcaacacaagtggacagatatgagggagttgctgatgaattgttccagaggtagtaaaattgttgtcactacacgtagtaagatggttgctttgattactggaacagttccgccttattatttgagaggccttggtaatgatgactgcttatcttatttttgaaatgtgcatttggagggcaggacaatttgtttcctaatctagtagaaataggaaaagaaattgtgaaaaagtgtggaggagtgcctttggctgtgaaaaccttgggaaggttgttgtacatgaaaacagacaagaacgaatggttgcagataagagacaatgagatatgggaaatcgaacagaataaatctgacatcttaccaatattgaggttgagctatgaacagatgccatcacatctaagacagtgctttgcctattgccccatgttacccaaaggtcaagaaattccgagggaggattttatcaatcgctgggctcaaggatttatccagagttcaaacagaaacaggaagttggaagatatcggtaatcagtactttgatgagttgctatcaaggttttgcttcctagatgtggtacaagcttttgatggagaaatattggcttgtaagatacacaatcttgtgcatgatcttgcacagtcagtatcaggtgcagagtgcttaaatgtgaaacccaatgctttcgtggtttctgagagagttcgccacttattcttccatgcagaagatatgtctaggaaatacttccccagatttttgcttcctttgcataaggtgaggtctttttcttattcgtttaacattggacctgtaaacaagttttttgtcaagacaatgttgtcaaatttcaaatgccttcggatgttagtcttgaacaatctagatcttgaggagttgccaacttcgataggtaacttgaatgaattaagataccttaaccttagtgacagtggtaatatcaagtttcttccaaggtctatgagcaaattagtaaatttgcacacccttaacctcattaactgtgaacagcttaaggagttgccaagagactttagaaagttaatcagcctgaagaccttgtatttgaccacacatcagatatttgcagggatcaagaatcaacattctttcacttctcttcaattttgcttcttttcaaatgttgtttcccaaaattgcagccagaactggtgcagcattttactgcacttcggttgttgcgtatctacgaatgcccaagtttatgttctcttccaagcagtattagatatcttacttcacttgaaaagctatggatctggaactgtgaagaacttgatttga

SEQ ID NO: 116 - protein, 823 aa, Solanum tuberosum
MAESFLFNIIERVLAKVSSIAVYEITLAWNVKIELRKLQSTLSTIKAVLLDANEQQAKNHEVRDWLEKLRDVVYDVDDLMDDLSTQLLLQMHFQKSFRKKVRKFFSSSNPIIYRFKIGRKVKEIRELLNEIADDRRNFHFTEHTFVISAENTSREQTHSFVRASDIIGRDDDQENIIKQLIGSHDEENISVIPIVGLGGLGKTTLVKLVYNNNRVVQNYDLRMWVSISEDFNLSKVIEKILRSATGESFDHLDMDQLQGCLGEVLQQKRYLLVLDDVWNEDQHKWTDLRELLMNCSRGSKIVVTTRSKMVALITGTVPPYYLGGLADDDCLSLFLKCAFGGQDNLFPNLVEIGKEIVKKCGGVPLAVKTLGRLLYMKTDENEWLQIRDNEIWEIEQNKSDILPILRLSYEQMPSHLRQCFAYCSMLSKGQEIPREDFINRWIAQGFIQSSNRNRKLEDIGNQYFDELLSRFCFLDVVQAFDGEILACKIHNLVHDLAQSVAGAECLNVKPNAFVVSERVRHLFFHAEDMSRKHFPRILLPLQKLRSFSYSFNIGPVNKFFVKTMLSNFKCLRMLVLNNLDLEELPTSIGHLKELRYLNLSNSGNIKFLPRSMSKLVNLHTLNLINCEQLKELPRDFRKLISLKTLYLTTHQISAGIKNQHVFTSLQFLLLFKCCFPKLQPELVQHFSALRVLRIYECPSLCSLPSSIRYLTSLEKLWIWNCEELDLIDGEGMSGLTSLHSLLLMGLPKLVTLPLELKDTAPTTLKYFRIADCPNLMELPEWLPNCSSLQRLYIEDCPVLASIPQGIYSHNANLHIIDCPLLGG

SEQ ID NO: 117 - protein, 828 aa, Capsicum annuum
MAESFLFNIIERVLAKVSSIAVYEISLAWNVKTDLRKLQSTLSTIKAVLLDANEKQAKNHEVRDWLEKLRDVVYDVDDLMDDLSTQLLLQMHFQKSFGKKVRRFFSSSNPIIYRFKIGRRVKEIRELLNEIADDRKSFHFTEHNYLIPAENTSREQTHSSVRASDIIGRDADQENIVKQLIDSHNEENISVIPIVGLGGLGKTTLVKLVYNDNRVVQNFDLRMWVSISEDFSLRKIIEKILRSATGESFDHLDMDQLQGCLGNVLRLKRYLLVLDDVWNEDQHKWTDLRELLMNCASGSKIVVTTRSKMAALITGTVPPYYLEGLANSDDCLSLFLKCAFGGQDNMFPNLVEIGKEIVKKCGGVPLAVKTLGRLLYTKTDENEWLQIRDNEIWEIEQKESDILPILRLSYEQMPSHLRQCFAYCSMLSKGQEIPREDFINRWIAQGFIQSSNRNSKLEDIGNQYFDELLSRFCFLDVVQAFDGEILACKIHSLVHDLAQSVAGAECLNVKPNAFVVPERVRHLFFHAEDMSGKHFPKFLLPLRKLRSFSYSFNVGPVNKFFVKTILSNFKRLRMLVLSNLDLEELPTSIGHLKELRYLNLSGSGNIKFLPRSMSKLVNLQTLNLINCEQLKELPRDFAKLISLKTLYLTTQQISVGIKCKNQHSFTSLQFLLLFKCCFPKLQPELVQHFTALRVLRIYECPSLCSLPSSIRYLTSLEKLWIWNCEELDLMDGEGMSGLSSLRSLLLMGLPKLVTLPLELKDTAPATSKYFRIADCPNLVELPEWLQNYSSLQRLYVEDCPALASIPPGIYNHNVNVHIIDCPLLSGGC

SEQ ID NO: 118 - protein, 827 aa, Capsicum baccatum
MAESFLFNIIERVLAKVSSIAVYEISLAWNVKTDLRKLQSTLSTIKAVLLDANEKQAKNHEVRDWLEKLRDVVYDVDDLMDDLSTQLLLQMHFQKSFGKKVRRFFSSSNPIIYRFKIGRRVKEIRELLNEIADDRKSFHFTEHNYLIPAENTSREQTHSFVRASDIIGRDADQENIVKQLIDSRNEENISVIPIVGLGGLGKTTLVKLVYNDNRVVQNFDLRMWVSISEDFSLRKIIEKILRSATGESFDHLDMDQLQGCLGNVLQLKRYLLVLDDVWNEDQHKWTDLRELLMNCARGSKIVVTTRSKMAALITGTVPPYYLEGLASDDCLSLFLKCAFGGQDNMFPNLVEIGKEIVKKCGGVPLAVKTLGRLLYTKTDENEWLQIRDNEIWEIEQKESDILPILRLSYEQMPSHLRQCFAYCSMLSKGQEIPREDFINRWIAQGFIQSSNRNSKLEDIGNQYFDELLSRFCFLDVVQAFDGEILACKIHSLVHDLAQSVAGAECLNVKPNAFVVPERVRHLFFHAEDMSGKHFPKFLLPLRKLRSFSYSFNVGPVNKFFVKTILSNFKRLRMLVLSNLDLEELPTSIGHLKELRYLNLSGSGNIKFLPRSMSKLVNLQTLNLINCEQLKELPRDFTKLISLKTLYLTTQQISVGIKFKNQHSFTSLQFLLLFKCCFPKLQPELVQHFTALRVLRIYECPSLCSLPSSIRYLTSLEKLWIWNCEELDLMDGEGMSGLSSLRSLLLMGLPKLVTLPLELKDTAPATSKYFRIADCPNLVELPEWLQNYSSLQRLYIEDCPALASIPPGIYNHNVNVHIIDCPLLSGGC

SEQ ID NO: 119 - protein, 827 aa, Capsicum chinense
MAESFLFNIIERVLAKVSSIAVYEISLAWNVKTDLRKLQSTLSTIKAVLLDANEKQAKNHEVRDWLEKLRDVVYDVDDLMDDLSTQLLLQMHFQKSFGKKVRRFFSSSNPIIYRFKIGRRVKEIRELLNEIADDRRSFHFTEHNYLIPAENTSREQTHSSVRASDIIGRDADQENIVKQLIDSHNEENISVIPIVGLGGLGKTTLVKLVYNDNRVVQNFDLRMWVSISEDFSLRKIIEKILRSATGESFDHLDMDQLQGCLGNVLQLKRYLLVLDDVWNEDQHKWTDLRELLMNCASGSKIVVTTRSKMAALITGTVPLYYLEGLASDDCLSLFLKCAFGGQDNMFPNLVEIGKEIVKKCGGVPLAVKTLGRLLYTKTDENEWLQIRDNEIWEIEQKESDILPILRLSYEQMPSHLRQCFAYCSMLSKGQEIPREDFINRWIAQGFIQSSNRNSKLEDIGNQYFDELLSRFCFLDVVQAFDGEILACKIHSLVHDLAQSVAGAECLNVKPNAFVVPERVRHLFFHAEDMSGKHFPKFLLPLRKLRSFSYSFNVGPVNKFFVKTILSNFKRLRMLVLSNLDLEELPTSIGHLKELRYLNLSGSGNIKFLPRSMSKLVNLQTLNLINCEQLKELPRDFAKLISLKTLYLTTQQISVGIKCKNQHSFTSLQFLLLFKCCFPKLQPELVQHFTALRVLRIYECPSLCSLPSSIRYLTSLEKLWIWNCEELDLMDGEGMSGLSSLRSLLLMGLPKLVTLPLELKDTAPATSKYFRIADCPNLVELPEWLQNYSSLQRLYIEDCPALASIPPGIYNHNVNVHIIDCPLLSGGC

SEQ ID NO: 120 - protein, 824 aa, Nicotiana attenuata
MAESFLFNIIERVLAKVSSVAVTEISLAWNVKTELRKLQSTLSTIKAVLLDANEQQAKNHEVRDWLEKLRDVVYDVDDVLDDLSTQLLLQIHFQKSLKKKVRKFFSSSNPIIYRFKIGRKVKEIRELLNEIADDRKSFHFTEHTCLNPVENICREQTHSFVRASDIIGRETDQENIVKQLIDARDEENISVIPIVGLGGLGKTTLVKLVYNYNTVVQNFDLRMWVSISEDFSLSKVIEKILRSATGESFGHLDMDQLQGHLSEVLRSKRYLLVLDDVWNEDQNKWTDLRELLMNCSKGSKIVVTTRSKMVALITGTVAPYYLGGLTDDACLLLFLKCAFVGEDKLLPNLVEIGKEIVKKCGGVPLAVKTLGRLLYMKTDENEWLRIRDNEIWEIEQKQSDILPILRLSYEQMPSHLRQCFAYCSMLSKGQEIPREDFINRWIAQGFIQSSNGSRKLEDIGNQYFDELLSRFCFLDVVQAFDGEILACKLHNLVHDLAQSVAGSECLNVKSNASVVSERVRHLFFHAEDMSRKHFPRFLLSLQKLRSFSYSFNIGPVNKFFVKTTLSNFKCLRVLVLNNLDFEELPTSIGHLKELRYLNLSDNGNIKFLPMSMSKLVNLQTFNLINCEQLKELPRDFGKLICLKTLYLTTYKISAGKNQQSFPSLQFLLLFKCCFPKLQPELVQQFTALRVLRIYECPSLCSLPSSIRYLTSLEKLWIWNCEELDLMDGEGMVGLTSLRSLLLMGLPKLVTLPLGLKDAAHATLKYFRVADCPNLVVLPEWLQNCSSLQRLYIEDCPVLATIPQGIYNHNANVHIIDCPLLSGGC

SEQ ID NO: 121 - protein, 824 aa, Nicotiana benthamiana
MAEFFLFNIIERVLSKVSSVSVNEISLAWNVKTELRKLQSTLSTIKAVLLDANERQAKNHEVRDWLEKLRDVVYDVDDVLDDLSTQLLLQIHFQESLKKKVRKFFSSSNPIIYRFKIGRKIKEIRELLNDIADDRKSFHFTEHTCINPVENICREQTHSFVRASDIIGRETDQENIVKQLIDSRDEENISVIPIVGLGGLGKTTLVKLVYNNNKVVQNFDLRMWVSISEDFSLSKVIEKILRSATGESFGHLDMDQLQGHLSEVLRSKRYLLVLDDVWNEDQNRWTDLRELLMNCSRGSKIVVTTRSKMVALITGTVAPYYLGGLTDDACLSLFLKCAFVGEDKLLPNLVEIGKEIVKKCGGVPLAVKTLGRLLYMKTDENEWLRIRDNEIWEIEQKQSDILPILRLSYEQMPSHLRQCFAYCSMLSKGQEIPREDFINRWIAQGFIQSSNGSRKLEDIGNQYFDELLSRFCFLDVVQAFDGEILACKLHNLVHDLAQSVAGSECSNVKSNASVVSERVRHLFFHAEDMSRKHFPRFLLSLQKLRSFSYAFNIGPANKFFVKTTLSNFKCLRVLVLNNLDFEELPTSIGHLKELRYLNLSDNGNIKFLPRSMSKLVNLQTLNLINCEQLKELPRDFGKLICLKTLYLTTYKISAGKNQQSFPSLQFLLLFKCCFPKLQPELVQQFTALRVLRIYECPSLCSLPSSIRYLTSLEKLWIWNCEELDLMDGEGMVGLTNLRSLLLMGLPKLVTLPLGLKDAAHATLNYFRVADCPNLVVLPEWLQDCSSLQRLYIEDCPVLATVPQGIYNHNANVHIIDCPLLSGGC

SEQ ID NO: 122 - protein, 829 aa, Nicotiana tabacum
MLLQSMAETFLFNIIERVLAKVSSVAVYEISLAWNVKTELRKLQSTLSTIKAVLLDANEQQAKNHEVRDWLEKLRDVVYDVDDLLDDLSTQLTLQMHFQKSLKKKVRKFFSSSNPIFYRFKIGRKVKEIRELLNEIAHDRKSFHFTEHTYLNPVENICREQTHSFVRASDIIGRETDQENIVKQLIDAREEENISVIPIVGLGGLGKTTLVKLVYNNNKVVQNFDLRMWVSISEDFSLSKVIEKILRSATGESFGHLDMDQLQSHLSEVLRSKRYLLVLDDVWNEDQNKWTDLRELLMNCSRGSKIVVTTRSKMVALITGTVAPYYLGGLADDECLSLFLKCAFVGEDKLLPNLVEIGKEIVKKCGGVPLAVKTLGRLLYMKTDENEWLRIRDNEIWEIEQKQSDILPILRLSYEQMPSHLRQCFAYCSMLSKGQEIPREDFINRWIAQGFIQSSNGSRKLEDIGNQYFDELLSRFCFLDVVQAFDGEILACKLHNLVHDLAQSVAGSECLNVKSNASVVSERVRHLFFHAEDMSRKHFPRFLLSLQKLRSFSYSFNIGPVNKFFVKTMLSNFKCLRVLVLNNLDFGELPTSIGHLKELRYLNLSDNGNIKFLPRSMSKLVNLQTLNLINCEQLKELPRDFEKLICLKTLYLTTYKISAGKNQQSFPSLQFLLLFKCCFPKLQPELVQQFTALRILRIYECRSLYSLPSSIRYLTSLEKLWIWNCEELDLMDGEGMVGLTSLRSLLLMGLPKLVTLPLGLKDTAHATLKYFRVADCPNLAVLPEWLQNCSSLQRLYIEDCPVLASIPQGIYNHNAEVHIIDCPLLSGGC

SEQ ID NO: 123 - protein, 824 aa, Nicotiana tomentosiformis
MAETFLFNIIERVLAKVSSVAVYEISLAWNVKTELRKFQSTLSTIKAVLLDANEQQAKNHEVRDWLEKLRDVVYDVDDLLDDLSTQLTLQMHFQKSLKKKVRKFFSSSNPIIYRFKIGRKVKEIRELLNEIAHDRKSFHFTEHTYLNPVENICREQTHSFVRASDIIGRETDQENIVKQLIDAREEENISVIPIVGLGGLGKTTLVKLVYNNNKVVQNFDLRMWVSISEDFSLSKVIEKILRSATGESFGHLDMDQLQSHLSEVLRSKRYLLVLDDVWNEDQNKWTDLRELLMNCSRGSKIVVTTRSKMVALITGTVAPYYLGGLADDECLSLFLKCAFVGEDKLLPNLVEIGKEIVKKCGGVPLAVKTLGRLLYMKTDENEWLRIRDNEIWEIEQKQSDILPILRLSYEQMPSHLRQCFAYCSMLSKGQEIPREDFINRWIAQGFIQSSNGSRKLEDIGNQYFDELLSRFCFLDVVQAFDGEILACKLHNLVHDLAQSVAGSECLNVKSNASVVSERVRHLFFHAEDMSRKHFPRFLLSLQKLRSFSYSFNIGPVNKFFVKTMLSNFKCLRVLVLNNLDFGELPTSIGHLKELRYLNLSDNGNIKFLPRSMSKLVNLQTLNLINCEQLKELPRDFEKLICLKTLYLTTYKISAGKNQQSFPSLQFLLLFKCCFPKLQPELVQQFTALRILRIYECRSLYSLPSSIRYLTSLEKLWIWNCEELDLMDGEGMVGLTSLRSLLLMGLPKLVTLPLGLKDTAHATLKYFRVADCPNLAVLPEWLQNCSSLQRLYIEDCPVLASITQGIYNHNAEVHIIDCPLLSGGC

SEQ ID NO: 124 - DNA, 2475 bp, Nicotiana benthamiana
  source 1..2475: mol_type = unassigned DNA
atggcagaatttttcttgttcaacatcattgaaagagttttgtcaaaagtttcttcagtttctgtaaatgagatcagtttagcgtggaatgttaaaacagagttaagaaaactccagagcactttatccacaatcaaagctgtacttttagatgcaaatgagcggcaggcaaagaaccatgaagtgagagattggctggaaaagctaagagatgttgtttatgatgtcgatgatgtgctcgatgatttgtcaacacagctattactacaaatacattttcaggaaagccttaagaagaaggtaagaaaattcttttcaagttcaaatccaattatttatagattcaagattggtagaaagataaaagagattagggaactattgaatgatattgcagatgatcggaaaagtttccactttactgaacatacttgtataaatccagttgaaaatatttgtagagaacaaacacattcctttgtaagggcctctgatattattggtagagaaactgatcaagaaaacatagtaaaacagctcatagattctcgcgatgaggaaaatatttctgtgattcctattgttggacttggagggcttggaaaaaccacacttgttaagttggtttataacaataataaggttgttcagaattttgacctgagaatgtgggtgagtatttcagaagattttagtctgagtaaggtaattgagaaaattctgcgatctgcaacaggagagagttttggccacctagatatggaccaattacaaggtcatttgagtgaggttttgcgatcgaaaaggtatttacttgtactggatgatgtgtggaatgaagatcaaaacaggtggacggacttgagggagttgctgatgaattgttctagaggtagtaagattgttgtcactacacgcagtaagatggttgctttgattactggaacagttgcaccttactatttgggtggtcttaccgatgatgcgtgcttatcgttatttttgaaatgtgcatttgtaggggaggacaaattgttgcctaatctagtagaaataggaaaagagattgtgaaaaagtgtggaggagtgcctttggctgtgaaaaccttgggaaggttattgtatatgaaaacagacgaaaatgaatggttgcggataagagataatgagatatgggagatcgaacagaaacaatcagacattttaccaatattgagattgagctatgaacagatgccatctcatctaagacagtgctttgcctattgctccatgttatccaaaggtcaagaaattccgagagaggacttcatcaaccggtggattgctcaaggatttatacagagttctaacggatccaggaagttggaagatattggtaatcagtactttgatgagttgctatcaaggttttgcttccttgatgtggtacaagcatttgatggagaaatattggcttgtaagttacacaatcttgtgcatgatcttgcacagtcagtggcaggttctgaatgttcaaatgtgaaatctaatgcttctgtggtttctgaaagagttcgccacttattttttcatgcagaagatatgtctaggaaacacttcccaagatttttactttctttgcaaaagttgaggtctttttcttacgcatttaacattggacctgcaaacaagttctttgtcaagacgacattatcaaatttcaaatgccttcgggtgttagtcttgaacaatttagattttgaggagttgccaacttcgataggtcacttgaaggaactaagatatcttaacctcagtgacaatggtaacatcaagtttctcccaaggtctatgagcaaattagtaaatctgcagactcttaacctcattaattgtgaacagcttaaggagttgccgagagactttggaaagttaatctgcttgaagaccttgtatttgactacatataagatatcagcagggaagaatcaacaatctttcccttctcttcaatttttgcttcttttcaagtgttgtttcccaaaattgcagccagaactggtgcagcagtttactgcacttcgggttttgcgtatctatgaatgcccgagtttatgttctcttccaagcagtattagatacctgacttcacttgaaaagctatggatttggaactgtgaagaacttgatttgatggatggagagggaatggtaggcctaacaaatttacggtcgttgcttctaatgggactccctaagttggtgactctaccattgggacttaaagatgctgctcatgcaacactgaactactttagagttgccgattgtcccaacctagtggtgcttccagaatggctgcaggattgctcttcccttcagaggctgtatatagaggattgtcctgtattggcaactgtacctcaaggaatctacaaccataatgccaatgtccatataatcgactgtccattgctaagtggaggatgctaa

SEQ ID NO: 125 - DNA, 2324 bp, Nicotiana benthamiana
  source 1..2324: mol_type = unassigned DNA
atggcagaatctttcttgttcaacatcattgaaagagttttggcaaaagtctcttcagttgctgtaaatgagatcagtttagcttggattgttaaaattaagaaaactccagagcactttatccacaatcaaagctgtacttttagatgcaaatgagcagtaggcaaagaaccatgaagtgagagattggctggaaaagctaagacatattgtttatgatgtcgatgatctgctcgatgatttgtctacacagttattactacaaatacattttcagaaaagccttaagaagaaggtaagaaaattcttttcaagttcaaatccaattatttatagattcaagattggtagaaaggtaaaagagattagggaactattgaatgagattgcagatgataggagaactttccactttactgaacatacttgtctaaatccagttgagaatatttgtagagaacaaacacattcctttgtaagggcctccgatattattagtagagaaactgatcaagaaaacatagtaaaacagctcatagatgctcgtgacgaggaaaatatttctgtgattcctgttgttggacttggtgggcttggaaaaaccacacttgttaagttggtttataacaataataaggttgttcagaattttgacctgagaatgtgggttagtatttcagaggattttagtctgagtaaggtaattgagaaaattctgcggtctgcaacaggagagagttttggccaccttgatatggatcaattacaaggtcatttaagtgaggttttgcgatcaaaatggtatttacttgtactggatgatgtgtggaatgaagatcaaaacaggtggacggacttgagggagttgctgatgaattgttctagaggtagtacgattgttgtcactacacgcaataagatggttgctttgattactggaacagttgcaccttactatttgggtggtcttactgatgtgcgtgcttatcgttatttttgaaatgtgcatttgtaggggtggaaaaattgttgcctaatctagtagaaataggaaaagaaattgtgaaaaagtgtggaggagtgcctttggctgtgaaaaccttgggaaggttgctgtacatgaaaacagacgaaaatgaatggttgcggatacgagataatgagatatgggagatcgaacagaaacaatctgacattttgccaatattgagattgagctatgaacagatgccatcacatctaagacagtgctttgcctattgctccatgttatccaaaggtcaagaaattccgagagaggacttcatcaaccggtggattgctcaaggatttatacagagttcaaacggatccaggaagttggaagatattggtaatcagtactttgatgagttactatcgaggttttgcttccttgatgtggtacaagcttttgatggagaaatattggcttgtaagttacacaatcttgtgcatgatcttgcacagtcagtggcaggttctgaatgtttaaatgtgaaatctaatgcttctgtggtttctgaaagagttcgccacttattttttcatgcagaagatatgtctaggaaacacttcccaatatttttactttctttgcaaaagttgaggtctttttcttactcatttaacattggacctgtaaacaagttctttgtcaaaacgacgttatcaaatttcaaatgccttcgggtgttagtcttgaacaatttagattttgaggagttgccaacttcgataggtcacttgaaggaattaagatatcttaaccttagtgacaatggcaacatcaagtttctcccaaggtctatgagcaaattagtaaatctgcagacttttaacctcattagttgggaacagcttaaggagttgccaagaggctttggaaagttaatctgtttgaagaccttgtatttgactacatataagatacggaaaagagtcatatgttctactagtcaaaataagctacatttgtcctccgttatactatttggccatatatgtccctaccgttcacatctctggccatatatgtccctctgacgttaacttttttaaaaaaataattaaaagccacgtgtcatagtcctattggttaaactaaacccacttcttttttttaatattaaaacgaaaatatttgtaaaaaatgtaaaaaaacttgtaaaaaatgtgtcaaaaataattttgtcaaaaataaaaatattttattaaaaaaattttatttttgtttttacaagaagaaaatcagttcattttcaaatttgaaaaatatttccaaaatgtgttttttgtttttaaaa

SEQ ID NO: 126 - DNA, 2472 bp, synthetic construct
  misc_feature 1..2472: note = Synthetic Ptr1
  source 1..2472: mol_type = other DNA
atggcagaatcattccttttcaacattattgagagggttcttgctaaggtttcatcaatcgctgtgtatgaaatctcactagcatggaacgttaaaacagaactaagaaaacttcaatctactctttccactatcaaggctgttcttcttgatgctaacgaacaaaaagcaaaaaaccacgaagttagagactggcttgaaaaactcagggatgtggtttacgatgttgatgaccttatggacgatctttcaactcaacttttgcttcagatgcacttccaaaaatcttttagaaagaaagtaaggagatttttttctagttctaatcctattatttatagattcaaaattggaagaaaagtaaaggaaattagggaactgcttaatgaaattgctgatgacaggaggaattttcactttacggagcatacatatgttattcctgctgagaatactagtagggaacagacacattccttcgtgagagcctctgatattattggaagagacgatgaccaagagaacatcgtaaagcagcttatagactctcacgatgaagaaaacatttcagtgatccctatcgttggtcttggtgggttgggaaagaccactcttgtgaagcttgtttacaacaacaatagagttgtgcagaactttgatcttaggatgtgggtgagtatctcagaggattttagtcttagcaaagtaatcgagaagattcttaggtctgcaactggagaaagtttcgaccatctagacatggatcaacttcaatgctgtcttggagaagttcttcaacaaaaaagatatcttcttgttctggacgatgtgtggaatgaagaccaacataagtggacagatcttagagagcttcttatgaactgttctagaggaagtaagattgtggtcacaacaagaagtaaaatggtggctcttattacaggaactgttccaccttactatcttggaggacttgcaaatgacgactgtttatcattattcttgaagtgtgcttttggtggacaagacaacttgttccctaacctagttgaaattggaaaggaaatcgtgaagaagtgcggaggtgtgccattggcagtgaagacccttggaagattgctttatatgaagacagatgagaacgagtggttgcaaataagggataacgagatttgggagatcgagcagaacaaatcagacatcttacctatacttagacttagctacgaacaaatgccttcacacctaaggcagtgttttgcttattgttctatgttgcccaagggtcaggaaatcccgagagaggactttattaatagatggatcgctcagggattcatacaaagttctaacaggaacagaaagcttgaagacatcggaaatcaatacttcgatgaattgctttcaagattttgtttccttgatgttgtacaggctttcgatggtgaaattttggcatgtaaaatacataatttggtgcacgatttggcacaatcagtttcaggagcagaatgccttaatgttaaacctaatgcattcgttgtctcagaaagggttagacaccttttttttcatgctgaagacatgtcaaggaagcactttcccaggtttcttcttccattgcagaagcttaggtcattctcatattcatttaatattggtcctgttaacaaattcttcgtcaaaactatgctttcaaacttcaagtgcttgagaatgcttgtccttaacaacctagaccttgaagagcttccaacatcgattggtcatttgaaagaacttagatatctcaatctttctgactctggtaaaatcaaatttttgccaagatcaatgtctaaacttgtaaacctgcataccctaaatctcatcaactgcgaacaacttaaagagcttccaagggatttcagaaaattaattagccttaagactttgtacttgacaacacaccaaatgtctgcaggtatcaaaaatcagcattcattcacatctttgcaattcttattgctttttaaatgctgttttccaaagttgcaaccagagctggttcagcactttactgcattgcgggtgttgagaatctacgaatgtccatcattatgctctttgccatctagtatcagatacctgacatcattggaaaaactttggatttggaattgtgaggaattggatcttattgacggagagggaatgtctggccttacatctcttcagtcccttcttcttatgggacttccaaagcttgtgacactacctttggagcttaaggatacagctccaacaactttaaaatactttagaattgccgactgtcctaaccttgtggaacttcctgaatggcttcctaactgctcttcattgcagaggctctacatagaagattgccctgtgttggcttcgattcctcagggaatttactctcacaacgccaatgtccacataattgactgcccacttctaggaggataa

SEQ ID NO: 127 - DNA, 2730 bp, Arabidopsis thaliana
  source 1..2730: mol_type = unassigned DNA
atggatttcatctcatctcttatcgttggctgtgctcaggtgttgtgtgaatctatgaatatggcggagagaagaggacataagactgatcttagacaagccatcactgatcttgaaacagccatcggtgacttgaaggccatacgtgatgacctgactttacggatccaacaagacggtctagagggacgaagctgctcaaatcgtgccagagagtggcttagtgcggtgcaagtaacggagactaaaacagccctacttttagtgaggtttaggcgtcgggaacagaggacgcgaatgaggaggagatacctcagttgtttcggttgtgccgactacaaactgtgcaagaaggtttctgccatattgaagagcattggtgagctgagagaacgctctgaagctatcaaaacagatggcgggtcaattcaagtaacttgtagagagatacccatcaagtccgttgtcggaaataccacgatgatggaacaggttttggaatttctcagtgaagaagaagaaagaggaatcattggtgtttatggacctggtggggttgggaagacaacgttaatgcagagcattaacaacgagctgatcacaaaaggacatcagtatgatgtactgatttgggttcaaatgtccagagaattcggcgagtgtacaattcagcaagccgttggagcacggttgggtttatcttgggacgagaaggagaccggcgaaaacagagctttgaagatatacagagctttgagacagaaacgtttcttgttgttgctagatgatgtctgggaagagatagacttggagaaaactggagttcctcgacctgacagggaaaacaaatgcaaggtgatgttcacgacacggtctatagcattatgcaacaatatgggtgcggaatacaagttgagagtggagtttctggagaagaaacacgcgtgggagctgttctgtagtaaggtatggagaaaagatcttttagagtcatcatcaattcgccggctcgcggagattatagtgagtaaatgtggaggattgccactagcgttgatcactttaggaggagccatggctcatagagagacagaagaagagtggatccatgctagtgaagttctgactagatttccagcagagatgaagggtatgaactatgtatttgcccttttgaaattcagctacgacaacctcgagagtgatctgcttcggtcttgtttcttgtactgcgctttattcccagaagaacattctatagagatcgagcagcttgttgagtactgggtcggcgaagggtttctcaccagctcccatggcgttaacaccatttacaagggatattttctcattggggatctgaaagcggcatgtttgttggaaaccggagatgagaaaacacaggtgaagatgcataatgtggtcagaagctttgcattgtggatggcatctgaacaggggacttataaggagctgatcctagttgagcctagcatgggacatactgaagctcctaaagcagaaaactggcgacaagcgttggtgatctcattgttagataacagaatccagaccttgcctgaaaaactcatatgcccgaaactgacaacactgatgctccaacagaacagctctttgaagaagattccaacagggtttttcatgcatatgcctgttctcagagtcttggacttgtcgttcacaagtatcactgagattccgttgtctatcaagtatttggtggagttgtatcatctgtctatgtcaggaacaaagataagtgtattgccacaggagcttgggaatcttagaaaactgaagcatctggacctacaaagaactcagtttcttcagacgatcccacgagatgccatatgttggctgagcaagctcgaggttctgaacttgtactacagttacgccggttgggaactgcagagctttggagaagatgaagcagaagaactcggattcgctgacttggaatacttggaaaacctaaccacactcggtatcactgttctctcattggagaccctaaaaactctcttcgagttcggtgctttgcataaacatatacagcatctccacgttgaagagtgcaatgaactcctctacttcaatctcccatcactcactaaccatggcaggaacctgagaagacttagcattaaaagttgccatgacttggagtacctggtcacacccgcagattttgaaaatgattggcttccgagtctagaggttctgacgttacacagccttcacaacttaaccagagtgtggggaaattctgtaagccaagattgtctgcggaatatccgttgcataaacatttcacactgcaacaagctgaagaatgtctcatgggttcagaaactcccaaagctagaggtgattgaactgttcgactgcagagagatagaggaattgataagcgaacacgagagtccatccgtcgaagatccaacattgttcccaagcctgaagaccttgagaactagggatctgccagaactaaacagcatcctcccatctcgattttcattccaaaaagttgaaacattagtcatcacaaattgccccagagttaagaaactgccgtttcaggagaggaggacccagatgaacttgccaacagtttattgtgaggagaaatggtggaaagcactggaaaaagatcaaccaaacgaagagctttgttatttaccgcgctttgttccaaattga

SEQ ID NO: 128 - DNA, 4167 bp, Malus domestica
  source 1..4167: mol_type = unassigned DNA
atggggggagaggcttttcttgtggcattcctccaagtgctggttgacaagttggcgcatcgcgaggtcttcaagtactttggacttgtaaagggcgtagatcaaaaactgaagaaatggagtgccaccttgtctgcgattggagcggtgctgaatgacgcagaggagaggcaactgacggctaagaacaacacactgaagctctggctcgaagatctcagggacttggcttttgatgtggaagacgtgttggacaaatatgctactaaaatgttgaaacgtcagatacaacatgctcattcccgcacaacaagcaaactatggaactcaattcctgatggtgttttcaacttcaacatgaactcggaaatacagaagattagtgagcgattacaagagatatctgaacaaaaagaccagcttaatttgaaaattgatactggggcgttgactacaagggcacgccgaaacatatcacctagttccagtcaaccagatggacctgtgattggaagggatgaggacaaaagaaagattgttgagctgctgtcgaaacaagagcatcgtactgtcaatttcgatgtagttgcaattgttggtatggctggagtcggaaagacaacacttgctggacaagtactcaatgatatggttgcaacccaaacgtttcaaccagctgtttgggcatgcgtatctgacgacttcaaccttgaaagagtgacaaagcaaattcttgaatcaatcacatctcggcaatgcaccacagaagattacaataaggttcaagattatctgcataaggagttagcggggaagaagtttttaattgttttagatgatgtgtggaaaacgtgtagctacggtgaatggatgaagttgcagtccccttttcgtgacggagcacaaggaagcaagataattgtgacaacacgtgatacagatgtttcaaaaatgatgggagctgccacgctcgttcacaatttggagcctatggaaagtagtgtttgtttgcaagtatttgagcagcatgcattcttaaattctaatgacgacaaaccaccaaattacgagttacttaaggaaaaaattgctgccaagtgtaggggattgcctttggccgcaaggacccttggtggtgttctacttcgtaaagatacatacgaatgggaagacatattgaacaacaaactgtggagtctatcaaatgagcacgacatacttccagtactgagattaacctacttttatcttccttcacatttgaaaagatgctttgcctattgctcaatacttccaaatgactatgaatttgaagagaagcaaatgatccttctatggatggccgaggggtttattcttccacgaccagaagataagaagcaaattgaagatttaggtgctgattattttcgggatctcgtatcaaggtcattgtttcaaaaatcaaccaaatgtatttcaaaatatgtgatgcatgaccttattggtgatttagcacggtgggcagcaggagaaatttgttttagattggaggataagcaaaatgatgatggtgaacaacttagatgttttcccaaggcacgccattcgtcttacatcaggggtctgtctgatggggtcaaaagatttgaggtattttctgaactgaaatatttgcgaaccttcttgccactaagaaaggattctttctggaattatttaagtcgtcaggttgcttttgatttattgccgaaattgcaatatttgcgggtgctctctttcaattgctataaaataactgagcttccagactcaatcggtgatttaaggtatctacggtatctcgacctttcctacacagatataacaagtttacctaaatcaacaagcactctttacaacttgcagacgttgatattggaaggctgttctaaattgaaggcattgcctatagacatgagtaatctagttaatttgcgtcatctcaacaactcaaatgtatctttgttggaagacatgcctccacaactaggtcgattggtgaatctccagtcattgactaagtttgtggtgagtggtggtggtggtggtgatcgatcagggatacgagagctggagttcctaatgcatctccgaggaacattgtgcatctcaagattggagaatgtgactgatgtcgaggatgctcagagggccaacttaaactgcaaagagaggcttgattcgttggtgctagaatggtctcattcaagcgacacaagagaaacagaatccgctgtgcttgacatgttacagcctcatacaaagcttaaggagctcaccatcaagagttatgcaggaaaagaattttcatcatgggttggagttccattgttctctaatatggtgctcgtgcgcttagaggaatgtaacaattgtctatctctaccacctctcggaaaattgcctcatctcaaagaactttatattagaggaatgaatgcagtggaaagtgttggagctgagttttatggagagtgctccttgccttttccgctattagagactcttgagtttgtggatatgcaacattggaaagtatggcttccatttcaaacggatcacagaggtagtgttttcccttgcctgaaaacactcttagtcagaaaatgttctaaactggagggtaagctgccagagaaccttgattcgctggcatcgcttgaaattgttaaatgtgaagaattattggtttcaattgccaattacaaacaacttcgtcagttaaacattgacggttgtaaaggggtggtgcatacagctgctaaggttgagtttgagttattagagtccttgtacctttcaaatatttcggagctgacgtctctacaaacaggagaattgtgcagaaatggattaaacatggtcagagatttgaagattaatggatgtgaggagctgacgtcttcattgaagaatgaggcaatattattgcaacagttgatttctcttggccgtttagagattgaagacaactctctcctagttgaagaactaggaaaagaagcagatgagttgttgcaattgcaaatattggggtgtaagcttgaatttctgaagttaaagaagtgcaaaaatcttttgaagctaccagaagggttaaatcagttgtcgtcacttcaagagcttcgcatacatgaatgttcaagtctagtttcttttccagatgttggtttgccaccttctcttaaagacatcgagattacagagtgccactcgttgatatattttgcaaaatcccagattccccaaaatctcagaagaatacagataagagattgcagaagtttgagatcactagtagacaatgaggctgttggttcttgttcttcgtcttctcacaattgtcttgagtacttgaatatcgagagatgtcaatctctaacgttgttatcattgagtgaccagcttgtcagggcacttagagaacttgacatatatgattgtgaacaactggagtttctcgcaccggacgggttgttctgcaacaacactaattactttctcgaaaattttaggatacggaggtgccaaaatctgaaatccttaccgagactgagtggggggataaggggctctaacctgagagagatccggatcaccgattgcgacagattggaggccttgcccgaagacatgcacaatttcaactctcttgagaaattgattatcgactaccgtgaaggtttgacttgctcctttcccgccaacctaacatcacttatgatttggaaggtcaagagctgtaagtcattgtgggagttggagtgggggttacacagactcacctctcttagatacttgtggatcggtggtgaagacccggatatggtatcgtttccaccggacatggtccgaatggagacgttgctccccaaatctctcactgaactctcaattggtggcttcccgaatctgaagaaactgagcagcaagggctttcaattcctaacctcccttgaatctttggaactctgggattgtccaaagctagcatccattccaaaggagggactgcctctttcacttacggaactttgcatctatgggtgtcctgttctaaaagagagatgtcaaccaggaaaaggacgctactggcacaaaatatcccacatcccttacatagatatagattggaagatgatttga

SEQ ID NO: 129 - protein, 909 aa, Arabidopsis thaliana
MDFISSLIVGCAQVLCESMNMAERRGHKTDLRQAITDLETAIGDLKAIRDDLTLRIQQDGLEGRSCSNRAREWLSAVQVTETKTALLLVRFRRREQRTRMRRRYLSCFGCADYKLCKKVSAILKSIGELRERSEAIKTDGGSIQVTCREIPIKSVVGNTTMMEQVLEFLSEEEERGIIGVYGPGGVGKTTLMQSINNELITKGHQYDVLIWVQMSREFGECTIQQAVGARLGLSWDEKETGENRALKIYRALRQKRFLLLLDDVWEEIDLEKTGVPRPDRENKCKVMFTTRSIALCNNMGAEYKLRVEFLEKKHAWELFCSKVWRKDLLESSSIRRLAEIIVSKCGGLPLALITLGGAMAHRETEEEWIHASEVLTRFPAEMKGMNYVFALLKFSYDNLESDLLRSCFLYCALFPEEHSIEIEQLVEYWVGEGFLTSSHGVNTIYKGYFLIGDLKAACLLETGDEKTQVKMHNVVRSFALWMASEQGTYKELILVEPSMGHTEAPKAENWRQALVISLLDNRIQTLPEKLICPKLTTLMLQQNSSLKKIPTGFFMHMPVLRVLDLSFTSITEIPLSIKYLVELYHLSMSGTKISVLPQELGNLRKLKHLDLQRTQFLQTIPRDAICWLSKLEVLNLYYSYAGWELQSFGEDEAEELGFADLEYLENLTTLGITVLSLETLKTLFEFGALHKHIQHLHVEECNELLYFNLPSLTNHGRNLRRLSIKSCHDLEYLVTPADFENDWLPSLEVLTLHSLHNLTRVWGNSVSQDCLRNIRCINISHCNKLKNVSWVQKLPKLEVIELFDCREIEELISEHESPSVEDPTLFPSLKTLRTRDLPELNSILPSRFSFQKVETLVITNCPRVKKLPFQERRTQMNLPTVYCEEKWWKALEKDQPNEELCYLPRFVPN

SEQ ID NO: 130 - protein, 1388 aa, Malus domestica
MGGEAFLVAFLQVLVDKLAHREVFKYFGLVKGVDQKLKKWSATLSAIGAVLNDAEERQLTAKNNTLKLWLEDLRDLAFDVEDVLDKYATKMLKRQIQHAHSRTTSKLWNSIPDGVFNFNMNSEIQKISERLQEISEQKDQLNLKIDTGALTTRARRNISPSSSQPDGPVIGRDEDKRKIVELLSKQEHRTVNFDVVAIVGMAGVGKTTLAGQVLNDMVATQTFQPAVWACVSDDFNLERVTKQILESITSRQCTTEDYNKVQDYLHKELAGKKFLIVLDDVWKTCSYGEWMKLQSPFRDGAQGSKIIVTTRDTDVSKMMGAATLVHNLEPMESSVCLQVFEQHAFLNSNDDKPPNYELLKEKIAAKCRGLPLAARTLGGVLLRKDTYEWEDILNNKLWSLSNEHDILPVLRLTYFYLPSHLKRCFAYCSILPNDYEFEEKQMILLWMAEGFILPRPEDKKQIEDLGADYFRDLVSRSLFQKSTKCISKYVMHDLIGDLARWAAGEICFRLEDKQNDDGEQLRCFPKARHSSYIRGLSDGVKRFEVFSELKYLRTFLPLRKDSFWNYLSRQVAFDLLPKLQYLRVLSFNCYKITELPDSIGDLRYLRYLDLSYTDITSLPKSTSTLYNLQTLILEGCSKLKALPIDMSNLVNLRHLNNSNVSLLEDMPPQLGRLVNLQSLTKFVVSGGGGGDRSGIRELEFLMHLRGTLCISRLENVTDVEDAQRANLNCKERLDSLVLEWSHSSDTRETESAVLDMLQPHTKLKELTIKSYAGKEFSSWVGVPLFSNMVLVRLEECNNCLSLPPLGKLPHLKELYIRGMNAVESVGAEFYGECSLPFPLLETLEFVDMQHWKVWLPFQTDHRGSVFPCLKTLLVRKCSKLEGKLPENLDSLASLEIVKCEELLVSIANYKQLRQLNIDGCKGVVHTAAKVEFELLESLYLSNISELTSLQTGELCRNGLNMVRDLKINGCEELTSSLKNEAILLQQLISLGRLEIEDNSLLVEELGKEADELLQLQILGCKLEFLKLKKCKNLLKLPEGLNQLSSLQELRIHECSSLVSFPDVGLPPSLKDIEITECHSLIYFAKSQIPQNLRRIQIRDCRSLRSLVDNEAVGSCSSSSHNCLEYLNIERCQSLTLLSLSDQLVRALRELDIYDCEQLEFLAPDGLFCNNTNYFLENFRIRRCQNLKSLPRLSGGIRGSNLREIRITDCDRLEALPEDMHNFNSLEKLIIDYREGLTCSFPANLTSLMIWKVKSCKSLWELEWGLHRLTSLRYLWIGGEDPDMVSFPPDMVRMETLLPKSLTELSIGGFPNLKKLSSKGFQFLTSLESLELWDCPKLASIPKEGLPLSLTELCIYGCPVLKERCQPGKGRYWHKISHIPYIDIDWKMI

SEQ ID NO: 131 - protein, 823 aa, Solanum tuberosum
MAESFLFNIIERVLAKVSSIAVYEITLAWNVKIELRKLQSTLSTIKAVLLDANEQQAKNHEVRDWLEKLRDVVYDVDDLMDDLSTQLLLQMHFQKSFRKKVRKFFSSSNPIIYRFKIGRKVKEIRELLNEIADDRRNFHFTEHTYVIPAENTSREQTHSFVRASDIIGRDDDQENIVKQLIDSHDEENISVIPIVGLGGLGKTTLVKLVYNNNRVVQNFDLRMWVSISEDFSLSKVIEKILRSATGESFDHLDMDQLQGCLGEVLQQKRYLLVLDDVWNEDQHKWTDLRELLMNCSRGSKIVVTTRSKMAALITGTVPPYYLEGLADDDCLSLFLKCAFGGQDNLFPNLVEIGKEIVKKCGGVPLAVKTLGRLLYMKTDENEWLQIRDNEIWEIEQNKSDILPILRLSYEQMPSHLRQCFAYCSMLSKGQEIPREDFINRWIAQGFIQSSNRNRKLEDIGNQYFDELLSRFCFLDVVQAFDGEILACKIHNLVHDLAQSVAGAECLNVKPNAFVVSERVRHLFFHAEDMSRKHFPRFLLPLQKLRSFSYSFNIGPVNKFFVKTMLSNFKCLRMLVLNNLDLEELPTSIGHLKELRYLNLSNSGNIKFLPRSMSKLVNLHTLNLINCEQLKELPRDFRKLISLKTLYLTTHQISEGIKNQHSFTSLQFLLLFKCCFPKLQPELVQHFTALRVLRIYECPSLCSLPSSIRYLTSLEKLWIWNCEELDLIDGEGMSGLTSLQSLLLMGLPKLVTLPLELKDTAPTTLKYFRIADCPNLMELPEWLPNCSSLQRLYIEDCPVLASIPQGIYSHNANLHIIDCPLLGG

SEQ ID NO: 132 - DNA, 2472 bp, Solanum tuberosum
  source 1..2472: mol_type = unassigned DNA
atggcggaatctttcttgttcaatatcattgaacgagttttggctaaagtttcttcaattgctgtatatgagatcactctagcttggaatgttaagatagagctaaggaaactccaaagtactctatccaccatcaaagctgtacttctagatgcaaacgagcaacaggcaaagaaccatgaagtgagagattggctggaaaagctcagagatgttgtttatgatgtcgatgatttgatggatgatttatcaacacaactgttgctgcaaatgcatttccagaaaagctttaggaagaaggtaagaaaattcttttcaagttcaaatccaattatatatcgattcaagattggcagaaaggtaaaagaaatcagggagctactgaatgagattgcagatgataggagaaatttccacttcacggaacatacgtatgtaattccagctgagaatacgagtagagaacaaacacactcctttgtgagggcatcagatatcattggtagagatgatgatcaagaaaacattgtaaaacagctgatagattctcatgatgaggaaaatatttctgtgattcctattgttggacttggagggcttggaaaaaccacacttgttaagttggtttataacaataatagggttgttcagaattttgaccttagaatgtgggttagtatttcagaagattttagtctgagcaaggtaattgagaaaattctgaggtctgcaacgggagagagttttgaccacctagatatggaccaattacaaggttgtttgggagaggttttgcaacagaaaaggtatttacttgtgctggatgatgtgtggaatgaagatcaacacaagtggacagatctgagggagttgctgatgaattgttccagaggtagtaaaattgttgtcactacacgtagtaagatggctgctttgattactggaacagttccgccttattatttggaaggccttgctgatgatgactgcttatctttatttttgaaatgtgcatttggagggcaggacaatttgtttcctaatctagtagaaataggaaaagaaattgtgaaaaagtgtggaggagtgcctttggctgtgaaaaccttgggaaggttattgtacatgaaaacagacgagaatgaatggttgcaaataagagataatgagatatgggaaatcgaacagaataaatctgacattttaccaatattgagattgagctatgaacagatgccatcacatctaagacagtgctttgcctattgctccatgttatccaaaggtcaagaaattccgagggaggattttatcaatcggtggattgctcaaggatttatacagagttcaaacagaaacaggaagctggaagatataggtaatcagtactttgatgagttgctatcaaggttttgcttcctagatgtggtacaagcttttgatggagaaatattggcttgtaagatacacaatcttgtgcatgatcttgcacagtcagtagcaggtgcagaatgcttaaatgtgaaacccaatgctttcgtggtttctgaaagagttcgccacttattcttccatgcagaagatatgtctaggaaacacttccccagatttttgcttcctttgcaaaagttgaggtctttctcttattcttttaacattggacctgtaaacaagttctttgtcaagacaatgttgtcaaatttcaaatgccttcggatgttagtcttgaacaatctagatcttgaggagttgccaacttcgataggtcacttgaaggaattaagataccttaaccttagcaacagtggtaatatcaagtttcttccaaggtctatgagcaaattagtaaatctgcacaccctaaacctcattaactgtgaacagcttaaggagttgccaagagactttagaaagttaatcagcctaaagaccttgtatttgactacacatcagatatcagaagggatcaagaatcaacattctttcacttctcttcaatttttgcttcttttcaaatgttgtttcccaaaattgcagccagaactggtgcagcattttactgcacttcgggttttgcgtatctatgaatgcccaagtttatgttctcttccaagcagtattagatatctgacttcacttgaaaagctatggatctggaactgtgaagaacttgatttgatagatggagaaggaatgtcaggcctaacaagtcttcaatccttgcttctaatggggcttcctaagttggtgactctaccattggaacttaaagatactgctcctacaacattaaagtacttcagaatcgccgattgtcccaacctgatggagcttccagagtggctgcccaattgctcctcacttcagagactgtatatagaggactgtcctgttttggcatcgatacctcaaggaatctacagccacaatgccaatctccatataatcgactgtccattgctaggtggatga

(19) United States
(12) Patent Application Publication
(10) Pub. No.: US 2025/0051795 A1
(43) Pub. Date: Feb. 13, 2025

(54) ENHANCED DISEASE RESISTANCE IN PLANTS

(71) Applicants: BOYCE THOMPSON INSTITUTE FOR PLANT RESEARCH, INC., Ithaca, NY (US); CORNELL UNIVERSITY, Ithaca, NY (US)

(72) Inventors: Gregory B. MARTIN, Ithaca, NY (US); Ari FEDER, Haifa (IL); Samantha MAINIERO, Ithaca, NY (US); Sarah HIND, Champaign, IL (US); Diana Carolina MAZO MOLINA, Ithaca, NY (US)

(21) Appl. No.: 18/919,922

(22) Filed: Oct. 18, 2024

Related U.S. Application Data
(62) Division of application No. 16/916,757, filed on Jun. 30, 2020, now Pat. No. 12,157,895.
(60) Provisional application No. 62/894,234, filed on Aug. 30, 2019.

(51) Int. Cl. C12N 15/82; A01H 1/00; A01H 5/08; A01H 6/82; C12N 15/00; C12N 15/74; C12Q 1/6895

(52) U.S. Cl. CPC C12N 15/8279; A01H 1/00; A01H 5/08; A01H 6/82; A01H 6/825; C12N 15/00; C12N 15/74; C12N 15/82; C12Q 1/6895; C12Q 2600/13

(57) ABSTRACT

Embodiments of the disclosure are directed to compositions and methods for enhancing disease resistance in plants. One aspect of embodiments of the disclosure relates to a nucleic acid construct comprising a nucleic acid molecule comprising a Pseudomonas tomato race 1 (Ptr1) polynucleotide, a 5′ heterologous DNA promoter sequence, and a 3′ terminator sequence, wherein the nucleic acid molecule, the DNA promoter sequence, and the terminator sequence are operatively coupled to permit transcription of the nucleic acid molecule. Methods of imparting disease resistance to a plant and methods of identifying a candidate plant suitable for breeding that displays enhanced disease resistance are also disclosed. Embodiments of the disclosure also include plant cells, plants, and plant seeds including a heterologous Pseudomonas tomato race 1 (Ptr1) polynucleotide.

This application is a division of U.S. patent application Ser. No. 16/916,757, filed Jun. 30, 2020, which claims the priority benefit of U.S. Provisional Application Ser. No. 62/894,234, filed Aug. 30, 2019, which are hereby incorporated by reference in their entirety.

This invention was made with government support under grant number 1546625 awarded by the National Science Foundation. The government has certain rights in the invention.

### SEQUENCE LISTING

This application contains a computer readable Sequence Listing which has been submitted electronically in XML format and is hereby incorporated by reference in its entirety. The XML file was created on Oct. 15, 2024, is named 147393000332.xml, and is 215,269 bytes in size.

### FIELD

Embodiments of the disclosure relate generally to disease resistance in plants.

### BACKGROUND

Bacterial speck disease of tomato, caused by Pseudomonas syringae pv. tomato (Pst), occurs in cool, wet environments that favor bacterial spreading and leaf colonization through stomata (Pedley et al., “Molecular Basis of Pto-Mediated Resistance to Bacterial Speck Disease in Tomato,” Ann. Rev. Phytopathol. 41:215-43 (2003)). The disease causes necrotic lesions (specks) on different parts of the plant including leaves, stems, flowers, and fruits. As a result, it can affect both fruit yield and quality, leading to significant economic losses (Jones et al., “Bacterial speck,” in COMPENDIUM OF TOMATO DISEASES 26-27 (J. B. Jones et al. eds, 1991). Two races of Pst are currently defined which differ in their ability to cause disease on tomato varieties expressing the resistance gene Pto. Race 0 strains express the type III effectors AvrPto or AvrPtoB, are recognized by Pto, and consequently are unable to cause disease on Pto-expressing tomato varieties. Race 1 strains do not have the avrPto or avrPtoB genes or do not express these effector proteins and are therefore not recognized by Pto (Lin et al., “Diverse AvrPtoB Homologs from Several Pseudomonas syringae Pathovars Elicit Pto-Dependent Resistance and Have Similar Virulence Activities,” Appl. Environ. Microbiol. 72(1):702-12 (2006); Kunkeaw et al., “Molecular and Evolutionary Analyses of Pseudomonas syringae pv. tomato Race 1,” Mol. Plant-Microbe. Interact. 23:415-24 (2010)). Recently, strains with virulence attributes intermediate between race 0 and race 1 strains have been discovered (Kraus et al, “Pseudomonas syringae pv. tomato strains from New York Exhibit Virulence Attributes Intermediate Between Typical race 0 and race 1 Strains,” Plant Dis. 101:1442-8 (2017)). These strains express AvrPto but nevertheless multiply to levels intermediate between race 0 and race 1 strains in tomato plants that express Pto.

In order to combat pathogens, plants have evolved a two-layered immune system. In an initial defense response, plants use extracellular pattern recognition receptors (PRRs) to detect the presence of microbe-associated molecular patterns (MAMPS) (Dangl et al., “Pivoting the Plant Immune System from Dissection to Deployment,” Science 341(6147):746-51 (2013)). In the second immune response, plants use intracellular proteins (R proteins or nucleotide-binding oligomerization domain-like (NOD-like) receptors, NLRs) to detect pathogen effector proteins translocated inside the host cell during the infection process. NLR-triggered immunity (NTI) is typically associated with programed cell death and significant inhibition of pathogen multiplication (Jones et al., “The Plant Immune System,” Nature 444(7117):323-29 (2006); Buttner, “Behind the Lines-Actions of Bacterial type III Effector Proteins in Plant Cells,” FEMS Microbiol. Rev. 40(6):894-937 (2016)).

Genetic resistance to Pst is conferred by the Pto and Prf genes which encode a serine/threonine cytoplasmic kinase and a nucleotide-binding leucine-rich repeat (NLR) protein, respectively (Pedley and Martin, “Molecular Basis of Pto-Mediated Resistance to Bacterial Speck Disease in Tomato,” Ann Rev Phytopathol 41:215-243 (2003)). The Pto/Prf proteins form a complex that recognizes the type III effectors AvrPto or AvrPtoB expressed by race 0 Pst strains (Kim et al, “Two Distinct Pseudomonas Effector Proteins Interact With the Pto Kinase and Activate Plant Immunity,” Cell 109:589-598 (2002); Mucyn et al., “Regulation of Tomato Prf by Pto-like Protein Kinases,” Mol Plant-Microbe Interact 22:391-401 (2009); Martin, “Suppression and Activation of the Plant Immune System by Pseudomonas syringae Effectors AvrPto and AvrPtoB,” in EFFECTORS IN PLANT-MICROBE INTERACTIONS 123-154 (F. Martin and S. Kamoun eds, 2012).; Ntoukakis et al., “The Tomato Prf Complex is a Molecular Trap for Bacterial Effectors Based on Pto Transphosphorylation,” PLoS Pathog, 9:e1003123 (2013); and Saur et al., “The N-terminal Domain of the Tomato Immune Protein Prf Contains Multiple Homotypic and Pto Kinase Interaction Sites,” J Biol Chem, 290:11258-11267 (2015)). After translocation of AvrPto or AvrPtoB into the plant cell, Pto, which encodes a serine/threonine protein kinase, physically interacts with either one of these effectors and acts in concert with Prf, an NLR, to activate NTI (Salmeron et al., “Tomato Prf is a Member of the Leucine-Rich Repeat Class of Plant Disease Resistance Genes and Lies Embedded Within the Pto Kinase Gene Cluster,” Cell 86(1):123-33 (1996); Pedley et al., “Molecular Basis of Pto-Mediated Resistance to Bacterial Speck Disease in Tomato,” Ann. Rev. Phytopathol. 41:215-43 (2003)). Pto was originally identified in a wild relative of tomato, Solanum pimpinellifolium, and it has been introgressed into many processing-type tomato varieties. For over 30 years, the gene has provided effective control of speck disease caused by Pst race 0 strains (Pitblado et al., “Resistance to Bacterial Speck (Pseudomonas tomato) in Tomato,” Acta Hort. 100:379-82 (1980)). The widespread use of the Pto/Prf genes since the 1980s appears to have exerted selection pressure on the pathogen and race 0 Pst strains are becoming less common. Instead, strains of race 1 Pst, which lack AvrPto and AvrPtoB, are becoming more prevalent and represent a new threat in tomato growing areas (Kunkeaw et al., “Molecular and Evolutionary Analyses of Pseudomonas syringae pv. tomato Race 1,” Mol Plant-Microbe Interact 23:415-424 (2010) and Thapa and Coaker, “Genome Sequences of Two Pseudomonas syringae pv. tomato Race 1 Strains, Isolated From Tomato Fields in California,” Genome Announc 4: e01671-01615 (2016). The increasing prevalence of race 1 Pst strains able to overcome Pto/Prf-mediated NTI has led to the search for sources of resistance to these virulent strains. Since no race 1 resistance has been identified in cultivated tomato, wild relatives of tomato are likely to be the best potential source for this trait (Peralta et al., “Taxonomy of Wild Tomatoes and Their Relatives (Solanum sect. Lycopersicoides, sect. Juglandifolia, sect. Lycopersicon; Solanaceaea),” Syst. Bot. Mon. 84:1-186 (2008)).

Wild relatives of tomato have been screened previously to identify resistance against race 1 Pst strains. One study reported a screen of introgression lines (ILs) derived from S. habrochaites LA1777 using the race 1 strain A9 from California (Thapa et al., “Identification of QTLs Controlling Resistance to Pseudomonas syringae pv. tomato Race 1 Strains From the Wild Tomato, Solanum habrochaites LA1777,” Theor. Appl. Genet. 128: 681-92 (2015)). The detection of four QTLs, on chromosomes 1, 2, and 12 (2 loci), explained the moderate resistance to this strain, however, overall they accounted for a small percentage of the variability observed (10.5-12.5% of the phenotypic variation) (Thapa et al., “Identification of QTLs Controlling Resistance to Pseudomonas syringae pv. tomato Race 1 Strains From the Wild Tomato, Solanum habrochaites LA1777,” Theor. Appl. Genet. 128: 681-92 (2015)). A second study identified two QTLs, on chromosomes 2 and 8, in S. habrochaites accession LA2109 that contributed to resistance to race 1 strain T1, which accounted for 24% and 26% of the phenotypic variability, respectively (Bao Z et al., “Identification of a Candidate Gene in Solanum habrochaites for Resistance to a Race 1 Strain of Pseudomonas syringae pv. tomato,” The Plant Genome 8:10.3835/plantgenome2015.3802.0006 (2015)). Recently, another study reported a screen of 96 wild accessions and identified two accessions that display resistance toward race 1 strain T1, S. neorickii LA1329 and S. habrochaites LA1253. Resistance in LA1253 appears to be a complex genetic trait and its inheritance remains unclear (Hassan et al., “A Rapid Seedling Resistance Assay Identifies Wild Tomato Lines that Aae Resistant to Pseudomonas syringae pv. tomato Race 1,” Mol. Plant Microbe. Interact. 30:701-9 (2017)). Although together these QTLs might contribute to the breeding of enhanced race 1 Pst resistance in tomato, their quantitative nature and relatively weak race 1 resistance limits their usefulness.

Embodiments of the disclosure are directed to overcoming these and other deficiencies in the art.

### SUMMARY

One aspect of embodiments of the disclosure is a nucleic acid construct comprising a nucleic acid molecule comprising a Pseudomonas tomato race 1 (Ptr1) polynucleotide, a 5′ heterologous DNA promoter sequence, and a 3′ terminator sequence, wherein the nucleic acid molecule, the DNA promoter sequence, and the terminator sequence are operatively coupled to permit transcription of the nucleic acid molecule.

Another aspect of embodiments of the disclosure relates to a plant or plant seed transformed with one or more nucleic acid constructs described herein.

Another aspect of embodiments of the disclosure is a method of expressing a nucleic acid molecule in a plant. The method involves providing a transgenic plant or transgenic plant cell transformed with a nucleic acid construct comprising a nucleic acid molecule comprising a Pseudomonas tomato race 1 (Ptr1) polynucleotide, a 5′ heterologous DNA promoter sequence, and a 3′ terminator sequence, wherein the nucleic acid molecule, the DNA promoter sequence, and the terminator sequence are operatively coupled to permit transcription of the nucleic acid molecule, and growing the transgenic plant or a plant grown from the transgenic plant seed under conditions effective to express the nucleic acid molecule in said transgenic plant or said plant grown from the transgenic plant seed.

Yet another aspect of embodiments of the disclosure is a method of imparting disease resistance to a plant. The method involves transforming a plant or a plant cell with a nucleic acid molecule that increases expression of Ptr1, wherein said expression is effective in imparting disease resistance to the transformed plant or to a transgenic plant produced from the transformed plant cell.

A further aspect of embodiments of the disclosure is a method of identifying the presence of a putative Ptr1 sequence based on sequence homology to one or more polynucleotide sequences set forth herein.

A further aspect of embodiments of the disclosure a method of identifying a candidate plant suitable for breeding that displays enhanced disease resistance. The method involves providing a candidate plant; analyzing the candidate plant for the presence, in its genome, of a Ptr1 polynucleotide; identifying, based on said analyzing, a candidate plant suitable for breeding that includes in its genome, a Ptr1 polynucleotide; and breeding the identified plant with at least one other plant.

A further aspect of embodiments of the disclosure a method of identifying a candidate plant suitable for breeding that comprises a putative Ptr1 polynucleotide sequence.

A further aspect of embodiments of the disclosure is a plant cell comprising a nucleic acid molecule comprising a heterologous Pseudomonas tomato race 1 (Ptr1) polynucleotide.

As described herein, it was surprisingly shown that Pseudomonas syringae pv. tomato race 1 resistance in a distantly-related relative of tomato, Solanum lycopersicoides. Experimental results indicate that the resistance is due to a single locus referred to here as Pseudomonas tomato race 1 (Ptr1). Ptr1 confers resistance to several race 1 Pst strains but not to the race 0 strain DC3000. A test of type III effectors that are present in race 1 strains but lacking in DC3000 identified AvrRpt2 as the effector recognized by Ptr1. AvrRpt2 is a cysteine protease that has been intensively studied because it cleaves RIN4 leading to activation of the Arabidopsis NLR protein RPS2 (Axtell et al., “Genetic and Molecular Evidence that the Pseudomonas Syringae Type III Effector Protein AvrRpt2 is a Cysteine Protease,” Mol. Microbiol. 49(6):1537-46 (2003); Mackey et al., “Arabidopsis RIN4 is a Target of the Type III Virulence Effector AvrRpt2 and Modulates RPS2-Mediated Resistance,” Cell 112(3):379-89 (2003), which are hereby incorporated by reference in their entirety). By using site-directed mutagenesis of AvrRpt2 it was discovered that, like RPS2, Ptr1 detects the proteolytic activities of this effector. Ptr1 also detects the activity of AvrRpt2 variants expressed by diverse bacterial phytopathogens and this recognition correlates with ability of the variants to cleave the Arabidopsis RIN4 protein. Notably, Ptr1 conferred strong resistance to a Ralstonia pseudosolanacearum strain that expresses an AvrRpt2 homolog (RipBN); no resistance gene that gives protection against this important pathogen, causative agent of bacterial wilt disease, has been reported previously.

Also described herein is the identification and phylogenetic analysis of the Ptr1 gene. A single recombinant among 585 F2 plants segregating for the Ptr1 locus was discovered that narrowed the Ptr1 candidates to eight nucleotide-binding leucine-rich repeat protein (NLR)-encoding genes. From analysis of the gene models in the S. lycopersicoides genome sequence and RNA-Seq data, two of the eight genes emerged as the strongest candidates for Ptr1. One of these two candidates was found to encode Ptr1 based on its ability to mediate recognition of AvrRpt2 and RipBN when it was transiently expressed with these effectors in leaves of Nicotiana glutinosa. The ortholog of Ptr1 in tomato and in Solanum pennellii is a pseudogene. However, a functional Ptr1 ortholog exists in N. benthamiana and potato and both mediate recognition of AvrRpt2 and RipBN. In apple and Arabidopsis, recognition of AvrRpt2 is mediated by the Mr5 and RPS2 proteins, respectively. Phylogenetic analysis places Ptr1 in a distinct clade compared to Mr5 and RPS2 and it therefore appears to have arisen by convergent evolution for recognition of AvrRpt2.

These findings are a significant advance in disease resistance in plants.

### BRIEF DESCRIPTION OF THE DRAWINGS

FIG. 1A and FIG. 1B provide results that demonstrate that LA4245-R confers resistance to certain strains of Pseudomonas syringae pv. tomato. FIG. 1A shows disease symptoms of LA4245-R and LA4245-S leaves vacuum infiltrated with T1, NY15125 or DC3000 at 3×10⁴ cfu/mL and photographed five days later. FIG. 1B is a graph showing bacterial populations measured two days after inoculation with the Pst strains indicated. Significance was determined by a pair wise t-test and indicated as: *P<0.02, **P<0.0001, not significant (n.s.) at P>0.05. Bars indicate the mean of three plants and error bars represent +/− SEM. Results shown are representative of three independent experiments.

FIG. 2A and FIG. 2B provide results that demonstrate that Pst race 1 resistance in LA4245-R plants is due to the recognition of the bacterial effector AvrRpt2. FIG. 2A shows measures of bacterial populations two days after LA4245-R and LA4245-S plants were vacuum infiltrated with DC3000 carrying avrRpt2_{NY15125} or an empty vector (EV) at 5×10⁴ cfu/mL. FIG. 2B shows measures of bacterial populations two days after LA4245-R and LA4245-S plants were inoculated with NY15125 wild-type, NY15125ΔavrRpt2, a complemented strain, or an empty vector (EV) at 5×10⁴ cfu/mL. Significance was determined by a pair wise t-test and is indicated as: * P<0.05, not significant (n.s.) at P>0.05. Bars indicate the mean of three plants and error bars represent +/− SEM. Both FIG. 5A and FIG. 5B are representative of three independent experiments.

FIG. 3 shows a comparison of the Pst NY15125 chromosome with the chromosomes of Pst strains DC3000 and T1. The innermost ring (solid line) represents the NY15125 chromosome; the next outermost ring (jagged line) represents GC percentage. The next outermost ring (short lines) represent the locations of the type III effectors. The final two most outermost rings (short lines) represent genome comparisons between NY15123 and T1 (second to outermost ring) or DC3000 (outermost ring), respectively. The location of avrRpt2 is shown with a dot on the innermost ring (solid line). Ring image was generated using the BLAST Ring Image Generator (BRIG) sourceforge.net/projects/brig/.

FIG. 4 provides results that demonstrate that deletion of avrRpt2 from Pst strain JL1065 abolishes recognition by Ptr1. LA4245-R and LA4245-S plants inoculated with JL1065 wild-type, JL1065ΔavrRpt2, a complemented strain, or an empty vector (EV) strain at 1×10⁴ cfu/mL. Bacterial populations were measured four days after inoculation. Significance was determined by a pair wise t-test and is indicated as: * P<0.05 or not significant (n.s.) at P>0.05. Bars indicate the mean of three plants and error bars represent +/− SEM. Data are representative of three independent experiments.

FIG. 5 provides results that demonstrate that Ptr1 confers resistance to several race 1 Pseudomonas syringae pv. tomato strains. Disease symptoms of LA4245-R and LA4245-S plants vacuum infiltrated with Pst strains CA-A9, CA-407 and NYT1 at 1×10⁴ cfu/mL and photographed eight days later.

FIG. 6 provides results of immunoblotting that confirms expression of each AvrRpt2 variant in Pst DC3000. Detection of protein accumulation of each AvrRpt2 variant by immunoblotting using ∝-HA. Bacteria were grown in Hrp-inducing minimal media (+) or KB media (−) to detect each effector protein. As expected, proteins were detectable only when the strains were grown in Hrp-inducing medium (+). Ponceau staining shows amount of protein loaded in each lane.

FIG. 7A and FIG. 7B demonstrate that Ptr1 detects the same features of AvrRpt2 as does RPS2. FIG. 7A depicts symptoms of LA4245-R plants vacuum infiltrated with DC3000 expressing avrRpt2 wild-type (WT), avrRpt2 variants or an empty vector (EV) at 5×10⁴ CFU/ml. Photographs were taken five days after inoculation. FIG. 7B shows bacterial populations measured two days after LA4245-R plants were inoculated with DC3000 expressing avrRpt2 wild-type (WT), avrRpt2 variants and empty vector (EV). Significance was determined using ANOVA with a Tukey's post hoc multiple comparison test, and different letters indicate significant differences between treatments (P<0.001). Bars indicate the mean of three plants and error bars represent +/− SEM. Results shown are representative of three independent experiments.

FIG. 8A and FIG. 8B provide results demonstrating that DC3000 strains expressing AvrRpt2 or the AvrRpt2 variants cause similar disease symptoms and grow to the same levels in LA4245-S plants. FIG. 8A shows whole-plant symptoms of LA4245-S plants inoculated with DC3000 carrying avrRpt2 wild-type (WT), avrRpt2 variants or an empty vector (EV) at 5×10⁴ CFU/ml five days after inoculation. FIG. 8B shows bacterial populations in leaves of LA4245-S plants inoculated with DC3000 expressing avrRpt2 wild-type (WT), avrRpt2 variants or an empty vector (EV), 2 days after inoculation. Significance was determined using ANOVA with a Tukey's post hoc multiple comparison test, and different letters indicate significant differences between treatments (P<0.001). Bars indicate the mean of three plants and error bars represent +/− SEM. Data are representative of three independent experiments.

FIG. 9 shows results demonstrating that AvrRpt2 variants F70R, E150S, Y191C and D216E are recognized by RPS2. Five week-old Arabidopsis Col-0 RPS2 plants were dip inoculated with DC3000 carrying avrRpt2 wild-type (WT), avrRpt2 variants or an empty vector (EV) at 3×10⁸ CFU/ml. Bacterial populations were measured three days after inoculation. Significance was determined using ANOVA with a Tukey's post hoc multiple comparison test, and different letters indicate significant differences between treatments (P<0.1). Bars indicate the mean of three plants and error bars represent +/− SEM. Data are representative of three independent experiments.

FIG. 10A and FIG. 10B provide results demonstrating that AvrRpt2 variants F70R, E150S, Y191C, D216E and AvrRpt2 wild-type which are recognized by Ptr1 are able to degrade tomato Rin4 proteins. FIG. 10A shows degradation of the endogenous tomato Rin4 proteins after vacuum infiltration of LA4245-S leaves with DC3000 strains carrying avrRpt2 wild-type (WT), avrRpt2 variants or an empty vector (EV). Plant tissue was harvested 0 and 6 hours post infiltration (hpi). Total protein extracted from infiltrated plants was subjected to immunoblotting using an ∝-RIN4 antibody. Locations of SlRin4-1, Rin4-2, and Rin4-3 are shown. The asterisk indicates an unknown cross-reacting protein. FIG. 10B shows total protein extracted from infiltrated leaves and subjected to immunoblotting using an ∝-RIN4 antibody. Arabidopsis Col-0 rps2 leaves were syringe-infiltrated with DC3000 carrying avrRpt2 wild-type (WT), avrRpt2 variants or an empty vector (EV). Leaf tissue was harvested four hours after infiltration. Total protein extracted from infiltrated leaves was subjected to immunoblotting using an ∝-RIN4 antibody. Ponceau staining shows amount of protein loaded in each lane.

FIG. 11 shows results demonstrating that AvrRpt2 homologs from diverse bacteria are recognized by Ptr1. Agroinfiltration of each avrRpt2 homolog and a yellow fluorescent protein (YFP) control into leaves of LA4245-R and LA4245-S plants. Detached leaves were cleared with ethanol to better visualize cell death associated with AvrRpt2 recognition. Photographs were taken four days after infiltration. Shown are the number of times cell death was observed over the total number of agroinfiltrations performed.

FIG. 12 depicts Agrobacterium-mediated transient expression of AvrRpt2 homologs in leaves of Nicotiana benthamiana. Agroinfiltration of AvrRpt2 homologs (Aa, A. avenae; Ac, A. citrulli; Bp, B. pyrrocinia; Cf, C. fungivorans; Ea, E. amylovora; Mh, M. huakuii; Rs, R. pseudosolanacearum; Sm, S. medicae; Pst, P. syringae pv tomato) at an OD₆₀₀ of 0.3 into N. benthamiana leaves. Samples were taken 43 hours after infiltration. Total proteins extracted from infiltrated leaves were subjected to immunoblotting using an ∝-HA antibody.

FIG. 13A and FIG. 13B show results demonstrating that Pst AvrRpt2(Y191S) variant, analog of the E. amylovora AvrRpt2(C156S) virulent variant, is recognized by Ptr1. FIG. 13A shows disease symptoms of LA4245-R and LA4245-S plants four days after inoculation with DC3000 expressing avrRpt2(Y191S), avrRpt2 wild-type (WT) or an empty vector (EV) at 5×10⁴ CFU/ml. FIG. 13B shows bacterial populations measured in LA4245-R plants two days after inoculation. Significance was determined using ANOVA with a Tukey's post hoc multiple comparison test, and different letters indicate significant differences between treatments (P<0.001). Bars indicate the mean of three plants and error bars represent +/−SD. Results shown are representative of three independent experiments.

FIG. 14A and FIG. 14B show results that demonstrate that Ptr1 confers resistance to Ralstonia pseudosolanacearum strain CMR15 expressing AvrRpt2 homolog RipBN. FIG. 14A shows LA4245-R plants (n=25) and LA4245-S plants (n=30) were soil drench-inoculated with 50 mL of 10⁸ cfu/mL of Ralstonia pseudosolanaceraum CMR15. The percentage of plants surviving (showing no wilt symptoms) or dying (showing severe wilting) 1 to 13 days post-inoculation (DPI) is shown. The two survival curves are significantly different at P<0.0001 using the Mantel-Cox test.

FIG. 14B is photographs taken 13 days after inoculation show extreme wilting of a LA4245-S(left) plant and no wilting of a LA4245-R plant (right). Results shown are representative of three independent experiments.

FIGS. 15A and 15B show results that demonstrate that the predicted proteins of the 14 NLR-encoding genes in the introgression segment of LA4245 bear little similarity to RPS2 or Mr5. FIG. 15A is a scheme of the introgression segments present in LA4245-R. Coordinates (in megabases, Mb) are based on SNP density mapped to S. lycopersicum Heinz 1706 SL2.50. FIG. 15B shows predicted NRL-like genes present in the large introgression segment of LA4245-R (no NLR genes are present in the small introgression segment). Shading indicates absence of this domain.

FIG. 16 shows an annotation of the amino acid sequence of Ptr1 (SEQ ID NO:19). Coiled-coil (CC) domains are shown in bold; nucleotide binding (NB-ARC) domains are shown in underline; leucine-rich repeat (LRR) domains are shaded.

FIG. 17 shows a summary of the type III effectors present in the P. syringae strains T1, NYT1, NY15125, DC3000, CA-407, and CA-A9. The type III effector genes are listed across the top and the P. syringae strains are listed at the left edge. “ORF Present” indicates presence of a full length open-reading frame (ORF). “ORF Present, protein not detectable by immunoblotting” indicates the gene is present but protein is not detectable by immunoblotting. “Partial ORF” indicates the genes has an ORF truncated by an insertion sequence element or the presence of a premature stop codon. “ORF absent” indicates the gene is not present in that strain. “Present in T1 and NY15125, but absent in DC3000” indicates the effectors that are present in T1, NYT1, NY15125, CA-407, and CA-A9 but absent in DC3000. AvrRpt2 is highlighted. DC3000 is the only strain that produce coronatine. References: Jones et al., “Genome-Assisted Development of a Diagnostic Protocol for Distinguishing High Virulence Pseudomonas Syringae pv. tomato Strains,” Plant Disease 99:527-34 (2015); Kraus et al, “Pseudomonas syringae pv. tomato strains from New York Exhibit Virulence Attributes Intermediate Between Typical race 0 and race 1 Strains,” Plant Dis. 101:1442-8 (2017); Kunkeaw et al., “Molecular and Evolutionary Analyses of Pseudomonas syringae pv. tomato Race 1,” Mol. Plant-Microbe. Interact. 23:415-24 (2010); Saha et al., “Bound to Succeed: Transcription Factor Binding-Site Prediction and its Contribution to Understanding Virulence and Environmental Adaptation in Bacterial Plant Pathogens,” Mol. Plant Microbe. Interact. 26(10):1123-30 (2013); and Thapa et al., “Identification of QTLs Controlling Resistance to Pseudomonas syringae pv. tomato Race 1 Strains From the Wild Tomato, Solanum habrochaites LA1777,” Theor. Appl. Genet. 128: 681-92 (2015), each of which is hereby incorporated by reference in its entirety.

FIG. 18 shows features and expression patterns of the four genes in tomato that encode proteins with similarity to Arabidopsis RIN4. The predicted protein mass is shown along with the percentage similarity to the Arabidopsis RIN4 protein (AtRIN4). RNA-Seq data for PTI (pattern-triggered immunity) are from Pombo et al., “Transcriptomic Analysis Reveals Tomato Genes Whose Expression is Induced Specifically During Effector-Triggered Immunity and Identifies the Epk1 Protein Kinase Which is Required for the Host Response to Three Bacterial Effector Proteins,” Genome Biology 15:492 (2014), which is hereby incorporated by reference in its entirety, and for effector-triggered immunity (ETI) are from Rosli et al., “Transcriptomics-Based Screen for Genes Induced by Flagellin and Repressed by Pathogen Effectors Identifies a cell wall-associated Kinase Involved in Plant Immunity,” Genome Biology 14:R139 (2013), which is hereby incorporated by reference in its entirety. Numbers under PTI and ETI are RPKMs (reads per kilobase of transcript per million mapped reads). FDR, false discovery rate. Data are available on the Tomato Functional Genomics Database (TGFD; ted.bti.cornell.edu/cgi-bin/TFGD/digital/home.cgi).

FIG. 19A, FIG. 19B, and FIG. 19C depict delimiting the region containing Ptr1 using progeny of selfed LA4277-R (Ptr1/ptr1) plants. FIGS. 19A and 19B each illustrate four boxed regions—three leftmost boxed regions that are individually shaded (respectively referred to herein as box 1, box 2, and box 3, from left to right) and one rightmost boxed region that appears unshaded or white (referred to herein as box 4). FIG. 19A is a schematic of S. lycopersicoides introgression on chromosome 4 in LA4245-R (box 2 and box 3) and LA4277-R (box 1, box 2, and box 3). The rightmost boxed region shown in white (box 4) is derived from the tomato parent VF36. Distribution of NLR-encoding genes unique to LA4277-R (dots above box 1, representing ˜50 NLR genes) and those shared with LA4245-R (dots above box 2 and box 3) is shown. Indicated are the locations of the DNA markers (megabases, Mb) used to identify recombinants. FIG. 19B depicts that LA4277-159 derived from a selfed LA4277-R plant has a recombination event between markers at 9.15 and 9.94 Mb (box 3). LA4277-R, LA4277-S, and LA4277-159 plants were vacuum infiltrated with Pst JL1065 or Pst JL1065ΔavrRpt2. Photos were taken 6 days after inoculation and are representative of three independent experiments. R, resistant; S, susceptible. FIG. 19C depicts the region defined as containing Ptr1 contains eight NLR gene models, A-H. Their positions and orientations within the Ptr1 region are shown with the coordinates (Mb) corresponding to the start codon of each gene model. Transcripts were not detectable in leaves from genes C, E, F, G, and H; candidate D is a pseudogene.

FIG. 20A, FIG. 20B, FIG. 20C, and FIG. 20D show that candidate A mediates recognition of AvrRpt2 and RipBN in Nicotiana glutinosa. FIG. 20A shows N. glutinosa leaves that were syringe-infiltrated with Agrobacterium strains carrying either A:HA or B:HA (OD₆₀₀=0.025), and AvrRpt2:Myc, AvrRpt2(C122A): Myc or RipBN:Myc (OD₆₀₀=0.05). Photographs were taken 48 hr after infiltration. FIG. 20B shows N. glutinosa leaves that were syringe-infiltrated with Agrobacterium strains expressing A:HA or B:HA (OD₆₀₀=0.1), Prf(D1416V):HA (OD₆₀₀=0.1) or Myc:SlRin4 (OD₆₀₀=0.2). Photographs for candidates A and B were taken 48 hr after infiltration and 4 days after infiltration for Prf(D1416V). FIG. 20C shows N. glutinosa leaves that were syringe-infiltrated with Agrobacterium strains carrying either A:HA (OD₆₀₀=0.1), AvrRpt2:Myc (OD₆₀₀=0.05), RipBN:Myc (OD₆₀₀=0.05), YFP:Myc (OD₆₀₀=0.05) or Myc:SlRin4-3 (OD₆₀₀=0.2). Photographs were taken 48 hr after infiltration. FIG. 20D shows immunoblot analysis of protein extracts isolated from N. benthamiana leaves agroinfiltrated with A:HA (OD₆₀₀=0.1), SlRin4-3:Myc (OD₆₀₀=0.2), AvrRpt2:Myc (OD₆₀₀=0.05), RipBN:Myc (OD₆₀₀=0.05), or YFP:Myc (OD₆₀₀=0.05). Samples were collected 28 hr after infiltration. Total proteins extracted from infiltrated leaves were subjected to immunoblotting using an ∝-HA antibody to detect candidate A and c-Myc antibody to detect SlRin4-3, AvrRpt2, RipBN, and YFP. +, construct was included in the experiment. Protein masses are indicated at the left of the blot. Ponceau staining shows amount of protein loaded in each lane. The asterisk indicates a nonspecific band across the immunoblot. All genes were expressed from the CaMV 35S promoter. Data shown are representative of three independent experiments, using three biological replicates and infiltrating two leaves per plant.

FIG. 21A and FIG. 21B depict Ptr1 orthologs in various solanaceous species. FIG. 21A is a schematic of Ptr1 gene structure in S. lycoperiscoides (Ptr1), S. lycoperiscum (SlPtr1), and S. pennellii (SpPtr1). Ptr1 has a single exon of 2,472 base pairs. SlPtr1 and SpPtr1 are pseudogenes that lack a start codon (asterisk) and have other nonsense mutations (dotted bars) and frameshift mutations (solid bars). The position of each mutation is indicated relative to the Ptr1 base pair (bp) position. FIG. 21B shows that Ptr1 is conserved in many species in the Solanaceae family. Maximum likelihood tree generated from the amino acid sequences of S. lycopersicoides Ptr1 and homologs from S. tuberosum (St), Capsicum chinense (Cc), Capsicum baccatum (Cb), Capsicum annuum (Ca), Nicotiana tomentosiformis (Ntom), Nicotiana tabacum (Ntab), Nicotiana benthamiana (Nb), and Nicotiana attenuata (Na). Bootstrapping 1000 replicates, with bootstrap values over 75 shown. The tree is drawn to scale, with branch lengths measured in the number of substitutions per site.

FIG. 22A and FIG. 22B show that the Ptr1 homolog in N. benthamiana mediates cell death response to AvrRpt2 and RipBN. FIG. 22A shows Agrobacterium strains carrying AvrRpt2:Myc (OD₆₀₀=0.2), AvrRpt2(C122A):Myc (OD₆₀₀=0.2), RipBN:Myc (OD₆₀₀=0.2), YFP:Myc (OD₆₀₀=0.2), or 35S:Prf(D1416V): HA (OD₆₀₀=0.2) were infiltrated into leaves of N. benthamiana (Nb1) or Nb1 plants silenced with either TRV:Ptr1 (Ptr1 VIGS) or TRV:EC1 (EC1 VIGS). Photographs were taken 48 hr after agroinfiltration (except 35S:Prf(D1416V):HA) in Nb1 leaves and after 72 hr for VIGS leaves. 35S:Prf(D1416V):HA treatment was photographed 4 days after infiltration for both Nb1 and VIGS plants. See FIGS. 30A-30B for details of NbPtr1 VIGS construct. Photographs are representative of three independent experiments. For FIG. 22B, Ptr1 gene was synthesized (synPtr1) with a divergent nucleotide sequence to avoid silencing but retaining the same amino acid sequence (FIGS. 31A-31B). Agrobacterium strains harboring AvrRpt2, AvrRpt2(C122A), RipBN, and YFP were infiltrated into leaves at OD₆₀₀=0.05 co-expressed with either 35S:Ptr1:HA or 35S:synPtr1:HA (both at OD₆₀₀=0.025). Photographs were taken 6 days after infiltration and are representative of three independent experiments. All genes were expressed from the CaMV 35S promoter.

FIG. 23A and FIG. 23B show that the Ptr1 ortholog in potato (StPtr1) mediates recognition of AvrRpt2 and RipBN. FIG. 23A shows Nicotiana glutinosa leaves that were syringe-infiltrated with Agrobacterium strains carrying the potato Ptr1 ortholog (OD₆₀₀=0.1) and Myc:SlRin4-3 (OD₆₀₀=0.2) both expressed from the CaMV 35S promoter. Photographs were taken 48 hr after agroinfiltration. FIG. 23B shows N. glutinosa leaves that were syringe-infiltrated with Agrobacterium strains carrying the potato Ptr1 ortholog (OD₆₀₀=0.025), AvrRpt2:Myc (OD₆₀₀=0.05), RipBN:Myc (OD₆₀₀=0.025), and YFP:Myc (OD₆₀₀=0.05) with all genes expressed from the CaMV 35S promoter. Photographs were taken 41 hr after agroinfiltration. Data shown are representative of three independent experiments, using three biological replicates and infiltrating two leaves per plant.

FIG. 24 is a phylogenetic tree of Ptr1, Mr5, RPS2 with their most closely related proteins in S. lycopersicoides. Maximum likelihood tree of the amino acid sequence of the NB-ARC domain only of Ptr1, Mr5, and RPS2 top BLAST hits with an intact NB-ARC domain, including the P-loop, kinase 2, kinase 3a, and GLPL motifs, from the S. lycopersicoides genome protein database. 1000 bootstrap replicates, with bootstraps 75 or above shown. The tree is drawn to scale, with branch lengths measured in the number of substitutions per site.

FIG. 25A and FIG. 25B show the amino acid sequences of Rin4 proteins in (among others) tomato and Arabidopsis and suppression of candidate A-mediated cell death by N- or C-terminal Myc-tagged tomato Rin4 proteins. FIG. 25A is an amino acid sequence alignment of Rin4 proteins from tomato (Solanum lycopersicum (Sl)); Arabidopsis thaliana (At); and Malus domestica (Md) (S1Rin4-1, SEQ ID NO:85; S1Rin4-2, SEQ ID NO:86; S1Rin4-2, SEQ ID NO:87; AtRIN4, SEQ ID NO:88; MdRIN4-1, SEQ ID NO:89, MdRIN4-2, SEQ ID NO:90) (Table 13). Black background indicates identical amino acid residues; grey background indicate similar amino acid residues; boxed regions RCS1 and RCS2 indicate regions cleaved by AvrRpt2 cysteine protease activity with arrows marking the exact cleavage site. AvrRpt2 cleavage products (ACP) are indicated. Boxed region PS2 indicates the palmitoylation (PS). The polymorphic residues in AtRIN4 and the two MdRIN4 proteins are shown. FIG. 25B shows N. glutinosa leaves that were syringe-infiltrated with Agrobacterium strains carrying A:HA (OD₆₀₀=0.1) and SlRin4:Myc (OD₆₀₀=0.2) with both being expressed by the CaMV 35S promoter. Tomato Rin4 proteins were c-Myc tagged at the N- or C-terminal as indicated. Photographs were taken 48 hr after agroinfiltration. Data shown are representative of three independent experiments, using three biological replicates and infiltrating two leaves per plant.

FIG. 26A and FIG. 26B show that candidate B does not mediate recognition of AvrRpt2 or RipBN in N. glutinosa. FIG. 26A shows N. glutinosa leaves that were syringe-infiltrated with Agrobacterium strains carrying either B:HA (OD₆₀₀=0.1), AvrRpt2:Myc (OD₆₀₀=0.05), RipBN:Myc (OD₆₀₀=0.05), YFP:Myc (OD₆₀₀=0.05) or Myc:SlRin4-3 (OD₆₀₀=0.2). Photographs were taken 48 hr after agroinfiltration. FIG. 26B shows immunoblot analysis of protein extracts isolated from N. benthamiana leaves agroinfiltrated with B:HA (OD₆₀₀=0.1), Myc:SlRin4-3 (OD₆₀₀=0.2), AvrRpt2:Myc (OD₆₀₀=0.05) or YFP:Myc (OD₆₀₀=0.05). Samples were collected 28 hr after agroinfiltration. Total proteins extracted from infiltrated leaves were subjected to immunoblotting using an ∝-HA antibody to detect candidate B or a c-Myc antibody to detect SlRin4-3, AvrRpt2, RipBN and YFP. Protein masses are indicated at the left of the blot. Ponceau staining shows amount of protein loaded in each lane. +, candidate B protein; *, a nonspecific bands across the immunoblot. All genes in this figure were expressed from the CaMV 35S promoter. Data shown are representative of three independent experiments, using three biological replicates and infiltrating two leaves per plant.

FIG. 27 shows the amino acid sequence of the predicted Ptr1 protein showing motifs characteristic of NLR immune receptors. The full sequence of Ptr1 (SEQ ID NO:19) is shown with annotations, and motifs are set forth below the full sequence: NB-ARC P-loop (Ptr1, SEQ ID NO:91; Consensus, SEQ ID NO:92); NB-ARC kinase 2 (Ptr1, SEQ ID NO:93; Consensus, SEQ ID NO:94); NB-ARC kinase 3a (Ptr1, SEQ ID NO:95; Consensus, SEQ ID NO:96); NB-ARC GLPL (Ptr1, SEQ ID NO:97; Consensus, SEQ ID NO:98); NBR-ARC MHD (Ptr1, SEQ ID NO:99; Consensus, SEQ ID NO: 100); Leucine-rich repeats (SEQ ID NOs:101-113) (Table 13). The LRR repeats (L_{XX}L_{XX}L_{XX}L_{X}L_{XX}L_{XX} motif) were identified using the LRRSearch Tool (Bej et al., “LRRsearch: An Asynchronous Server-Based Application for the Prediction of Leucine-Rich Repeat Motifs and an Integrative Database of NOD-like Receptors,” Comp. Biol. Med. 53:164-70 (2014), which is hereby incorporated by reference in its entirety) and adjusted manually.

FIG. 28A and FIG. 28B show that the ortholog of Ptr1 is a pseudogene in tomato and S. pennellii. FIG. 28A and FIG. 28B show a ClustalW alignment of the nucleotide sequence (including 20 nucleotides upstream of start codon) of candidate A (Ptr1, SEQ ID NO:19) and its ortholog in S. lycopersicum (SlPtr1, SEQ ID NO:114) and S. pennellii (SpPtr1, SEQ ID NO:115) (Table 13). The asterisk denotes the start codon unique to S. lycopersicoides Ptr1. Numbers on right correspond to the sequence of Ptr1. FIG. 28A shows positions 1-1,260 of the alignment. FIG. 28B shows positions 1,261-2,500 of the alignment.

FIG. 29A, FIG. 29B, and FIG. 29C show Ptr1 orthologs in various Solanaceae species. FIG. 29A shows a comparison of Ptr1 nucleotide and amino acid sequences with its orthologs in potato and Nicotiana benthamiana. FIG. 29B and FIG. 29C show an amino acid sequence alignment of Ptr1 proteins from various Solanaceae species. S. lycopersicoides Ptr1 (Ptr1, SEQ ID NO:19), S. tuberosum Ptr1 (SEQ ID NO:116), C. anuum Ptr1 (SEQ ID NO:117), C. baccatum Ptr1 (SEQ ID NO:118), C. chinense Ptr1 (SEQ ID NO:119), N. attenuata Ptr1 (SEQ ID NO:120), N. benthamiana Ptr1a (SEQ ID NO:121), N. tabacum Ptr1 (SEQ ID NO:122), N. tomentosiformis Ptr1 (SEQ ID NO:123) (Table 13). FIG. 29B shows positions 1-400 of the alignment. FIG. 29C shows positions 401-833 of the alignment.

FIG. 30A and FIG. 30B show that Ptr1 has extensive sequence similarity with the two Ptr1 homologs in N. benthamiana. Nucleotide alignment of Ptr1 (SEQ ID NO:18), NbPtr1a (SEQ ID NO:124) and NbPtr1b (SEQ ID NO:125) (Table 13). FIG. 30A shows positions 1-1,170 of the alignment. FIG. 30B shows positions 1,171-2,482 of the alignment. A five base pair deletion in NbPtr1b (box at positions 98-102) leads to a premature stop codon (box at positions 166-168), although the nucleotide sequence remains conserved at the region targeted by VIGS (line above positions 1,213-1,538).

FIG. 31A and FIG. 31B show a nucleotide alignment of Ptr1 (SEQ ID NO:18) and synthetic Ptr1 (synPtr1, SEQ ID NO:126) (Table 13). FIG. 31A shows positions 1-1,236 of the alignment. FIG. 31B shows positions 1,237-2,472 of the alignment. Identical nucleotides are shown in black. The amino acid sequence is identical for both Ptr1 and synPtr1.

FIG. 32 shows immunoblot analysis of protein extracts isolated from N. benthamiana leaves transiently expressing 35S:Ptr1:HA or 35S:synPtr1:HA. Proteins were extracted from leaves collected 46 hr after Agrobacterium-mediated transient transformation of Ptr1 and synPtr1 (agroinfiltration; OD₆₀₀=0.1) and subjected to immunoblotting using an ∝-HA antibody to detect Ptr1 and synPtr1. Protein masses are indicated at the left of each blot. Ponceau staining shows amount of protein loaded in each lane.

FIG. 33A, FIG. 33B, FIG. 33C, and FIG. 33D show that Ptr1 has little nucleotide similarity with RPS2 or Mr5. FIGS. 33A-33B show an alignment of the nucleotide sequences of Ptr1 (SEQ ID NO:18) and RPS2 (SEQ ID NO:127 (Arabidopsis thaliana)) (Table 13) generated using MUSCLE. FIG. 33A shows positions 1-1,484 of the alignment. FIG. 33B shows positions 1,485-2,966 of the alignment. Identical nucleotides are shown in black. FIGS. 33C-33D show an alignment of the nucleotide sequences of Ptr1 (SEQ ID NO:18) and Mr5 (SEQ ID NO:128 (Malus domestica)) (Table 13) generated using MUSCLE. FIG. 33C shows positions 1-2,144 of the alignment. FIG. 33D shows positions 2,145-4,265 of the alignment. Identical nucleotides are shown in black.

FIG. 34A, FIG. 34B, FIG. 34C, and FIG. 34D show that Ptr1, StPtr1, and NbPtr1 share little amino acid sequence similarity with RPS2 (FIG. 34A-34B) or Mr5 (FIGS. 34C-34D). FIGS. 34A-34B show an amino acid alignment of the Ptr1 protein (SEQ ID NO:19) with StPtr1 (GenBank Accession No. XP_006340095.1, which is hereby incorporated by reference in its entirety) (SEQ ID NO:131), NbPtr1a (SEQ ID NO:121), and RPS2 (SEQ ID NO:129 (Arabidopsis thaliana)) (Table 13). FIG. 34A shows positions 1-480 of the alignment. FIG. 34B shows positions 481-959 of the alignment. FIGS. 34C-34D show an amino acid alignment of the Ptr1 protein (SEQ ID NO:19) with StPtr1 (SEQ ID NO:131), NbPtr1a (SEQ ID NO:121), and Mr5 (SEQ ID NO:130 (Malus domestica)) (Table 13). FIG. 34C shows positions 1-720 of the alignment. FIG. 34D shows positions 721-1,402 of the alignment. Darker shading indicates all four of the proteins have the same residue at that position, lighter shading indicates that three of the four proteins have the same residue at that position. Lines indicates leucine-rich repeats (LRRs).

FIG. 35A and FIG. 35B show phylogenetic trees of Ptr1, Mr5, and RPS2 with their most closely related proteins in Arabidopsis and apple. Maximum likelihood trees of top BLAST hits of Ptr1, Mr5, and RPS2 NB-ARC domains from: Arabidopsis (AT; Araport 11 protein database) (FIG. 35A) and Malus domestica (MD; GDDH13 V1.1 protein database) (FIG. 35B). Only hits with an intact NB-ARC domain, including the P-loop, kinase 2, kinase 3a, and GLPL motifs were included in the analysis. Bootstrapping 1000 replicates, with bootstraps over 75 shown. The tree is drawn to scale, with branch lengths measured in the number of substitutions per site.

FIG. 36 shows a maximum likelihood phylogenetic tree of AvrRpt2 homologs in GenBank. Ptr1 is known to mediate recognition of AvrRpt2 proteins indicated with asterisks (all are cysteine protease-active). Bootstrapping 1000 replicates, with bootstraps over 75 shown. The tree is drawn to scale, with branch lengths measured in the number of substitutions per site.

### DETAILED DESCRIPTION

Unless otherwise indicated, the definitions and embodiments described in this and other sections are intended to be applicable to all embodiments and aspects of the present application herein described for which they are suitable as would be understood by a person skilled in the art.

As used in this application, the singular forms “a”, “an” and “the” include plural references unless the content clearly dictates otherwise.

The term “and/of” as used herein means that the listed items are present, or used, individually or in combination. In effect, this term means that “at least one of” or “one or more” of the listed items is used or present.

Terms of degree such as “substantially”, “about” and “approximately” as used herein mean a reasonable amount of deviation of the modified term such that the end result is not significantly changed. These terms of degree should be construed as including a deviation of at least +/−5% of the modified term if this deviation would not negate the meaning of the word it modifies.

Where a range of values is provided, it is intended that each intervening value between the upper and lower limit of that range and any other stated or intervening value in that stated range is encompassed within the disclosure.

As used herein, the terms “nucleic acid”, “polynucleotide”, and “DNA” are used interchangeably, unless indicated otherwise by context. In some embodiments, a nucleic acid or protein of the invention is “isolated”. As used herein, the term “isolated” refers to a synthesized, cloned, and/or truncated sequence from the naturally-occurring sequence.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “comprising” and its derivatives, as used herein, are intended to encompass “consisting of” and “consisting essentially of”. The term “consisting of” and its derivatives are intended to be closed terms that specify the presence of the stated features, elements, components, groups, integers, and/or steps, but exclude the presence of other unstated features, elements, components, groups, integers and/or steps. The term “consisting essentially of” is intended to specify the presence of the stated features, elements, components, groups, integers, and/or steps as well as those that do not materially affect the basic and novel characteristic(s) of features, elements, components, groups, integers, and/or steps.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

Embodiments of the disclosure relate to disease resistance in plants and compositions useful in imparting or enhancing disease resistance in plants.

Accordingly, a first aspect of embodiments of the disclosure is a nucleic acid construct comprising a nucleic acid molecule comprising a Pseudomonas tomato race 1 (Ptr1) polynucleotide, a 5′ heterologous DNA promoter sequence, and a 3′ terminator sequence, wherein the nucleic acid molecule, the DNA promoter sequence, and the terminator sequence are operatively coupled to permit transcription of the nucleic acid molecule.

In some embodiments, the nucleic acid molecule comprises the nucleotide sequence of SEQ ID NO:18, as set forth below:

ATGGCGGAATCTTTCTTGTTCAATATCATTGAACGGGTTTTGGCTAAAGT
TTCTTCAATTGCTGTATATGAGATCAGTCTAGCTTGGAATGTTAAGACAG
AGCTAAGGAAACTCCAAAGTACTCTATCCACCATCAAAGCTGTACTTCTA
GATGCAAACGAGCAAAAGGCAAAGAACCATGAAGTGAGAGATTGGCTGGA
AAAGCTCAGAGATGTTGTTTATGATGTCGATGATTTGATGGATGATTTAT
CAACACAACTGTTGCTGCAAATGCATTTCCAGAAAAGCTTTAGGAAGAAG
GTAAGAAGATTCTTTTCCAGTTCAAATCCAATTATATATCGATTCAAGAT
TGGCAGAAAGGTAAAAGAAATCAGGGAGCTGCTGAATGAGATTGCAGATG
ATAGGAGAAATTTCCACTTCACGGAACATACTTATGTAATTCCAGCTGAG
AATACGAGTAGAGAACAAACACACTCCTTTGTGAGGGCCTCAGATATCAT
TGGTAGAGATGATGATCAAGAAAACATTGTAAAACAGCTGATAGATTCTC
ATGATGAGGAAAATATTTCTGTGATTCCTATTGTTGGACTTGGAGGGCTT
GGAAAAACCACACTTGTTAAGTTGGTTTATAACAATAATAGGGTTGTTCA
GAATTTTGACCTTAGAATGTGGGTTAGTATTTCAGAAGATTTCAGTCTGA
GCAAGGTAATTGAGAAAATTCTGAGGTCCGCAACAGGAGAGAGTTTTGAC
CACCTAGATATGGACCAATTACAATGTTGTTTGGGAGAGGTTTTGCAACA
GAAAAGGTATTTACTTGTGCTGGATGATGTTTGGAACGAAGATCAACACA
AGTGGACGGATCTGAGGGAGTTGCTGATGAATTGTTCCAGAGGTAGTAAA
ATTGTTGTCACTACACGTAGTAAGATGGTTGCTTTGATTACTGGAACAGT
TCCGCCTTATTATTTGGGAGGCCTTGCTAATGATGACTGCTTATCTTTAT
TTTTGAAATGTGCATTTGGAGGACAGGACAATTTGTTTCCTAATCTAGTA
GAAATAGGAAAAGAAATTGTGAAAAAGTGTGGAGGAGTGCCTTTGGCTGT
GAAAACCTTGGGAAGGTTGTTGTACATGAAAACAGACGAGAATGAATGGT
TGCAGATAAGAGATAATGAGATATGGGAAATCGAACAGAATAAATCTGAC
ATTTTACCAATATTGAGATTGAGCTATGAACAGATGCCATCACATCTAAG
ACAGTGCTTTGCCTATTGCTCCATGTTACCCAAAGGTCAAGAAATTCCGA
GGGAGGATTTTATCAATCGCTGGATTGCTCAAGGATTTATACAGAGTTCC
AACAGAAACAGGAAGCTGGAAGATATCGGTAATCAGTACTTTGATGAGTT
GCTATCAAGGTTTTGCTTCCTAGATGTGGTACAAGCTTTTGATGGAGAAA
TATTGGCTTGTAAGATACACAATCTTGTGCATGATCTTGCACAGTCAGTA
TCAGGTGCAGAGTGCTTAAATGTGAAACCCAATGCTTTCGTGGTCTCTGA
AAGAGTTCGCCACTTATTTTTCCATGCAGAAGATATGTCTAGGAAACACT
TCCCCAGATTTTTGCTTCCTTTGCAAAAGTTGAGGTCTTTCTCTTATTCT
TTTAACATTGGACCTGTAAACAAGTTCTTTGTCAAGACAATGTTGTCAAA
TTTCAAATGCCTTCGGATGCTAGTCTTGAACAATCTAGATCTTGAGGAGT
TGCCAACTTCGATAGGTCACTTGAAGGAATTAAGATACCTCAACCTTAGT
GACAGTGGTAAGATCAAGTTTCTTCCAAGGTCTATGAGCAAATTAGTAAA
TCTGCACACCCTAAACCTCATTAACTGTGAACAGCTTAAGGAGTTGCCAA
GAGATTTTAGAAAGTTAATCAGCCTGAAGACCTTGTATTTGACTACACAT
CAGATGTCAGCAGGGATCAAGAATCAACATTCTTTCACTTCTCTTCAATT
TTTACTTCTTTTCAAATGTTGTTTCCCAAAATTGCAGCCAGAACTGGTGC
AGCATTTTACYGCACTTCGGGTTTTGCGTATCTATGAATGCCCAAGTTTA
TGTTCTCTTCCAAGCAGTATTAGATATCTGACTTCACTTGAAAAGCTATG
GATCTGGAACTGTGAAGAACTTGATTTGATTGATGGAGAAGGGATGTCAG
GCCTAACAAGTCTTCAATCCTTGCTTCTAATGGGGCTTCCTAAGTTGGTG
ACTCTACCATTGGAACTTAAAGATACTGCTCCTACAACATTAAAGTACTT
CAGAATCGCCGATTGTCCCAACCTGGTGGAGCTTCCAGAGTGGCTGCCTA
ATTGCTCCTCACTTCAGAGACTCTATATAGAGGATTGTCCTGTTTTGGCA
TCGATACCTCAAGGAATCTACAGCCACAATGCCAACGTCCATATAATCGA
CTGTCCATTGCTAGGTGGATGA

In some embodiments, the nucleic acid molecule may include the nucleotide sequence of SEQ ID NO:18. Also encompassed are nucleic acid molecules at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical with the entire sequence of SEQ ID NO:18.

In some embodiments, the invention provides nucleic acids capable of improving disease resistance in a plant. A person of ordinary skill in the art would be readily able to predict a resulting protein sequence based on the nucleic acid molecules of embodiments of the disclosure. Thus, where protein coding nucleic acid sequences are disclosed, the resulting amino acid sequence or sequences (i.e. a polypeptide or protein sequence) are also contemplated as embodiments of the disclosure.

Accordingly, polypeptides or protein sequences of embodiments of the disclosure include SEQ ID NO:19, as set forth below:

MAESFLFNIIERVLAKVSSIAVYEISLAWNVKTELRKLQSTLSTIKAVLL
DANEQKAKNHEVRDWLEKLRDVVYDVDDLMDDLSTQLLLQMHFQKSFRKK
VRRFFSSSNPIIYRFKIGRKVKEIRELLNEIADDRRNFHFTEHTYVIPAE
NTSREQTHSFVRASDIIGRDDDQENIVKQLIDSHDEENISVIPIVGLGGL
GKTTLVKLVYNNNRVVQNFDLRMWVSISEDFSLSKVIEKILRSATGESFD
HLDMDQLQCCLGEVLQQKRYLLVLDDVWNEDQHKWTDLRELLMNCSRGSK
IVVTTRSKMVALITGTVPPYYLGGLANDDCLSLFLKCAFGGQDNLFPNLV
EIGKEIVKKCGGVPLAVKTLGRLLYMKTDENEWLQIRDNEIWEIEQNKSD
ILPILRLSYEQMPSHLRQCFAYCSMLPKGQEIPREDFINRWIAQGFIQSS
NRNRKLEDIGNQYFDELLSRFCFLDVVQAFDGEILACKIHNLVHDLAQSV
SGAECLNVKPNAFVVSERVRHLFFHAEDMSRKHFPRFLLPLQKLRSFSYS
FNIGPVNKFFVKTMLSNFKCLRMLVLNNLDLEELPTSIGHLKELRYLNLS
DSGKIKFLPRSMSKLVNLHTLNLINCEQLKELPRDFRKLISLKTLYLTTH
QMSAGIKNQHSFTSLQFLLLFKCCFPKLQPELVQHFTALRVLRIYECPSL
CSLPSSIRYLTSLEKLWIWNCEELDLIDGEGMSGLTSLQSLLLMGLPKLV
TLPLELKDTAPTTLKYFRIADCPNLVELPEWLPNCSSLQRLYIEDCPVLA
SIPQGIYSHNANVHIIDCPLLGG*.

In some embodiments, the polypeptides or proteins according to this or any other embodiment described herein comprise one or more (e.g., 1, 2, 3, 4, 5 or more) amino acid insertions, deletions, or modifications (e.g., substitution of one amino acid for another) compared to SEQ ID NO:19, or are otherwise substantially identical (e.g., having a sequence at least 80%, 81%, 82%, 83%, 84%, 85%, 86%, 87%, 88%, 89%, 90%, 91%, 92%, 93%, 94%, 95%, 96%, 97%, 98%, 99% or more identical) with the entire sequence of SEQ ID NO:19. It is contemplated that such variants retain the function of, for example, SEQ ID NO:19 (e.g., in imparting or enhancing disease resistance). For example, polypeptides or proteins comprising an amino acid sequence having one or more (e.g., 1, 2, 3, 4, 5, or more) conservative amino acid substitutions relative to SEQ ID NO:19, but retaining the function of SEQ ID NO:19 (e.g., in imparting or enhancing disease resistance) are encompassed. Nucleic acid molecules encoding such variants of the polypeptides of embodiments of the disclosure are also contemplated. Such nucleic acid molecules may have, for example, a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identical with the entire sequence of SEQ ID NO:18.

In some embodiments, the polynucleotide, polypeptide, or protein (or a fragment or variant thereof) of embodiments of the disclosure confers resistance to an organism expressing the bacterial effector AvrRpt2 (or fragments or variants thereof) or a homolog of AvrRpt2 (or fragments or variants thereof), the sequence of which SE ID NO:16 is as follows:

MKIAPVAINHSPLSREVPSHAAPTQAKQTNLQSEAGDLDARKSSASSPET
RALLATKTVLGRHKIEVPAFGGWFKKKSSKHETGGSSANADSSSVASDST
EKPLFRLTHVPYVSQGNERMGCWYACARMVGHSVEAGPRLGLPELYEGRE
GPAGLQDFSDVERFIHNEGLTRVDLPDNERFTHEELGALLYKHGPIIFGW
KTPNDSWHMSVLTGVDKETSSITFHDPRQGPDLAMPLDYFNQRLAWQVPH
AMLYR

Thus, it is contemplated that such a polynucleotide, polypeptide, or protein (or fragment or variant thereof) confers resistance to an organism expressing the effector encoded by SEQ ID NO:16. In some embodiments, the polynucleotide or protein (or fragment or variant thereof) confers resistance to a fragment or variant of SEQ ID NO:16, including those that comprise one or more (e.g., 1, 2, 3, 4, 5 or more) amino acid insertions, deletions, or modifications (e.g., substitution of one amino acid for another) compared to SEQ ID NO:16, or are otherwise substantially identical (e.g., having a sequence at least 80%, 85%, 90%, 95%, 96%, 97%, 98%, 99% or more identical) with the entire sequence of SEQ ID NO:16).

In some embodiments, a polynucleotide, polypeptide, or protein (or fragment or variant thereof) of embodiments of the disclosure confers resistance to an organism expressing the bacterial effector AvrRpt2 (SEQ ID NO:16) comprising mutations selected from F70R, E150S, Y191C, D216E, and combinations thereof.

In some embodiments, a polynucleotide, polypeptide, or protein (or fragment or variant thereof) of embodiments of the disclosure confers resistance to an organism expressing a pathogen effector protein (e.g., AvrRpt2 as described herein) capable of degrading a RIN4 expressed by the cell (e.g., host cell) or plant of embodiments of the disclosure. Accordingly, in some embodiments, a polynucleotide (e.g., Ptr1 or a variant or ortholog thereof as described herein) or polypeptide or protein (e.g., encoded by a polynucleotide described herein or fragment or variant thereof) of embodiments of the disclosure confers resistance to an organism by responding to degradation of a target (e.g., RIN4) by a pathogen-associated protease (e.g., AvrRpt2). In some embodiments, the response to degradation is a conformational change in the polynucleotide, polypeptide, or protein. In some embodiments, the method involves co-expressing one or more RIN4 proteins and Ptr1 (or a variant or ortholog thereof) as described herein. Accordingly, in some embodiments of the disclosure, the nucleic acid molecule described herein further comprises one or more RIN4 polynucleotides. See Kim et al., “Using Decoys to Expand the Recognition Specificity of a Plant Disease Resistance Protein,” Research Reports 351 (6274): 684-687 (2016), which is hereby incorporated by reference in its entirety. In some embodiments, RIN4 is Arabidopsis thaliana RIN4 (see Mackey et al., “Arabidopsis RIN4 is a Target of the Type III Virulence Effector AvrRpt2 and Modulates RPS2-Mediated Resistance,” Cell 112(3):379-89 (2003), which is hereby incorporated by reference in its entirety) or a variant or ortholog thereof. Exemplary RIN4 proteins are described in the Examples below. For example, in some embodiments, RIN4 is encoded by RIN4-1, RIN4-2, RIN4-3, and/or RIN4-4 (corresponding, respectively, to Solyc identifiers Solyc09g059430, Solyc06g083390, Solyc12g098440, and Solyc11g012010, each of which are hereby incorporated by reference in its entirety (see FIG. 18) or a variant or ortholog thereof. Accordingly, another aspect of the disclosure is a method of imparting or enhancing disease resistance in plants expressing a pathogen effector protein (e.g., AvrRpt2 as described herein) capable of degrading a RIN4 expressed by the plant.

In some embodiments, the nucleic acid molecule further comprises a Pseudomonas tomato race (Pto) polynucleotide, the amino acid sequence of which (SEQ ID NO:17) is as follows:

MGSKYSKATNSINDALSSSYLVPFESYRVPLVDLEEATNNFDHKFLIGHG
VFGKVYKGVLRDGAKVALKRRTPESSQGIEEFETEIETLSFCRHPHLVSL
IGFCDERNEMILIYKYMENGNLKRHLYGSDLPTMSMSWEQRLEICIGAAR
GLHYLHTRAIIHRDVKSINILLDENFVPKITDFGISKKGTELDQTHLSTV
VKGTLGYIDPEYFIKGRLTEKSDVYSFGVVLFEVLCARSAIVQSLPREMV
NLAEWAVESHNNGQLEQIVDPNLADKIRPESLRKFGDTAVKCLALSSEDR
PSMGDVLWKLEYALRLQESVI

The Ptr1 nucleic acid molecules of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire Ptr1 sequences set forth herein or to variants and fragments thereof are encompassed by embodiments of the disclosure. Accordingly, another aspect of embodiments of the disclosure is a method of identifying the presence of a putative Ptr1 sequence based on sequence homology to the sequences set forth herein by, for example, using next generation sequencing, TaqMan assays, UniTaq assays, real-time PCR assays, digital PCR, microarray, hybridization or other detection methods. Accordingly, a further aspect of embodiments of the disclosure a method of identifying a candidate plant suitable for breeding that comprises a putative Ptr1 sequence. The method involves providing a candidate plant; analyzing the candidate plant for the presence, in its genome, of a putative Ptr1 polynucleotide; identifying, based on said analyzing, a candidate plant suitable for breeding that includes in its genome, a Ptr1 polynucleotide; and breeding the identified plant with at least one other plant.

Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode for an Ptr1 protein or polypeptide and which hybridize under stringent conditions to at least one of the Ptr1 nucleic acid molecules disclosed herein, or to variants or fragments thereof, are encompassed by embodiments of the disclosure. In some embodiments, the orthologous Ptr1 to those described herein may be from a member of the Solanaceae family. Plants that are members of the Solanaceae family include, but not limited to, tomato, potato, pepper, tobacco, eggplant, tomatillo, and petunia.

Accordingly, in embodiments, the polynucleotide, polypeptide, or protein (or a fragment or variant thereof) of embodiments of the disclosure is an ortholog of Ptr1. In some embodiments, the polynucleotide, polypeptide, or protein (or a fragment or variant thereof) of embodiments of the disclosure is a modified form of an ortholog of Ptr1.

In some embodiments, the ortholog of Ptr1 is from a species of Solanum, Capsicum, or Nicotiana. In some embodiments, the ortholog of Ptr1 is from S. lycopersicum, S. pennellii, S. tuberosum, C. annuum, C. baccatum, C. chinense, N. attenuata, N. benthamiana, N. tabacum, or N. tomentosiformis.

In some embodiments, the ortholog of Ptr1 is RGA1 of Solanum tuberosum (St). For example, in some embodiments the ortholog of Ptr1 has the amino acid sequence of SEQ ID NO: 131 (GenBank Accession No. XP_006340095.1). In some embodiments the ortholog of Ptr1 has the polynucleotide sequence of SEQ ID NO:132 (coding sequence from GenBank Accession No. XM_006340033.2).

In some embodiments, the ortholog of Ptr1 is selected from S. lycopersicum (SlPtr1, SEQ ID NO:114), S. pennellii (SpPtr1, SEQ ID NO:115), S. tuberosum Ptr1 (SEQ ID NO:116), C. anuum Ptr1 (SEQ ID NO:117), C. baccatum Ptr1 (SEQ ID NO:118), C. chinense Ptr1 (SEQ ID NO:119), N. attenuata Ptr1 (SEQ ID NO:120), N. benthamiana Ptr1a (SEQ ID NO:121), N. tabacum Ptr1 (SEQ ID NO:122), or N. tomentosiformis Ptr1 (SEQ ID NO:123).

In some embodiments, the ortholog of Ptr1 is selected from S. lycopersicum (SlPtr1, SEQ ID NO:114) or S. pennellii (SpPtr1, SEQ ID NO:115).

In some embodiments, the ortholog of Ptr1 is S. lycopersicum (SlPtr1, SEQ ID NO:114).

Components of the plant cells and nucleic acid constructs according to embodiments of the disclosure may be heterologous. A polynucleotide sequence is “heterologous to” an organism or a second polynucleotide sequence if it is synthetic or originates from a different organism, or, if from the same organism, is modified from its original form. For example, a promoter operably linked to a heterologous coding sequence (or vice versa) refers to a coding sequence from an organism or species different from that from which the promoter was derived, or, if from the same organism or species, a coding sequence which is not naturally associated with the promoter (e.g., a genetically engineered coding sequence or an allele from a different ecotype or variety).

In some embodiments, a plant or cell provided by the invention incorporates a heterologous polynucleotide sequence not native to the plant or cell, e.g., an exogenous nucleic acid derived from another plant, strain, or cultivar. In some embodiments, a plant or cell of the invention incorporates modifications to its own native polynucleotide sequence (e.g., by genome modification as described herein) resulting in a heterologous polynucleotide sequence. The resulting heterologous polynucleotide sequence may result in a change in the expression or functionality of the gene product of the modified polynucleotide sequence or of another native polynucleotide sequence. Modifications can be incorporated, for example, by genome modification. The specific mutations can be based on information such as the use of known techniques to identify variations in the gene that correlate with improved function. In some embodiments, modifications may be introduced to produce a heterologous polynucleotide that encodes a protein that imparts or enhances disease resistance in a plant as described herein. Such modifications include modifications of the native polynucleotide sequence to increase sequence similarity to SEQ ID NO: 18 or to produce an expression product with similarity (or increased similarity) to SEQ ID NO:19. For example, in comparing the native polynucleotide sequence to that of SEQ ID NO:18, differences (or mutations) in the native polynucleotide sequence versus SEQ ID NO:18 may be identified. For example, functional start and/or stop codons, missense mutations, nonsense mutations, splice junction mutations, insertion mutations, deletion mutations, or frameshift mutations may be identified in the native polynucleotide sequence as compared to SEQ ID NO: 18. See Example 15. Accordingly, in some embodiments, modifications to a native polynucleotide sequence to produce a heterologous polynucleotide include modifications made to introduce or repair a start and/or stop codon or modifications to repair missense mutations, nonsense mutations, splice junction mutations, insertion mutations, deletion mutations, or frameshift mutations to correspond to, e.g., the polynucleotide sequence of SEQ ID NO:18.

Genome modification can be achieved, for example, by use of known techniques or systems for site directed mutagenesis or genome editing. Such techniques or systems include, for example, zinc finger nucleases (“ZFNs”) (Urnov et al., “Genome Editing with Engineered Zinc Finger Nucleases,” Nat Rev Genet. 11: 636-646 (2010), which is hereby incorporated by reference in its entirety), transcription activator-like effector nucleases (“TALENs”) (Joung & Sander, “TALENs: A Widely Applicable Technology for Targeted Genome Editing,” Nat Rev Mol Cell Biol. 14: 49-55 (2013), which is hereby incorporated by reference in its entirety), clustered regularly interspaced short palindromic repeat (“CRISPR”)-associated endonucleases (e.g., CRISPR/CRISPR-associated (“Cas”) 9 systems and variants thereof) (Wiedenheft et al., “RNA-Guided Genetic Silencing Systems in Bacteria and Archaea,” Nat 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013); Chen et al., “CRISPR/Cas Genome Editing and Precision Plant Breeding in Agriculture,” Ann. Rev. Plant Biol. 70:667-97 (2019), each of which is hereby incorporated by reference in its entirety), or DNA free genome editing (Metje-Sprink et al., “DNA-Free Genome Editing: Past, Present, and Future,” Front. Plant. Sci. 9:1957 (2019), which is hereby incorporated by reference in its entirety).

The polypeptide or protein sequences, fragments, and variants thereof as described herein include, for example, modified forms of native polynucleotide sequences naturally present in the organism or species prior to modification. For example, in some embodiments, the native polynucleotide sequence naturally present in the organism or species prior to modification is that of an ortholog of Ptr1 as described herein. In some embodiments, the native polynucleotide sequence is from a species of Solanum, Capsicum, or Nicotiana. In some embodiments, the native polynucleotide sequence naturally present in the organism or species prior to modification is from S. lycopersicum, S. pennellii, S. tuberosum, C. annuum, C. baccatum, C. chinense, N. attenuata, N. benthamiana, N. tabacum, or N. tomentosiformis.

In embodiments, the polynucleotide, polypeptide, or protein (or a fragment or variant thereof) of embodiments of the disclosure is a modified form of the RGA1 gene of Solanum tuberosum. In some embodiments, the RGA1 gene of Solanum tuberosum encodes a protein having the amino acid sequence of SEQ ID NO: 131. In some embodiments, the RGA1 gene of Solanum tuberosum has the polynucleotide coding sequence of SEQ ID NO:132.

In some embodiments, the polynucleotide, polypeptide, or protein (or a fragment or variant thereof) of embodiments of the disclosure is a modified form of S. lycopersicum (SlPtr1, SEQ ID NO:114), S. pennellii (SpPtr1, SEQ ID NO:115), S. tuberosum Ptr1 (SEQ ID NO:116), C. anuum Ptr1 (SEQ ID NO:117), C. baccatum Ptr1 (SEQ ID NO:118), C. chinense Ptr1 (SEQ ID NO:119), N. attenuata Ptr1 (SEQ ID NO:120), N. benthamiana Ptr1a (SEQ ID NO:121), N. tabacum Ptr1 (SEQ ID NO:122), or N. tomentosiformis Ptr1 (SEQ ID NO:123).

In some embodiments, the polynucleotide, polypeptide, or protein (or a fragment or variant thereof) of embodiments of the disclosure is a modified form of S. lycopersicum (SlPtr1, SEQ ID NO:114) or S. pennellii (SpPtr1, SEQ ID NO:115). In some embodiments, the polynucleotide, polypeptide, or protein (or a fragment or variant thereof) of embodiments of the disclosure is a modified form of S. lycopersicum (SlPtr1, SEQ ID NO:114).

Heterologous polynucleotides may be introduced into the cells of embodiments of the disclosure by use of known techniques or systems. For example, by traditional breeding, transgenic methods, or direct editing. A person of skill in the art will recognize that some of these techniques or systems will produce transgenic cells, while others produce non-transgenic cells. Thus, both transgenic and non-transgenic cells and plants comprising the heterologous polynucleotide sequences of embodiments of the disclosure are contemplated.

Methods of producing recombinant nucleic acids for purposes of, e.g., making transgenic plants are well-known. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Second Edition, Cold Spring Harbor, NY, Cold Spring Harbor Press (1989), and Ausubel et al., Current Protocols in Molecular Biology, New York, NY, John Wiley & Sons (1989), which are hereby incorporated by reference in their entirety.

In preparing a nucleic acid vector for expression, the various nucleic acid sequences may normally be inserted or substituted into a bacterial plasmid. Any convenient plasmid may be employed, which will be characterized by having a bacterial replication system, a marker which allows for selection in a bacterium, and generally one or more unique, conveniently located restriction sites. Numerous plasmids, referred to as transformation vectors, are available for plant transformation. The selection of a vector will depend on the preferred transformation technique. A variety of vectors are available for stable transformation using Agrobacterium tumefaciens, a soilborne bacterium that causes crown gall. Crown gall are characterized by tumors or galls that develop on the lower stem and main roots of the infected plant. These tumors are due to the transfer and incorporation of part of the bacterium plasmid DNA into the plant chromosomal DNA. This transfer DNA (“T-DNA”) is expressed along with the normal genes of the plant cell. The plasmid DNA, pTi, or Ti-DNA, for “tumor inducing plasmid,” contains the vir genes necessary for movement of the T-DNA into the plant. The T-DNA carries genes that encode proteins involved in the biosynthesis of plant regulatory factors, and bacterial nutrients (opines). The T-DNA is delimited by two 25 bp imperfect direct repeat sequences called the “border sequences.” By removing the oncogene and opine genes, and replacing them with a gene of interest, it is possible to transfer foreign DNA into the plant without the formation of tumors or the multiplication of Agrobacterium tumefaciens (Fraley et al., “Expression of Bacterial Genes in Plant Cells,” Proc. Nat'l Acad. Sci. 80:4803-4807 (1983), which is hereby incorporated by reference in its entirety).

Further improvement of this technique led to the development of the binary vector system (Bevan, “Binary Agrobacterium Vectors for Plant Transformation,” Nucleic Acids Res. 12:8711-8721 (1984), which is hereby incorporated by reference in its entirety). In this system, all the T-DNA sequences (including the borders) are removed from the pTi, and a second vector containing T-DNA is introduced into Agrobacterium tumefaciens. This second vector has the advantage of being replicable in E. coli as well as A. tumefaciens, and contains a multiclonal site that facilitates the cloning of a transgene. An example of a commonly-used vector is pBin19 (Frisch et al., “Complete Sequence of the Binary Vector Bin19,” Plant Molec. Biol. 27:405-409 (1995), which is hereby incorporated by reference in its entirety). Any appropriate vectors now known or later described for genetic transformation are suitable for use with embodiments of the disclosure.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Certain “control elements” or “regulatory sequences” are also incorporated into the vector-construct. These include non-translated regions of the vector, promoters, and 5′ and 3′ untranslated regions which interact with host cellular proteins to carry out transcription and translation. Such elements may vary in their strength and specificity. Depending on the vector system and host utilized, any number of suitable transcription and translation elements, including constitutive and inducible promoters, may be used. Tissue-specific and organ-specific promoters can also be used.

A constitutive promoter is a promoter that directs expression of a gene throughout the development and life of an organism. Examples of some constitutive promoters that are widely used for inducing expression of transgenes include the nopaline synthase (“NOS”) gene promoter, from Agrobacterium tumefaciens (U.S. Pat. No. 5,034,322 to Rogers et al., which is hereby incorporated by reference in its entirety), the cauliflower mosaic virus (CaMV) 35S and 19S promoters (U.S. Pat. No. 5,352,605 to Fraley et al., which is hereby incorporated by reference in its entirety), those derived from any of the several actin genes, which are known to be expressed in most cells types (U.S. Pat. No. 6,002,068 to Privalle et al., which is hereby incorporated by reference in its entirety), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, growth regulator, herbicide, or phenolic compound, or a physiological stress directly imposed upon the plant such as cold, heat, salt, toxins, or through the action of a pathogen or disease agent such as a virus or fungus. A plant cell containing an inducible promoter may be exposed to an inducer by externally applying the inducer to the cell or plant such as by spraying, watering, heating, or by exposure to the operative pathogen. An example of an appropriate inducible promoter is a glucocorticoid-inducible promoter (Schena et al., “A Steroid-Inducible Gene Expression System for Plant Cells,” Proc. Natd. Acad. Sci. 88:10421-5 (1991), which is hereby incorporated by reference in its entirety). Expression of the transgene-encoded protein is induced in the transformed plants when the transgenic plants are brought into contact with nanomolar concentrations of a glucocorticoid, or by contact with dexamethasone, a glucocorticoid analog (Schena et al., “A Steroid-Inducible Gene Expression System for Plant Cells,” Proc. Natl. Acad. Sci. USA 88:10421-5 (1991); Aoyama et al., “A Glucocorticoid-Mediated Transcriptional Induction System in Transgenic Plants,” Plant J. 11:605-612 (1997); and McNellis et al., “Glucocorticoid-Inducible Expression of a Bacterial Avirulence Gene in Transgenic Arabidopsis Induces Hypersensitive Cell Death,” Plant J. 14(2):247-57 (1998), which are hereby incorporated by reference in their entirety). In addition, inducible promoters include promoters that function in a tissue specific manner to regulate the gene of interest within selected tissues of the plant. Examples of such tissue specific or developmentally regulated promoters include seed, flower, fruit, or root specific promoters as are well known by those of ordinary skill in the art (U.S. Pat. No. 5,750,385 to Shewmaker et al., which is hereby incorporated by reference in its entirety).

A number of tissue- and organ-specific promoters have been developed for use in genetic engineering of plants (Potenza et al., “Targeting Transgene Expression in Research, Agricultural, and Environmental Applications: Promoters Used in Plant Transformation,” In Vitro Cell. Dev. Biol. Plant 40:1-22 (2004), which is hereby incorporated by reference in its entirety). Examples of such promoters include those that are floral-specific (Annadana et al., “Cloning of the Chrysanthemum UEP1 Promoter and Comparative Expression in Florets and Leaves of Dendranthema grandiflora,” Transgenic Res. 11:437-445 (2002), which is hereby incorporated by reference in its entirety), seed-specific (Kluth et al., “5′ Deletion of a gbss1 Promoter Region Leads to Changes in Tissue and Developmental Specificities,” Plant Mol. Biol. 49:669-682 (2002), which is hereby incorporated by reference in its entirety), root-specific (Yamamoto et al., “Characterization of cis-acting Sequences Regulating Root-Specific Gene Expression in Tobacco,” Plant Cell 3:371-382 (1991), which is hereby incorporated by reference in its entirety), fruit-specific (Fraser et al., “Evaluation of Transgenic Tomato Plants Expressing an Additional Phytoene Synthase in a Fruit-Specific Manner,” Proc. Natl. Acad. Sci. USA 99:1092-1097 (2002), which is hereby incorporated by reference in its entirety), and tuber/storage organ-specific (Visser et al., “Expression of a Chimeric Granule-Bound Starch Synthase-GUS Gene in Transgenic Potato Plants,” Plant Mol. Biol. 17:691-699 (1991), which is hereby incorporated by reference in its entirety). Targeted expression of an introduced gene (transgene) is necessary when expression of the transgene could have detrimental effects if expressed throughout the plant. On the other hand, silencing a gene throughout a plant could also have negative effects. However, this problem could be avoided by localizing the silencing to a region by a tissue-specific promoter.

Nucleic acid constructs of embodiments of the disclosure include an operable 3′ regulatory region, selected from among those which are capable of providing correct transcription termination and polyadenylation of mRNA for expression in the host cell of choice, operably linked to a nucleic acid molecule configured to silence BBTV. A number of 3′ regulatory regions are known to be operable in plants. Exemplary 3′ regulatory regions include, without limitation, the nopaline synthase (“nos”) 3′ regulatory region (Fraley et al., “Expression of Bacterial Genes in Plant Cells,” Proc. Nat'l Acad. Sci. USA 80:4803-4807 (1983), which is hereby incorporated by reference in its entirety) and the cauliflower mosaic virus (“CaMV”) 3′ regulatory region (Odell et al., “Identification of DNA Sequences Required for Activity of the Cauliflower Mosaic Virus 35S Promoter,” Nature 313(6005):810-812 (1985), which is hereby incorporated by reference in its entirety). Virtually any 3′ regulatory region known to be operable in plants would be suitable for use in conjunction with embodiments of the disclosure.

The different components described supra can be ligated together to produce the expression systems which contain the nucleic acid constructs of embodiments of the disclosure, using well known molecular cloning techniques as described in Sambrook et al., Molecular Cloning: A Laboratory Manual, Second Edition Cold Spring Harbor, NY, Cold Spring Harbor Press (1989), and Ausubel et al. Current Protocols in Molecular Biology, New York, NY, John Wiley & Sons (1989), which are hereby incorporated by reference in their entirety.

Once the nucleic acid construct of embodiments of the disclosure has been prepared, it is ready to be incorporated into a host cell. Accordingly, another aspect of embodiments of the disclosure relates to a recombinant host cell containing one or more of the nucleic acid constructs of embodiments of the disclosure. Basically, this method is carried out by transforming a host cell with a nucleic acid construct of embodiments of the disclosure under conditions effective to achieve transcription of the nucleic acid molecule in the host cell. This is achieved with standard cloning procedures known in the art, such as described by Sambrook et al., Molecular Cloning: A Laboratory Manual, Second Edition, Cold Springs Laboratory, Cold Springs Harbor, NY (1989), which is hereby incorporated by reference in its entirety. Suitable host cells include, but are not limited to, bacteria, virus, yeast, mammalian cells, insect, plant, and the like. Preferably the host cells are either a bacterial cell or a plant cell. Methods of transformation may result in transient or stable expression of the nucleic acid under control of the promoter. Preferably, a nucleic acid construct of embodiments of the disclosure is stably inserted into the genome of the recombinant plant cell as a result of the transformation, although transient expression can serve an important purpose.

Plant tissue suitable for transformation includes leaf tissue, root tissue, meristems, zygotic and somatic embryos, callus, protoplasts, tassels, pollen, embryos, anthers, and the like. The means of transformation chosen is that most suited to the tissue to be transformed.

Transient expression in plant tissue can be achieved by particle bombardment (Klein et al., “High-Velocity Microprojectiles for Delivering Nucleic Acids Into Living Cells,” Nature 327:70-73 (1987), which is hereby incorporated by reference in its entirety), also known as biolistic transformation of the host cell, as disclosed in U.S. Pat. Nos. 4,945,050; 5,036,006; and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety.

In particle bombardment, tungsten or gold microparticles (1 to 2 μm in diameter) are coated with the DNA of interest and then bombarded at the tissue using high pressure gas. In this way, it is possible to deliver foreign DNA into the nucleus and obtain a temporal expression of the gene under the current conditions of the tissue. Biologically active particles (e.g., dried bacterial cells containing the vector and heterologous DNA) can also be propelled into plant cells. Other variations of particle bombardment, now known or hereafter developed, can also be used.

An appropriate method of stably introducing the nucleic acid construct into plant cells is to infect a plant cell with Agrobacterium tumefaciens or Agrobacterium rhizogenes previously transformed with the nucleic acid construct of embodiments of the disclosure. As described supra, the Ti (or RI) plasmid of Agrobacterium enables the highly successful transfer of a foreign nucleic acid molecule into plant cells. A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety).

Yet another method of introduction is fusion of protoplasts with other entities, either minicells, cells, lysosomes, or other fusible lipid-surfaced bodies (Fraley et al., “Liposome-mediated Delivery of Tobacco Mosaic Virus RNA Into Tobacco Protoplasts: A Sensitive Assay for Monitoring Liposome-protoplast Interactions,” Proc. Natl. Acad. Sci. USA 79:1859-63 (1982), which is hereby incorporated by reference in its entirety). The nucleic acid molecule may also be introduced into the plant cells by electroporation (Fromm et al., “Expression of Genes Transferred into Monocot and Dicot Plant Cells by Electroporation,” Proc. Natl. Acad. Sci. USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate. Other methods of transformation include polyethylene-mediated plant transformation, micro-injection, physical abrasives, and laser beams (Senior, “Uses of Plant Gene Silencing,” Biotechnology and Genetic Engineering Reviews 15:79-119 (1998), which is hereby incorporated by reference in its entirety). The precise method of transformation is not critical to the practice of embodiments of the disclosure. Any method that results in efficient transformation of the host cell of choice is appropriate for practicing embodiments of the disclosure.

After transformation, the transformed plant cells must be regenerated. Plant regeneration from cultured protoplasts is described in Evans et al., Handbook of Plant Cell Cultures, Vol. 1, New York, NY, MacMillan Publishing Co. (1983); Vasil, ed., Cell Culture and Somatic Cell Genetics of Plants, Vol. I (1984) and Vol. III (1986), Orlando, Acad. Press; and Fitch et al., “Somatic Embryogenesis and Plant Regeneration from Immature Zygotic Embryos of Papaya (Carica papaya L.),” Plant Cell Rep. 9:320 (1990), which are hereby incorporated by reference in their entirety.

Means for regeneration vary from species to species of plants, but generally a suspension of transformed protoplasts or a petri plate containing explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. Efficient regeneration will depend on the medium, on the genotype, and on the history of the culture. If these three variables are controlled, then regeneration is usually reproducible and repeatable.

Preferably, transformed cells are first identified using a selection marker simultaneously introduced into the host cells along with the nucleic acid construct of embodiments of the disclosure. Suitable selection markers include, without limitation, markers encoding for antibiotic resistance, such as the neomycin phosphotransferae II (“nptII”) gene which confers kanamycin resistance (Fraley et al., “Expression of Bacterial Genes in Plant Cells,” Proc. Natl. Acad. Sci. USA 80:4803-4807 (1983), which is hereby incorporated by reference in its entirety), and the genes which confer resistance to gentamycin, G418, hygromycin, streptomycin, spectinomycin, tetracycline, chloramphenicol, and the like. Cells or tissues are grown on a selection medium containing the appropriate antibiotic, whereby generally only those transformants expressing the antibiotic resistance marker continue to grow. Other types of markers are also suitable for inclusion in the expression cassette of embodiments of the disclosure. For example, a gene encoding for herbicide tolerance, such as tolerance to sulfonylurea is useful, or the dhfr gene, which confers resistance to methotrexate (Bourouis et al., EMBO J. 2:1099-1104 (1983), which is hereby incorporated by reference in its entirety). Similarly, “reporter genes,” which encode for enzymes providing for production of an identifiable compound are suitable. The most widely used reporter gene for gene fusion experiments has been uidA, a gene from Escherichia coli that encodes the β-glucuronidase protein, also known as GUS (Jefferson et al., “GUS Fusions: β Glucuronidase as a Sensitive and Versatile Gene Fusion Marker in Higher Plants,” EMBO J. 6:3901-3907 (1987), which is hereby incorporated by reference in its entirety). Similarly, enzymes providing for production of a compound identifiable by luminescence, such as luciferase, are useful. The selection marker employed will depend on the target species; for certain target species, different antibiotics, herbicide, or biosynthesis selection markers are preferred.

Plant cells and tissues selected by means of an inhibitory agent or other selection marker are then tested for the acquisition of the transgene (Sambrook et al., Molecular Cloning: A Laboratory Manual, Cold Spring Harbor, NY, Cold Spring Harbor Press (1989), which is hereby incorporated by reference in its entirety).

Accordingly, another aspect of embodiments of the disclosure relates to a plant or plant seed transformed with one or more nucleic acid constructs described herein. Embodiments of the disclosure also encompasses the whole plant, or a component part of a plant, including shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same.

In some embodiments, a transgenic plant or cell of embodiments of the disclosure is modified, for example by outcrossing, to remove the transgene (e.g., removal of Cas9 following genomic introduction of heterologous polynucleotide sequences). Thus, also contemplated are non-transgenic cells and plants comprising heterologous polynucleotide sequences of embodiments of the disclosure, derived from transgenic cells and plants comprising heterologous polynucleotide sequences of embodiments of the disclosure.

Suitable plants for use in accordance with embodiments of the disclosure include both monocots and dicots. Suitable plants for use in accordance with embodiments of the disclosure also include both crop plants and ornamentals. For example, suitable plants include rice, corn, soybean, canola, potato, wheat, mung bean, alfalfa, barley, rye, cotton, sunflower, peanut, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, tobacco, tomato, sorghum, sugarcane, banana, Arabidopsis thaliana, Saintpaulia, petunia, pelargonium, poinsettia, chrysanthemum, carnation, crocus, marigold, daffodil, pine, Medicago truncatula, Sandersonia aurantiaca, and zinnia.

In embodiments, the plant is a Solanum spp. In embodiments, the plant is Solanum lycopersicum. In other embodiments, the plant is Solanum tuberosum.

In embodiments, the plant is a Cucurbita spp. In embodiments, the plant is Cucurbita argyrosperma, Cucurbita ficifolia, Cucurbita moschata, Cucurbita maxima, or Cucurbita pepo.

In embodiments, the plant is a Pyrus spp. In embodiments, the plant is Pyrus communis, Pyris pyrifolia, Pyris bretschneideri, Pyrus sinkiangensis, or Pyrus ussuriensis.

Another aspect of embodiments of the disclosure is a method of expressing a nucleic acid molecule in a plant. The method involves providing a transgenic plant or transgenic plant cell transformed with a nucleic acid construct comprising a nucleic acid molecule comprising a Pseudomonas tomato race 1 (Ptr1) polynucleotide, a 5′ heterologous DNA promoter sequence, and a 3′ terminator sequence, wherein the nucleic acid molecule, the DNA promoter sequence, and the terminator sequence are operatively coupled to permit transcription of the nucleic acid molecule, and growing the transgenic plant or a plant grown from the transgenic plant seed under conditions effective to express the nucleic acid molecule in said transgenic plant or said plant grown from the transgenic plant seed. Suitable nucleic acid molecules are described above.

In some embodiments a transgenic plant is provided. In some embodiments, a transgenic plant cell is provided. In some embodiments, a transgenic plant seed comprising a transgenic plant cell is provided.

In some embodiments, providing a transgenic plant, plant cell, or plant seed involves transforming a non-transgenic plant, non-transgenic plant cell, or a non-transgenic plant seed with the nucleic acid construct to yield the transgenic plant or plant seed. Transformation is described above and may include Agrobacterium-mediated transformation, whisker method transformation, vacuum infiltration, biolistic transformation, electroporation, micro-injection, polyethylene-mediated transformation, or laser-beam transformation.

Another aspect of embodiments of the disclosure is a method of imparting disease resistance to a plant. The method involves transforming a plant or a plant cell with a nucleic acid molecule that increases expression of Ptr1, wherein the expression is effective in imparting disease resistance to the transformed plant or to a transgenic plant produced from the transformed plant cell.

In some embodiments, a plant is transformed. In another embodiment, a plant seed is transformed and the method also involves planting the transformed plant seed under conditions effective for a plant to grow from the planted plant seed. Suitable nucleic acid molecules and are described above.

Imparting disease resistance also includes enhancing disease resistance in a plant. Imparting disease resistance or enhancing disease resistance refers to an increase in the ability of a plant to prevent pathogen infection or pathogen-induced symptoms. In some embodiments, disease resistance is increased compared to a control plant (for example, an unmodified or non-transgenic plant). In some embodiments, the level of resistance in a non-naturally occurring transgenic plant of the invention is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the resistance exhibited by a control plant. The level of resistance may be measured using conventional methods. For example, the level of resistance to a pathogen may be determined by comparing physical features and characteristics (for example, plant height and weight) or by comparing disease symptoms (for example, delayed lesion development, reduced lesion size, leaf wilting and curling, water-soaked spots, amount of pathogen growth, and discoloration of cells) of the non-naturally occurring plant (e.g., a modified plant or transgenic plant).

Disease resistance can be increased or enhanced resistance to a particular pathogen species or genus or can be increased or enhanced resistance to a broad range of pathogens (e.g., pattern-triggered immunity, systemic acquired resistance, etc.). In some embodiments, the pathogen is a bacterial plant pathogen. By way of example, bacterial pathogens may belong to Acidovorax, Agrobacterium, Burkholderia, Candidatus Liberibacter, Clavibacter, Curtobacterium, Dickeya, Erwinia, Pantoea, Pectobacterium, Phytoplasma, Pseudomonas, Ralstonia, Spiroplasma, Streptomyces, Xanthomonas, and Xylella. See PLANT PATHOGENIC BACTERIA: GENOMICS AND MOLECULAR BIOLOGY, Robert W. Jackson Ed., Norfolk, UK, Caister Academic Press (2009), which is hereby incorporated by reference in its entirety. By way of example, the bacterial parasite may belong to Pseudomonas syringae pv. tomato. In some embodiments, the pathogen is a fungal parasite. By way of example, the fungal parasite may be to Collimonas fungivorans.

In some embodiments, the pathogen expresses an AvrRpt2 protein (or a variant, homolog, or ortholog thereof). In some embodiments, the AvrRpt2 protein is one of those identified in Table 10.

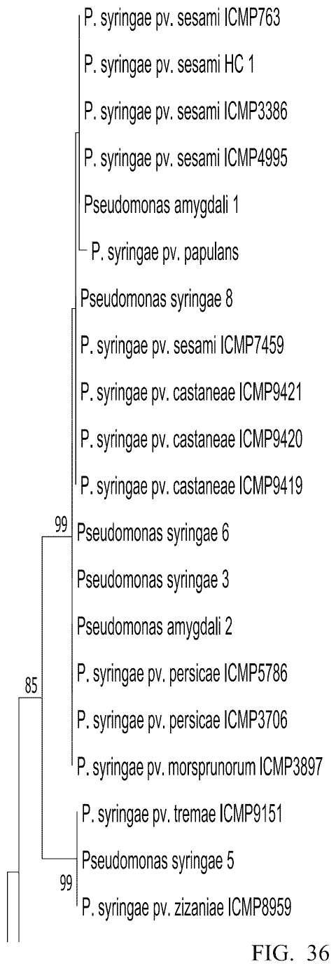

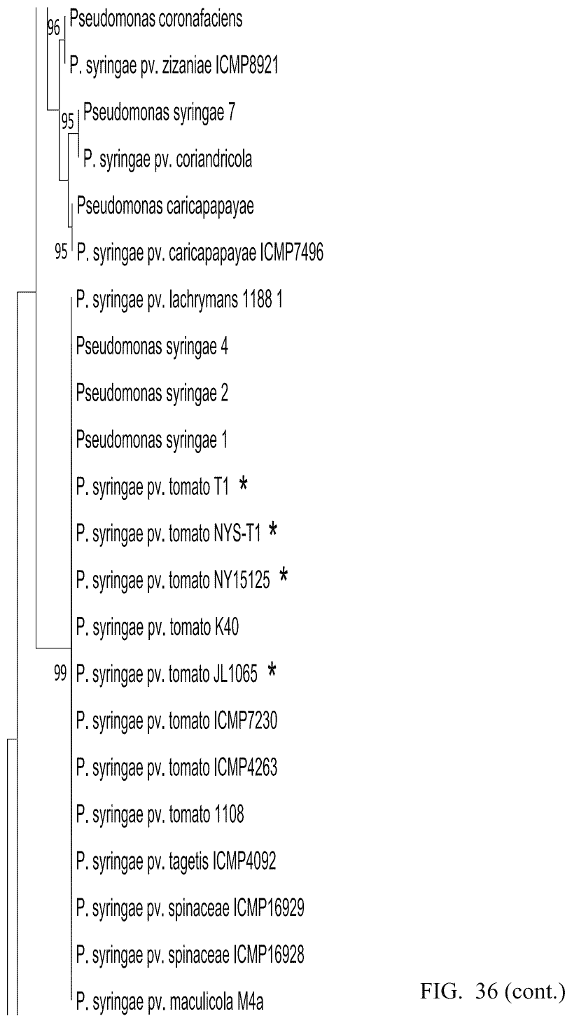

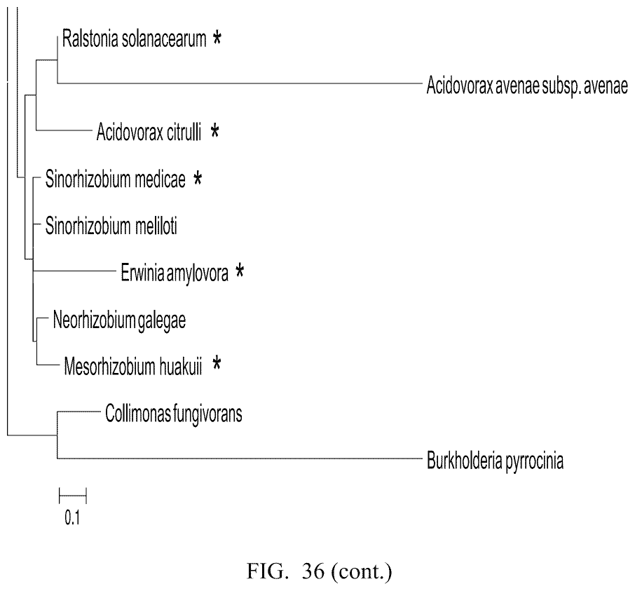

In some embodiments, disease resistance to a Pseudomonas, Ralstonia, Acidovoraxavenae, Acidovorax, Sinorhizobium, Erwinia, Neorhizobium, Meshorhizobium, Collimonas, or Burkholderia species, such as those identified in FIG. 36 and Table 10 is increased.

In some embodiments, disease resistance to one or more of Erwinia amylovora, Ralstonia pseudosolanacearum, Acidovorax citrulli, Acidovorax avenae, Burkholderia pyrrocinia, Collimonas fungivorans, Mezorhizobium huakuii, and Sinorhizobium medicae is increased.

Another aspect of embodiments of the disclosure is a method of imparting disease resistance to a plant. The method involves providing a plant, plant cell, or plant seed. The plant, plant cell, or plant seed comprises a heterologous Ptr1 as described herein. The expression of the heterologous Ptr1 is effective in imparting or enhancing disease resistance in the plant or plant produced from the plant cell or plant seed.

Suitable heterologous Ptr1 polynucleotides are described herein above.

For example, in some embodiments, the heterologous Ptr1 polynucleotide is a heterologous polynucleotide sequence not native to the plant or cell. In some embodiments, the heterologous Ptr1 polynucleotide incorporates modifications to its own native polynucleotide sequence (e.g., by genome modification as described herein) resulting in a heterologous polynucleotide sequence, as described herein above.

In some embodiments, the heterologous Ptr1 polynucleotide is a transgene. Accordingly, in some embodiments, providing the plant includes transforming a plant, plant cell, or plant seed with a nucleic acid molecule that increases expression of an Ptr1 protein or polypeptide according to embodiments of the disclosure. Suitable nucleic acid molecules are described above.

Another aspect of embodiments of the disclosure is a method of identifying a candidate plant suitable for breeding that displays enhanced disease resistance. The method involves providing a candidate plant; analyzing the candidate plant for the presence, in its genome, of a Ptr1 polynucleotide (or a variant or ortholog thereof); identifying, based on said analyzing, a candidate plant suitable for breeding that includes in its genome, a Ptr1 polynucleotide (or a variant or ortholog thereof); and breeding the identified plant with at least one other plant. In some embodiments, identifying the presence of a Ptr1 sequence is achieved based on sequence homology to the sequences set forth herein by, for example, using next generation sequencing, TaqMan assays, UniTaq assays, real-time PCR assays, digital PCR, microarray, hybridization or other detection methods.

In some embodiments, analyzing the candidate plant for the presence, in its genome, of a gene encoding an Ptr1 polynucleotide involves isolating genomic DNA from the plant, germplasm, pollen, or seed of the plant; analyzing genomic DNA from the plant, germplasm, pollen, or seed of the plant for the presence of the gene encoding the Ptr1 polynucleotide; and detecting the gene encoding the Ptr1 polynucleotide.

In some embodiments, the breeding involves crossing, making hybrids, backcrossing, self-crossing, double haploid breeding, and/or combinations thereof.

In some embodiments, a transgenic plant transformed with a nucleic acid molecule that encodes a Ptr1 polynucleotide is provided as the candidate plant. In some embodiments, providing the transgenic plant involves transforming a plant or plant cell with a nucleic acid construct according to embodiments of the disclosure and growing the transgenic plant or a plant grown from the transgenic plant cell under conditions effective to express the nucleic acid molecule in the transgenic plant or a plant grown from the transgenic plant cell.

Another aspect of embodiments of the disclosure is a plant cell comprising a nucleic acid molecule comprising a heterologous Pseudomonas tomato race 1 (Ptr1) polynucleotide.

In embodiments, the plant cell comprising a nucleic acid molecule comprising a heterologous Pseudomonas tomato race 1 (Ptr1) polynucleotide is transgenic; in other embodiments the plant cell comprising a nucleic acid molecule comprising a heterologous Pseudomonas tomato race 1 (Ptr1) polynucleotide is non-transgenic.

The following examples are provided to illustrate embodiments of the present disclosure but are by no means intended to limit its scope.

### EXAMPLES

### Example 1: Materials and Methods for Examples 2-11

### Bacterial Strains and Plasmids

Pseudomonas syringae pv. tomato strains DC3000 (Buell et al., “The Complete Genome Sequence of the Arabidopsis and Tomato Pathogen Pseudomonas Syringae pv. Tomato DC3000,” PNAS 100(18):10181-6 (2003), which is hereby incorporated by reference in its entirety), NY15125 (Kraus et al, “Pseudomonas syringae pv. tomato strains from New York Exhibit Virulence Attributes Intermediate Between Typical race 0 and race 1 Strains,” Plant Dis. 101:1442-8 (2017), which is hereby incorporated by reference in its entirety), T1 (Almeida et al., “A Draft Genome Sequence of Pseudomonas Syringae pv. tomato T1 Reveals a Type III Effector Repertoire Significantly Divergent from that of Pseudomonas syringae pv. tomato DC3000,” Mol. Plant-Microbe Interact. 22(1): 52-62 (2009), which is hereby incorporated by reference in its entirety), JL1065 (Whalen et al., “Identification of Pseudomonas syringae Pathogens of Arabidopsis and a Bacterial Locus Determining Avirulence on Both Arabidopsis and Soybean,” Plant Cell 3:49-59 (1991), which is hereby incorporated by reference in its entirety), NYT1 (Jones et al., “Genome-Assisted Development of a Diagnostic Protocol for Distinguishing High Virulence Pseudomonas Syringae pv. tomato Strains,” Plant Disease 99:527-34 (2015), which is hereby incorporated by reference in its entirety), CA-A9, and CA-407 (Kunkeaw et al., “Molecular and Evolutionary Analyses of Pseudomonas syringae pv. tomato Race 1,” Mol. Plant-Microbe. Interact. 23:415-24 (2010), which is hereby incorporated by reference in its entirety) were grown on King's B (KB) (King et al., “Two Simple Media for the Demonstration of Pycocyanin and Fluorescin,” J. Lab. Clin. Med. 44:301-7 (1954), which is hereby incorporated by reference in its entirety) semi-selective media at 30° C. (Table 1). Ralstonia pseudosolanacearum CMR15 (Remenant et al., “Genomes of Three Tomato Pathogens Within the Ralstonia solanacearum Species Complex Reveal Significant Evolutionary Divergence,” BMC Genomics 11:379 (2010), which is hereby incorporated by reference in its entirety) was grown on rich B medium (27.7 mM glucose, 10 g 1⁻¹ bacto-peptone, 1 g 1⁻¹ yeast extract, 1 g 1⁻¹ casamino acids). Plasmids pCPP5372 (Oh et al., “Pseudomonas syringae Lytic Transglycosylases Coregulated with the Type III Secretion System Contribute to the Translocation of Effector Proteins into Plant Cells,” J. Bacteriol. 189: 8277-89 (2007), which is hereby incorporated by reference in its entirety) carrying wildtype avrRpt2_{NY15125}, avrRpt2 variants or the empty vector were introduced into DC3000 by electroporation (Tables 2 and 3). All P. syringae pv. tomato strains were stored in 20% glycerol+60 mM sucrose at −80° C. Escherichia coli TOP 10 was used for plasmid maintenance and grown in LB medium at 37° C.

TABLE 1
Bacterial strains used in this study.

| | | | | Relevant |
|---|---|---|---|---|
| Name | Genotype | Race | Source | efectors |
| P. syringae pv. tomato | Wild type | 0 | Beull et al., 2003^{a} | AvrPto |
| DC3000 | | | | |
| P. syringae pv. tomato | Wild type | 0 | Kraus et al., 2017^{b} | AvrPto, |
| NY15125 | | | | AvrRpt2 |
| P. syringae pv. tomato | Wild type | 0 | Whalen et al., 1991^{c} | AvrPto, |
| JL1065 | | | | AvrRpt2 |
| P. syringae pv. tomato | Wild type | 1 | Almeida et al., | AvrRpt2 |
| T1 | | | 2009^{d} | |
| P. syringae pv. tomato | Wild type | 1 | Jones et al., 2015^{e} | AvrRpt2 |
| NYT1 | | | | |
| P. syringae pv. tomato | Wild type | 1 | Kunkeaw et al., | AvrRpt2 |
| CA-A9 | | | 2010^{f} | |
| P. syringae pv. tomato | Wild type | 1 | Kunkeaw et al., | AvrRpt2 |
| CA-407 | | | 2010^{f} | |
| P. pseudosolanacearum | Wild type | — | Mahbou Somo | RipBN |
| CMR15 | | | Toukam et al., 2009^{g} | |
| A. tumefaciens 1D1249 | Wild type | — | Wroblewski et al., | — |
| | | | 2005^{h} | |
| E. coli TOP 10 | Wild type | — | Thermo Fisher Co. | — |
| E. coli S17-1 | Wild type | — | A. Collmer, Cornell | — |
| | | | University | |

Table 1 source references include:
^{a}Buell et al., “The Complete Genome Sequence of the Arabidopsis and Tomato Pathogen Pseudomonas Syringae pv. Tomato DC3000,” PNAS 100(18):10181-6 (2003);
^{b}Kraus et al, “Pseudomonas syringae” pv. tomato strains from New York Exhibit Virulence Attributes Intermediate Between Typical race 0 and race 1 Strains,” Plant Dis. 101:1442-8 (2017);
^{c}Whalen et al., “Identification of Pseudomonas syringae Pathogens of Arabidopsis and a Bacterial Locus Determining Avirulence on Both Arabidopsis and Soybean,” Plant Cell 3:49-59 (1991);
^{d}Almeida et al., “A Draft Genome Sequence of Pseudomonas Syringae pv. Tomato T1 Reveals a Type III Effector Repertoire Significantly Divergent from that of Pseudomonas syringae pv. tomato DC3000,” Mol. Plant-Microbe Interact. 22(1): 52-62 (2009);
^{e}Jones et al., “Genome-Assisted Development of a Diagnostic Protocol for Distinguishing High Virulence Pseudomonas Syringae pv. tomato Strains,” Plant Disease 99:527-34 (2015);
^{f}Kunkeaw et al., “Molecular and Evolutionary Analyses of Pseudomonas syringae pv. tomato Race 1,” Mol. Plant-Microbe. Interact. 23:415-24 (2010);
^{g}Mahbou et al., “Broad Diversity of Ralstonia solanacearum Strains in Cameroon,” Plant Dis. 93:1123-30 (2009); and
^{h}Wroblewski et al., “Optimization of Agrobacterium-Mediated Transient Assays of Gene Expression in Lettuce, Tomato, and Arabidopsis,” Plant Biotech. J. 3:259-73 (2005), each of which is hereby incorporated by reference in its entirety.

TABLE 2
Vectors and plasmids used in this study.

| Vector | Insert | Identifier | Purpose | Source |
|---|---|---|---|---|
| pK18mobsac | — | — | Biparental Mating | Kvitko et al., |
| | | | | 2011^{a} |
| pk18mobsac | ΔavrRpt2 | pCM3 | For deletion of avrRpt2 in | This work |
| | | | NY15125 biparental mating | |
| pENTR SD | — | — | Gateway entry vector | ThermoFisher |
| TOPO | | | | |
| pENTR SD | avrRpt2 (no stop) | 7208 | For LR recombination into | This work |
| TOPO | | | destination vector | |
| pENTR SD | avrRpt2 F70R (no stop) | pCM4 | For LR recombination into | This work |
| TOPO | | | destination vector | |
| pENTR SD | avrRpt2 C122A (no stop) | pCM5 | For LR recombination into | This work |
| TOPO | | | destination vector | |
| pENTR SD | avrRpt2 C122Y (no stop) | pCM17 | For LR recombination into | This work |
| TOPO | | | destination vector | |
| pENTR SD | avrRpt2 G131D (no stop) | pCM18 | For LR recombination into | This work |
| TOPO | | | destination vector | |
| pENTR SD | avrRpt2 G141R (no stop) | pCM30 | For LR recombination into | This work |
| TOPO | | | destination vector | |
| pENTR SD | avrRpt2 E150S (no stop) | pCM27 | For LR recombination into | This work |
| TOPO | | | destination vector | |
| pENTR SD | avrRpt2 Y191C (no stop) | pCM6 | For LR recombination into | This work |
| TOPO | | | destination vector | |
| pENTR SD | avrRpt2 Y1915 (no stop) | pCM53 | For LR recombination into | This work |
| TOPO | | | destination vector | |
| pENTR SD | avrRpt2 G194R (no stop) | pCM19 | For LR recombination into | This work |
| TOPO | | | destination vector | |
| pENTR SD | avrRpt2 H208A (no stop) | pCM31 | For LR recombination into | This work |
| TOPO | | | destination vector | |
| pENTR SD | avrRpt2 D216E (no stop) | pCM7 | For LR recombination into | This work |
| TOPO | | | destination vector | |
| pCPP5372 | — | — | Binary Gateway destination | Oh et al., |
| | | | vector for type three | 2007^{b} |
| | | | effector protein expression | |
| | | | with C-terminal HA-tag | |
| pCPP5372 | avrRpt2 | 7200 | Generating hrp protein | This work |
| | | | fusion with C-terminal HA- | |
| | | | tag | |
| pCPP5372 | avrRpt2 F70R | pCM9 | Generating hrp protein | This work |
| | | | fusion with C-terminal HA- | |
| | | | tag | |
| pCPP5372 | avrRpt2 C122A | pCM10 | Generating hrp protein | This work |
| | | | fusion with C-terminal HA- | |
| | | | tag | |
| pCPP5372 | avrRpt2 C122Y | pCM20 | Generating hrp protein | This work |
| | | | fusion with C-terminal HA- | |
| | | | tag | |
| pCPP5372 | avrRpt2 G131D | pCM21 | Generating hrp protein | This work |
| | | | fusion with C-terminal HA- | |
| | | | tag | |
| pCPP5372 | avrRpt2 G141R | pCM32 | Generating hrp protein | This work |
| | | | fusion with C-terminal HA- | |
| | | | tag | |
| pCPP5372 | avrRpt2 E150S | pCM28 | Generating hrp protein | This work |
| | | | fusion with C-terminal HA- | |
| | | | tag | |
| pCPP5372 | avrRpt2 Y191C | pCM11 | Generating hrp protein | This work |
| | | | fusion with C-terminal HA- | |
| | | | tag | |
| pCPP5372 | avrRpt2 Y191S | pCM55 | Generating hrp protein | This work |
| | | | fusion with C-terminal HA- | |
| | | | tag | |
| pCPP5372 | avrRpt2 G194R | pCM22 | Generating hrp protein | This work |
| | | | fusion with C-terminal HA- | |
| | | | tag | |
| pCPP5372 | avrRpt2 H208A | pCM33 | Generating hrp protein | This work |
| | | | fusion with C-terminal HA- | |
| | | | tag | |
| pCPP5372 | avrRpt2 D216E | pCM33 | Generating hrp protein | This work |
| | | | fusion with C-terminal HA- | |
| | | | tag | |
| pGWB417 | — | — | Binary Gateway destination | Nakagawa et |
| | | | vector for plant | al., 2007^{c} |
| | | | transformation with C- | |
| | | | terminal Myo-tag | |
| pGWB417 | avrRpt2_Acidovorax | pTK187 | Generating C-terminal | Eschen- |
| | avenae subsp. avenae | | Myc-tag fusion | Lippold et al., |
| | ATCC 19860 | | | 2016^{d} |
| pGWB417 | avrRpt2_Acidovorax | pTK188 | Generating C-terminal | Eschen- |
| | citrulli tw6 | | Myc-tag fusion | Lippold et al., |
| | | | | 2016^{d} |
| pGWB417 | avrRpt2_Burkholderia | pTK189 | Generating C-terminal | Eschen- |
| | pyrrocinia Lyc2 | | Myc-tag fusion | Lippold et al., |
| | | | | 2016^{d} |
| pGWB417 | avrRpt2_Collimonas | pTK190 | Generating C-terminal | Eschen- |
| | fungivorans | | Myc-tag fusion | Lippold et al., |
| | | | | 2016^{d} |
| pGWB417 | avrRpt2_Erwinia | pTK191 | Generating C-terminal | Eschen- |
| | amylovora ATCC 49946 | | Myc-tag fusion | Lippold et al., |
| | | | | 2016^{d} |
| pGWB417 | avrRpt2_Mesorhizobium | pTK192 | Generating C-terminal | Eschen- |
| | huakuii 7653R | | Myc-tag fusion | Lippold et al., |
| | | | | 2016^{d} |
| pGWB417 | avrRpt2_Pseudomonas | pCM1 | Generating C-terminal | This work |
| | syringae pv. tomato | | Myc-tag fusion | |
| | NY15125 | | | |
| pGWB417 | avrRpt2_Ralstonia | pTK194 | Generating C-terminal | Eschen- |
| | solanacearum CMR15 | | Myc-tag fusion | Lippold et al., |
| | | | | 2016^{d} |
| pGWB417 | avrRpt2_Sinorhizobium | pTK195 | Generating C-terminal | Eschen- |
| | medicae WSM1369 | | Myc-tag fusion | Lippold et al., |
| | | | | 2016^{d} |

Table 2 source references include:
^{a}Kvitko et al., “Construction of Pseudomonas syringae pv. tomato DC3000 Mutant and Polymutant Strains,” Methods Mol. Biol. 712:109-28 (2011);
^{b}Oh et al., “Pseudomonas syringae Lytic Transglycosylases Coregulated with the Type III Secretion System Contribute to the Translocation of Effector Proteins into Plant Cells,” J. Bacteriol. 189: 8277-89 (2007);
^{c}Nakagawa et al., “Improved Gateway Binary Vectors: High-Performance Vectors for Creation of Fusion Constructs in Transgenic Analysis of Plants,” Biosci. Biotechnol. Biochem. 71:2095-100 (2007); and
^{d}Eschen-Lippold et al., “Bacterial AvrRpt2-Like Cysteine Proteases Block Activation of the Arabidopsis Mitogen-Activated Protein Kinases, MPK4 and MPK11,” Plant Physiol. 171:2223-8 (2016), each of which is herey incorporated by reference in its entirety.

TABLE 3
Oligonucleotides used in this study.

| | | | | SEQ | |
|---|---|---|---|---|---|
| | | | | ID | |
| Gene | Direction | Identifier | Sequence (5′-3′) | NO: | Source |
| Introgressed | Forward | Spenn- | taatgaggcagagcaagttt | 20 | This |
| region in Chr4 | | ch04_541669 | | | work |
| Introgressed | Reverse | Spenn- | ccctcaagaaccatgaatca | 21 | |
| region in Chr4 | | ch04_5416620 | | | |
| avrRpt2 | Forward | oCM26 | CACCatgaaaattgctccagttgccataaat | 22 | This |
| | | | cacag | | work |
| avrRpt2 | Reverse | oCM27 | cacacgcaatgctctaccgc | 23 | |
| 5′UTR | Forward | oCM20 | atcggGAATTCgtgctgatggatgctgc | 24 | |
| avrRpt2 | | | agg | | |
| 5′UTR | Reverse | oCM21 | ccacgtgaagataccttgctgcttgttaagtcgt | 25 | |
| avrRpt2 | | | ccgttgg | | |
| 3′UTR | Forward | oCM22 | agcaaggtatcttcacgtggcgg | 26 | |
| avrRpt2 | | | | | |
| 3′UTR | Reverse | oCM23 | atcggCCCGGGttctgcgagcgatttgcg | 27 | |
| avrRpt2 | | | gg | | |
| UTR avrRpt2 | Forward | oCM38 | CTGATCATGTGTGCCTTGACCCC | 28 | |
| UTR avrRpt2 | Reverse | oCM39 | GGACTGCAGGGTGTTTATCGGG | 29 | |
| UTR avrRpt2 | Forward | 3719 | TTAGCTCACTCATTAGGCAC | 30 | Kraus |
| | | | C | | etal., |
| UTR avrRpt2 | Reverse | 3720 | CCTCTTCGCTATTACGCCA | 31 | 2017^{a} |
| avrRpt2 F70R | Forward | oCM28 | atagaggttccagccCGTggagggtggttc | 32 | This |
| avrRpt2 F70R | Reverse | oCM29 | ccctccACGggctggaacctctatcttg | 33 | work |
| avrRpt2 | Forward | oCM30 | gcgaatgggaGCTtggtatgcctgcg | 34 | This |
| C122A | | | | | work |
| avrRpt2 | Reverse | oCM31 | cataccaAGCtcccattcgctcattac | 35 | |
| C122A | | | | | |
| avrRpt2 | Forward | oCM74 | gcgaatgggaTATtggtatgcctgc | 36 | This |
| C122Y | | | | | work |
| avrRpt2 | Reverse | oCM75 | gcataccaATAtcccattcgctcattacc | 37 | |
| C122Y | | | | | |
| avrRpt2 | Forward | oCM76 | agaatggttGACcattctgtcgaagc | 38 | This |
| G131D | | | | | work |
| avrRpt2 | Reverse | oCM77 | agcttcgacagaatgGTCaaccattc | 39 | |
| G131D | | | | | |
| avrRpt2 | Forward | oCM104 | cctaAGActgccggagctctatgagggaa | 40 | This |
| G141R | | | g | | work |
| avrRpt2 | Reverse | oCM105 | agagctccggcagTCTtaggcgaggccca | 41 | |
| G141R | | | gcttc | | |
| avrRpt2 E150S | Forward | oCM86 | ctatgagggaaggTCAggcccagctgggc | 42 | This |
| | | | tac | | work |
| avrRpt2 E150S | Reverse | oCM87 | gtagcccagctgggccTGAccttccctcat | 43 | |
| | | | ag | | |
| avrRpt2 | Forward | oCM32 | gtgcactgttgTGTaagcacgggccg | 44 | This |
| Y191C | | | | | work |
| avrRpt2 | Reverse | oCM33 | ccgtgatACAcaacagtgcacccaactc | 45 | |
| Y191C | | | | | |
| avrRpt2 | Forward | oCM109 | gttgggtgcactgttgTCTaagcacg | 46 | This |
| Y191C | | | | | work |
| avrRpt2 | Reverse | oCM110 | ccgtgatAGAcaacagtgcaccc | 47 | |
| Y191C | | | | | |
| avrRpt2 | Forward | oCM80 | gttgtataagcacAGGccgattatatttggg | 48 | This |
| G194R | | | | | work |
| avrRpt2 | Reverse | oCM81 | cccaaatataatcggCCTgtgatatacaac | 49 | |
| G194R | | | | | |
| avrRpt2 | Forward | oCM106 | gctggGCTatgteggtectcactggtgtcg | 50 | This |
| H208A | | | | | work |
| avrRpt2 | Reverse | oCM107 | gaggaccgacatAGCccagctgtcattcgg | 51 | |
| H208A | | | agttttc | | |
| avrRpt2 | Forward | oCM34 | ctcactggtgtcGAAaaagagacgtcgtcc | 52 | This |
| D216E | | | | | work |
| avrRpt2 | Reverse | oCM35 | gacgtctattTTCgacaccagtgaggacc | 53 | |
| D216E | | | | | |

Table 3 source references include:
^{a}Kraus et al, “Pseudomonas syringae pv. tomato strains from New York Exhibit Virulence Attributes Intermediate Between Typical race 0 and race 1 Strains,” Plant Dis. 101:1442-8 (2017), which is hereby incorporated by reference in its entirety.

### Plant Material

Solanum lycopersicoides introgression lines (ILs) seeds of LA4245 and LA4277 were obtained from the Tomato Genetics Resource Center (tgrc.ucdavis.edu/lycopersicoides_ils.aspx). The genotype of LA4245 can be heterozygous for the presence of Ptr1 (Ptr1/ptr1) (LA4245-R) or homozygous for the lack of the gene (ptr1/ptr1) (LA4245-S). S. lycopersicoides introgression lines were grown in a greenhouse at 24° C. during daylight and 22° C. at night. Nicotiana benthamiana Nb-1 (Bombarely et al., “A Draft Genome Sequence of Nicotiana benthamiana to Enhance Molecular Plant-Microbe Biology Research,” Mol. Plant Microbe Interact. 25(12):1523-30 (2012), which is hereby incorporated by reference in its entirety) was maintained in a growth chamber with 16 h: 8h, light: dark at 24° C. with light and 20° C. in the dark and 50% humidity. Tomatoes and N. benthamiana plants were grown in Cornell Osmocote Mix soil (0.16 m³ peat moss, 0.34 m³ vermiculite, 2.27 kg lime, 2.27 kg Osmocote Plus 15-9-12 and 0.54 kg Uni-Mix 11-5-11; Everris, Israeli Chemicals Ltd). After pathogen inoculation, plants were moved to a growth chamber with 25° C., 50% humidity, and 16 h light. Arabidopsis thaliana accession Columbia (Col-0) seeds were ethanol-sterilized, suspended in 1 ml of water and cold stratified for 2 days at 4° C. A. thaliana was grown in Fafard Mix (Sungro Horticulture) in a growth chamber under fluorescent lighting (100 mol m⁻² s⁻¹) with a 12 h:12 h; light:dark cycle at 21° C. and 40% humidity.

### Genome Sequencing, Assembly, and Type III Effector Annotation of NY15125

The NY15125 genome was sequenced to 163× coverage with long reads from the Pacbio RSII platform. A Canu assembly was performed with a stringent error rate (corrected Error Rate=0.035) (Koren et al., “Canu: Scalable and Accurate Long-Read Assembly via Adaptive k-mer Weighting and Repeat Separation,” Genome Res. 27(5):722-36 (2017), which is hereby incorporated by reference in its entirety). Illumina sequencing was also done to generate paired-end reads for a coverage of 114×. Adapter clipping and quality filtering of the Illumina reads was done with Trimmomatic (Bolger et al., “Trimmomatic: a Flexible Trimmer for Illumina Sequence Data,” Bioinformatics 30(15):2114-20 (2014), which is hereby incorporated by reference in its entirety). Concordant read mapping of the Illumina paired-end reads was used to evaluate the quality of the assembly. Two rounds of base level error corrections were done with Pacbio reads using Arrow (github.com/PacificBiosciences/pbbioconda), followed by two rounds of error correction with Illumina reads using Pilon (Walker et al., “Pilon: an Integrated Tool for Comprehensive Microbial Variant Detection and Genome Assembly Improvement,” PloS ONE 9(11):e112963 (2014), which is hereby incorporated by reference in its entirety).

The polished assembly includes a 6.2 Mb chromosome and three putative plasmids (88 Kb, 116 Kb, 122 Kb). It was annotated with Prokka (Seemann, “Prokka: Rapid Prokaryotic Genome Annotation,” Bioinformatics 30(14):2068-9 (2014), which is hereby incorporated by reference in its entirety) using proteins from the T1 genome (Almeida et al., “A Draft Genome Sequence of Pseudomonas Syringae pv. tomato T1 Reveals a Type III Effector Repertoire Significantly Divergent from that of Pseudomonas syringae pv. tomato DC3000,” Mol. Plant-Microbe Interact. 22(1): 52-62 (2009), which is hereby incorporated by reference in its entirety) as supporting evidence. The NY15125 chromosome was compared with the DC3000 and T1 genomes using BRIGG (Alikhan et al., “BLAST Ring Image Generator (BRIG): Simple Prokaryote Genome Comparisons,” BMC Genomics 12:402 (2011), which is hereby incorporated by reference in its entirety). Pseudomolecules were constructed from the deposited contigs for P. syringae pv. tomato NY15125 and annotated using MG-RAST (Meyer et al., “The Metagenomics RAST Server—a Public Resource for the Automatic Phylogenetic and Functional Analysis of Metagenomes,” BMC Bioinf 9:386 (2008), which is hereby incorporated by reference in its entirety). Effector genes were identified from the MG-RAST annotation, by alignment with other P. syringae pv. tomato sequences, and based on proximity to HrpL binding sites, predicted using the methods described previously (Saha et al., “Bound to Succeed: Transcription Factor Binding-Site Prediction and its Contribution to Understanding Virulence and Environmental Adaptation in Bacterial Plant Pathogens,” Mol. Plant Microbe. Interact. 26(10):1123-30 (2013), which is hereby incorporated by reference in its entirety). The NY15125 genome sequence and plasmid sequences are available from GenBank (accession numbers: CP034558-CP034561).

Development of a P. syringae pv. Tomato NY15125ΔavrRpt2 Strain

A 1,024-bp promoter fragment and a 841-bp fragment downstream of the avrRpt2 gene sequence were PCR amplified and EcoRI or XmaI restriction sites were added, respectively. Fusion of both DNA fragments was cloned into the suicide vector pK18mobsacB and transformed into E. coli S17-1. Deletion of avrRpt2 in NY15125 was performed by biparental mating as described previously (Kraus et al, “Pseudomonas syringae pv. tomato strains from New York Exhibit Virulence Attributes Intermediate Between Typical race 0 and race 1 Strains,” Plant Dis. 101:1442-8 (2017), which is hereby incorporated by reference in its entirety) with modifications (Kvitko et al., “Construction of Pseudomonas syringae pv. tomato DC3000 Mutant and Polymutant Strains,” Methods Mol. Biol. 712:109-28 (2011), which is hereby incorporated by reference in its entirety).

P. syringae pv. Tomato Inoculation and Population Assays in Tomato

P. syringae pv. tomato strains were grown on KB plates for 2 days at 30° C. Strains were diluted in 10 mM MgCl₂+0.002% Silwet L-77 at a final concentration of 5×10⁴ cfu ml⁻¹. Four-week-old plants were vacuum infiltrated, and three leaf disk samples (7 mm in diameter) were collected at 2 h (day 0) and 2 days post inoculation (dpi) to quantify bacterial populations. The experiments were repeated three times. Results shown are the mean of three independent experiments using three biological replicates per strain, including standard error of the mean. Photographs for each technical replicate were taken 7 dpi. Statistical analyses were performed using Prism 6.0 (GraphPad Software).

P. syringae pv. Tomato Inoculation and Population Assays in Arabidopsis thaliana

Five-week-old plants were dip inoculated for 20 seconds in a bacterial suspension (1×10⁸ cfu ml⁻¹ of Pst) containing 10 mM MgCl₂+0.02% Silwet L-77. Bacterial populations were measured 3 dpi by submerging the aerial plant tissue in 10 mM MgCl₂+0.2% Silwet L-77 for 2 h at 28° C. The bathing solution was serially diluted and plated (Tornero et al., “A high-Throughput Method for Quantifying Growth of Phytopathogenic Bacteria in Arabidopsis thaliana,” Plant J. 28(4):475-81 (2001), which is hereby incorporated by reference in its entirety). The experiments were repeated three times. Results shown are the mean of three independent experiments using three biological replicates per strain, including standard error of the mean. Statistical analyses were performed using Prism 6.0 (GraphPad Software).

Immunodetection of AvrRpt2 Proteins in P. syringae pv. Tomato

Strains of P. syringae pv. tomato grown on KB plates for 2 days at 30° C. were resuspended in hrp-inducing liquid minimal media (50 mM KH2PO4, 7.6 mM (NH4)2SO4, 1.7 mM NaCl, 1.7 mM MgCl₂, 10 mM fructose, pH 5.7) or KB liquid media containing the appropriate antibiotics at an OD₆₀₀ of 0.4 and 0.1 respectively. Bacterial cultures were grown at 28° C. for 16 hours shaking at 220 RPM and OD₆₀₀ was adjusted to a final concentration of 0.5. One ml of each bacterial culture was washed with water and centrifuged. Bacterial pellets were resuspended in 100 μl of Laemmli buffer (20 mM Tris, 1% sodium dodecyl sulfate [SDS], 0.05% bromphenol blue, and 10% glycerol, pH 6.8), boiled for 5 minutes, and 5 μl of each was used for immunoblot analysis. To detect AvrRpt2 proteins, membranes were probed with ∝-HA antibody (Roche, Indianapolis, IN) conjugated with HRP.

### Agrobacterium-Mediated Transient Protein Expression in Leaves

Agrobacterium tumefaciens 1D1249 (Wroblewski et al., “Optimization of Agrobacterium-Mediated Transient Assays of Gene Expression in Lettuce, Tomato, and Arabidopsis,” Plant Biotech. J. 3:259-73 (2005), which is hereby incorporated by reference in its entirety) harboring the various expression vectors were grown on LB media with the appropriate antibiotics for 2 days at 30° C. Bacteria were scraped from the plate, resuspended in infiltration buffer (10 mM MgCl₂, 10 mM MES [pH 5.6], and 200 mM acetosyringone) and maintained for 4 h in the dark at room temperature on a nutator rocker. Bacterial cultures were then washed, centrifuged, and the pellet was resuspended in fresh infiltration buffer before diluting cultures at a final OD₆₀₀ of 0.15. Tomato or N. benthamiana leaves were infiltrated using a needle-less syringe and placed to a growth chamber (24° C. day and 22° C. night). Leaf samples for protein expression were taken 32 hours later.

### Immunoblot Detection of Plant-Expressed Proteins

Protein samples were analyzed by grinding three leaf disks (9 mm in diameter) in protein sample buffer (50 mM Tris HCl [pH 7.5], 10% glycerol, 2% SDS, 2 mM EDTA, 1 mM DTT and 1% protease inhibitor [Sigma-Aldrich]). Samples were separated by SDS-PAGE on 4-20% gradient polyacrylamide gels and transferred to Immobilon-P PVDF membranes (Millipore) according to standard procedures (Taylor, “Using Antibodies: A Laboratory Manual,” Quarterly Rev. Biol. 74:1-374 (2015), which is hereby incorporated by reference in its entirety). To detect AvrRpt2 proteins, membranes were probed with ∝-c-Myc (GeneScript, Piscataway, NJ) antibody conjugated with HRP. For tomato Rin4 detection, AtRin4 polyclonal antiserum (G. Coaker, UC-Davis) was used at a concentration of 1:2.000. Secondary goat anti-rabbit IgG conjugated with HRP was used at a dilution of 1:10,000 (Promega, Madison, WI).

Ralstonia pseudosolanacearum Disease Assays

For survival assays with R. pseudosolanacearum CMR15, 4-week-old tomato plants (grown in peat pots) were transferred in potting mixture in 3-liter pots to the Toulouse Plant-Microbe Phenotyping facility, (28° C., 16 hours light). Each tomato plant was soil-drench inoculated with 50 ml of 10⁸ cfu ml⁻¹. Disease scoring was performed daily using a visual index in which the numbers 1, 2, 3 and 4 corresponded to 25%, 50%, 75% and 100% wilted leaves, respectively. Disease scores were transformed into binary data for the purpose of statistical comparison between disease curves (Remigi et al., “Functional Diversification of the GALA Type III Effector Family Contributes to Ralstonia solanacearum Adaptation on Different Plant Hosts,” New Phytol. 192(4):976-87 (2011), which is hereby incorporated by reference in its entirety).

### Tomato Genome Sequencing

Genomic DNA was extracted from a single LA4245-R plant using a DNeasy Plant Mini Kit (QIAGEN). DNA was mechanically sheared using the Covaris S2 Adaptative Focused Acoustic Disruptor (Covaris, Inc., Woburn, MA, USA) to an average size of 500-600 bp, and used to prepare a library. Single-end 100 bp DNA reads were sequenced using the Illumina HiSeq 2000 platform. The reads from LA4245-R were mapped to the S. lycopersicum Heinz 1706 genome sequence SL2.50 (Tomato Genome Consortium, 2012) using hisat2 version 2.1.0 (Kim et al., “HISAT: a Fast Spliced Aligner with Low Memory Requirements,” Nat. Methods 12(4):357-60 (2015), which is hereby incorporated by reference in its entirety), and SNPs were called using GATK version 4.0. (McKenna et al., “The Genome Analysis Toolkit: a MapReduce Framework for Analyzing Next-Generation DNA Sequencing Data,” Genome Res. 20(9):1297-303 (2010), which is hereby incorporated by reference in its entirety). SNPs were plotted in 10 kb bins using R. Synteny between SL2.50 and LA2951 version 0.6 was determined using SynMap at CoGe: genomevolution.org/coge/SynMap.pl. Protein similarity for RPS2 and MR5 were calculated with Geneious R11: geneious.com. The genome sequence of LA4245 is available at NCBI SRA under BioProject ID PRJNA516877 and is hereby incorporated by reference in its entirety. The genome sequence of LA2951 is available at: solgenomics.net/organism/Solanum_lycopersicoides/genome and is hereby incorporated by reference in its entirety.

### Example 2: A Locus on Chromosome 4 from S. lycopersicoides LA2951 Confers Resistance to Pst Race 1 Strains

During the summer of 2015 in upstate New York, a research plot of 110 S. lycopersicum VF36×S. lycopersicoides LA2951 introgression lines (ILs; (Canady et al., “A Library of Solanum lycopersicoides Introgression Lines in Cultivated Tomato,” Genome 48(4):685-97 (2005), which is hereby incorporated by reference in its entirety) became naturally infected by Pseudomonas syringae pv. tomato (Pst) resulting in severe symptoms of bacterial speck disease. However, two ILs, LA4245 and LA4277, remained essentially free of disease. LA4245 and LA4277 have large overlapping introgressed segments from chromosome 4 of S. lycopersicoides (Canady et al., “A Library of Solanum lycopersicoides Introgression Lines in Cultivated Tomato,” Genome 48(4):685-97 (2005), which is hereby incorporated by reference in its entirety). The introgression in LA4245 is smaller and so that line was further characterized. In order to determine the race and other characteristics of the Pst strain involved in the outbreak, isolates were collected from the field and analyzed. The presence of both avrPto and avrPtoB genes, immunoblot detection of the AvrPto protein, and subsequent inoculation of tomato plants with and without the Pto gene indicated the field isolates were race 0 Pst strains (Kraus et al, “Pseudomonas syringae pv. tomato strains from New York Exhibit Virulence Attributes Intermediate Between Typical race 0 and race 1 Strains,” Plant Dis. 101:1442-8 (2017), which is hereby incorporated by reference in its entirety). One Pst strain, referred to as NY15125, was chosen for further analysis.

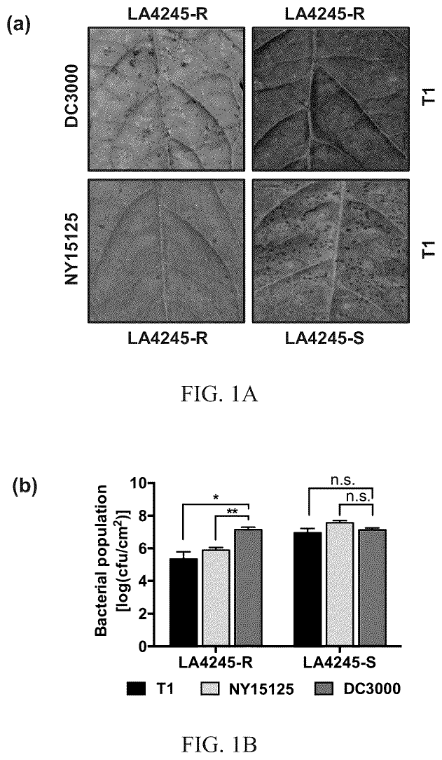

The chromosome 4 introgression segment in LA4245 is maintained in heterozygous condition because homozygotes are very rarely obtained, as noted previously (Canady et al., “A Library of Solanum lycopersicoides Introgression Lines in Cultivated Tomato,” Genome 48(4):685-97 (2005), which is hereby incorporated by reference in its entirety). The putative LA4245 resistance locus was named Ptr1 (Pseudomonas s. pv. tomato race 1) and a nomenclature was used in which its presence or absence is denoted as LA4245-R (Ptr1/ptr1) or LA4245-S(ptr1/ptr1), respectively. To follow up the field observations, LA4245 plants were inoculated with Pst strains DC3000, NY15125 and T1 in the greenhouse. Pathogen assays showed that DC3000 caused severe symptoms on LA4245-R plants, whereas NY15125 and T1 caused the appearance of few or no specks on LA4245-R, respectively (FIG. 1A). All of the Pst strains caused more disease on LA4245-S plants. Consistent with these observations, bacterial population assays showed that T1 attained levels ˜65-fold lower than DC3000 on LA4245-R plants (FIG. 1B). NY15125 grew to a level intermediate between T1 and DC3000 on LA4245-R. Such intermediate growth was observed previously for NY15125 on Pto-expressing plants (Kraus et al, “Pseudomonas syringae pv. tomato strains from New York Exhibit Virulence Attributes Intermediate Between Typical race 0 and race 1 Strains,” Plant Dis. 101:1442-8 (2017), which is hereby incorporated by reference in its entirety). The three Pst strains all attained similar population levels on LA4245-S plants (FIG. 1B). Therefore, the putative Ptr1 locus from S. lycopersicoides LA2951 confers resistance to Pst strains NY15125 and notably T1, which is a race 1 strain.

### Example 3: Pst Resistance in LA4245-R Plants is Due to Recognition of the Bacterial Effector AvrRpt2

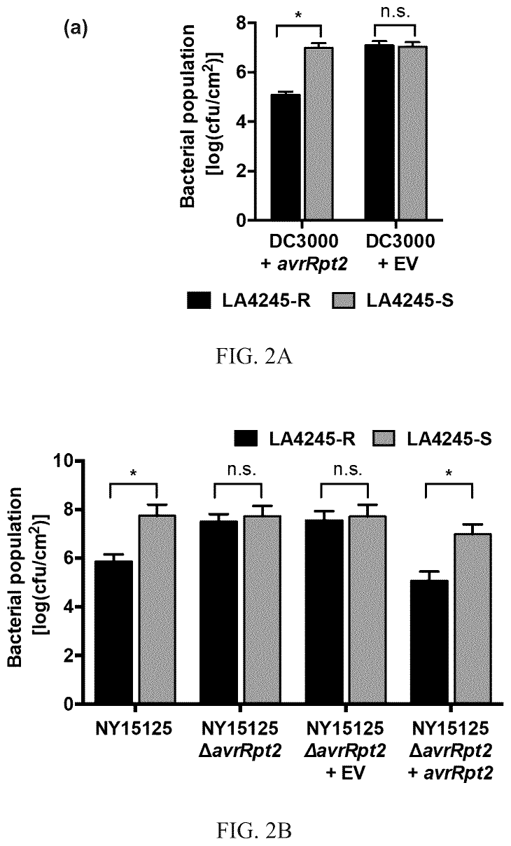

A comparison of the type III effector genes in DC3000 and T1 identified eight that are present exclusively in Ti (avrA1, avrRpt2, hopAE1, hopAG1, hopAI1, hopAS1, hopS1, and hopW1) (Jones et al., “Genome-Assisted Development of a Diagnostic Protocol for Distinguishing High Virulence Pseudomonas Syringae pv. tomato Strains,” Plant Disease 99:527-34 (2015), which is hereby incorporated by reference in its entirety). To determine whether LA4245-R resistance involves the recognition of any of these effectors, T1-specific effectors were individually cloned into the expression vector pCPP5372, the plasmids were introduced via electroporation into DC3000 ΔavrPtoΔavrPtoB and the strains were inoculated onto LA4245-R plants. All of the strains, except the one expressing AvrRpt2, caused disease on LA4245-R plants. Subsequent experiments showed that the DC3000 strain expressing AvrRpt2 reached a population size 80-fold less in leaves of LA4245-R plants compared to LA4245-S plants; a DC3000 strain carrying an empty vector grew to the same level in the two plant lines (FIG. 2A).

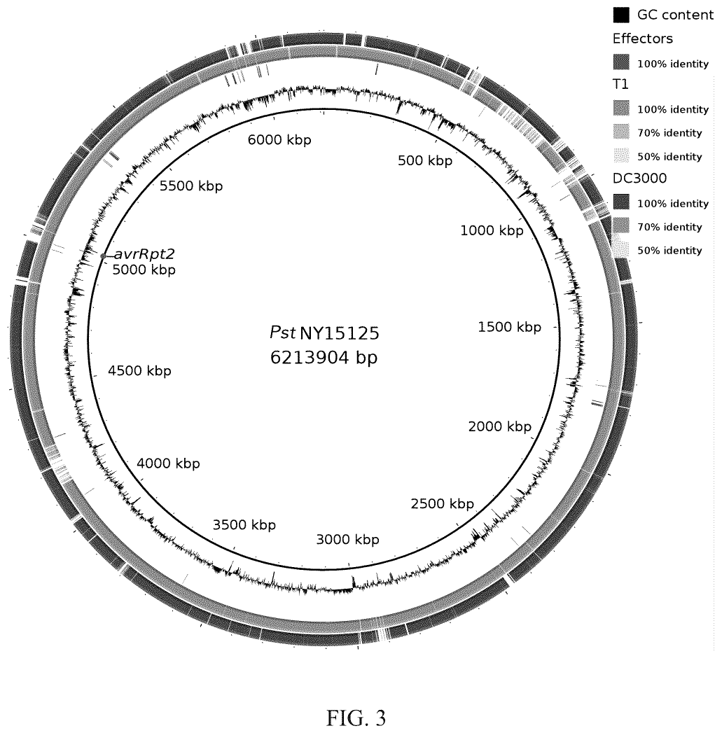

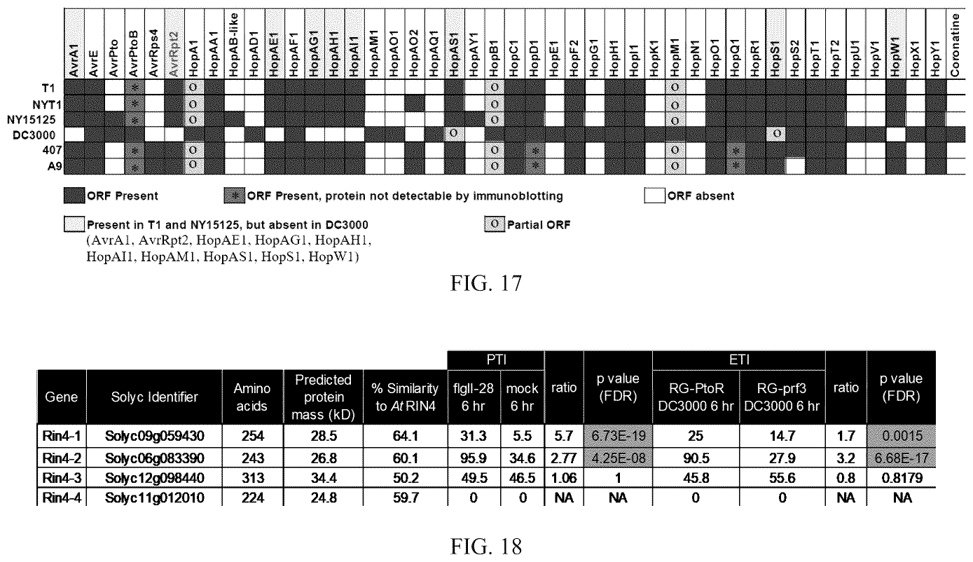

Next, it was determined whether Pst isolate NY15125, which was collected from the naturally-infected tomato field, has the avrRpt2 gene. Genome sequencing and PCR analysis confirmed the presence of avrRpt2 in this isolate (FIG. 3, FIG. 17). A comparison of AvrRpt2 protein sequences from NY15125 and T1 showed they are 100% identical. To investigate the activity of avrRpt2 in NY15125, a NY15125ΔavrRpt2 deletion mutant was generated and tested with regard to whether the strain was altered in its ability to grow on LA4245-R or LA4245-S plants. LA4245-R plants inoculated with the NY15125 wild-type strain supported a ˜60-fold lower bacterial population compared to LA4245-S plants (FIG. 2B). In contrast, this difference was not observed when LA4245-R and LA4245-S plants were inoculated with NY15125ΔavrRpt2. Importantly, complementation of the mutant strain with avrRpt2 restored the wild-type growth difference in LA4245-R plants (FIG. 2B).

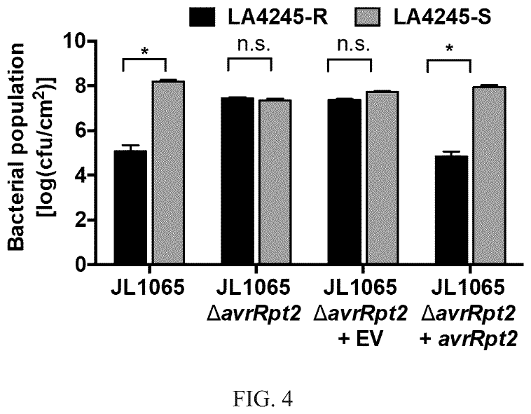

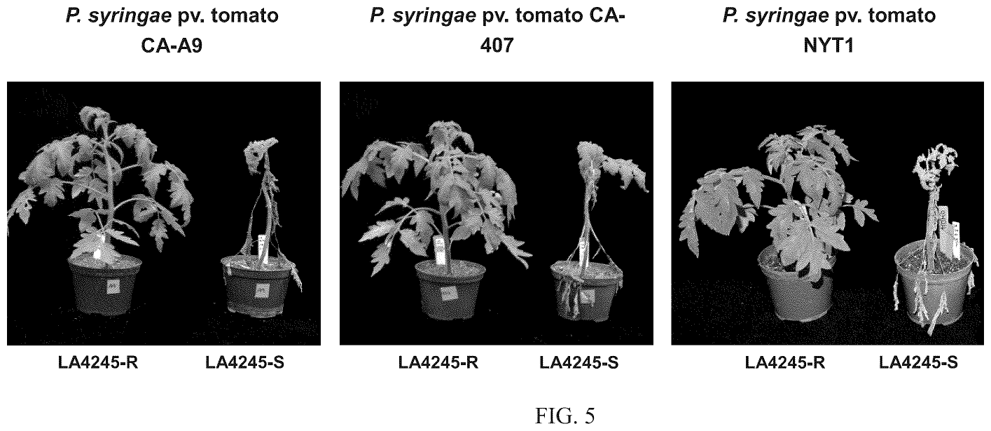

AvrRpt2 was originally identified in Pst JL1065 (Whalen et al., “Identification of Pseudomonas syringae Pathogens of Arabidopsis and a Bacterial Locus Determining Avirulence on Both Arabidopsis and Soybean,” Plant Cell 3:49-59 (1991), which is hereby incorporated by reference in its entirety). A comparison between the AvrRpt2 protein sequences from JL1065 and NY15125 showed that the proteins have just two divergent amino acids: proline-24 and alanine-152 in AvrRpt2_{JL1065} are replaced by threonine and glycine, respectively in AvrRpt2_{NY15125}. Difference in effector recognition on LA4245-R plants when infected with JL1065 wild-type and JL1065ΔavrRpt2 strains (Lim et al. “The Pseudomonas syringae avrRpt2 Gene Contributes to Virulence on Tomato,” Mol. Plant Microbe. Interact. 18:626-33 (2005), which is hereby incorporated by reference in its entirety) was investigated. Bacterial population assays indicated that AvrRpt2_{JL1065} is also recognized by LA4245-R (FIG. 4). Finally, three additional race 1 Pst strains which all carry the avrRpt2 gene (NYT1, CA-A9, and CA-407) were tested; it was found that LA4245-R is resistant to all of them, whereas LA4245-S was susceptible to these strains (FIG. 5). Thus, Ptr1 confers AvrRpt2-mediated resistance to multiple Pst race 1 strains.

### Example 4: The Ptr1 Locus and RPS2 Detect the Same Variants of AvrRpt2

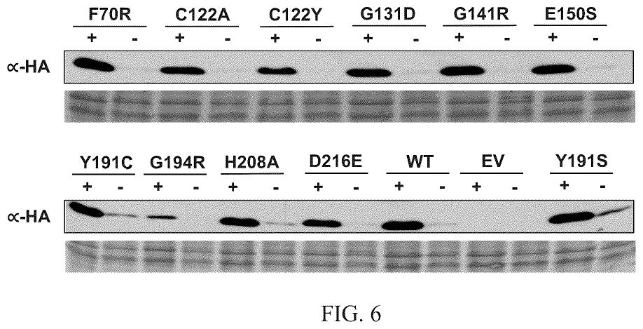

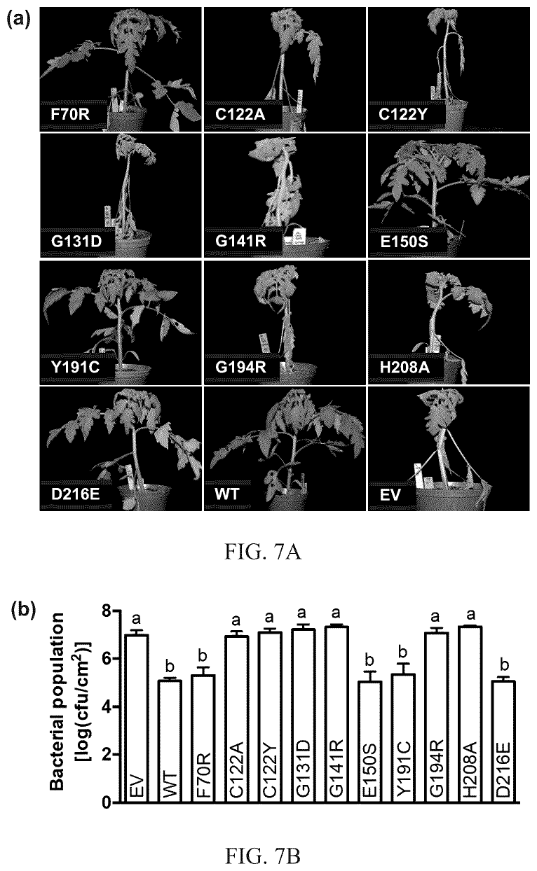

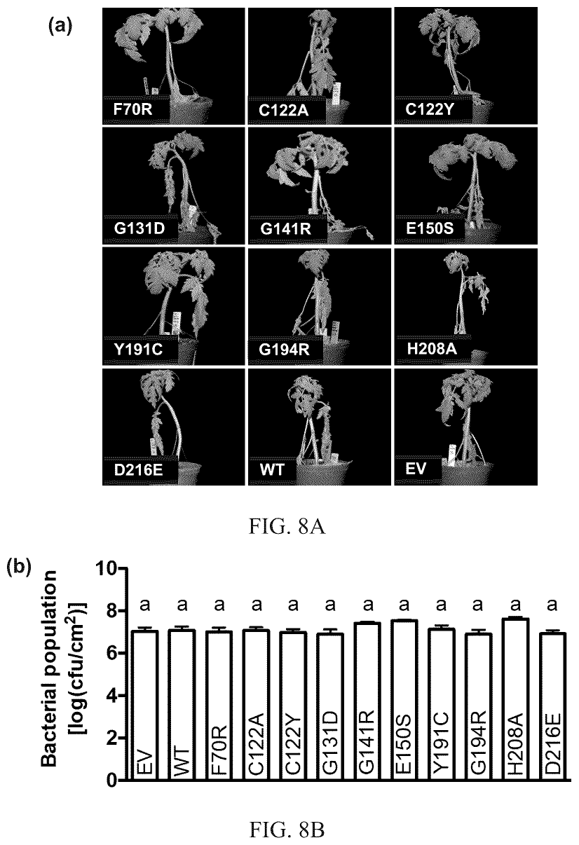

To gain insight into the mechanism of Ptr1 recognition of AvrRpt2, site-directed mutagenesis was performed to alter amino acids in the effector that have been reported to be essential for its recognition by RPS2 in Arabidopsis (Jin et al., “Cleavage of the Pseudomonas syringae Type III Effector AvrRpt2 Requires a Host Factor(s) Common Among Eukaryotes and is Important for AvrRpt2 Localization in the Host Cell,” Plant Physiol. 133(3):1072-82 (2003); Lim et al., “The Pseudomonas syringae Type III Effector AvrRpt2 Promotes Virulence Independently of RIN4, a Predicted Virulence Target in Arabidopsis thaliana,” Plant J. 40(5):790-8 (2004); Lim et al., “Mutations in the Pseudomonas syringae avrRpt2 gene that Dissociate its Virulence and Avirulence Activities Lead to Decreased Efficiency in AvrRpt2-Induced Disappearance of RIN4,” Mol. Plant Microbe. Interact. 17(3):313-21 (2004); Chisholm et al., “Molecular Characterization of Proteolytic Cleavage Sites of the Pseudomonas Syringae Effector AvrRpt2,” PNAS 102:2087-92 (2005), which are hereby incorporated by reference in their entirety). Ten AvrRpt2 variants were generated, of which eight have been reported to abolish recognition by RPS2 (Axtell et al., “Mutational Analysis of the Arabidopsis RPS2 Disease Resistance Gene and the Corresponding Pseudomonas syringae avrRpt2 Avirulence Gene,” Mol. Plant-Microbe Interact. 14(2):181-8 (2001); Axtell et al., “Genetic and Molecular Evidence that the Pseudomonas Syringae Type III Effector Protein AvrRpt2 is a Cysteine Protease,” Mol. Microbiol. 49(6):1537-46 (2003); Jin et al., “Cleavage of the Pseudomonas syringae Type III Effector AvrRpt2 Requires a Host Factor(s) Common Among Eukaryotes and is Important for AvrRpt2 Localization in the Host Cell,” Plant Physiol. 133(3):1072-82 (2003); Lim et al., “The Pseudomonas syringae Type III Effector AvrRpt2 Promotes Virulence Independently of RIN4, a Predicted Virulence Target in Arabidopsis thaliana,” Plant J 40(5):790-8 (2004), which are hereby incorporated by reference in their entirety); two variants, F70R, which disrupts the AvrRpt2 autocleavage site, and E150S are still recognized by RPS2 (Jin et al., “Cleavage of the Pseudomonas syringae Type III Effector AvrRpt2 Requires a Host Factor(s) Common Among Eukaryotes and is Important for AvrRpt2 Localization in the Host Cell,” Plant Physiol. 133(3):1072-82 (2003); Chisholm et al., “Molecular Characterization of Proteolytic Cleavage Sites of the Pseudomonas Syringae Effector AvrRpt2,” PNAS 102:2087-92 (2005), which are hereby incorporated by reference in their entirety). Each AvrRpt2 variant was introduced into DC3000 on a plasmid and shown to be expressed by immunoblotting (FIG. 6). The strains were then vacuum infiltrated into LA4245-R and LA4245-S plants and bacterial populations in leaves were measured and plants scored for disease symptoms. It was observed that DC3000 expressing AvrRpt2 variants with the substitutions C122A, C122Y, G131D, G141D, G194R or H208A grew to high levels and caused severe disease on LA4245-R plants, indicating a loss of Ptr1 recognition (FIGS. 7A and 7B). In contrast, similar to wild-type AvrRpt2, strains with variants F70R, E150S, Y191C or D216E reached population levels on average ˜60-fold lower than the empty vector control strain and did not cause any disease symptoms on LA4245-R plants (FIGS. 7A and 7B). All of the DC3000 strains caused disease and reached similar population levels in LA4245-S plants (FIGS. 8A-8B).

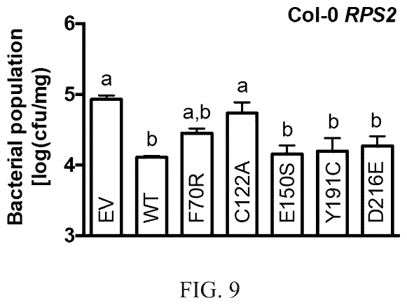

Interestingly, AvrRpt2 variants with the substitutions Y191C and D216E were recognized by Ptr1 but have been reported earlier not to be recognized by RPS2 and to be unable to induce Arabidopsis RIN4 degradation (Lim et al., “Mutations in the Pseudomonas syringae avrRpt2 gene that Dissociate its Virulence and Avirulence Activities Lead to Decreased Efficiency in AvrRpt2-Induced Disappearance of RIN4,” Mol. Plant Microbe. Interact. 17(3):313-21 (2004), which is hereby incorporated by reference in its entirety). However, recently it was shown that Arabidopsis RIN4 is cleaved by variants AvrRpt2(Y191C) and AvrRpt2(D216E) (Eschen-Lippold et al., “Bacterial AvrRpt2-like Cysteine Proteases Block Activation of the Arabidopsis Mitogen-Activated Protein Kinases, MPK4 and MPK11,” Plant Physiol. 171: 2223-38 (2016), which is hereby incorporated by reference in its entirety). To investigate this discrepancy, Arabidopsis Col-0 RPS2 plants were inoculated with DC3000 expressing AvrRpt2 and several of the variants, including Y191C and D216E, and bacterial population were measured three days later. A significant reduction in bacterial growth and an absence of disease symptoms was observed in Col-0 RPS2 plants inoculated with DC3000 carrying AvrRpt2 and the variants E150S, Y191C, and D216E; the F70R variant appeared to be weakly detected by RPS2 (FIG. 9). Thus Ptr1 and RPS2 detect the same variants of AvrRpt2.

### Example 5: Ptr1 Recognition of the AvrRpt2 Variants Correlates with the Effector's Ability to Cleave Tomato RIN4 Proteins

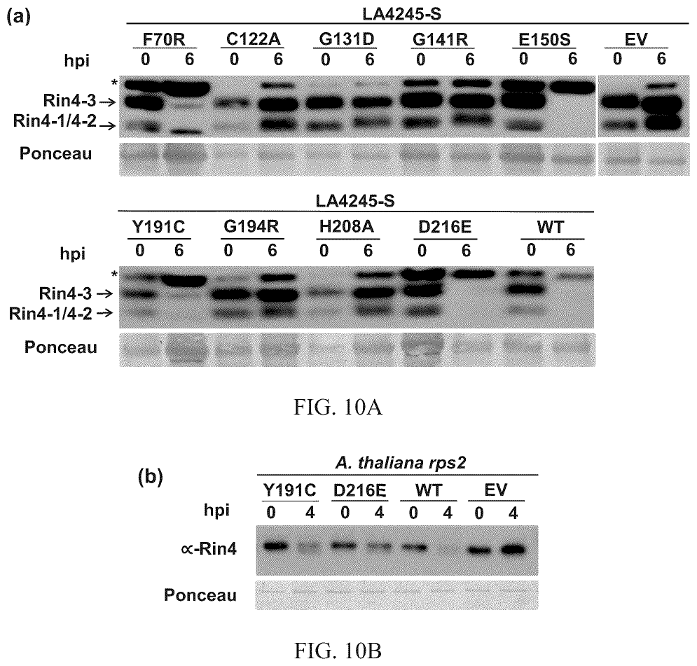

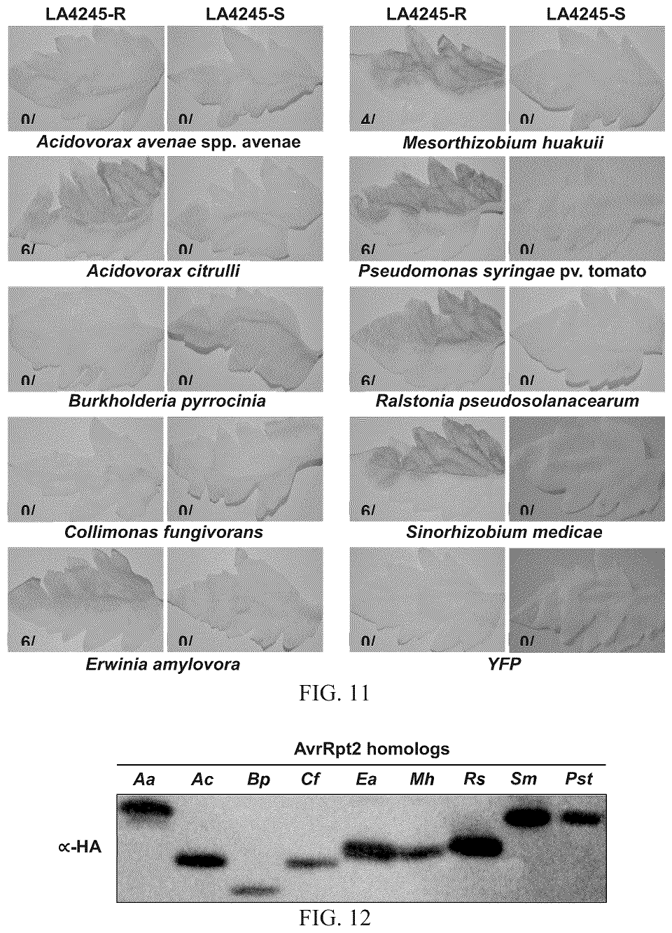

In Arabidopsis, AvrRpt2-mediated degradation of RIN4 leads to the activation of RPS2 (Mackey et al., “Arabidopsis RIN4 is a Target of the Type III Virulence Effector AvrRpt2 and Modulates RPS2-Mediated Resistance,” Cell 112(3):379-89 (2003), which is hereby incorporated by reference in its entirety). Accordingly, it was hypothesized that AvrRpt2 variants that are recognized by Ptr1 will also be capable of degrading RIN4 in tomato. Tomato has three genes with similarity to AtRIN4 that are expressed in leaves and two of these are induced during Pto-mediated NTI (SlRin4-1, SlRin4-2, and SlRin4-3; FIG. 18). To avoid the Ptr1-mediated defense response, LA4245-S plants with the DC3000 strains expressing each AvrRpt2 variant and wildtype AvrRpt2 were vacuum infiltrated and SlRin4 abundance was detected by immunoblotting 6 hours later. Wild-type AvrRpt2 and the variants F70R, E150S, Y191S and, D216A each induced a reduction in tomato RIN4 abundance which correlates with Ptr1 recognition of these proteins in LA4245-R plants (FIG. 10A). DC3000 strains with AvrRpt2 variants C122A, G131D, G141D, G194R, H208A, or an empty vector, all of which caused disease on LA4245-R plants, failed to induce tomato Rin4 elimination (FIG. 10A). Similar experiments in Arabidopsis, supported our earlier observations in that both AvrRpt2(Y191C) and AvrRpt2(D216E) were able to degrade RIN4 in rps2 plants (FIG. 10B). Therefore, there is a perfect correlation between the recognition of each AvrRpt2 variant by Ptr1 and the effector's ability to induce tomato Rin4 disappearance.

### Example 6: AvrRpt2 Proteins from Different Bacterial Species are Recognized by Ptr1

Homologs of AvrRpt2 are found in diverse bacterial species including the plant pathogens Erwinia amylovora, Ralstonia pseudosolanacearum, Acidovorax citrulli and Acidovorax avenae, the soil bacterium Burkholderia pyrrocinia, the fungal parasite Collimonas fungivorans, and the symbiotic bacteria Mezorhizobium huakuii and Sinorhizobium medicae (Zhao et al., “The Erwinia Amylovora avrRpt2EA Gene Contributes to Virulence on Pear and AvrRpt2EA is Recognized by Arabidopsis RPS2 When Expressed in Pseudomonas syringae,” Mol. Plant Microbe. Interact. 19(6):644-54 (2006); Eschen-Lippold et al., “Bacterial AvrRpt2-like Cysteine Proteases Block Activation of the Arabidopsis Mitogen-Activated Protein Kinases, MPK4 and MPK11,” Plant Physiol. 171: 2223-38 (2016), which are hereby incorporated by reference in their entirety). Some of these AvrRpt2 proteins have very divergent amino acid sequences or are truncated in comparison to Pst AvrRpt2, and it was investigated whether Ptr1 would recognize them. Agrobacterium-mediated expression (agroinfiltration) was used to express each AvrRpt2 protein or a YFP control in leaves of LA4245-R and LA4245-S. The AvrRpt2 homologs from five of the eight bacterial species induced cell death in LA4245-R leaves but not in LA4245-S leaves indicating their activity is recognized by Ptr1 (FIG. 11). Detection of protein expression of each AvrRpt2 homolog was done via agroinfiltration in LA4245-S leaves (FIG. 12).

### Example 7: An Amino Acid Substitution in AvrRpt2 that Abolishes its Recognition by the Apple Mr5 NLR Resistance Protein does not Affect Recognition by Ptr1

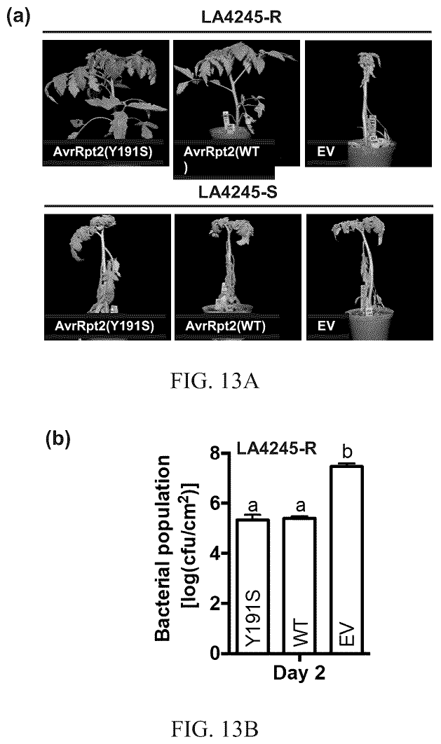

Mr5 is an NLR fire blight resistance protein in apple that recognizes strains of Erwinia amylovora that express AvrRpt2 (Fahrentrapp et al., “A Candidate Gene for Fire Blight Resistance in Malus×Robusta 5 is Coding for a CC-NBS-LRR,” Tree Genet. Genomes 9:237-51 (2012); Vogt et al., “Gene-for-Gene Relationship in the Host-Pathogen System Malus×Robusta 5-Erwinia Amylovora,” New Phytol. 197:1262-75 (2013), which are hereby incorporated by reference in their entirety). A single amino acid substitution in AvrRpt2 at position 156 (cysteine-to-serine) abolishes recognition of the effector by Mr5. In AvrRpt2_{NY15125} the comparable residue is tyrosine-191. Since a Y191C substitution in AvrRpt2 was shown previously to be recognized by Ptr1 (FIGS. 7A-7B), it was investigated whether a Y191S substitution in AvrRpt2 would abolish Ptr1 recognition as it does for Mr5 recognition. Inoculation of LA4245-R plants with a DC3000 strain expressing AvrRpt2(Y191S) revealed this variant is recognized by Ptr1, as it induced disease resistance and reduced bacterial growth compared to an empty vector DC3000 control strain (FIGS. 13A-13B). Mr5 and Ptr1 therefore appear to use different mechanisms to detect AvrRpt2.

### Example 8: Ptr1 Confers Resistance to Bacterial Wilt Disease Caused by Ralstonia Pseudosolanacearum

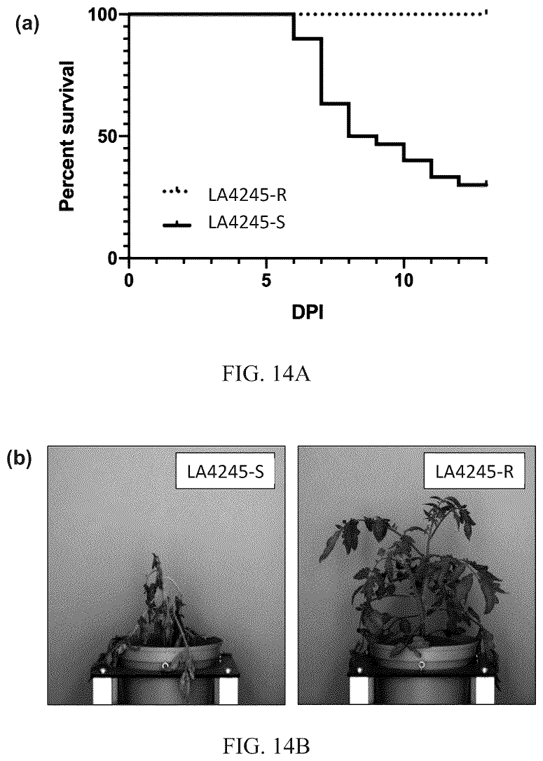

The agroinfiltration experiments indicated that RipBN, the AvrRpt2 homolog from R. pseudosolanacearum, is recognized by Ptr1 (FIG. 11). The ripBN gene is present in a strain of R. pseudosolanacearum, CMR15, which was collected from tomato in Cameroon in 2009 (Mahbou et al., “Broad Diversity of Ralstonia solanacearum Strains in Cameroon,” Plant Dis. 93:1123-30 (2009), which is hereby incorporated by reference in its entirety) and later sequenced (Remenant et al., “Genomes of Three Tomato Pathogens Within the Ralstonia solanacearum Species Complex Reveal Significant Evolutionary Divergence,” BMC Genomics 11:379 (2010), which is hereby incorporated by reference in its entirety). Bacterial wilt, caused by R. pseudosolanacearum, is a devastating disease for which no NLR-mediated resistance in tomato has been reported (Huet, “Breeding for Resistances to Ralstonia solanacearum,” Frontiers Plant Sci. 5:715 (2014), which is hereby incorporated by reference in its entirety). LA4245-R and LA4245-S plants with CMR15 were soil-drench inoculated and the number of plants showing symptoms of bacterial wilt over a 13-day time period were scored. Beginning at 6 days after inoculation LA4245-S plants started to wilt, and 13 days after inoculation ˜70% of the plants were dead (FIGS. 14A-14B). Remarkably, at this timepoint there was 100% survival of the LA4245-R plants. Ptr1-mediated resistance therefore can be effective against bacterial wilt and might be useful for controlling bacterial wilt disease in tomato-growing areas that have R. pseudosolanacearum strains expressing RipBN.

### Example 9: No RPS2, Mr5 or RIN4 Orthologs are Present in the S. lycopersicoides Chromosome 4 Introgression Segments of LA4245-R

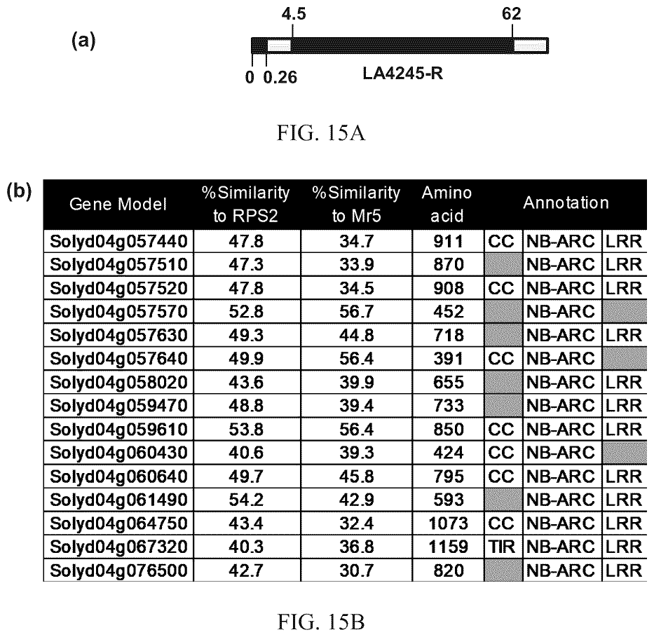

To initiate the map-based cloning of Ptr1, sequence data was generated at 14× coverage of the Heinz 1706 reference genome from genomic DNA of LA4245-R using an Illumina HiSeq2000. The reads were mapped to the S. lycopersicum Heinz 1706 genome sequence to identify areas of high sequence polymorphism. This analysis revealed that LA4245-R contains two S. lycopersicoides introgression segments on chromosome 4 in the background of the tomato parent VF36. One small segment lies within coordinates 1-260,000 bp (260 kb) and the other lies between coordinates 4,480,000 bp and 62,030,000 bp (˜57.5 mb) (FIG. 15A). A high-quality genome sequence of S. lycopersicoides LA2951, a parent of the introgression lines, has been generated recently (see Example 1). Synteny between Heinz 1706 and LA2951 was determined and gene models predicted to be in the introgressed regions of LA4245-R were identified in the S. lycopersicoides annotation. Ptr1 recognition of AvrRpt2 suggests it likely encodes an NLR protein or possibly a guardee such as Rin4. No NLR-encoding genes are annotated within the small introgression segment and just 15 NLR genes are annotated within the large S. lycopersicoides introgression segment. Interestingly, none of these 15 genes encode a predicted protein with similarity to RPS2 or Mr5 (FIG. 15B). In fact, the S. lycopersicoides genes with predicted proteins having the highest similarity to RPS2 and Mr5 are all located on other chromosomes (Table 4). None of the four Rin4 genes in tomato are located on chromosome 4 (FIG. 18).

The sequences of the predicted NRL-like genes present in the large introgression segment of LA4245-R are as follows:

The coding sequence for Solyd04g057440.1, which is hereby incorporated by reference in its entirety, (SEQ ID NO: 1) is as follows:

ATGGAGGCTACAATCGTTGCAGCGTTGAGTCCGGCGGCGACAAAAGCGGT
AAGCTTTCTGGTGGACAGTCTATCGCAGTTACTATCGGAAAATGTGGAAC
TGATAAGAGGTGCAGATAGAGATTTCCAGCAATTACTGGATGAAATTACA
CCCATAAATGAGTTACTATCTGGAGATTATGCACAATTGAAAAGCAACAA
CAACAACGATTTGGATAAATTGTTCCAGAATATTCAACGAACAGTATATA
AAGCTGAAGATGCGATCGATAAATTCCTAATTCAGGCGAAAATTAACGAA
GCTAACGTGTTCAATAAGTTCGGTCCGTTTGTCAAGTGGAACAACAATAG
GAAAATTACACCGGAATTCAGAAAAATTCTCGAACAAGTGACTGAAATTC
GCCAACAGACTCAACAGGTTTTGGAGAAAACCGGTATACAGAGTTCTGCT
TTCCAGCCTGGAGAAACTACCCGGCCACAGGGTCCTGCTGAGGAGGATAG
CGAAGTGGTTGGTTTTAATAAGCCTGCAGAAGATGTGAAAAAGCGACTTT
GTGAAGGATCAAAAGATCTTGATGTTATACCTATTGTGGGTATGCCAGGA
CTTGGAAAGACCACACTTTCAAGAAAAGTTTACAATGATTCTTCCATTGA
TTTTTATTTTTACCATAAAATGTGGATTTATGTTGGGACATCAAAGAAAC
CAAAGGATATTCTTGTTGAGATTGTGAAAGATGTCGCGCAAAGCAATAGT
AAGGAACTAATTAAAGACAAGGATGAGGACCAATTAGCTCATATCATACG
TGATTTTCTTGTTAAAAGAGGTAAACATCTCATTGTCTTGGATGATGTGT
GGGACACACAAGTTGTAGATTTTGTCAAGAAAGCTTTCCCAAACAACAAA
TCCTGGCCCCGAGGGGACAGAATCATGTTGACAACTCGCCAACGACGTGT
GGCTGAAGCTGTCAGCGCTCGTCCTCACTATCTGGAAAACTTGTCAAAAG
AGGATAGTATAAAGTTGTTGGAACAGAGAGTTTTTGCCAATAAAAGGACA
TGTCCTATTGAGTTAGAAGGATATCGAGATGGGATTGTAGATAAATGTTG
TGGTGTACCACTTGCCATAGTGGTGATTTCAGGAGCTTTGAGAGGTGTTA
TGGACGAAAGTGAATGGAGAGTAGTCGAGGAAAATGTGGGGAAGCACCTT
ATAAACAAGGACGACCATAAAAGCTGCTTGAAATTTGTTGAAACGAGTTA
CAATCATTTGCTGCAAGAGAAAAAGGCAGCCTTCTTGTATTTTGGAGTAT
TTCCTCAAGGTTTTGATATTCCTGCTTGGAACCTTATTCGCTTATGGGTT
GCTGAGGGGCTAATAAAGTCCGATCATAAAGACAGTGAAATCGAGAAGGT
TGCGGAGACTTACTTGAGCGACTTTGCTAGTAGGAATTTAGTGATGGTGA
TGCAAAAGAGATCTAACGGTCAAATCAAAACGTGTCGTCTTCATGACATG
TTGCATGAGTTCTGCATTATTGAGGCTCAAAGGATAAGTCTCTTTCAACA
AGTATATCTCCAACCTGGTGTTCGAGTTTTTCCTTCTATAGAAGATCCAA
ATAATTCTCGTCGATTATGTATTCAATCCTCTATTCCGTATAATTTTATC
CCTAAAGATAGAATTGTACAGCACGTTAGGTCTCTCTTATGTTTTTCCTC
AGACCAAAAGCAAATTGACTTGTCTAATCTAGATGTCCAACTCATCCCCA
ATGCCTTTCCACTCATCAGGGTGTTGGACATTCAATCCCTCATATTTGAA
TTCTCCAAGATGTTTTATGGTCTATTTCACTTGAGGTATATTGCCATCAA
GGGCGACTTCACTGTCATTCCTTCACTCTTTCGTAATTTTTGGTATTTAC
AAACCCTTATACTTCGTAATGATGATACACATACTTCAAGATCCACCCTC
GAGATAAAAGAGGACATATGGAAACTGTTACAATTGAGACATCTGCACTC
CGACCTTCATGTGAAATTGCCTCCTCCTCCTACCCCAACAAGCAATAGTC
GTACTTCTTGTCTACAAACTCTTTCTAAGGTTACACCAGATAGTTGCAAA
AAAACTGTGTTTGCAAAGGCTTGTCATCTCAAAAAATTGGGTATTGAAGG
GCAATTGGCACTTCTTCTTGGAAAATCTACCAAGGGACGTGGATTCGACA
GTTTCCAAGAGCTAAGGTGCGTCGAAAAATTGAAATTGTTGAACAATGAT
TTTAGTGAAGAGCTTCACCTTCCTCCACACTTTTTCAGCTTACAAAAAAC
GCTGAACAAGTTAACTTTGTCAAGTACAAGGTTTGAGTGGAGTGAGGCAG
ATATGTTGGGGAAGTTGGAATGCCTTAAGGTACTAAAATTGAAATATAAT
GCATTCATAGGGAAGAATTGGAAGCCCAAGAAAGGAAGTTTTAGCAAGCT
CCAAGTCCTGCACATTGTCTGGGCAGGAGACTGGCAAACTTGGGATGCAT
CAAATCGTCCGTTCCAAAGTCTTACACACCTTGTTCTTATTTCCTGTAAT
CATCTCAAGGCTGTGCCACATGAGCTGGCTGATTTACCTTATCTTCAAGA
GATGAAGCTGACGCGCACATACGACGCAGTCAGTTCTGCCATAGAAATCA
AAAGAAAGATACTACAAAGGCAAGATCCCGAAAGCAACATCAAATTCAAT
CTCATTACATTTCCCCCTACCTCGTCCACAAATTAA

The coding sequence for Solyd04g057510.1, which is hereby incorporated by reference in its entirety, (SEQ ID NO: 2) is as follows:

ATGGCGGCTACTTACGGTGCACTAACTTCTGTGTTGGGAACCATAGAGAA
GGTTTTACGGTCCAACCATAGAGAATATGGTTGGACCGTAAAATTGAAAT
CACTGTATGGGTTGTTTGACACTCTAATTGTGAAATGTGATGTCGGAGAA
GCAATTATTAGCAAGGATTTGCTAAGAAGAATCAAAGATGTTGCAGTTTA
TGCAGAAGATGAAGTTGAATCACTAATGAAACAACTTATTATTATTGGGA
TGAATGATGAATGTTGTGGTGCGAAGCTTGATAAAGTCTCTCAACAGGTA
ATACAAGTAACTCATTCAGTCAACAATATTAATTGTCCAAAAAAAGTTGT
TTCTCCACGATTAGATGATGATTCAATCCATGAGAACTTTATGGAAGGGT
ACAATGAAGAAAGAGTAAATATGGTTCAAAGACTTACCAAAGGATCAAAT
AGACTGCAAGTGGTTTCTGTTGTGGGGATGGCGGGCATAGGTAAGACAAC
TTTTGCCAAAACTACATTATTACATATTAACAAGGAGGACTTTCCTATTC
GTGGTTGGATTACGGTGTCTAAAAACTATGATTTAAGAAAGTTGCTCCTA
CTCCTCCTTCATGATGTTATTGAAATGAAAGGTAAGAATTTTGATGGAAT
GGATAATGGACAACTAGCTGCTCATCTAAAGCAAGTTTTGCAGGGAAAAA
GGTATTTGATTGTTGTGGATGACATATGGAGCAAAGAAGATTGGGATGAA
ATTAAACATTGGTTTCCAGATTGTGGTGACAAAAGTCGAATTTTGTTGAC
TTCTCGAGACAAAGAGGTTGGTGAGTATGCTGCTAAAGAAGGTTTGGTAC
TGATGCGTCCTCTGACGCAAGATGAAAGTCGGAATCTGTTTTACCATAGG
GCATTTGGGAAAAATTACACTATTCGAGGGTCAGATATTGATGAATTCGA
AAAGGTTGGAGAAAAAGTTATAACAAATTGCAAAGGATTACCACTAATGA
TTACTGCAGTTGCTGGTATACTCCGTAGTAAGAGTAAACTGGATGAGTGG
ATGGAAGTAGCTCAAAGTGTAAGCTCATTAGTAAATGATGATGATTACAA
ACAATGCTTACAAGTTGTTGCTTTGAGCTACAATAATCTTCCTTCTCTAA
TGAAAGCTTGCTTTTTGCATTTCGGAGTTTTTCCAAAAGCCCATGTCATT
TCTGTGAAGAAGTTGATCAGATTATGGATTGCAGAAGGACTCGTAAATCT
AAAGGGAGTTGAGGAATTCGAACAAGCAGCTGATCGTGTTTTACATGATC
TTATTGGTAAAAGTGTAGTTATTGTTGACAAGCGAAGTTTGGATGGGAAA
ATTAAGACATGTAGGATTCATGATCTTTTTCATGATTTCTGCTCCAAGGA
AGCTGAAAGCGAGAATCTTCTGTATGTTGTTGGTTCAGATTCTCAAGTAC
ACACAAATTTCCGTCAAGGTTGTAGGTGGGTGTCAGTTCAATCAAAACTT
GATTATCGTCACCGCAGACGGTATACTCCTATTGAAATATCCTCTTTTTA
CATGCTATATAATATTGTTATTTTTGACAAAAAAGTTTTTCATTTCAAAC
TACTAAGGGTATTGGACTTGGAGGTACATGATATCATAGGACTCAGTGAA
ATTACTAGAGACCTTGTTTGTTTAAGGTATTTGGCTGTCGGGATTAAGTA
TGTAAGTTTTGTGGATCTCCCGATTACCAATCTTTGGAATCTACACACTC
TCATTTTAGGTAAATCTTTCACTAATAGTCTTATACATAAAGTCTTCAGT
TTTCCAAAAGATATTTGGCAAATGTCACAATTAAGGCATCTTTATGCAAA
AGGCATTTATCTATCTTCGCCTGGAGAAAAGGTTCTCGAAAACTTACAGA
GTGTTTCTGGTTTGAGTCCTTCTTGTTGTACAAAGGAAATATTTGAAGGG
ATTAAGAATGTGAAAAAATTGATCATTCGTGGAAAGAGGGAGGATTATCC
TACAGATATTAAATGGATAGATAATCTTAAATATTTGCAACATCTCGAGT
CACTGAGTATTGAAACTACCGATTTTCTATTTCATAAGAATAAAACCAGG
TTTTTTAGTCTTACAAGTCCAGATTCTTTTCCACAAAAACTCAAGAAATT
GAAACTTAGCTACACATATCTACCGTGGGAATACATGTCCATTATCAGCA
AGTTGCCCGAACTTGAGGTACTCAAACTGAAGTGTGGTTCCTTAATTGGC
GACGAGTGGAAAGCAACAGACCAGATTGGGTTCCCGAAGTTGAGGTTCTT
GCTCCTTGAGAATCTCTTCCTTGGAAAATGGGAAACCACCACGGGCTCTC
ATGATCATTTCCCCAGCCTTGAGCGCTTAATTATCACAAATTTCAGTTTC
TTAAAAGAGATTCCTCAAGGATTTGCTGATAGCAAGAAACTGGAGCTGAT
TGAGTTACACAATTGTGACCCTTCCTTGGTGGCTTTTGCTAAGCAGATAC
AGGTTGAACACGAGGATTTGGGGAGGAACAAACTTAAAGTTACTGCCTTC
GATACAGGTCGAGGATGGAACAGAGGAATCTTAGCTTCAAGAAATAACAG
GATTTCCAGCTAA

The coding sequence for Solyd04g057520.1, which is hereby incorporated by reference in its entirety, (SEQ ID NO: 3) is as follows:

ATGGAGGCTACAATCGTTGCAGCGGTGAGTCCGGCGGCGACAAAAGCGGT
AAGCTTTCTGGTGGACAGTCTATCGCAGCTACTATCGGAAAATGTGGAAC
TGATAAGAGGTGCAGATAGAGATTTCCAGCGATTACTGGATGAAATTAAA
CCCATAAATGAGTTACTAGCTGGAGATTATGCACAATTGAAAAGCAACAA
CAACAACGATTTGGATAAATTGTTCCAGAATATTCAACGAACAGTATATA
AAGCTGAAGATGCGATCGATAAATTCCTAATTCAGGCTAAAATTGACGAA
GCTAACCTGTTCAATAAGTTCGGTCCGTTCGTCAAGTGGAACAACAATCG
GAAAATTGCACCGGAATTCGAGAAAATTTTAAAACAAGTGGCCGGAATTC
GCCAACAGACTCAACAGGTTTTGGACAAAACCGGTATACAGAGTTCTGCT
TTCCAGCCTGGAGAAACTACGGGGACACAGGGTCCTGCTGAGGAGGATAT
CGAAGTGGTTGGTTTTAATAAGCCTGCAGAAGATGTGAAAAAGCGACTCT
GTGAAGGATCAAAGGATGTTGATGTTATACCTATTGTGGGTATGCCAGGA
CTTGGAAAGACCACTCTGTCAAGAAAAGTTTACAATGATTGTTCCCTTGA
TTTTTATTTTTACCATAAAATGTGGATTTATGTTGGGACATCAAAGAAAC
CAAAGGATATTCTTATTGAGATTGTGAAAGAAGTCGCGCAAAGCAATAGT
AAGGAACTAATTATAGACAAGGATGAGGACCAATTAGCTCATATCATACG
TGGTTTTCTTGTTGAAAGAGGTAAACATCTCATTGTCTTGGATGATGTGT
GGGACACACAAGTTGTAGATTTTGTCAAGAAAGCTTTCCCAAACAACAAA
TCCCGGCCCCGAGGGGACAGAATCATGTTGACAACTCGCCAACGACGTGT
GGCTGAAGCTGTCAGCGCTCGTCCTCACTATCTAGAAAACTTGTCAAAAA
AGGATAGTATAAAGTTGTTGGAACAGAGAGTTTTTGCCGATAAAAGGACA
TGTCCTATTGAGTTAGAAGTATATCGAGATGGGATTGTAGATAAATGTTG
TGGTGTACCACTTGCCATAGTGGTGATTTCAGGAGCTTTGAGAGGTTGTA
TGGGTGTGGGAGTAGTGCAAGAAAATATGGGGAAGCACCTTATAAACAAG
GACGACCATAAAAGCTGCTTGAAATTTGTTGAAACGAGTTACAATCATTT
GCCGCAAGAGAAAAAGGCAGCCTTCTTGTATTTTGGAGTATTTCCTCAAG
GTTTTGATATTCCTGCTTGGAACCTTATTCGCTTATGGGTTGCTGAGGGG
CTAATAAAGTCCAGTCATAAAGACAGTGAAATCGAGAAGGTTGCGGAGAC
TTACTTGAGCGACTTTGCTAGTAGGAATTTAGTGATGGTGATGCAAAAGA
GATCTAACGGTCAAATCAAAACGTGTCGTCTCCATGACATGTTGCATGAG
TTCTGCATTATTGAGGCTCAAAGGATAAGTCTCTTTCAACAAGTATATCT
CCAACCTGGTGTTCGAGTTTTTCCTTCTATAGAAGATCCAAATACTTCTC
GCCGATTATGTATTCAATCCTCTATTCCGTATAATTTTATCACTAAAGAT
AGAATTGTACAGCATGTTAGGTCTCTCTTATGTTTTTCCTCAGACCAAAA
GCAAATTGACTTGTCTAATCTAGATGTCCAACTTATCCCCAATGCCTTTC
CACTCATCAGGGTGTTGGACATTCAATCCCTCATATTTGAATTCTCCAAG
ATGTTTTATGGTCTATTTCACTTGAGGTATATTGCCATCAAAGGCGACTT
CACTGTCATTCCTTCACTCTTTCGTAATTTTTGGTATTTACAAACCCTTA
TACTTCGTAATGATGATACACATACTTCAAGCTCCACCCTTGAGATAAAA
GAGGACATATGGAAACTGTTACAATTGAGACATCTGCACTCCGACCTTCC
TGTGAAATTGCCTCCCCCTCCTACCCCAACAAGCGAGAGTCGTACTTCTT
GTCTACAAACTCTTTCTAAGGTTACGCCAGATAGTTGCAAAAAAACTGTG
TTTGCAAAGGCTTGTCATCTCAAAAAATTGGGTATTGAAGGGCAATTGGC
ACTTCTTCTTGGAAAATCTACCAAGGGAAGTGGATTCGACAGTTTCCAAG
AGCTAAGGTGCGTCGAAAAATTGAAATTGTTGAACAATGATTTTAGTGAA
GAGCTTCACCTTCCTCCACACTTTTTCAGCTTACAAAAAACACTGAACAA
GTTAACTTTGTCAAGTACAAGGTTTGAGTGGAGTGAGGCAGATATGTTGG
GGAAGTTGGAATGCCTTAAGGTACTAAAATTGAAAGATAATGCATTCATA
GGGAAGAATTGGAAGCCCAAGAAAGGAAGTTTTAGCAAGCTCCAAGTCCT
GCACATTGTCTGGGCAGGAGACTGGCAAACTTGGGATGCATCAAATCGTC
CGTTCCTAAGTCTTACACACCTTGTTCTTATTTCCTGTTATGATCTCAAG
GCTGTGCCACACGAGCTGGCTGATTTACCTTATCTTCAAGAGATGAAGCT
GACGCGCACATTCCAGGCAGTCAGTTCTGCCATAGAAATCAAAAGAAAGA
TACTACAAAGGCAAGATCCCGAAAGCAGCATCAAATTCAATCTCATTACA
TTTCCCCCTAACTCGTCCACAAATTAA

The coding sequence for Solyd04g057570.1, which is hereby incorporated by reference in its entirety, (SEQ ID NO: 4) is as follows:

ATGGCGGCTACTTACGGTGCACTAACTTCTGTGTTGGGAACCATAGAGAA
GGTTTTACGGTCCAACCATAGAGAATGTGGTTGGACCGTAAAATTGAAAT
CACTGTATGAGTTATTTGACACTCTACTTGGCAAATGTGATGTCGGAGAA
GCAATTATTAGCAAGGATTTGCTAAGAAGAATCAAAGATGTTGCAGTTTA
TGCAGAAGATGAAGTTGAATCACTAATGAAACAACTTATTGTTATTGGGC
TGAATGATGAATGTTGTGGTGCGAAGCTTGATAAAGTCTCTCAACAGGTA
ATACAAGTAACTCATTATGTCAACAATATTAATTGTCCAAAAAAAATTGT
TTCTCCACGATTAGATGATGATTCAATCTATGAGAACTTTATGGAAGGGT
ACAATGAAGAAAGAGTAAATATGGTTCAAAGACTTACCAAAGGATCAAAT
AGACTGCAAGTGGTTTCTGTTGTGGGGATGGCGGGCATAGGTAAGACAAC
TTTTGCCAAAACTATATTATTTCATGACCATCTTAAGAAGGAGGACTTTC
CTATTCGTGGTTGGATTACTGTGTCTAACAACTATGATTTAAGAAAGTTG
CTCCTACTCCTCCTTCACGATGTTATTGAAATGAAAGGCAAGAATTTTGA
TGAAATGGATAATGGAGAACTATCTGGTCACGTAAAGCAAGGTTTGCAGG
GAAAAAGGTATTTGATTGTTGTGGATGACATATGGAGCAATAAAGATTGG
GATAGTATTAAACATTTGTTTCCAGATTTTGGTGACAGAAGTCGAATTTT
GTTGACTTCTCGAGACAGGGAGGTTGGTGAGTATGCTGCTACCAATCCTA
AAGATGGTTTGGTACTGATGCGTCCTCTGACGCAAGATGAAAGTCGGAAT
CTGTTTTACCATAGGGTATTTGGGAAAAATTACACTATTCGAGGGTCAGA
TATTGATGAATTCAAAAAGGTTGGAGAAAAGGTTATAACAAATTGCAAAG
GATTACCACTAATGATTACTGCAGTTGCTGGTATACTCCGTAGTAAGAGT
AAACTGGATGAGTGGATGGAAGTAGCTCAAAGTGTAAGCTCATTAGTAAA
TGATGATGATTACAAACAATGCTTACAAGTTGTTGCTTTGAGCTACAATA
ATGTTCCTTCTCTTATGAAAGCTTGCTTTTTGCATTTCGGAGTTTTTCCA
AAAGCCCATGTCATTTCTGTGAAGAAGTTGATCAGATTATGGATTGCAGA
AGGACTCGTAAATCTAAAGGGAGTTGAGGAATTCGAACAAGCAGCTGATC
GTGTTTTACATGATCTTATTGGTAAAAGTGTAGTTATTGTTGACAGCTGT
CATGACTGA

The coding sequence for Solyd04g057630.1, which is hereby incorporated by reference in its entirety, (SEQ ID NO: 5) is as follows:

ATGGAAGGGTACAATGAAGAAAGAGTAAATATGGTTCAAAGACTTACCAA
AGGATCAAATAAACTGCAAGTGGTTTCTGTTGTGGGGATGGCGGGCATAG
TTAAGACAACTTTTGTCAAAACTATATTATTACATATTAACAAGGAGGAC
TTTCCTATTCGTGGTAGGATTACTGTGTCTAAAAACTATGATTTAAGAAA
GTTGCTCCAACTCCTCCTCCATGATGTTATTGAAATGAAAGGTAAGAATT
TTGATGGAATGGATACTGGAAAAAGGTATTTGATTGTTGTGGATGACATA
TGGAGCAAAAAAGATTGGGATGAAATTAAACATTGGTTTCCAGATTGTGG
TGACAGAAGTCGAATTTTGTTGACTTCTCGAGACAGGGAGGTTGGTGAGT
ATGCTGCTAAAGATGGTTTGGTACCGATGCGTCCTCTGACGCAAGATGAA
AGTCGGAATCTGTTTTACCATAGGGCATTTGGGAAAAATTACACTATTCG
AGGGTCAGATATTGATGAATTCGAAAAGGTTGGAGAAAAAGTTATAACAA
ATTGCAAAGGATTACCACTAATGATTACTGCAGTTGCTGGTATACTCCGT
AGTAAGAGTAAACTGGATGAGTGGATGGAAGTAGCTCAAAGTGTAAGCTC
ATTAGTAAATGATGATGATTACAAACAATGCTTACAAGTTGTTGCTTTGA
GCTACAATAATCTTCCTTCTCTAATGAAAGCTTGCTTTTTGCATTTCGGA
GTTTTTCCAAAAGCCCATGTCATTTCTGTGAAGAAGTTGATCAGATTATG
GATTGCAGAAGGACTCGTAAATCTAAAGGGAGTTGAGGAATTCGAACAAG
CAGCTGATCGTGTTTTACATGATCTTATTGGTAAAAGTGTAGTTATTGTT
GACAAGCGAAGTTTGGATGGGAAAATTAAGACATGTAGGATTCATGATCT
TTTTCATGATTTCTGCTCCAAGGAAGCTGAAAGCGAGAATCTTCTGTATG
TTGTTGGTTCAGATTCTCAAGTACACACAAATTTCCGTCAAGGTTGTAGG
TGGGTGTCAGTTCAATCAAAACTTGATTATCGTCACCGCAGACGGTATAC
TCCTATTGAAATATCCTCTTTTTACATGCTATATAATATTGTTATTTTTG
ACAAAAAAGTTTTTCATTTCAAACTACTAAGGGTATTGGACTTGGAGGTA
CATGATATCATAGGAATCAGTGAAATTACTAGAGACCTTGTTTGTTTAAG
GTATTTGGCTGTCGGGATTAAGTATGTAGATTTTGTGGATCTCCCAATTA
CCAATCTTTGGAATCTACACACTCTCATTTTAGGTAAATATTTTATTCAT
AATAGTCTTATGCATAAAGTCTTCACTTTTCCAAAAGATATTTGGCAAAT
GTCACAATTAAGGCATCTTTATGCAAAAGGCATTCATCTATGTTCGCCTG
GAGATAATAAGGTTCTCGAAAACTTACTGAGTGTTTCTGGTTTGAGTCCT
TCTTGTTGTACAAAGGAAATATTTGAAGGGATTAAGAAAGTGAAAAAATT
GGTCATTCGTGGAACGAGGGAGGAATGTCCTACAGATGTTAAATGGATAG
ATAATCTTAAATATTTGCAACATCTCGAGTCACTAAGTATTGAAAATATC
AAATTTATATTTCATGAGAATGAAACCAGGTTTTTTAGTCTTACAAGTCC
AGATTCTTTTCCACAAAAACTCAAGAAGTTGAAACTTAGCTACACATATC
TACCGTGGGAATACATGTCCATTATCAGCAAGTTGCCCGAACTTGAGGTA
CTCAAACTGAAGCGTAGTTCCTTAATTGGCAACGAGTGGAAAGCAACAGA
ACAGATTGGGTTCCCGAAGTTGAGGTTCTTGCTCCTTGAGAATCTCTTCC
TTAGAAAATGGGAAAGCACCACGGGCTATCATGATCATTTCCCCAGCCTT
GAGCGCTTAATTATCACAAATTTCAAATTCTTAAAAGAGATTCCTCAAGG
ATTTGCTGATAGCAAGAAACTGGAGCTGATTGAGTTACACAATTGTGACC
CTTCCTTGGTGGATTTTGCTAAGAAGGTGCAGCTTGAACACGAGGAGGTT
TTGGGGAGGAACAAACTTAAAGTTACTGCCTTCAATACAGGTAAATATAT
CTATTAG

The coding sequence for Solyd04g057640.1, which is hereby incorporated by reference in its entirety, (SEQ ID NO: 6) is as follows:

ATGGCCGCTACTTACGCTGCACTAACTTCTGTGTTGGGAACCATAGACAA
GCTTTTACGGTCCAACTTATTAGTTGGCGTAGAAGAGGTTCATAAGCAAC
AATTAGAATCACTCGACAAGATGTTTGGCACTCTGCAAGTGTCTCTAATT
GGCAAATGTGATGGCGAAGAAGCAATTATTAGCAAGGATTTGCAAAGAAG
AATCAAAGATGTTGCAGTTGATGCAGAAGATGAAGTTGAATCACTAATGA
GACAACTTATTATTATTGAGCTGAATGATGAATGTTGTGGTGCGAAGCTT
GATAAAGTCTCTCAACAGGTTATACAAGTAACTCATTCTGTCAATGAAGA
GCTGATCATCATCAACAATATTAATTGTCCAGAAAAAGCTGATGAAAATA
GTGCTTCTTCTCCACGATTAGGTGATTCAATCCGTGAGAACGTTATGGAA
GGGTACAATGAAGAAAGAGAAAGGATGGTGCAAAGACTTACCAGGGGCTC
AGGATCAAATAGACGGGAAGTGGTCTCTGTTGTGGGGATGCCGGGCATAG
GTAAGACAACTTTTGCCAAAACTATATTATTCGATAACTCTATCAAGAGG
GTCTTTCGTATTCGTGGTTGGATTACTGTGTCTAACAAGTATGATTTAAA
AAAGTTGCTTCTACTGCTCCTTCATGATGTTATTGAAATGAAAGGCAGCA
ATAATTATGATGAAATGGATATTGGACAACTATCTGGTCACGTAAAGCAA
GGTTTGCAGGGACAAAGGTATTTGATTGTTGTGGATGACATATGGAGCAA
TAAAGATTGGGATACAATTTCACATTGGTTTCCAGATTGTGGTGACAGAA
GTCGAATTTTGTTGACTTCTCGAGACTACAAGGTTGGTGAGTATGCTGCA
ACCAATCCTAAAGATGGTTTGGTACTGATGCGTCCTCTGACGCAAGATGA
AAGTCGGAATCTGTTTTACCATAGGGCATTTGGGAAAAATTACAGTATTC
GAGGGTCAGATATTGATGAATTCGAGAAAGTTGGAGAAAAAGTTATAACA
AATTGCAAAGGATTACCACTAATGATTACTGCGGTTGCTGGTATACTCTG
TAGCAAGAGTAAACTGGATGAGTGGATGGAAATAGCTCAAAGTGTAAGCT
CATTAGTAAATGATGATGCTTACTAA

The coding sequence for Solyd04g058020.1, which is hereby incorporated by reference in its entirety, (SEQ ID NO: 7) is as follows:

ATGCTGTATGCGACAACAGATGAACGGAAAATGAAGCATTTCAAGATTCA
AGTACTTGAGGGTGTTAGATTTACGCCATTCAAGATTCTATTAGCATCTA
AGATAGGATGTTATGGAGGCACGGATTATGATAGAGGGTTAGTCGAAAGA
TATGGTACATTGAGTCATGAGGCAGCTGAGATGCTTAAACTTGCTTGTTA
TGATGCTGAACAAGTCATTGACAAGGACGTTGATAGAGATACCGTTATCG
AAGCACTTTTAAAATCAGGAAATGAAGCAACCCTTTTTGTATATCCCATT
GTTGGAGTTGAAGGCATTGGAAAAACTACTCTTGCCAAATTGGTGTATAA
TGATCCAAGGATAGTTAGTCAGTTTCAGCTGCGTATATGGGTTCGTGTGT
CTCGTGTGTTTAAAGTAGAGGAAGTATTAGAACAAATTGTGATTCAGTTA
GAGAAGATTTATGTGAGAAACTTGATATGAATGAGCTGAAGAATCTAGTT
CATGAGACTTTGTATGGAAAGAATTATTTGATTGTGTTAGATGATGTGTG
GAATGAGGACCCAGTGAAGTGGGATGAACTTAAGAAGTTGTTGATGGTAG
GTGGTTGTGGAAGTAAGATTCTTGTAACTACTCGGAAAAAGGAAGTAGCT
TCGATAATGGGGACGGTTCCTGCATACTGTTTGGAGGGTTTGTTCCATGA
AGATAGTCTGACTTTGTTCTTGAGTAAGGCATCTGAACAAGGATCTTCAT
TGCGCAAGAAAACTGAGAGACGGGAGTGGGAGATAGTTAACAATCACAGC
GGGTGGAACTCAACTCAGAATGACGAAATTTCATCTGCACTAAGAGTCGC
AGAAGGTCTCATTAGTAAGTCTAATGAATCAGAAGATCTTGAGGACGTTG
CCATTCAACATTTCCGAGAGTTATTGTCGAGATCCTTCTTTCAAGACGTT
GAAGAATATCGTTCCGTTTATACTTCAATCTGTACAATGCATGACCTTGT
ACATGATCTTGCACTGTCAGCAGCAGGGGTTGAATTCTGTACAGTAAATT
CTCACATACAAAACATTTCTGATGAGGTCAGACATGTGGTGTTTTCTGAC
TATGATTTATCAGGCAAGGAACTGCCAGCATCCCTTCTCGGTAACCAGGC
ATTAAGGACCATATCCTTCTCCGTTGATGGAGTAGGGCCGATGAGTACAA
TGTTCGTTGAGAATTGCTTAGCAAGATTCATGCAACTTAAGGTGCTAGAT
ATCAGTGATTCATGTTTCGATGAGCTGCCTAGCTCTGTTGGCGAATTGAA
GTATTTAAGATATCTTGATGTAAGTTCCAATGGAAGCATTAAAGAATTAC
CTGATTCGATTAACAAGTTGCTGAGCTTACAGACACTTCGAGTTTCTCAT
TGTCCACAACTTGAAGGTCTGCCTAAAGATATTGGAAATTTGATCAGCCT
AAGACACCTGTATATAACCACCAAGCAAGCATGTTTTCCTGAAAAGCAAT
TGGCTGCTTATCATCTCTTCGTTCTTTGTACATTCATAGCTGCAACAATC
TCGTATCTTTGTCTGAAGCACCTTACTGCTTTAAAGAATCTGTTGATTGT
TGACTGTAAAGAGCTTACATTGTTGGAGTGGCAAGATATTGAAGGACTTA
GGATGCTTCGGTCATTGGTTATCGGAGGCTTACCTGAATTGGAGTCAAAA
GATGTTCACTGCCTCGGGAGCCTTCAGATGTTGGTACTTGCTGGTTTACC
AGAGTTAGTTACTTTGCCGCGATGGCTTGAAGGTGCTAGTGCTACTCTAC
AATACCTGAGGGTGGAAAGGTGCCTGAATTTTGCAGCATTGCCAAACTGG
CTGGCAAATCTTACTGCACTTGAAAAACTTGAAATTTCCAAGTGCCGTAA
ATCATTTTCATTGCCCGAGGGTATGAGTTGCCTCACGAATCTGAAGGTAC
TTAAGAACGACAATTGA

The coding sequence for Solyd04g059470.1, which is hereby incorporated by reference in its entirety, (SEQ ID NO: 8) is as follows:

ATGCATTTCCAGAAAAGCTTTAGGAAGAAGGTAAGAAGATTCTTTTCCAG
TTCAAATCCAATTATATATCGATTCAAGATTGGCAGAAAGGTAAAAGAAA
TCAGGGAGCTGCTGAATGAGATTGCAGATGATAGGAGAAATTTCCACTTC
ACGGAACATACTTATGTAATTCCAGCTGAGAATACGAGTAGAGAACAAAC
ACACTCCTTTGTGAGGGCCTCAGATATCATTGGTAGAGATGATGATCAAG
AAAACATTGTAAAACAGCTGATAGATTCTCATGATGAGGAAAATATTTCT
GTGATTCCTATTGTTGGACTTGGAGGGCTTGGAAAAACCACACTTGTTAA
GTTGGTTTATAACAATAATAGGGTTGTTCAGAATTTTGACCTTAGAATGT
GGGTTAGTATTTCAGAAGATTTCAGTCTGAGCAAGGTAATTGAGAAAATT
CTGAGGTCCGCAACAGGAGAGAGTTTTGACCACCTAGATATGGACCAATT
ACAATGTTGTTTGGGAGAGGTTTTGCAACAGAAAAGGTATTTACTTGTGC
TGGATGATGTTTGGAACGAAGATCAACACAAGTGGACGGATCTGAGGGAG
TTGCTGATGAATTGTTCCAGAGGTAGTAAAATTGTTGTCACTACACGTAG
TAAGATGGTTGCTTTGATTACTGGAACAGTTCCGCCTTATTATTTGGGAG
GCCTTGCTAATGATGACTGCTTATCTTTATTTTTGAAATGTGCATTTGGA
GGACAGGACAATTTGTTTCCTAATCTAGTAGAAATAGGAAAAGAAATTGT
GAAAAAGTGTGGAGGAGTGCCTTTGGCTGTGAAAACCTTGGGAAGGTTGT
TGTACATGAAAACAGACGAGAATGAATGGTTGCAGATAAGAGATAATGAG
ATATGGGAAATCGAACAGAATAAATCTGACATTTTACCAATATTGAGATT
GAGCTATGAACAGATGCCATCACATCTAAGACAGTGCTTTGCCTATTGCT
CCATGTTACCCAAAGGTCAAGAAATTCCGAGGGAGGATTTTATCAATCGC
TGGATTGCTCAAGGATTTATACAGAGTTCCAACAGAAACAGGAAGCTGGA
AGATATCGGTAATCAGTACTTTGATGAGTTGCTATCAAGGTTTTGCTTCC
TAGATGTGGTACAAGCTTTTGATGGAGAAATATTGGCTTGTAAGATACAC
AATCTTGTGCATGATCTTGCACAGTCAGTATCAGGTGCAGAGTGCTTAAA
TGTGAAACCCAATGCTTTCGTGGTCTCTGAAAGAGTTCGCCACTTATTTT
TCCATGCAGAAGATATGTCTAGGAAACACTTCCCCAGATTTTTGCTTCCT
TTGCAAAAGTTGAGGTCTTTCTCTTATTCTTTTAACATTGGACCTGTAAA
CAAGTTCTTTGTCAAGACAATGTTGTCAAATTTCAAATGCCTTCGGATGC
TAGTCTTGAACAATCTAGATCTTGAGGAGTTGCCAACTTCGATAGGTCAC
TTGAAGGAATTAAGATACCTCAACCTTAGTGACAGTGGTAAGATCAAGTT
TCTTCCAAGGTCTATGAGCAAATTAGTAAATCTGCACACCCTAAACCTCA
TTAACTGTGAACAGCTTAAGGAGTTGCCAAGAGATTTTAGAAAGTTAATC
AGCCTGAAGACCTTGTATTTGACTACACATCAGATGTCAGCAGGGATCAA
GAATCAACATTCTTTCACTTCTCTTCAATTTTTACTTCTTTTCAAATGTT
GTTTCCCAAAATTGCAGCCAGAACTGGTGCAGCATTTTACTGCACTTCGG
GTTTTGCGTATCTATGAATGCCCAAGTTTATGTTCTCTTCCAAGCAGTAT
TAGATATCTGACTTCACTTGAAAAGCTATGGATCTGGAACTGTGAAGAAC
TTGATTTGATTGATGGAGAAGGGATGTCAGGCCTAACAAGTCTTCAATCC
TTGCTTCTAATGGGGCTTCCTAAGTTGGTGACTCTACCATTGGAACTTAA
AGATACTGCTCCTACAACATTAAAGTACTTCAGAATCGCCGATTGTCCCA
ACCTGGTGGAGCTTCCAGAGTGGCTGCCTAATTGCTCCTCACTTCAGAGA
CTCTATATAGAGGATTGTCCTGTTTTGGCATCGATACCTCAAGGAATCTA
CAGCCACAATGCCAACGTCCATATAATCGACTGTCCATTGCTAGGTGGAT
GA

The coding sequence for Solyd04g059610.1, which is hereby incorporated by reference in its entirety, (SEQ ID NO: 9) is as follows:

ATGGCTGATGCCTTTGTGTCATTTGCAGTTAAAAAATTGGGTGATTTCCT
AATACAACAAGTTTCCCTGAGAAAAAATATGAGAGATGAAGTTACATGGC
TGAGAAATGAGCTACTCTTCATGCAGTCTTTCCTCAGAGATGCCGAACAA
AAGCAATATGGAGATCAAAGAGTTCAACAATGGGTGTTTGAGATCAACTC
TCTTGCTAATGATGCTGTCGCTATACTCGAGACTTACAGCTTCGAGGCTT
GTAAAGGTGATGAGGATGGATTTGCTAGTCGTCTCAAGGCTTGCGCTTAC
ATCTGTAGGAAGGAGAAGAAATTCTACAATGTCGCCAAGGAGATTCAATC
ACTCAAGCAACGAATCATGGATATCTCTCGCAAACGAGATACTTATGGTA
TTACAAATATCAATAGTACTAATTCAGGAGAAGGGCCAAGTAATCAGTCT
GCCATGGTTAGAACATTGAGGAGAACTACCTCCTATGTGGATGACCAGGA
CTACATATTTGTTGGACTTCAAGATGTTGTACGAACATTGCTAGATGAAC
TTCTCAAAGCAGAGCCTCGTCGAAGTGTCCTCTCCATTTATGGCATGGGC
GGATTAGGCAAGACCACTCTTGCAAGAAACCTCTACAACAGTCCTAACAT
AGTCAATAGCTTCCCTACACGCGCTTGGATATGTGTTTCTCAAGAGTACA
ACACAATGGATCTCCTTAGGAATATCATAAAATCCATCCAAGGTTGCACC
AAGGAAACTTTAGATATGTTGGAAAGGATGACAGAAAGAGATCTAGAAAT
ATACCTTCGTGATCTATTAAAAGAACGCAAATACCTTGTGGTGGTTGATG
ATGTATGGAGGAGAGAAGCATGGGAGAGTTTGAAAAGAGCATTTCCAGAT
AGCAAGAATGGAAGCAGAGTTATTATTACCACGCGCAAAGAGGATGTCGC
TGAAAGAGCAGATGACAGAGGTTTTGTCCATAAACTTCGCTTCCTAAGCC
AAGAAGAAAGTTGGGATCTCTTTTGTAGGAAACTACTTGATGTTCAAGCA
ATGGCCTCCACAATGGAGAGGCTAGCTAAAGATATGGTGGACAAGTGTGG
AGGCTTACCTCTTGCAATTGTTGTACTGAGCGGACTACTTTCGCATAAAA
GGGGGCAAGACGAATGGAAAAAGGTGAAAGATCACCTTTGGCAGAACATT
GAAAACAACTCTATTGAAATCTCCTACATACTATCATTGAGCTATAATGA
TTTGTCAATTGCACTCAAGCAGTGTTTTCTGTACTTTGGCCTTTTTCCAG
AAGATGAAGAGGTCGATGCTGAATACATAATATGGTTGTGGATGGCCGAG
GGTTTCATACCTAATGGAGAAGAAAGAATGGAAGATGTCGCTGAAGGCTT
CTTGAATGAGCTGATAAGACGAAGCTTAGTTCAAGTGGCTGGAACACTTT
GGGAGAAAGTTATTCTATGTAGGGTTCATGATGCAGTTCGTGATCTTTCC
ATACAAAAGGCAATGGAGGTAAACTTCTATGACATTTATAATCCAAGAAA
CCATTCCATATCCTTCTTACCTATTAGACATGCCATTCATAGTCAAGGAG
AAAGGTACCTCTCACTTGATCTTTCTAATTTAAAGTTGAGGTCAATTATG
TTCTTCGGTCCAGATTTTCGTAACATGAATCTTATAAACTTTAGTAGTGC
GTTCCAACATATATATGTGTTGTACTTGGATAATCGTGGTGGTTCTATAT
CTATAGTACCTGATGCGATAGGAAGTTTGTACCACCTCAAGTTCTTAAGC
TTGAGAGGCATCCATGATCTTCCCTCCTCCATTGGCAACCTCAAGAATTT
ACAGACACTTTCTGTCGTGAATGAAATTGGACACCCATTCAAACTACCCT
GTGAGTCAGTTGATCTAATAAATCTAAGACATTTAGTTGCTCCGTATACA
AAACCTTTGAAACGTATAAGCAAACTCAAAAATCTTCAAGTTCTTAAAGG
CACTGCTTGTGATCAGTGGAAAGATGTTGACCCTGTTGATTTAGTCAATC
TTCGAGAATTAAGCATGCATGATATTACCAAAATTTACGCCCTGAACAAC
ATTAGCAACTTGAAAAACCTTAGCACACTCAGATTGTTTTGTAGAGGTGG
TGAATCATTCGCTGCCCTTGAATTTCTTAGTTCTTGTCAAAAGCTCGAGA
AATTGTTGTTAAAAGGGAGAATAGAGAAACTTCCTTTGTTTGTAAATTCC
ATCACAATGATGGTTTTTTGGAACTCAAAACTCATGGAAGATCCCATGCC
TATCTTGGGAATGTTGCCAAACCTAAGGGATCTCTATTTAGTAGCAACTT
ATGAAGGAAAAGAAATAATGTGCGGTGACAACAGCTTCAGGCAACTCGAG
TTCCTTCGTCTTCATCGTCTTGAGAACCTAGAAACATGGCATTTAGCCAC
AGGTTCCTTGCCTCTGATTAAAGGTCTTCGTATCTATGATTGTCCAAAGC
TGAAGGAGATTCCAGATAGATTGAAACATGTGAAGCTTTTCAACCATATA
TGA

The coding sequence for Solyd04g060430.1, which is hereby incorporated by reference in its entirety, (SEQ ID NO: 10) is as follows:

ATGTCTTTTGCACTTGGAAAATTGGGTGATTTACTCTTCATAAAATCTTT
CCTCAAAGATGCAGAACTAAAGCAATGTGGAGATCAAAGAGTTGGAGAAT
GGGTATTTGAGATCAACTCTATAGCTAATGATGTTGTTGCTACACTCGAG
CCTTGCATCTTTGAGGTTGGATGTTGTACAAACATTGCTAGCTCACCTTC
TCAAAGCAGAGCCTCGTCGAAGCGTCCTCTCCATTTATGGTATGGGGGGT
TAGGCAAGACCACTCTTGCCAGAAAACTTTACATTAGTCCTAATATAGCC
TCTAGTTTCCCTACACGTGCTTGGATATGTGTTTCGCAAGAGTACAACAC
CATTGATCTTCTTAGGAATATCATGAAATCCCTCCAAGGTCGCACCAAGG
AAACTCTAGATTTGTTGGAAAGGATGATCGAAGGAGATCTAGAAATTTAT
CTTCGTGATCGATTAAAAAAACACAAATGCCTTGTGGTGGTTGATGATGT
GTGGCAGAAAGAAGCATGGGAGAGTTTGAAAAGAGAATTCCCGGATAGCA
AGAATGGAAGCGGAGTCATTATTACCACGCCCAAAAGGGAAGTCGCTGAA
AGAGCAGATGACAGAGAAGATCAAGTGCTCAAGGCTGATAACATAATACG
GTTGTGGACGGCCGAGGGTTTCATACCCAGAGGAGAAGAAAGAATGGAGG
ATACCGCTGAAGGCTTCTTCAATGAGCTTATAATACGAAGCTTGGTTCAA
GTGGCTAAAACATTTTGGGGAAGAGTTACTGAATGTAGGGTTCATGATTT
ACTCCATGATCTTGCGATACAAAAGGCATTGGAGGTAAACATCTTTTACA
TTTATGATCCAAAAAGCCACTCCATATCCTCTTTATGTATCAGACATGTC
ATTCATAGTCAAGGAGAAAGGTACCCCTCACTTGATCTTTCTAACTTAAA
GTTGAGGTCACTTATGTTCTTCGATCCAGATTTTCGTAAGATGAGTCTTA
TAAACTTCAGGAGTGTGTTCCAACATCTATATGTGTTGTACTTGGAGATG
CGTGTTGACAATATGTCTATTGTATTAGTATCTGATGCCATAGGAAGTTT
GTACCACCTCAAGTTCTTAAGCTTGAGAGCTCCAGAAATTGAGGTTAGAA
GGGGTGATAGAGAAACTGCCTCATCTGTTTCCAAATTCCATCACAATGAT
GGTTCTGAGGGACTCAAGACTGACAGAAGATCTGATGCCTATTTTGGAAA
CGTTGCCAAACTTAAGGAATCATGA

The coding sequence for Solyd04g060640.1, which is hereby incorporated by reference in its entirety, (SEQ ID NO: 11) is as follows:

ATGAGAGATGAAGTTACATGGCTGAGAAATGAGCTACTCTTTATACAATC
TTTCCTCAAAGATGCAGAACAAAAGCAAAGTAGAGATCAAAGAGTTCAAC
AATGGGTATTTGAGATCAACTCTATTGCTAATGATGTTGTTGCTATACTC
GAGACTTATAGCATTGAGGCTGGTAAACGTGCTAGTCGTCTCAAAGCTTG
CGCTTGCATATGTAGCAAGGAGAAGAAATTCTACAGTGTTGCCGAGGAGA
TTCAATCACTCAAGCAACGAATCATGGATATCTCTCGCAAACCAGAGACT
TATGGTATTACAAATATCAATTATAATTCAGGAGAAGGGCCAAGTAATCA
TGATTACATTTTTGTTGGCTTTAACGATGTTGTACAAACATTGCTAGCTC
AACTTCTCAAAGCAGAGCCTCGTCGAAGTGTCCTCTCCATTTATGGCATG
GGCGGATTAGGGAAGACCACTCTTGCCAGAAACCTCTACAACAGTCATGA
TATATTCAATAGCTTCCATACACGCGCTTGGATATGTGTCTCTCAAGAGT
ACAACACAATGGATCTTCTTAGGAATACCATAAATCCATCCAAGGAAACT
CTAGATTTGTTGGAATGGGTGACAGAAGGAGATCTAGAAATTTATCTTCG
TGATCTATTAAAAGAACGCAAATACCTTGTGGTGGTTGATGATGTATGGA
ATAGAGAAGCATGGGAGCGTTTGAAGAGAGCATTCCCGGATAGCAAGAAT
GGCAGCAGAGTCATTATTACCACGCGCAAAGAGGATGTCGCTGAAAAGCA
AACGATAGAGGTTTTTCAAGAAGAAAGTTGGGATCTCTTTTGTAGGAAAC
TACTTGATGTTCGAGCAATGGTTTCAGAAATGGGAAGTCTAGCTAAGGAT
ATGGTGGAAAAGTGTAGAGGCTTACCTCTTGCAATTGTTGTATTGAGCGG
ACTACTTTCGCATAAAAAAGGGCTAAACGAATGGCAAAAGGTGAAAGACC
ACCTTTGGAAGAATATTAAAGAAAATAAATCTATTGAAATCTCTAACATA
CTATCTTTAAGCTACAATGATTTGTCAACTGCTCTCAAGCAGTTTTTTCT
CTACTTTGGTATTTTTCCAGAAGATCAAGTGGTAAAGGCTGATAACATAA
TACGGTTGTGGATGGTCGAGGGTTTCATACCCAGAGGAGAAGAAAGAATG
GAGGATGTCGCTGAAGGCTTGTTGAATGAACTGATAAGACGAAGCTTGGT
TCAAGTGGCTAATACATTTTGGGGAAGAGTTACTGAATGTAGTGTTCATG
ATTTACTCGATCTTGCGATACAAAAGGCATCGGAGGTAAACTTCTTTGAC
ATTTATGATCCAAGAAGCCACTCCATATCCTCTTTATGTATCAGACACTT
CATTCATAGTCAAGGAGAAAGGTACCTCTCACTTGATCTTTCTAACTTAA
AGTTGAGGTCAATTATGTTCTTCGATCGAGATTTTTGTAAGACGAGTCTT
ATAAACTTCAGGAGTGTGTTCCTACATCTATATGTGTTTCACTTGGATAT
GAATGTTGGGAATATGTCTATAGATGTACCTGATGCCATTGGAAGTTTGT
ACCACCTCAAGTTCTTAAGCTTGAGAGGTATCCGTGATCTTCCCTCTTCC
ATTGGCAACCTCAAGAATCTACAGACACTTGTTGTCGATGTGGGAGGATA
CACTTTCCAACTACCCCGCGAGACAGTTTACCTGATAAATTTAAGACATT
TAGTTGCTCGGTATTCAAAACCTCTGGTACATATAAGCAAACTAACTAGT
CTTCAAGTTCTTGAAGGTGTTGGTTGTGATCAATGGAAAGATGTTGACCC
TGTTGATTTAGTCAATCTTGGAGAATTAAGTATGTTTGATATTAGCAAAT
CTTACTCCCTAAACAACATTAGGAGCTTGAAAAACCTTAGCACTCTCACA
TTGTCGGGGGGGTATCGGAACTCATCTCCATTTCCAGACCTTGAATTTGT
TAATTGTTGTGAAAAGCTCCAGAAATTGAGGTTAGAAGGGGTGATAGAGA
AACTGCCTCATCTCTTTCCAAATTCCATCACAATGATGCTTCTTTGGAAG
TCAAGACTGACAGAAGATCCGATGCTTATTTTGGGAATGTTGCCAAACCT
AAGGAATCTCGATTTGGTTAGTGCTTATGAAGGAAAAGAAATAATTTGCA
GTGATAACAGTTTCAATCAACTAGAGTTCCTTCAACTTGAGGATCTTCGA
AATCTAGAAAGATGGTATTTAGGTACAAGTGCCATGCCTCTAATTAAAGG
TCTTGGTTTCCATGACTGTCCAAATTTAAAGGAGATTCCTATGAGAATGA
AAGACGTGGAGCTGCTGAAGAGGAATTATATGTGGTGA

The coding sequence for Solyd04g061490.1, which is hereby incorporated by reference in its entirety, (SEQ ID NO: 12) is as follows:

ATGGAAAATGAATATCAGAAGCTGGAGGATATCAGAATTGGGTTGCATCA
AAGAGCGGAGGCTGATCGGAGAAACTTACTAGTCATTTCACCTAATGTTG
AGGCTTGGTTTACTCGTGTTGATACTACTACTGCAGATGTGGAAGCTGTA
ATGCGACGAGAGGTTGAAAGATATGGCTGGTGCCCAAAATTGAAGTCACG
TTACTCGCTGAGCAGGAAAGCTAAAAAATTTGCACTGGAATTGATTGAAC
TTCGAAATCAAGGCAATGATTATGCTGTTTTCTCCTATCCTGCAGTCAAA
ATTGAAGTTCTACCTAGTAACAGTGGTGAGGAGTTTGACTCCAGAAAATT
GCAAGAGGAAGAGGTTATGGCAGCTTTGAGAGATGATGGGGTCACTATGA
TTGGGATATGTGGTATGGGTGGTTTTGGTAAGACAACACTGGCTGAGAAA
ATCAGGCAAAAGGCGAAACAAGGAAGTTTGTTTGATGAGGTTGTCATGGT
AACTATCAGTCAACAACCAGACTTGAAAAGAATTCAAGGTGAGGTAGCAG
GGGGTGTTGGTCTGACGTTGCAAGGGGACAATTTCTGGAATCGTGGAGAT
CAGCTGCGCTCAAGGTTAATGGTTCAGAACAGCTGTGTCCTAGTAATCTT
GGATGATGTTTGGGAGGCTCTTCATGAACTAGAGAAACTTGGAATTCCCA
GATGTAGCAACCACAACCAACGTTGTAAAGTGACATTGACAACGCGATTC
CGAGATGTTTGTGAAGCTATGGAAGCTCAAAAGATCATGGAAGTTGGAAC
TTTACCTGAAGAGGAAGCATGGATCCTTTTCAGGGAGAAAGTTGGTAATA
TAATCGACGACGATCCTTCTGTACTTGACATAGCAAAAGTTGTTGCCAAA
GAATGCAAGGGGTTGCCGCTTGCAATTATTACAGTTGCAAGAGCACTTAA
GCGTAAAACCAAGCCTTCGTGGGAGGATGCCCTTGTACAATTACAAAGAT
CAACACCAATAAATATTCCAGGAGTAATTAAAAATGTGTATCAATCTCTC
AAACTTAGCTATGATTACTTGGAAAGTAATGAAGTCAGGTCCCTGTTTTT
GCTTTGTTCCTTGTTCGAGGAAGATAGTAATATCTGGACTGAACAATTAC
TTAGATATGGAATGGGGCTTGACATCTTTTCAGAAATTAAAAACTTAGAA
GAAGCAAGGAAGCGGGCGTGTCATCTCTTAGAAACATTGAAAGATCGTTT
CTTCTTGTCCTTAGGTTCAACAGAAAATTATGTCAAAATGCATGATATGG
TCCGTGATGTGGCTATAAGTATTGCCTCTATGGGAGAGTATAGCTTTATG
GTAAGTCACCATGTGAACTCGCAAGAGCTCCTAAGAAGAACTTCTTACAA
GCAATACAGTCACATTTCAATTGTTGCAAATAAATTTGATGAGCTTCCTA
ACCCAATATTTTGCAAAAAACTGAAGCTTCTAATGTTGACTCTCGATTTT
AAAAATCCATTTAAATTACAGGAAGATTTTTTTGATGAAATGGCTGAACT
CAATGTCTTAAGTTTGAGTGGATATGGATCCATTCTGCCTTTTCCAACAT
CCATTGAGAGGTTGTCAAGTTTGAAGACATTATGTCTGAGTCATTTAAGG
TTAGATGACATATCCATTATTGGGATACTTGTCACTTTAGAAATTCTCAG
CATAAGACATTCTGACATAGAGGAGCTGCCAGTTGAGATAGGAAATTTGA
CCAATCTAATTATGTTAAGAGTTTCGGAATAG

The coding sequence for Solyd04g064750.1, which is hereby incorporated by reference in its entirety, (SEQ ID NO: 13) is as follows:

ATGGATCATCTTATGCCTTACCTGCTGAAAGAAATCAAAGATTACTATTA
CAAGGAGTATGAGATCTCTCAATGCTCTTCCGAGATCTCTTCTCTACTTA
TCGACATAATGCCTCTTGAGCTGGAGGTTCTATACATTTTTACTTCTAAG
CTCATGAAAGAATCAAGGTCAACAGAACTAGAAGTGTTTGTTAAGCAAAT
CCTAAAAGCATCTCCGAAGATTCTTCAAAATTATCTGATTCTTATGAAAG
GATGCATGGAAGGTTCAGTAGCTGTCAGTTACGCTCCAATTCAATGCATT
AATGTCATGATGGAGTTCCTATTGATCTTTCTCACTTATATACCAAAGCG
CTATATCCGTCGTGACAAACTGAATGATATGTTGGCACATGTTGGAGTAC
TTACAAGGAAGATATCTATTCTGGTGAGCAAGCTGATGGAGGAGAGCTCT
GAGAATAATATCAACGAAGCGGACTTTTCAGCTGCATACTTGTTGCAAGA
AATTGAACAAATGAAGGGAGATATCAGACAGCTTTTTTTGAAAGCCCCAG
AGTCATCTCAAATTCGCTTTTCTATGGATGATGGTTTACTCTTCATGAAT
CTTCTACTCGGACATTTAAATGATTTGCTCATTTCCAATGCTTATTCAGT
TGCTCTGATAAAAAAAGAAATTGGGATAGTGAAAGAAAGCCTTGAATTCC
TAAGATCATCTTTCGGGGAAGTCAGGCAAACATTGGATGACACTAGCGGA
TTAGTTAAAGATTGTTGGGTGCGTGCTTTAGATGTGGCATATGAAGCAGA
ACATATCATTAATTCCATTCTTGTCAGAGATAATGCTCTCACACATCTCA
TATTCTCACTTCCGAATGTCACAGATAAGATCAAGCTTATCGTGGCAGAA
GTCACCTGCAGTGTACATCTGGAGGATAAAAATGGGGATGACCCCCTTGA
TGCAAAGTCTTTCGACGAGTCAATTGAGTCAACCTCATCATCTTTTGTTG
AGGTAACAGTTGGTCATGAGGAAGACGAAGCCTGGATCGTTGACCAGCTC
CTTGATAAGCATGAATCCAAGCTTGATGTCATTTCGATTGTCGGAATGCC
AGGAGTCGGTAAAACTACTCTAGCCAACAAAGTTCATTTCAATGTTCATG
TTTGGTGCACTGTTTCCCAAAAGTTTAACAAGTCAAAGGTGTTGCGGGTG
ATTCTTCAGCAAGTTACAGGGTCGGAAGACAAGAAACAAAGTAATGAGGG
TGATAAAGTTGTTGATCTTGCTGAAAAGCTACGAGAAGAACTATACGATA
AAAGGTACCTCATCGTCTTGGATGATGTGTGGGATATTGCAACAGTGGAG
ATGTTAATAGCATGCTTTCCGAGGGTTGAGAGAGGAAATAGAATTATCTT
AACTAGCCGAAGTAGTGAGGTAGGTTTGCAAGTTAAATGTCGTAATGATC
TTCTCTACCTTCAACTTCTAACACATGAAAAAAGTTGGAATTTATTTGAA
AAAAGGATCAAGGAAGCTGCCCTGCTGAACTGTCAGAAGTTGGACACCAA
ATATATAGTTGAGAAATGTCAAGGGCTTCCTCTGGCTGTTGTGTTGATTG
CTGGAGTAATTGTTAGAGGAAAGAAAAATGAAAAAGATTTGTGGCTTAAG
ATTCAACATAATATGGATTCCGTTATTTCTGCCAACAACAATTTGCAGAT
GATGAAGGTTATGCAATTAAGTTATGACCACTTACCATACCACCTAAAGC
CGTTGTTGCTTTACTTTGGAAGATCTCAAAAGAACAAACTAACTCCAGTC
TCTAAGTTGATGCAATTGTGGATGGCCGAAGGGTTTGTGGATCATTGTAT
CCCGTCTAAGAGTAGTTTAGAGGAAATAACCCAAAGTTACTTGGAAGCTT
TAATTTCCAGTAGCCTGGTAATGGTGGATCGTAGCGTGTCCAAGAGTAGT
CACCCTTTTTCTGTTACTATCAAGGTTTGCTATGTGCATGATGTTGTGCA
TGATTTTTGTTCAGTAAAAGCAAAGAAGGAAATGTTTTTCAAGTTAATCA
ATTCAGGTGCTCCATTTCATGCTTCGGATTTCATACATCGTCGTCTAACC
ATTTATACTGACAAATCCCAACTCCACAAAAGATGTGTTTTGTTTAATTC
TAATAAGTGTTCAGCTGGTAGTAAGCATCTCATATCTTTGAAAGTGAAAA
ATTGGCTTGATCCCTTCAGTCACATTAGACACTTTGGACTTGTTAGAGTG
TTGCAACTTGGTAACATTATTCTGGAAGAGTCATCAATGGAAGAAATAGG
CTCCCTATTTCATTTGAGGTTTTTGAGGTTTCAGACATATGAAAAATCTC
TCCCGCTTTCGTGGTTGAACCTCCAGAATCTGGAAACTCTGTGGATCCTA
AACAGGCATTCCACCATGGTACTACTCGACGAACTGTCAAAGCTGAAACA
TGTGAGCATCGACGCTAGATCTTTCTTTGAAGAGGACAAGGACAACATTA
TGCATCAGCCAAGTAGAATATTGGAAGCTGAGTGTTCAAAGTTAGAATAC
TTGACAACTTTATCCCGAGTTGATATCTCATATTCTCAAGGCACAACTGA
TGCTCTGGGGAAGTTCCCAAATCTTCAGGACTTTGATTGCAACATTCTGG
TACCGAATGATCCTCCTGCAAACGGCGATTGGTTTCCCAAGTTTGATGTC
CTTAATAAACTTGAATCACTCATTCTAAGTTACAGTTATGTCTGGAGCAG
TACTGATAAATGTCTGGATTATTCCAAGTTTGAAAAAATCCGAAATCCCA
ATGAATATCACTTCCCTACCAGTTTGCAAGAGTTACGGTTGCATAGGTTT
CCCCTGAGACCTGCTTTGTTGTTAGCAATCGCGACATTGTCTGAGCTTGA
AATTCTGGAGATTATAGAATCTAATTTCCTCGAGGATGAGTGGGACGCAA
GTAAGGACATCTATCAAAGTCTAAAGACTTTGCATTTAGCAGATGTCTTT
CTTACAGAATGGCAAGTTGATAAGGGAACTTTTCCCAAGCTTGTGGAATT
AAAACTAGAACATTATCACGGGCTTATGGACATCCCTTATGCATTTGGGG
ATATAGACACTTTAAAGTCCATTCGAGTGGTTCAAACAAGCCGTCACCTT
GGAAATTCAGCCACAAAAATTAAGGAAGATGTAGAAGCTTACACGGGAAA
GGAAAGACTTAATGTCCAC

The coding sequence for Solyd04g067320.1, which is hereby incorporated by reference in its entirety, (SEQ ID NO: 14) is as follows:

ATGGATGGATTGGCTGAAACTGGATCTTCTTCTTCTTCTTCTTCTTCTTC
TTTGTGGCCATGCACTTATGATGTTTTCTTAAGTTTTAGAGGAGACGATG
TACGGAAGAATTTCGTCGATCATCTATATACAGCTTTGCAGCAAAGAGGA
ATTCACACTTTCAAAGATGATGAAAAACTTGAAAGAGGGAAATCTATTTC
ACCTTCACTTTTCAAAGCTATCGAAGAATCGATGATTTCCATCATCATAT
TCTCTCAAAACTATGCTTCTTCTTCGTGGTGTCTAGATGAGCTTGTTAAG
ATCACTCAATGCATGAAACTCAGGGGACAGATTGTTCTTCCCGTCTTCTA
TGACGTGGATCCATCTGTCGTAAGAAAACAAAAGGCAAATGTTGGTGAGC
TCTTCGCTAAACATGAGTTAGATTTCAAAGATGATGAAGAAAGGGTGAAG
AGATGGCGTACTGCTATGACAGAAGCAGCAAATGTATCTGGTTGGGATTT
GCCAAATATAGCTAACGGGCACGAATCAAAGTGTATCGATCAAGTTGTAG
AATGTGTCATGGAGATATTAGGTCATACTGCTTCTGATGCTACTGAAAGT
CTTATTGGGATACGCTCAAGAATGGGGACGGTGTATTCCTTGTTGAATCT
GGAGTCTGATAAAGTTCAATTCGTTGGAATATGGGGAATGAGCGGAATAG
GAAAAACAACTATAGCAAGAGCCATCTATGACAAGATTTTCCGTTACTTT
CAAGGTGCTACTTTCCTTCATGAAGTTGGAGAAACTTCAGCCAAACATGG
TATCCAACATTTGCAGAAGATACTTCTTTCTGAACTACTTCTGTTAAAAG
ATCTAAGAATAAACAACGTATTTGAAGGAACCAGCTTGATAAGAAGACGA
CTAAAAGGGAAACGAGTCCTAATTGTTCTTGATGATGTCAATCATGGAAA
CCAGTTAGATGCCCTAGCTAAAAGCCATGACTGGTTTGGTGCAGGCAGTG
TAATCATCATAACAACAAAGGATAAGCAGTTGCTTCGTCAATATAACGTG
GACAAAATGTATAAAGTGAGTCTGTTAAACACTGATGAAAGTATTGAACT
CCTTAGTTCGTATGCATTCCAGAATCGTCATCCCAAAAGTGGATATGGAG
AGATTATAGCTGAAGTTGTTCGGTATGCTGGTGGTCTTCCTTTAGCTCTT
AAAGTTTTGGGTTGCTCTCTGTATGGCGGAGGCATGATTGAATGGAGAGA
AACAGTGGAGAGACTAAAACAAATTCCAGAAGGCGAAATTGTAGAAAAGC
TCAAAGTAAGTTTCAATGGACTAAGTGAGACTGACCAAAAGATCTTCTTA
GATATTGCATGTTTCTTTAAAGGGAAGAAGAAAGGTTCTGTCATTAGAAT
TCTTCGTAGTTTCAGTTTTACTCCTGTCGTTGGCATAAGGAATCTCATCG
AAAAATCTCTTGTAACTGTTTCAAAAGGTAGGATTGTGATGCATCAGTTG
ATCCAAGAGATGGGTTGGCATATTGTTCGCAAAGAAGCTTCAAACAATCT
TGGCAAGTATACTAGGCTCTGGTCTACCGATGATATTCTTCAGGTACTAT
CTGAAAATACGGCCACAGAAGCTGTGGAAGGCATATGGTTGCACTTGCCT
ATACCGAAAGACATAAATGTTGGTGCAGAAGCCTTCAAACAAACGGACAA
CCTGAGGCTGCTCAAGATACACAATGCAAGTGTCTCTGTAGCTCCAGATT
GTCTTCCTAATAAATTGATATGGCTTCATTGGCATGGCTACCCAATGAAG
TCACTTCCAGCAAGTTTTCGAGCAGAAAGGCTTGTTTGTCTGAAAATGCA
GTATAGCCGCGTTGTACACTTGTGGAAGGGAGTAAAATTCCTACACAAAC
TGAAGTTTCTCAACCTTAGTCACTCCCAAAAGCTAGTCAGCTGTCCAGAT
TTCACAGGGGTGCCCAATCTCGAAAAGTTGGTTCTTGAAGATTGTTCGAG
TATAACTGAGATCCATCCTTCTGTGGGATATCTCAAAAATCTTGTTCTAC
TAAACCTGAAGAACTGCAAGAATCTTAAGACCCTTCCAAACATTATTCGA
TTGGATAATCTTGAGACTTTAATTCTTTCTGGCTGCTTGAAACTCGCGAA
TTTCCCAGAAATCATGAGTGACATGAATTGCTTATCTGAGGTCTACTTGG
AAGCTACAGATGTAAAAGAGTTGCCTTCATCAATTGAACGCCTCCCTGGC
CTTCGATTGATGAATCTAGGCTACTGCAGGAATCTTACAAATTTACCAAA
AACCATAGGTAGATTAAAATCTCTTAGGATTCTTATTCTTTCTGGATGTT
CAAAGCTAGAAAAGTTGCCAGAGGAACTGGGACATATAGAAATCTTGGAG
GAACTCTATTGCGACGAAACTTCCATTCAAAGCCCACCATCATCCATTAC
ACTGTTGAAGAACCTTAAGACCTTATCTTTTCATGGATGTAAAGGCATGG
TATCTCAATCATGGAGTTCACTTTTCTATGCATGGCTTCAGCCAAGAAAA
CATAATCACAAGCCAACAAGTCTGATGTTTACTTCCTTTTCTGGTTTATT
TTCTTTGAGGAAATTGGATCTTAGTGACTGTTGTATGTTGGATGAAGGAA
TTCCTAGTGATCTTGGATGCTTGTCTTCTTTGGTTGAGCTAAATCTTAGT
GGGAATAATTTTGTGGATCTCTCTCAAGCAAGTCTCAACATGCTTCCGCG
GCTCAGAATCCTTGAGCTAGTTGGTTGTGAGAGGCTTGAAAGGTTGCCAG
AACTTCCAACAACAATAGAGGAAGTTTTTGCAGATAATTGTACATACCTG
ATGACTGATAACATGGGAATATTGACCAACTACAAGATGTTACAGCGAAT
ATCGTTCACTAATTGTGTTGGACTACTGCAGAATCAGCAGACGCGTGACA
TGGCTACCTCATTGTGGCTTCACCTATTCAAGAAGTGCATTGTTAAAAGT
GGTCACTTTAGCATTTACCTACCTGGAGAACAAGTTCCAGAATGGTTTGG
CTACAAACTCAACGGAACTTCAGTTTCAATGCAGCTGCCAAACGATTGGT
ACAATGATAAATTCATGGGATTTGCCATCTGTGTTGTGTCTGAACTTGAA
ACAACATGGTTATCAGTTCATGAAGGTTACCTACAGGAAATGCCAGGCAT
TTCGATTGAGTTTACAATCAAAAGCCACCTCCGGAGGAGCACGAGTTGCC
TAATGAACATTGGTTTTGTAGGGACAAACAAGAATGTTGCTTCAGATCAC
ACATGCCTTGCCTATGTGCCATTTGAAGAATATTGGTCAATGTACAAAAA
CCACTTAGACAGCCCAAACAATTGGTATCAGATCGATTTTTCTGCAAACT
CTTTGAGGAAACATATATTCCTAAAAAGTTGGGGAATTCGTCTCGTGTAC
ACTGATGACTTACAGATTTTCACATCTTGA

The coding sequence for Solyd04g076500.1, which is hereby incorporated by reference in its entirety, (SEQ ID NO: 15) is as follows:

ATGGCGGTGACGGACTTTTTCGCCGGCGAAATTACCACCGAACTCATAAA
ATACCTGCTGTTAATAGTTAAAAAATCCACTTTATGCCGTTCAAGCGCAG
AGAATCTCATTGACAATATCAATGGTCTCCTTCCAATCATCCAAGAAATC
AAACAAACCGGTGTTGAACTTCCACAGATACGGCAAACTCAGCTCGACGA
TTTCTCCAAACTTCTCCGAGATGGCTACGAACTCGCCGGAAAAGTTCTGC
ACTCCGGCCGTTGGAACATGTACAGGAACCTACAGTTGGCTAGGAAAATG
GAGAGGTTAGAGAAAAGAGTAGCGAGGTTCATGCAAGTTACAATGCAAGC
TCATGTACTAGCTGATGTTCATCATGTTAGGTTTAATATGGAGCAGAGAT
TTGATGTGCTTGAGCATAGGCTTAAAGCTATAAAAATCGGAGTTGACGAT
AGAAGTGGTGGAGGAGGAGGGTGTTTAGGAGAAGCTGTGAAAAGAATGGA
AGAAGATGAGAAATGGTTTGAGGATAGTTTTGTAAATTTAGGTGCTGGGA
TTGAATTGGGGAAGAGGAAGGTGAAGGAGATGCTGATGGGTGAACAAGAT
AGGGGTGTGTTTGAGATTTGTGGAATTGGGGGTAGTGGCAAAACTACCTT
GGCTAAGGAGATTTGTAAAGATGATCAAGTTAAAAGTTATTTCAAGGACA
AGATTTTCTTTTTCACTGTTTCTCAATCTCCAAATGTGGAGCAATTAAGG
AAAATGATTTGGGAAAAGATATCAGGGTGCAATCTCCATGGTTATGGATA
CGGGGAGATGTTTCCCCAGTGGAACCTACAGTACCAATGGAATACGAAAT
GTGCATCCCCGGTACTCTTGATTCTTGATGATGTGTGGTCTGCATCTGTT
CTAGAGCCACTAGTTTTCAAGATCCCCGGATGCAAGATCCTAGTTGTATC
GCGCATCAAGTTTCCTCTATCGATCATTGACTGTATTTATGATTTAGAGT
TGTTAAGGAAGATGAAGCTATGTCCTTATTTTGCCATTTTGCTTTTGGAC
ACAATTCCTTTCCGCGTGATTGTGGATGAATGTGAAGGGCTTCCTTTGGC
TCTTAAGGTCATTGGATCTTCATTGAAGGGAAAACCTGAGATGTTATGGA
CAAGTGCAAAAAACAGATTATCACGATGCCAACCTGTCTGCGAGTCTCAT
GAACTGCAGTTGCTTGAGCGAATGAAATTGAGTATTGACTGTTTGCCTGA
GAAGGTGCGAGAGTGTTTCCTGGACTTGGGTGCTTTCCCGGAGGACAAAA
GGATTCCTGTTGATGTTCTAATTAACATGTGGGTGGAGCTACATGATATT
GATGAGGAGGAGGCTTTTCACATTCTTGTTGAACTTTCAGACAAAAATCT
CCTAAATCTAGTCAAAGATGCACGAGCCGGAGACATGTATACAAGTTACT
ATGAGATATCGGTGTTTCAGCATGATGTATTACGAGACCTAGCAATTCAT
ATGAGCAACTGTGATGATATAAATCAGAGAAAGCGATTGGTTATGCCGCG
GAGAGACACAAGCTTCCCAAGAGAATGGGAAAGAAATGTGGACGAACCTT
TCCATGCACGAGTTATCTCTGTGCATACAGATGAAATGAGAGAAATGGAC
TGGTTCAGAATGGATTGCCCGAAAGCTGAAGTACTGATTCTCAACTTTGC
CTCATCTGAGTACTTCTTGCCTCCTTTTCTGGAGAACATGCCAAAGCTAA
GGGCATTGATAATCATAAACTATAGTGCTGGCAATTCAGTTCTTCATAAC
ATGTCTGTATTCAGTCATTTAACCAACTTGAGAAGCCTTTGGTTCGAGAA
GATATCCATCACTCACTTATCTGACTCCACAAATCCTCTCAATAACCTGC
GAAAGATATCTCTAGTGCTTTGTGACATGAAAAACAGCTTCGATGAGTCA
GATGTAGACCTCCCTGGTTTGTTCCCACAGCTCTCGGAGTTCACAATGGA
TCATTGCATCAACTTCAACAAGCTACCATCGAGCATTTGCCGGTTGCATA
AGCTCAACAGCCTTAGTATCACTAATTGTGATAGTCTTTGTGAACTTCCA
TCTGATTTAGGTGAATTACAAACTCTACAAGTTTTAAGGATATATGCCTG
TCCACATCTGAAAAGGCTTCCCCCGGGAATTGGTCATCTGGTAAAGCTAA
AGTACCTTGACATTTCACAATGTGTTGGTTTGAGATGTCTCCCTGAAGCA
ATTGGTTGCTGTAGAAACTTAGAGAAGATTGATATGAGGGAGTGCCCTCA
AATCGACAGTTTGCCTAGCGCTCTATCCTTTCTTGAATCATTACGTTGTG
TTATCTGTGACGATGAAGTTTTTTGTCAATGGAAAGACGTTGAGAAGGCT
GTACCAGGTCTCTGTGTACAGGTTGCCGAGGAGTGCCATACTCTTGACTG
GCTATCTCAATAA

TABLE 4
Gene models with the highest similarity to RPS2 and Mr5 in
S. lycopersicoides. A genome-wide comparison between all
proteins from S. lycopersicoides LA2951 and the amino acid
sequences from RPS2 and Mr5 was performed to identify the
most similar proteins present in this tomato accession. A pairwise
alignment between each gene model and RPS2 (a) or Mr5 (b) amino
acids sequences was done to calculate the percentage of similarity.
References: Fahrentrapp et al., “A Candidate Gene for Fire
Blight Resistance in Malus × Robusta 5 is Coding for a CC-
NBS-LRR,” Tree Genet. Genomes 9:237-51 (2012); Bent et
al., “RPS2 of Arabidopsis thaliana: a leucine-rich repeat class
of plant disease resistance genes,” Science 265:1856-60
(1994), which are hereby incorporated by reference in their entirety.

| | % Similarity | Amino Acid | | |
|---|---|---|---|---|
| Gene Model | RPS2 | Residues | Annotation | |
| Solyd08g052660 | 56.7 | 1,314 | NB-ARC | LRR |
| Solyd11g052900 | 56.1 | 743 | NB-ARC | LRR |
| Solyd10g073720 | 56.1 | 996 | NB-ARC | LRR |
| Solyd06g059160 | 54.8 | 740 | NB-ARC | LRR |
| Solyd02g054510 | 54.6 | 1027 | NB-ARC | LRR |
| | % Similarity | Amino Acid | | |
| Gene Model | Mr5 | Residues | Annotation | |
| Solyd11g070490 | 69.3 | 916 | NB-ARC | LRR |
| Solyd11g069520 | 62.5 | 1329 | NB-ARC | LRR |
| Solyd03g059570 | 62.3 | 1226 | NB-ARC | LRR |
| Solyd11g069260 | 62.3 | 1431 | NB-ARC | LRR |
| Solyd11g070530 | 61.9 | 1244 | NB-ARC | LRR |

### Example 10: Identification of Ptr1

Further analysis confirmed that the gene conferring AvrRpt2-mediated Pst race 1 resistance (Ptr1) is Solyd04g059470, the coding sequence of which (SEQ ID NO:18) and amino acid sequence of which (SEQ ID NO:19) are as follows:

>Solyd04g059470 (2472 bp nucleotide)
(SEQ ID NO: 18):
ATGGCGGAATCTTTCTTGTTCAATATCATTGAACGGGTTTTGGCTAAAGT
TTCTTCAATTGCTGTATATGAGATCAGTCTAGCTTGGAATGTTAAGACAG
AGCTAAGGAAACTCCAAAGTACTCTATCCACCATCAAAGCTGTACTTCTA
GATGCAAACGAGCAAAAGGCAAAGAACCATGAAGTGAGAGATTGGCTGGA
AAAGCTCAGAGATGTTGTTTATGATGTCGATGATTTGATGGATGATTTAT
CAACACAACTGTTGCTGCAAATGCATTTCCAGAAAAGCTTTAGGAAGAAG
GTAAGAAGATTCTTTTCCAGTTCAAATCCAATTATATATCGATTCAAGAT
TGGCAGAAAGGTAAAAGAAATCAGGGAGCTGCTGAATGAGATTGCAGATG
ATAGGAGAAATTTCCACTTCACGGAACATACTTATGTAATTCCAGCTGAG
AATACGAGTAGAGAACAAACACACTCCTTTGTGAGGGCCTCAGATATCAT
TGGTAGAGATGATGATCAAGAAAACATTGTAAAACAGCTGATAGATTCTC
ATGATGAGGAAAATATTTCTGTGATTCCTATTGTTGGACTTGGAGGGCTT
GGAAAAACCACACTTGTTAAGTTGGTTTATAACAATAATAGGGTTGTTCA
GAATTTTGACCTTAGAATGTGGGTTAGTATTTCAGAAGATTTCAGTCTGA
GCAAGGTAATTGAGAAAATTCTGAGGTCCGCAACAGGAGAGAGTTTTGAC
CACCTAGATATGGACCAATTACAATGTTGTTTGGGAGAGGTTTTGCAACA
GAAAAGGTATTTACTTGTGCTGGATGATGTTTGGAACGAAGATCAACACA
AGTGGACGGATCTGAGGGAGTTGCTGATGAATTGTTCCAGAGGTAGTAAA
ATTGTTGTCACTACACGTAGTAAGATGGTTGCTTTGATTACTGGAACAGT
TCCGCCTTATTATTTGGGAGGCCTTGCTAATGATGACTGCTTATCTTTAT
TTTTGAAATGTGCATTTGGAGGACAGGACAATTTGTTTCCTAATCTAGTA
GAAATAGGAAAAGAAATTGTGAAAAAGTGTGGAGGAGTGCCTTTGGCTGT
GAAAACCTTGGGAAGGTTGTTGTACATGAAAACAGACGAGAATGAATGGT
TGCAGATAAGAGATAATGAGATATGGGAAATCGAACAGAATAAATCTGAC
ATTTTACCAATATTGAGATTGAGCTATGAACAGATGCCATCACATCTAAG
ACAGTGCTTTGCCTATTGCTCCATGTTACCCAAAGGTCAAGAAATTCCGA
GGGAGGATTTTATCAATCGCTGGATTGCTCAAGGATTTATACAGAGTTCC
AACAGAAACAGGAAGCTGGAAGATATCGGTAATCAGTACTTTGATGAGTT
GCTATCAAGGTTTTGCTTCCTAGATGTGGTACAAGCTTTTGATGGAGAAA
TATTGGCTTGTAAGATACACAATCTTGTGCATGATCTTGCACAGTCAGTA
TCAGGTGCAGAGTGCTTAAATGTGAAACCCAATGCTTTCGTGGTCTCTGA
AAGAGTTCGCCACTTATTTTTCCATGCAGAAGATATGTCTAGGAAACACT
TCCCCAGATTTTTGCTTCCTTTGCAAAAGTTGAGGTCTTTCTCTTATTCT
TTTAACATTGGACCTGTAAACAAGTTCTTTGTCAAGACAATGTTGTCAAA
TTTCAAATGCCTTCGGATGCTAGTCTTGAACAATCTAGATCTTGAGGAGT
TGCCAACTTCGATAGGTCACTTGAAGGAATTAAGATACCTCAACCTTAGT
GACAGTGGTAAGATCAAGTTTCTTCCAAGGTCTATGAGCAAATTAGTAAA
TCTGCACACCCTAAACCTCATTAACTGTGAACAGCTTAAGGAGTTGCCAA
GAGATTTTAGAAAGTTAATCAGCCTGAAGACCTTGTATTTGACTACACAT
CAGATGTCAGCAGGGATCAAGAATCAACATTCTTTCACTTCTCTTCAATT
TTTACTTCTTTTCAAATGTTGTTTCCCAAAATTGCAGCCAGAACTGGTGC
AGCATTTTACYGCACTTCGGGTTTTGCGTATCTATGAATGCCCAAGTTTA
TGTTCTCTTCCAAGCAGTATTAGATATCTGACTTCACTTGAAAAGCTATG
GATCTGGAACTGTGAAGAACTTGATTTGATTGATGGAGAAGGGATGTCAG
GCCTAACAAGTCTTCAATCCTTGCTTCTAATGGGGCTTCCTAAGTTGGTG
ACTCTACCATTGGAACTTAAAGATACTGCTCCTACAACATTAAAGTACTT
CAGAATCGCCGATTGTCCCAACCTGGTGGAGCTTCCAGAGTGGCTGCCTA
ATTGCTCCTCACTTCAGAGACTCTATATAGAGGATTGTCCTGTTTTGGCA
TCGATACCTCAAGGAATCTACAGCCACAATGCCAACGTCCATATAATCGA
CTGTCCATTGCTAGGTGGATGA
>Solyd04g059470 (823 amino acid 94.9 kDa)
(SEQ ID NO: 19):
MAESFLFNIIERVLAKVSSIAVYEISLAWNVKTELRKLQSTLSTIKAVLL
DANEQKAKNHEVRDWLEKLRDVVYDVDDLMDDLSTQLLLQMHFQKSFRKK
VRRFFSSSNPIIYRFKIGRKVKEIRELLNEIADDRRNFHFTEHTYVIPAE
NTSREQTHSFVRASDIIGRDDDQENIVKQLIDSHDEENISVIPIVGLGGL
GKTTLVKLVYNNNRVVQNFDLRMWVSISEDFSLSKVIEKILRSATGESFD
HLDMDQLQCCLGEVLQQKRYLLVLDDVWNEDQHKWTDLRELLMNCSRGSK
IVVTTRSKMVALITGTVPPYYLGGLANDDCLSLFLKCAFGGQDNLFPNLV
EIGKEIVKKCGGVPLAVKTLGRLLYMKTDENEWLQIRDNEIWEIEQNKSD
ILPILRLSYEQMPSHLRQCFAYCSMLPKGQEIPREDFINRWIAQGFIQSS
NRNRKLEDIGNQYFDELLSRFCFLDVVQAFDGEILACKIHNLVHDLAQSV
SGAECLNVKPNAFVVSERVRHLFFHAEDMSRKHFPRFLLPLQKLRSFSYS
FNIGPVNKFFVKTMLSNFKCLRMLVLNNLDLEELPTSIGHLKELRYLNLS
DSGKIKFLPRSMSKLVNLHTLNLINCEQLKELPRDFRKLISLKTLYLTTH
QMSAGIKNQHSFTSLQFLLLFKCCFPKLQPELVQHFTALRVLRIYECPSL
CSLPSSIRYLTSLEKLWIWNCEELDLIDGEGMSGLTSLQSLLLMGLPKLV
TLPLELKDTAPTTLKYFRIADCPNLVELPEWLPNCSSLQRLYIEDCPVLA
SIPQGIYSHNANVHIIDCPLLGG*

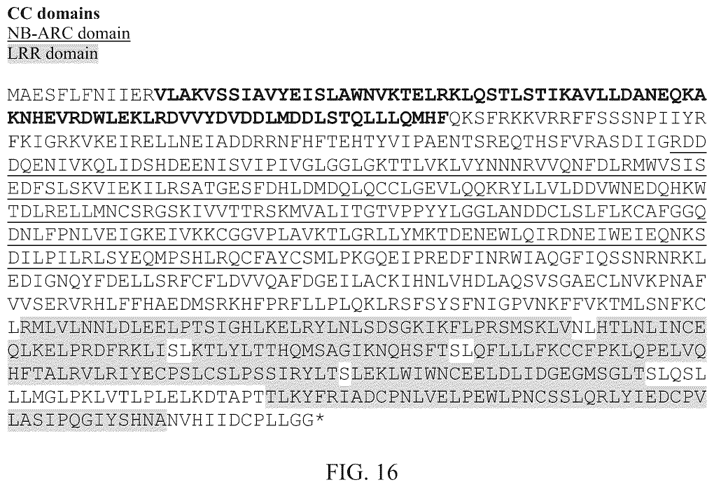

An annotation of SEQ ID NO:19, showing the location of coiled-coil (CC), nucleotide binding (NB-ARC), and leucine-rich repeat (LRR) domains is depicted in FIG. 16.

### Example 11: Discussion of Examples 1-10

From a serendipitous observation during a natural occurrence of bacterial speck disease two S. lycopersicoides introgression lines (ILs) that have strong resistance to multiple race 1 strains of Pst were identified and further characterized. Such resistance is important since race 1 Pst strains are becoming increasingly common throughout the world and yet no simply-inherited genetic resistance to these strains is known. The two ILs contain a large overlapping introgression segment from S. lycopersicoides chromosome 4 which carries the locus which is referred to here as Pseudomonas tomato race 1 (Ptr1), that recognizes the effector AvrRpt2.

This effector is present in Pst strains collected from diverse tomato-growing regions including all race 1 Pst strains for which a genome sequence is available (T1, K40, NY-T1, CA-A9, CA-407) and some race 0 Pst strains (JL1065, NY15125). The Ptr1 locus, if combined with Pto in the same tomato variety, has the potential to become an important component of bacterial speck disease control. Here the activity of Ptr1 to Arabidopsis RPS2 and apple Mr5, two genes that also detect AvrRpt2, was compared, the potential utility of Ptr1 and approaches to identifying the Ptr1 gene was discussed, and the definition of additional Pst races to account for the fact that two NTI loci are now known to confer resistance to bacterial speck disease was proposed.

Cleavage of Rin4 by AvrRpt2 in Arabidopsis leads to activation of RPS2 and resistance to P. syringae pv. tomato, although the specific mechanism by which Rin4 degradation activates RPS2 is unknown (Day et al., “Molecular Basis for the RIN4 Negative Regulation of RPS2 Disease Resistance,” Plant Cell 17(4):1292-1305 (2005); Toruno et al., “Regulated Disorder: Posttranslational Modifications Control the RIN4 Plant Immune Signaling Hub,” Mol. Plant Microbe. Interact. 32(1):56-64 (2018), which are hereby incorporated by reference in their entirety). Our analysis of AvrRpt2 variants revealed a perfect correlation between the ability of a variant to degrade tomato Rin4 proteins and its recognition by Ptr1 and RPS2; AvrRpt2 variants that do not degrade Rin4 are not recognized by Ptr1 or RPS2. It is possible therefore that the Ptr1 and RPS2 proteins are activated via the same mechanism subsequent to Rin4 degradation, although the possibility that a mechanism specific to tomato or Arabidopsis exists cannot currently be ruled out. The apple Mr5 protein detects activity of AvrRpt2 from Erwinia amylovora (Ea). The AvrRpt2_{Ea} effector does not induce Arabidopsis Rin4 degradation when the proteins are transiently co-overexpressed in N. benthamiana, although it is possible that this method masks moderate degradation; whether Rin4 degradation in apple is correlated with Mr5 activation has not been reported (Vogt et al., “Gene-for-Gene Relationship in the Host-Pathogen System Malus×Robusta 5-Erwinia Amylovora,” New Phytol. 197:1262-75 (2013), which is hereby incorporated by reference in its entirety). Interestingly, an AvrRpt2_{Ea} variant that is not detected by Mr5 is detected by Ptr1 and is able to degrade Arabidopsis RIN4, suggesting that the recognition mechanism of these two proteins is different.

Using an anti-AtRin4 antibody, it was observed that all three tomato Rin4-like proteins that are expressed in leaves were degraded in the presence of AvrRpt2. Soybean also has four Rin4 proteins and virus-induced gene silencing experiments showed that just two of them, GmRIN4a and GmRIN4b, play a role in the HR induced by AvrB and the NLR protein Rpg1b (Selote and Kachroo, 2010). A subsequent study showed that over-expression of any one of the four soybean RIN4s with Rpg1b/AvrB in leaves of Nicotiana glutinosa caused an HR; the authors concluded that the expression level differences in these two studies might account for these different observations (Kessens et al., “Determining the GmRIN4 Requirements of the soybean Disease Resistance Proteins Rpg1b and Rpg1r Using a Nicotiana Glutinosa-Based Agroinfiltration System,” PloS ONE 9(9):e108159 (2014), which is hereby incorporated by reference in its entirety). Apple contains two Rin4-like genes although, as noted above, any specific roles they might have in activation of Mr5 have not been reported yet (Fahrentrapp et al., “A Candidate Gene for Fire Blight Resistance in Malus×Robusta 5 is Coding for a CC-NBS-LRR,” Tree Genet. Genomes 9:237-51 (2012); Vogt et al., “Gene-for-Gene Relationship in the Host-Pathogen System Malus×Robusta 5-Erwinia Amylovora,” New Phytol. 197:1262-75 (2013), which are hereby incorporated by reference in their entirety). The requirement of the three tomato Rin4 proteins for activation of Ptr1 using both CRISPR-generated mutations in the corresponding genes and transient co-expression in N. benthamiana will be investigated.

Ptr1 is able to recognize AvrRpt2 homologs from a diverse array of different bacteria including several plant pathogens. Each of the AvrRpt2 proteins recognized by Ptr1 had been previously shown to also to induce AtRin4 degradation in Arabidopsis, although their ability to activate RPS2-mediated resistance was not reported (Eschen-Lippold et al., “Bacterial AvrRpt2-like Cysteine Proteases Block Activation of the Arabidopsis Mitogen-Activated Protein Kinases, MPK4 and MPK11,” Plant Physiol. 171: 2223-38 (2016), which is hereby incorporated by reference in its entirety). If avrRpt2 homologs are widespread in field isolates of Acidovorax citrulli and Erwinia amylovora then our results suggest that Ptr1 might be useful if expressed transgenically in cucurbits to control bacterial fruit blotch caused by A. citrulli and in apple or pear to confer resistance to fire blight caused by E. amylovora. Despite extensive screening of tomato germplasm, no single R gene has been identified which confers resistance to bacterial wilt disease caused by R. pseudosolanacearum (Huet, “Breeding for Resistances to Ralstonia solanacearum,” Frontiers Plant Sci. 5:715 (2014), which is hereby incorporated by reference in its entirety). It was found that Ptr1 was remarkably effective in preventing symptoms of bacterial wilt in a growth chamber assay using strain CMR15. Unfortunately, CMR15 seems to be a rare example of a R. pseudosolanacearum strain that expresses an AvrRpt2 homolog (RipBN) (Peeters et al., “Repertoire, Unified Nomenclature and Evolution of the Type III Effector Gene set in the Ralstonia solanacearum Species Complex,” BMC Genomics 14:859 (2013); (iant.toulouse.inra.fr//bacteria/annotation/site/prj/T3Ev3/), each of which are hereby incorporated by reference in their entirety), and Ptr1 will therefore likely not be of broad utility for controlling bacterial wilt disease. Nevertheless, further study of interaction between CMR15 and LA4245-R might provide some useful insights into the molecular basis of the NTI response against R. pseudosolanacearum that is induced by Ptr1.

Considering that Ptr1 plays a role in detecting the type III effector AvrRpt2 it seems most likely that it encodes an NLR, although it could encode a decoy or guardee protein which is monitored by an NLR. In either case, one of these genes must be located in the introgression segments although the other one may or may not lie in the introgressions. Examples of each possibility are known: the Pto (decoy) and Prf (NLR) genes are located in a 20 kb region of chromosome 5, whereas the RIN4 (guardee) and RPS2 (NLR) genes are located on different chromosome in Arabidopsis. In the Heinz 1706 tomato reference genome sequence chromosome 4 has 56 NLR-encoding genes, the most of any chromosome; 43 of these genes are clustered tightly within a 100-kilobase region with the other 13 located throughout the rest of the chromosome. S. lycopersicoides has 66 NLR-encoding genes on chromosome 4 with a similar distribution as seen in Heinz 1706-58 are tightly clustered at one end and 8 are distributed along the chromosome. One boundary of the large introgression segment occurs in the middle of the large NLR cluster which potentially eliminates as Ptr1 candidates 51 NLR-encoding genes. Each of the remaining 15 NLR genes is a candidate for Ptr1. None of these candidates encode proteins with obvious similarity to RPS2 or Mr5, and if one of these is Ptr1, it will be just the third known example of convergent evolution in different plant species for recognition of the same effector (Ashfield et al., “Convergent Evolution of Disease Resistance Gene Specificity in Two Flowering Plant Families,” Plant Cell 16:309-18 (2004); Carter et al., “Convergent Evolution of Effector Protease Recognition by Arabidopsis and Barley,” Mol. Plant Microbe. Interact. 32(5):550-65 (2019), which are hereby incorporated by reference in their entirety). The obvious decoy/guardee proteins that might be involved with AvrRpt2 recognition in tomato are the three Rin4-like proteins expressed in leaves. None of these genes are located on chromosome 4 of tomato or S. lycopersicoides. Another, perhaps less likely, scenario that should be considered is that a gene in the introgression segment encodes a novel host protein that acts with Rin4 to activate an NLR located on another chromosome in VF36. All of these possibilities will be investigated in the future to identify the genes involved in AvrRpt2 recognition and understand the associated molecular mechanisms.

Although LA4245-R plants are morphologically very similar to VF36, the IL line has two introgression segments on chromosome 4, including one large (˜57.5 mb) segment encompassing the majority of the 66.56 mb chromosome; however, this IL line cannot be maintained in a homozygous condition. If Ptr1 is to be useful for control of speck disease, it will need to be introgressed into other tomato breeding lines and it will be necessary to reduce the size of the introgressed segment in order to reduce ‘linkage drag’ (i.e., deleterious alleles). However, recombination is often severely suppressed in plants carrying chromosomal regions from a wild relative of tomato, and S. lycopersicoides is particularly distant from cultivated tomato (Grandillo et al., “Solanum sect. Lycopersicon,” in Ch. 9 WILD CROP RELATIVES: GENOMICS AND BREEDING RESOURCES, VEGETABLES 129-215 (Chittaranjan Kole ed., 2011), each of which is hereby incorporated by reference in its entirety). One approach that might be used in this case is to cross LA4245-R with a species which is phylogenetically intermediate between tomato and S. lycopersicoides. A good candidate for such a ‘bridge’ species is S. pennellii which has been shown previously to be useful in increasing recombination frequency in S. lycopersicoides introgression regions (Canady et al., “Homeologous Recombination in Solanum lycopersicoides Introgression Lines of Cultivated Tomato,” Genetics 174(4):1775-88 (2006), which is hereby incorporated by reference in its entirety). Importantly, Ptr1 and Pto are located on different chromosomes (4 and 5, respectively) which will facilitate introgressing them both into advanced breeding lines.

The identification of a second R gene that confers resistance to bacterial speck disease presents the opportunity to extend the currently defined race structure of P. syringae pv. tomato. The two current races were defined based on the Pto gene in which race 0 strains express either or both of the type III effectors AvrPto and AvrPtoB and race 1 strains evade Pto detection by either losing or mutating these effector genes, or in the case of AvrPtoB suppressing its protein accumulation (Pedley et al., “Molecular Basis of Pto-Mediated Resistance to Bacterial Speck Disease in Tomato,” Ann. Rev. Phytopathol. 41:215-43 (2003); Lin et al., “Diverse AvrPtoB Homologs from Several Pseudomonas syringae Pathovars Elicit Pto-Dependent Resistance and Have Similar Virulence Activities,” Appl. Environ. Microbiol. 72(1):702-12 (2006); Kunkeaw et al., “Molecular and Evolutionary Analyses of Pseudomonas syringae pv. tomato Race 1,” Mol. Plant-Microbe. Interact. 23:415-24 (2010), which are hereby incorporated by reference in their entirety). Based on the discovery of Ptr1, and focusing on Pst strains related to T1, it is proposed that race 0 strains might be considered the original state of the pathogen and refer to those strains that express avrPto or avrPtoB along with avrRpt2 (Table 5). Race 1 can then refer to strains that express avrRpt2, but lack avrPto or avrPtoB. A newly defined race 2 would refer to strains that have avrPto or avrPtoB, but lack avrRpt2. Finally, a hypothetical strain that lacks all three of these effectors would be defined as race 3. Examples of race 0, 1, and 2 Pst strains are provided in Table 5 along with the ability of Ptr1 and/or Pto to recognize them.

TABLE 5
Proposed races for P. syringae pv. tomato based on the Pto and Ptr1 genes.

| | | | | R gene present | | |
|---|---|---|---|---|---|---|
| Pst race | Example | References | Relevant effectors | Pto | Ptr1 | none |
| 0 | JL1065 | Whalen et al., 1991; | AvrPto and/or | R | R | S |
| | NY15125 | Kraus et al., 2017 | AvrPtoB, AvrRpt2 | | | |
| 1 | aT1 | Almeida et al., 2009; | AvrRpt2 | S | R | S |
| | NYT1 | Jones et al., 2015; | | | | |
| | CA-A9 | Kunkeaw et al., 2010 | | | | |
| | CA-407 | | | | | |
| 2 | Max13 | | AvrPto and/or AvrPtoB | R | S | S |
| 3 | unknown | | No AvrPto, AvrPtoB, | S | S | S |
| | | | or AvrRpt2 | | | |

Resistance and susceptibility are denoted as R and S, respectively.
^{a}Race 1 strains T1, NYT1, CA-A9 and CA-407 are positive for the presence of the avrPtoB gene, but AvrPtoB protein does not accumulate for unknown reasons.

References cited in Table 5 include: Almeida et al., “A Draft Genome Sequence of Pseudomonas Syringae pv. tomato T1 Reveals a Type III Effector Repertoire Significantly Divergent from that of Pseudomonas syringae pv. tomato DC3000,” Mol. Plant-Microbe Interact. 22(1): 52-62 (2009); ^{e}Jones et al., “Genome-Assisted Development of a Diagnostic Protocol for Distinguishing High Virulence Pseudomonas Syringae pv. tomato Strains,” Plant Disease 99:527-34 (2015); and Kunkeaw et al., “Molecular and Evolutionary Analyses of Pseudomonas syringae pv. tomato Race 1,” Mol. Plant-Microbe. Interact. 23:415-24 (2010), each of which is hereby incorporated by reference in its entirety.

In addition, the DNA sequence and predicted amino acid sequence for the Ptr1 gene from Solanum lycopersicoides have been confirmed as SEQ ID Nos 18 and 19, respectively. Evidence confirms that this gene confers AvrRpt2-mediated Pst race 1 resistance. In summary, Ptr1 has the potential to become an important component (along with Pto) for the control of bacterial speck disease in tomato.

### Example 12: Materials and Methods for Examples 13-18

### Bacterial Strains

Pseudomonas syringae pv. tomato strains JL1065 (Whalen et al., “Identification of Pseudomonas syringae Pathogens of Arabidopsis and a Bacterial Locus Determining Avirulence on Both Arabidopsis and Soybean,” Plant Cell 3:49-59 (1991), which is hereby incorporated by reference in its entirety) and JL1065ΔavrRpt2 (Lim et al., “The Pseudomonas syringae avrRpt2 Gene Contributes to Virulence on Tomato,” Mol. Plant Microbe. Interact. 18:626-33 (2005), which is hereby incorporated by reference in its entirety) were grown on King's B (KB) semi-selective media at 30° C. Agrobacterium tumefaciens strains GV3101 and GV2260 (Holsters et al., “The Functional Organization of the Nopaline A. tumefaciens Plasmid pTiC58,” Plasmid 3:212-30 (1980), which is hereby incorporated by reference in its entirety) were grown on LB with appropriate antibiotics at 30° C. (Table 6). All strains were stored in 20% glycerol+60 mM sucrose at −80° C. Escherichia coli was used for plasmid maintenance and grown in LB medium at 37° C.

TABLE 6
Bacterial strains used in this study

| Strain | Genotype | Source | Selection |
|---|---|---|---|
| P. syringae pv. tomato | Wild type | Whalen et al. | Rifampicin |
| JL1065 | | (1991)^{a} | |
| P. syringae pv. tomato | ΔavrRpt2 | Lim et al. | Rifampicin |
| JL1065 | | (2005)^{b} | Spectinomycin |
| A. tumefaciens | Wild type | Holsters et al. | Rifampicin |
| GV3101 + pMP90 | | (1980)^{c} | Gentamycin |
| A. tumefaciens GV2260 | Wild type | | Rifampicin |
| E. coli Stellar | Wild type | Clontech | — |

Table 6 source references include:
^{a}Whalen et al., “Identification of Pseudomonas syringae Pathogens of Arabidopsis and a Bacterial Locus Determining Avirulence on Both Arabidopsis and Soybean,” Plant Cell 3:49-59 (1991);
^{b}Lim et al., “The Pseudomonas syringae avrRpt2 Gene Contributes to Virulence on Tomato,” Mol. Plant Microbe. Interact. 18:626-33 (2005); and
^{c}Holsters et al., “The Functional Organization of the Nopaline A. Tumefaciens Plasmid pTiC58. Plasmid,” 3:212-30 (1980), each of which is hereby incorporated by reference in its entirety.

### Plant Materials

Seeds of Solanum lycopersicoides introgression lines were obtained from the Tomato Genetics Resource Center (tgrc.ucdavis.edu/lycopersicoides_ils.aspx). LA4245-R and LA4277-R are maintained as heterozygotes (Ptr1/ptr1). Progeny derived from selfing these plants are rarely homozygous Ptr1/Ptr1 (around 3% of the progeny) and such plants grow more slowly than Ptr1/ptr1 or ptr1/ptr1 plants. Tomato plants were grown in a greenhouse at 24° C. during daylight and 22° C. at night. Nicotiana benthamiana Nb1 (Bombarely et al, “A draft Genome Sequence of Nicotiana benthamiana to Enhance Molecular Plant-Microbe Biology Research,” Mol. Plant-Microbe Interact. 25:1523-30 (2012), which is hereby incorporated by reference in its entirety) and Nicotiana glutinosa plants were maintained in a growth chamber with 16 hr:8 hr, light:dark at 24° C. with light and 20° C. in the dark and 50% humidity. Tomato and Nicotiana plants were grown in Cornell Osmocote Mix soil (0.16 m³ peat moss, 0.34 m³ vermiculite, 2.27 kg lime, 2.27 kg Osmocote Plus 15-9-12 and 0.54 kg Uni-Mix 11-5-11; Everris, Israeli Chemicals Ltd). After pathogen inoculation, tomato plants were moved to a growth chamber with 25° C., 50% humidity, and 16 hr light.

### Mapping of Ptr1

DNA from progeny of selfed LA4277-R (Ptr1/ptr1) plants was isolated with DNA extraction buffer (200 mM Tris-HCl pH 8.0, 250 mM NaCl, 25 mM EDTA pH 8, 0.5% w/v sodium dodecyl sulfate) and resuspended in distilled water. Simple sequence repeat (SSR) markers were designed by mapping the S. lycopersicoides genome to S. lycopersicum Heinz 1706 v 3.0 using nucmer 4.0.0beta (Delcher et al., “Alignment of Whole Genomes,” Nucleic Acids Res. 27:2369-76 (1999); and Marcais et al., “MUMmer4: A Fast and Versatile Genome Alignment System,” PLoS Comput. Biol. 14:e1005944 (2018), which are hereby incorporated by reference in their entirety). A pipeline was developed to identify indels between 30-200 bp and develop flanking primers in order to amplify PCR products of different sizes based on insertions or deletions (Untergasser et al., “Primer3: New Capabilities and Interfaces,” Nucleic Acids Res. 40(15):e115 (2012), which is hereby incorporated by reference in its entirety) (Table 7). Potential recombinants were selfed and the resulting progeny were genotyped to confirm the segregating recombination event. Recombinant progeny were phenotyped by vacuum infiltration with Pst JL1065 or Pst JL1065ΔavrRpt2 as described previously (Examples 1-11; Mazo-Molina et al., “The Ptr1 Locus of Solanum lycopersicoides Confers Resistance to Race 1 Strains of Pseudomonas syringae pv. tomato and to Ralstonia Pseudosolanacearum by Recognizing the Type III effectors AvrRpt2 and RipBN,” Mol. Plant-Microbe. Interact. 32:949-60 (2019), which is hereby incorporated by reference in its entirety) and visually monitored for the absence or presence of disease symptoms,

TABLE 7
Oligonucleotides used in this study

| | | | | SEQ | |
|---|---|---|---|---|---|
| Gene | Direction | Identifier | Sequence (5′→3′) | ID NO: | Source |
| Introgressed | Forward | Chr4_4976565F | gaagccaatgtctgcagctg | 54 | This work |
| region in Chr4 | | | | | |
| 7.14 Mb | | | | | |
| Introgressed | Reverse | Chr4_4976565R | gccggtcatcagctaggtac | 55 | This work |
| region in Chr4 | | | | | |
| 7.14 Mb | | | | | |
| Introgressed | Forward | Spennch04_6606838F | ctggcctcattcttatggtt | 56 | This work |
| region in Chr4 | | | | | |
| 9.15 Mb | | | | | |
| Introgressed | Reverse | Spennch04_6606838R | taaagggagctttcgaacaa | 57 | This work |
| region in Chr4 | | | | | |
| 9.15 Mb | | | | | |
| Introgressed | Forward | Spennch04_7503010F | ctaagcatggattttagcgc | 58 | This work |
| region in Chr4 | | | | | |
| 9.94 Mb | | | | | |
| Introgressed | Reverse | Spennch04_7503010R | caacccttgaatcatggaga | 59 | This work |
| region in Chr4 | | | | | |
| 9.94 Mb | | | | | |
| Introgressed | Forward | Spennch04_5416619 | taatgaggcagagcaagttt | 60 | Examples |
| region in Chr4 | | | | | 1-11; |
| 69.13 Mb | | | | | Mazo- |
| Introgressed | Reverse | Spennch04_5416620 | ccctcaagaaccatgaatca | 61 | Molina et |
| region in Chr4 | | | | | al. (2019)^{a} |
| 69.13 Mb | | | | | |
| avrRpt2 | Forward | oCM26 | caccatgaaaattgctccagttg | 62 | This work |
| | | | ccataaatcacag | | |
| avrRpt2 | Reverse | oCM27 | cacacgcaatgctctaccgc | 63 | This work |
| S/Rin4- | Forward | KS12 | atggctcgtccaaatgtccc | 64 | This work |
| 1_Solyc09g059430 | | | | | |
| S/Rin4- | Reverse | KS13 | gccagggacaacaacaccac | 65 | This work |
| 1_Solyc09g059430 | | | | | |
| [no stop] | | | | | |
| S/Rin4- | Reverse | KS14 | ttaccagggacaacaacaccac | 66 | This work |
| 1_Solyc09g059430 | | | | | |
| S/Rin4- | Forward | KS15 | atggctcgtgcaaatgtgc | 67 | This work |
| 2_Solyc06g083390 | | | | | |
| S/Rin4- | Reverse | KS16 | gccacaaaggaaagcagcag | 68 | This work |
| 2_Solyc06g083390 | | | | | |
| [no stop] | | | | | |
| S/Rin4- | Reverse | KS17 | tcaccacaaaggaaagcagca | 69 | This work |
| 2_Solyc06g083390 | | | g | | |
| S/Rin4- | Forward | KS18 | atggcaaaacactcacaagtac | 70 | This work |
| 3_Solyc12g098440 | | | c | | |
| S/Rin4- | Reverse | KS19 | gactccgaccccatggaaag | 71 | This work |
| 3_Solycl2g098440 | | | | | |
| [no stop] | | | | | |
| S/Rin4- | Reverse | KS20 | tcaactccgaccccatggaaag | 72 | This work |
| 3_Solycl2g098440 | | | | | |
| AtRIN4_At3G25070.1 | Forward | KS24 | atggcacgttcgaatgtaccaa | 73 | This work |
| AtRIN4_At3G25070.1 | Reverse | KS25 | tcattttcctccaaagccaaagc | 74 | This work |
| Ptr1_Solyd04g059470 | Forward | oCM206 | tttggagaggacagggtaccat | 75 | This work |
| | | | ggcggaatctttcttgttcaatatc | | |
| Ptr1_Solyd04g059470 | Reverse | oCM207 | gtcgtatgggtaaggtccaccta | 76 | This work |
| | | | gcaatggacagtc | | |
| Candidate | Forward | oCM208 | tttggagaggacagggtaccat | 77 | This work |
| B_Solyd04g059610 | | | ggctgatgcctttgtgtc | | |
| Candidate- | Reverse | oCM209 | gtcgtatgggtaagggattgaaa | 78 | This work |
| B_Solyd04g059610 | | | catgtgaagcttttcaaccatata | | |
| Ptr1-VIGS1 | Forward | oSM03 | ggggacaagtttgtacaaaaaa | 79 | This work |
| | | | gcaggctttgagctatgaacaga | | |
| | | | tgccatcacatc | | |
| Ptr1-VIGS1 | Reverse | oSM04 | ggggaccactttgtacaagaaa | 80 | This work |
| | | | gctgggtaaagcattgggtttcac | | |
| | | | atttaagcactctg | | |
| synPtr1 | Forward | oSM15 | ggggacaagtttgtacaaaaaa | 81 | This work |
| | | | gcaggctttgagctatgaacaga | | |
| | | | tgccatcacatc | | |
| synPtr1 | Reverse | oSM16 | ggggaccactttgtacaagaaa | 82 | This work |
| | | | gctgggtaaagcattgggtttcac | | |
| | | | atttaagcactctg | | |
| StPtr1 | Forward | oCM206 | tttggagaggacagggtaccat | 83 | This work |
| | | | ggcggaatctttcttgttcaatatc | | |
| StPtr1 | Reverse | oCM207 | gtcgtatgggtaaggtccaccta | 84 | This work |
| | | | gcaatggacagtc | | |

^{a}Mazo-Molina et al., “The Ptrl Locus of Solanum lycopersicoides Confers Resistance to Race 1 Strains of Pseudomonas syringae pv. Tomato and to Ralstonia Pseudosolanacearum by Recognizing the Type III effectors AvrRpt2 and RipBN,” Mol. Plant-Microbe. Interact. 32:949-60 (2019), which is hereby incorporated by reference in its entirety. See Examples 1-11.

### RNA-Seq

Seven-week-old LA4277-Ro (Ptr1/Ptr1) plants were identified by markers and vacuum-infiltrated with a suspension of Pst JL1065 at 2×10⁷ cfu/mL. Four biological replicates were performed for each treatment. Tissue samples were collected at 5 hr after infiltration. Total RNA was isolated using RNeasy Plant Mini Kit (Qiagen) with additional in-column DNase digestion using the RNase-Free DNase Kit (Qiagen). Libraries for 3′ RNA-Seq were prepared by the Cornell Biotechnology Resource Center's Genomics Facility using the Quantseq FWD kit (Lexogen). Raw RNA-Seq reads were processed, removing adaptors and low-quality sequences using Trimmomatic (Bolger et al., “Trimmomatic: A Flexible Trimmer for Illumina Sequence Data,” Bioinf 30:2114-20 (2014), which is hereby incorporated by reference in its entirety). Low quality sequences were removed from leading and trailing read ends, and trimmed reads shorter than 10 bases were discarded. Clean reads were then aligned to the SILVA rRNA database (Quast et al., “The SILVA Ribosomal RNA Gene Database Project: Improved Data Processing and Web-Based Tools,” Nucleic Acids Res. 41:D590-96 (2013), which is hereby incorporated by reference in its entirety) using Bowtie (Langmead et al., “Fast Gapped-Read Alignment with Bowtie 2,” Nat. Methods 9:357-9 (2012), which is hereby incorporated by reference in its entirety) allowing for up to three mismatches. Reads that mapped to rRNA sequence were discarded. The final high-quality reads for each library were aligned to the S. lycopersicoides LA2951 reference genome (solgenomics.net/organismSolanumIycopersicoides/genome) using STAR default parameters (Dobin et al., “STAR: Ultrafast Universal RNA-Seq Aligner,” Bioinformatics 29:15-21 (2013), which is hereby incorporated by reference in its entirety). Raw counts for each LA2951 gene model were generated by counting the total number of reads that mapped between the gene region and 500 bp downstream of its stop codon. Reads were normalized to reads per million (RPM).

### Cloning of Ptr1 Candidates and Tomato Rin4 Genes

Ptr1 candidate genes were cloned from LA4277-Ro (Ptr1/Ptr1) cDNA into pBTEX (Table 8) using the In-fusion cloning kit manufacturer's instructions (Takara). StPtr1 was cloned from Dakota Crisp potato plants (Solanum tuberosum) into pBTEX via the In-fusion cloning kit (Takara). Nucleotide and amino acid sequences have been deposited in GenBank for Ptr1 from S. lycopersicoides (GenBank accession no. MT134103, which is hereby incorporated by reference in its entirety), N. benthamiana, NbPt1a (MT134102, which is hereby incorporated by reference in its entirety) and potato StPtr1 (MT134101, which is hereby incorporated by reference in its entirety) (Table 9).

TABLE 8
Vectors and plasmids used in this study

| Vector | Insert | Identifier | Purpose | Source |
|---|---|---|---|---|
| pBTEX | — | — | Binary expression vector | R. Bressan, |
| | | | with N-terminal HA-tag | Purdue University |
| pBTEX | Ptr1-A_ | pCM64 | Generating N-terminal | This work |
| | Solyd04g059470 | | HA-tag fusion | |
| pBTEX | Ptrl-B_ | pCM66 | Generating N-terminal | This work |
| | Solyd04g059610 | | HA-tag fusion | |
| | RPS2_At4g26090 | — | N-terminal HA-tagged | B. Staskawicz, |
| | | | RPS2 gene | UC Berkeley |
| pJLSmart | — | — | Binary Gateway entry | Mathieu et al. |
| | | | vector | (2014)^{a} |
| pJLSmart | SlRin4- | 10347 | Entry clone for N- | This work |
| | 1_Solyc09g059430 | | terminal Myc-tag fusion | |
| pJLSmart | SlRin4- | 10346 | Entry clone for C- | This work |
| | 1_Solyc09g059430 | | terminal Myc-tag fusion | |
| | [no stop] | | | |
| pJLSmart | SlRin4- | 10352 | Entry clone for N- | This work |
| | 2 _Solyc06g083390 | | terminal Myc-tag fusion | |
| pJLSmart | SlRin4- | 10348 | Entry clone for C- | This work |
| | 2_Solyc06g083390 | | terminal Myc-tag fusion | |
| | [no stop] | | | |
| pJLSmart | SlRin4- | 10350 | Entry clone for N- | This work |
| | 3_Solyc12g098440 | | terminal Myc-tag fusion | |
| pJLSmart | SlRin4- | 10349 | Entry clone for C- | This work |
| | 3_Solyc12g098440 | | terminal Myc-tag fusion | |
| | [no stop] | | | |
| pGWB417 | — | — | Binary Gateway | Nakagawa et al. |
| | | | destination vector for | (2007)^{b} |
| | | | plant expression with C- | |
| | | | terminal Myc-tag. | |
| pGWB518 | — | — | Binary Gateway | Nakagawa et al. |
| | | | destination vector for | (2007)^{b} |
| | | | plant expression with C- | |
| | | | terminal Myc-tag. | |
| pGWB518 | SlRin4- | pKS13 | Generating N-terminal | This work |
| | 1_Solyc09g059430 | | Myc-tag fusion | |
| pGWB518 | SlRin4- | pKS15 | Generating N-terminal | This work |
| | 2_Solyc06g083390 | | Myc-tag fusion | |
| pGWB518 | SlRin4- | pKS17 | Generating N-terminal | This work |
| | 3_Solyc12g098440 | | Myc-tag fusion | |
| pGWB518 | AtRIN4_At3G25070.1 | pKS18 | Generating N-terminal | This work |
| | | | Myc-tag fusion | |
| pGWB417 | SlRin4- | pKS12 | Generating C-terminal | This work |
| | 1_Solyc09g059430 | | Myc-tag fusion | |
| pGWB417 | SlRin4- | pKS14 | Generating C-terminal | This work |
| | 2_Solyc06g083390 | | Myc-tag fusion | |
| pGWB417 | SlRin4- | pKS16 | Generating C-terminal | This work |
| | 3_Solyc12g098440 | | Myc-tag fusion | |
| pGWB417 | avrRpt2_Pseudomonas | pCM1 | Generating C-terminal | Examples 1-11; |
| | syringae pv. tomato | | Myc-tag fusion | Mazo-Molina et |
| | NY15125 | | | al. (2019)^{c} |
| pGWB417 | AvrRpt2(C122A) | — | Generating C-terminal | Examples 1-11; |
| | | | Myc-tag fusion | Mazo-Molina et |
| | | | | al. (2019)^{c} |
| pGWB417 | RipBN | — | Generating C-terminal | Examples 1-11; |
| | | | Myc-tag fusion | Mazo-Molina et |
| | | | | al. (2019)^{c} |
| pGWB417 | YFP | — | Generating C-terminal | Roberts et al. |
| | | | Myc-tag fusion | (2019)^{d} |
| pBTEX | Prf(D1416V) | — | Generating N-terminal | Du et al. (2012)^{e} |
| | | | HA-tag fusion | |
| pDONR/Zeo | — | — | Binary Gateway entry | Invitrogen |
| | | | vector | |
| pDONR/Zeo | Ptr1-TRV2 | pSM1 | Entry clone for Ptr1- | This work |
| | | | TRV2 construct | |
| pQ11 | TRV1 | — | VIGS TRV1 construct | Liu et at. (2002)^{f} |
| pQ11 | — | — | empty TRV2 constrcut | Liu et at. (2002)^{f} |
| pQ11 | EC1-TRV2 | — | VIGS TRV2 construct to | Rosli et at. |
| | | | silence EC1 | (2013)^{g} |
| pQ11 | Ptrl-TRV2 | pSM2 | VIGS TRV2 construct to | This work |
| | | | silence Ptr1 | |
| pBTEX | synPtr1 | pSM3 | Generating N-terminal | This work |
| | | | HA-tag fusion | |
| pBTEX | StPtr1/RGA1 | pCM68 | Generating N-terminal | This work |
| | | | HA-tag fusion | |

Table 8 source references include:
^{a}Mathieu et al., “Pto Kinase Binds two Domains of AvrPtoB and its Proximity to the Effector E3 Ligase Determines if it Evades Degradation and Activates Plant Immunity,” PLoS Pathog. 10:e1004227 (2014);
^{b}Nakagawa et al., “Improved Gateway Binary Vectors: High-Performance Vectors for Creation of Fusion Constructs in Transgenic Analysis of Plants,” Biosci. Biotechnol. Biochem. 71:2095-100 (2007);
^{c}Mazo-Molina et al., “The Ptr1 Locus of Solanum lycopersicoides Confers Resistance to Race 1 Strains of Pseudomonas syringae pv. Tomato and to Ralstonia pseudosolanacearum by Recognizing the Type III Effectors AvrRpt2 and RipBN,” Mol. Plant Microbe. Interact. 32:949-60 (2019);
^{d}Roberts et al., “Mai1 Protein Acts Between Host Recognition of Pathogen Effectors and Mitogen-Activated Protein Kinase Signaling,” Mol. Plant-Microbe. Interact. 32:1496-507 (2019);
^{e}Du et al., “Plant Programmed Cell Death Caused by an Autoactive Form of Prf is Suppressed by Co-Expression of the Prf LRR Domain,” Mol. Plant 5:1058-67 (2012);
^{f}Du et al., “Plant Programmed Cell Death Caused by an Autoactive Form of Prf is Suppressed by Co-Expression of the Prf LRR Domain,” Mol. Plant 5:1058-67 (2012); and
^{g}Rosli et al., “Transcriptomics-Based Screen for Genes Induced by Flagellin and Repressed by Pathogen Effectors Identifies a Cell Wall-Associated Kinase Involved in Plant Immunity,” Genome Biol.
14:R139 (2013), each of which is hereby incorporated by reference in its entirety.

TABLE 9
Details of each Ptr1 gene in various solanaceous species. Each sequence identified in
Table 9 is hereby incorporated by reference in its entirety.

| | | GenBank ID/ | |
|---|---|---|---|
| | | NCBI reference | |
| Species | Gene ID | sequence | Comments |
| S. lycopersicoides | Solyd04g059470 | MT134103 | Ptr1 |
| S. tuberosum | | MT134101 | StrPtr1; cv. Dakota Crisp |
| S. lycopersicum | Solyc04g039980/ | | no open reading frame |
| | Solyc04g039990 | | |
| S. pennellii | Sopen04g016200 | | no open reading frame |
| C. annuum | CA05g00030 | PHT80575.1 | cv. CM334; annotated |
| | | | previously as RGA1 |
| N. tabacum | Nitab4.5_0002085g0050.1 | XP_016435110.1 | gene misannotated. Intron |
| | | | should be exon; correct in |
| | | | GenBank; annotated previously |
| | | | as RGA3 |
| N. attenuata | NIATv7_g26201.t1 | O1T07149.1 | annotated previously as RGA3 |
| N. tomentosiformis | mRNA_17526 | XP_018629570.1 | annotated previously as RGA3 |
| C. chinense | | PHU16668.1 | hypothetical protein |
| C. baccatum | | PHT54558.1 | hypothetical protein |
| N. benthamiana | Niben101Scf07061g00004.1 | MT134102 | NbPtr1a; gene misannotated in |
| | | | Nb1 genome sequence. Intron |
| | | | should be exon |
| N. benthamiana | Niben101Scf00597g00011.1/ | | NbPtr1b; no open reading frame |
| | Niben101Scf00597g00010.1 | | |
| S. melongena | SMEL011g377860 | | missing NB-ARC domain; |
| | | | annotated previously as RPP13- |
| | | | like |
| P. axillaris | Peaxi162Scf00970g00162.1 | | no open reading frame |
| P. inflata | Peinf101Scf01668g00022.1 | | no open reading frame |
| P. inflata | Peinf101Scf14222g00007.1 | | no open reading frame |

SlRin4 genes were cloned from LA4245 cDNA into pJLSmart (Mathieu et al., “Pto Kinase Binds two Domains of AvrPtoB and its Proximity to the Effector E3 Ligase Determines if it Evades Degradation and Activates Plant Immunity,” PLoS Pathog. 10:e1004227 (2014), which is hereby incorporated by reference in its entirety) and recombined into the Gateway expression vectors pGWB518 and pGWB417 (Nakagawa et al., “Improved Gateway Binary Vectors: High-Performance Vectors for Creation of Fusion Constructs in Transgenic Analysis of Plants,” Biosci. Biotechnol. Biochem. 71:2095-100 (2007), which is hereby incorporated by reference in its entirety) using LR Clonase II according to the manufacturer's instructions (Thermo Fisher Scientific) to generate N-tagged and C-tagged proteins, respectively.

Constructs were transformed into E. coli Stellar competent cells (Clontech). Inserts of all plasmids were sequenced from E. coli and then transformed into Agrobacterium tumefaciens GV2260. See Table 7 and Table 8 for a list of all oligo and constructs used in this study.

### LA4277 Genomic DNA Library Preparation and Oxford Nanopore Sequencing

The LA4277 genome sequence data are available at NCBI as BioProject No. PRJNA610286. To extract high molecular weight DNA, nuclei were enriched from 2 g of LA4277-Ro (Ptr1/Ptr1) leaves using a method modified from (Gendrel et al., “Profiling Histone Modification Patterns in Plants Using Genomic Tiling Microarrays,” Nat. Methods 2:213-8 (2005), which is hereby incorporated by reference in its entirety). Two g of leaf tissue was harvested and ground in liquid nitrogen and incubated with 90 mL of 0.4 M sucrose, 10 mM Tris-HCl pH 8, 10 mM MgCl₂, 5 mM BME (β-mercaptoethanol) for 10 min on ice with shaking. Large leaf debris was removed by filtering through 8 layers of filter paper (Fisher Scientific) and one layer of miracloth (Fisher Scientific). The solution was centrifuged at 3000×g at 4° C. for 20 min. The resulting pellet was resuspended in 500 uL of 0.25 M sucrose, 10 mM Tris-HCl pH 8, 10 mM MgCl₂, 1% Triton X-100, 5 mM BME. The suspension was then centrifuged at 12,000×g at 4° C. for 10 min. The pellet was resuspended in 300 uL 0.25 sucrose, 20 mM Tris-HCl pH 8, 4 mM MgCl₂, 0.3% Triton X-100 and 500 μL of 2.5 M sucrose was then mixed with the nuclei suspension. This mixture was then overlayed on top of 800 μL of 1.7 M sucrose, 10 mM Tris-HCl pH 8, 2 mM MgCl₂, 0.15% Triton X-100, 5 mM BME and centrifuged at 16,000×g at 4° C. for 1 hr. The pelleted nuclei were washed 2× in 500 μL of 25% glycerol, 20 mM Tris-HCl pH 7.4, 2.5 mM MgCl₂, 0.2% Triton X-100. DNA was isolated from the enriched nuclei suspension as described previously (Bernatzky et al., “Genetics of Actin-Related Sequences in Tomato,” Theory Appl. Genet. 72:314-21 (1986), which is hereby incorporated by reference in its entirety).

Covaris g-tube was used in accordance with the manufacturer's protocol to shear 8 ug DNA to 20 kb fragments. Following shearing, small fragments were removed with a size-selection step using 1× AMPure XP (Beckman Coulter, Brea, CA, USA) in NaCl/PEG buffer (10 mM Tris-HCl, 1 mM EDTA pH 8, 1.6 M NaCl, 0.25% Tween-20, 11% PEG-800) (Nagar et al., “DNA Size Selection (>3-4 kb) and Purification of DNA Using an Improved Homemade Spribeads Solution,” Protocols.io doi.org/10.17504/protocols.io.n7hdhj6 (2018), which is hereby incorporated by reference in its entirety). Large fragments were eluted from beads in 50 uL H₂O. A library was prepared using 1 μg of DNA as input using the SQK-LSK109 kit (Oxford Nanopore Technologies, Oxford, UK) according to manufacturer's protocol, except incubation times were increased to 30 min for DNA repair, end-prep and 1 hr for adapter ligation, and 10 min for all elutions from beads. The finished library was loaded on to a single FLO-Min106D R9 Spot-ON flowcell (Oxford Nanopore Technologies, Oxford, UK). MinION sequencing was performed using the standard script for a 60 hr run.

### LA4277 Genomic DNA Library Preparation and Illumina Sequencing

Illumina libraries for whole genome sequencing of LA4277-Ro were prepared as described previously (Examples 1-11; Mazo-Molina et al., “The Ptr1 Locus of Solanum lycopersicoides Confers Resistance to Race 1 Strains of Pseudomonas syringae pv. tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety) using nuclear-enriched DNA. Paired-end 150-bp DNA reads were sequenced using the Illumina HiSeq 2000 platform by Genewiz (South Plainfield, NJ).

### LA4277 Genome Assembly

Nanopore sequence was base-called using Guppy v 2.3.5+53a11 if (Wick et al., “Performance Of Neural Network Basecalling Tools for Oxford Nanopore Sequencing,” Genome Biol. 20:129 (2019), which is hereby incorporated by reference in its entirety). A hybrid approach implemented in MaSuRCA v3.3.2 (Zimin et al., “The MaSuRCA Genome Assembler,” Bioinformatics 29:2669-77 (2013), which is hereby incorporated by reference in its entirety) was used to de novo assemble the LA4277 Nanopore and Illumina genome sequence. The assembly was polished with Illumina reads and 3 rounds of Pilon correction. The assembly had a total length of 840.2 Mbp, N50 of 1.3 Mbp, and captured over 97% of the BUSCO set.

### Agrobacterium-Mediated Transient Protein Expression

Cell death assays in five-week-old N. benthamiana and N. glutinosa plants were performed as described previously (Oh et al., “Tomato 14-3-3 Protein 7 Positively Regulates Immunity-Associated Programmed Cell Death by Enhancing Protein Abundance and Signaling Ability of MAPKKKα,” Plant Cell 22:260-72 (2010), which is hereby incorporated by reference in its entirety), after dilution of the Agrobacterium strains to a final OD₆₀₀ of 0.025 or 0.1 as indicated in the figure legend for Ptr1, 0.05 for AvrRpt2, 0.2 for Rin4, and 0.1 for Prf. For all of the Agrobacterium assays, cell death in the infiltrated areas, when it occurred, was strong and reproducible and was documented as plus (100% cell death) or minus (no cell death). Cell death started to appear 2 days after Agrobacterium infiltration, except for Prf where it started 4 days after infiltration. To detect protein expression in N. benthamiana, a final OD₆₀₀ of 0.1 for Ptr1, 0.05 for AvrRpt2, and 0.2 for Rin4 was used and leaf tissue was sampled 28 hr after infiltration.

For the VIGS experiments, Agrobacterium strains were diluted to a final OD₆₀₀ of 0.2 for AvrRpt2, AvrRpt2(C122A), RipBN, YFP, and Prf(D1416V). Cell death started to appear 2 days after infiltration at which time the Nb1 plants were scored and photographed. TRV:EC1 and TRV:Ptr1 silenced plants were scored for cell death and photographed 72 hr after infiltration for all constructs except Prf(D1416V), which was scored and photographed 4 days after infiltration. The experiment was repeated three times, using six TRV:EC1 and TRV:Ptr1 plants in each experiment and infiltrating two leaves per plant.

For the synPtr1 complementation assays, Agrobacterium containing the Ptr1 and synPtr1 constructs were infiltrated into TRV:EC1 and TRV:Ptr1 silenced plants at a final OD₆₀₀ of 0.025 and co-infiltrated with Agrobacterium strains carrying AvrRpt2, AvrRpt2(C122A), RipBN, or YFP at a final OD₆₀₀ of 0.05. Cell death started to appear 48 hr after infiltration. Plants were scored for cell death and photographed 6 days after infiltration. The complementation experiment was repeated three times, using 3-6 TRV:EC1 and TRV:Ptr1 plants in each experiment and infiltrating two leaves per plant. Photos shown in the figures are representative of all replicates. To detect protein expression of Ptr1 and synPtr1 in silenced plants, Agrobacterium strains were infiltrated at a final OD₆₀₀ of 0.1. Leaf samples for protein expression were collected 46 hr after infiltration.

### Immunoblot Detection of Plant-Expressed Proteins

To detect protein expression in N. benthamiana, total proteins were extracted as previously described (Examples 1-11; Mazo-Molina et al., “The Ptr1 Locus of Solanum lycopersicoides Confers Resistance to Race 1 Strains of Pseudomonas syringae pv. tomato and to Ralstonia Pseudosolanacearum by Recognizing the Type III effectors AvrRpt2 and RipBN,” Mol. Plant-Microbe. Interact. 32:949-60 (2019), which is hereby incorporated by reference in its entirety). To detect AvrRpt2 and Rin4, membranes were probed with anti-c-Myc (GeneScript) antibody at a concentration of 1/7000. Secondary ∝-mouse-HRP was used at a dilution of 1/10,000 (Sigma-Aldrich). For detection of Ptr1, synPtr1, and RPS2 proteins, membranes were probed with anti-HA antibody at a concentration of 1/2,000 (Roche). Secondary ∝-rat-HRP was used at a dilution of 1/10,000 (Cell Signaling Technology).

### Phylogenetic Analyses

Alignments were generated using MUSCLE except where noted otherwise. Phylogenetic trees were constructed in MEGA7 (Kumar et al., “MEGA7: Molecular Evolutionary Genetics Analysis Version 7.0 for Bigger Datasets,” Mol. Biol. Evol. 33:1870-4 (2016), which is hereby incorporated by reference in its entirety) using the maximum likelihood with a JTT matrix-based model method (Jones et al., “The Rapid Generation of Mutation Data Matrices From Protein Sequences,” Comput. Appl. Biosci. 8:275-82 (1992), which is hereby incorporated by reference in its entirety). Positions containing gaps and missing data were eliminated. A bootstrap analysis with 1,000 replicates was used to determine the confidence probability of each branch (Felsenstein, “Confidence Limits on Phylogenies: An Approach Using the Bootstrap,” Evolution 39:783-91 (1985), which is hereby incorporated by reference in its entirety). Data on which AvrRpt2-like proteins are recognized by Ptr1 are from Examples 1-11, Mazo-Molina et al., “The Ptr1 Locus of Solanum lycopersicoides Confers Resistance to Race 1 Strains of Pseudomonas syringae pv. Tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety, and the current study.

The top 10 BLASTp hits of the Ptr1, Mr5, and RPS2 NB-ARC domains were identified in the S. lycopersicoides v.0.6 genome sequence (Powell et al., “A Solanum lycopersicoides Reference Genome Facilitates Biological Discovery in Tomato,” bioRxiv 2020.2004.2016.039636 (2020), which is hereby incorporated by reference in its entirety), M. domestica (GDR GDDH13 V1.1) (Jung et al., “15 years of GDR: New Data and Functionality in the Genome Database for Rosaceae,” Nucleic Acids Res. 47:D1137-45 (2019), which is hereby incorporated by reference in its entirety), and A. thaliana (TAIR Araport 11) genome protein databases. The NB-ARC domain of each protein hit was determined using Interpro scan and the amino acid sequences of the NB-ARC domain for each species were aligned to check for the presence of the NB-ARC conserved motifs. To ensure alignment of the sequences, NB-ARC domains missing any of the motifs were removed from further analysis. AvrRpt2 homologs in GenBank were identified through NCBI BLAST, and were aligned with MUSCLE, along with sequences of avrRpt2 homologs from Examples 1-11 and recent papers (Eschen-Lippold et al., “Bacterial AvrRpt2-like Cysteine Proteases Block Activation of the Arabidopsis Mitogen-Activated Protein Kinases, MPK4 and MPK11,” Plant Physiol 171:2223-8 (2016); Dillon et al., “Molecular Evolution of Pseudomonas syringae Type III Secreted Effector Proteins,” Front Plant Sci. 10(418):10.3389/fpls.2019.00418 (2019); and Mazo-Molina et al., “The Ptr1 Locus of Solanum lycopersicoides Confers Resistance to Race 1 Strains of Pseudomonas syringae pv. tomato and to Ralstonia pseudosolanacearum by Recognizing the Type III Effectors AvrRpt2 and RipBN,” Mol. Plant Microbe. Interact. 32:949-60 (2019), which are hereby incorporated by reference in their entirety) (Table 10).

TABLE 10
AvrRpt2 sequences used in the phylogenetic tree in FIG. 36.

| Species | Annotation | ID | Reference |
|---|---|---|---|
| Acidovorax avenae subsp. | AvrRpt2 | ADX44172.1 | Eschen-Lippold et |
| avenae ATCC 19860 | | | al. (2016)^{a} |
| Acidovorax citrulli | hypothetical | WP_082091296.1 | |
| | protein | | |
| Burkholderia pyrrocinia Lyc2 | AvrRpt2 | KFL50402.1 | Eschen-Lippold et |
| | | | al. (2016)^{a} |
| Collimonas fungivorans | cysteine protease | WP_082814589.1 | |
| Erwinia amylovora ATCC | AvrRpt2 | CBJ45097.1 | Eschen-Lippold et |
| 49946 | | | al. (2016)^{a} |
| Mesorhizobium huakuii 7653R | AvrRpt2 | AID34449.1 | Eschen-Lippold et |
| | | | al. (2016)^{a} |
| Neorhizobium galegae | cysteine protease | WP_151045106.1 | |
| P syringae pv. castaneae | AvrRpt2 | PcsICMP9421 | Dillon et al. (2019)^{b} |
| P syringae pv. morsprunorum | AvrRpt2 | PmpICMP3897 | Dillon et al. (2019)^{b} |
| P. syringae pv. castaneae | AvrRpt2 | PcsICMP9419 | Dillon et al. (2019)^{b} |
| P. syringae pv. castaneae | AvrRpt2 | PcsICMP9420 | Dillon et al. (2019)^{b} |
| P. syringae pv. caricapapayae | AvrRpt2 | PcaICMP7496 | Dillon et al. (2019)^{b} |
| P. syringae pv. lachrymans | AvrRpt2 | Pla1188_1 | Dillon et al. (2019)^{b} |
| P. syringae pv. maculicola | AvrRpt2 | PmaM4a | Dillon et al. (2019)^{b} |
| P. syringae pv. persicae | AvrRpt2 | PpeICMP3706 | Dillon et al. (2019)^{b} |
| P. syringae pv. persicae | AvrRpt2 | PpeICMP5786 | Dillon et al. (2019)^{b} |
| P. syringae pv. sesami | AvrRpt2 | PseHC_1 | Dillon et al. (2019)^{b} |
| P. syringae pv. sesami | AvrRpt2 | PseICMP3386 | Dillon et al. (2019)^{b} |
| P. syringae pv. sesami | AvrRpt2 | PseICMP4995 | Dillon et al. (2019)^{b} |
| P. syringae pv. sesami | AvrRpt2 | PseICMP7459 | Dillon et al. (2019)^{b} |
| P. syringae pv. sesami | AvrRpt2 | PseICMP763 | Dillon et al. (2019)^{b} |
| P. syringae pv. spinaceae | AvrRpt2 | PspICMP16928 | Dillon et al. (2019)^{b} |
| P. syringae pv. spinaceae | AvrRpt2 | PspICMP16929 | Dillon et al. (2019)^{b} |
| P. syringae pv. tagetis | AvrRpt2 | PtgICMP4092 | Dillon et al. (2019)^{b} |
| P. syringae pv. tremae | AvrRpt2 | PtrICMP9151 | Dillon et al. (2019)^{b} |
| P. syringae pv. zizaniae | AvrRpt2 | PziICMP8959 | Dillon et al. (2019)^{b} |
| P. syringae pv. zizaniae | AvrRpt2 | PziICMP8921 | Dillon et al. (2019)^{b} |
| Pseudomonas amygdali | cysteine protease | WP_103379467.1 | |
| | avirulence protein | | |
| | AvrRpt2 | | |
| Pseudomonas amygdali | cysteine protease | WP_081003941.1 | |
| | avirulence protein | | |
| | AvrRpt2 | | |
| Pseudomonas caricapapayae | cysteine protease | WP_122340504.1 | |
| Pseudomonas coronafaciens | cysteine protease | WP_081021999.1 | |
| | avirulence protein | | |
| | AvrRpt2 | | |
| Pseudomonas syringae | cysteine protease | WP_080397204.1 | |
| | avirulence protein | | |
| | AvrRpt2 | | |
| Pseudomonas syringae group | cysteine protease | WP_081012136.1 | |
| | avirulence protein | | |
| | AvrRpt2 | | |
| Pseudomonas syringae group | peptidase C70 | WP_007247525.1 | |
| Pseudomonas syringae group | cysteine protease | WP_083492254.1 | |
| | avirulence protein | | |
| | AvrRpt2 | | |
| Pseudomonas syringae group | cysteine protease | WP_080392394.1 | |
| | avirulence protein | | |
| | AvrRpt2 | | |
| Pseudomonas syringae group | cysteine protease | WP_099264889.1 | |
| | avirulence protein | | |
| | AvrRpt2 | | |
| Pseudomonas syringae group | cysteine protease | WP_080378320.1 | |
| Pseudomonas syringae group | cysteine protease, | WP_122290586.1 | |
| genomosp. 3 | partial | | |
| Pseudomonas syringae pv. | cysteine protease | KPW75402.1 | |
| coriandricola | avirulence protein | | |
| | AvrRpt2 | | |
| Pseudomonas syringae pv. | hypothetical | RMN57113.1 | Dillon et al. (2019)^{b} |
| papulans | protein | | |
| | ALQ56_200124 | | |
| Pseudomonas syringae pv. | AvrRpt2 | Pto1108 | Dillon et al. (2019)^{b} |
| tomato | | | |
| Pseudomonas syringae pv. | AvrRpt2 | PtoICMP4263 | Dillon et at. (2019)^{b} |
| tomato | | | |
| Pseudomonas syringae pv. | AvrRpt2 | PtoICMP7230 | Dillon et al. (2019)^{b} |
| tomato | | | |
| Pseudomonas syringae pv. | AvrRpt2 | PtoK40 | Dillon et al. (2019)^{b} |
| tomato | | | |
| Pseudomonas syringae pv. | AvrRpt2 | PtoT1 | Dillon et al. (2019)^{b} |
| tomato | | | |
| Pseudomonas syringae pv. | AvrRpt2 | CAA79815.2 | Eschen-Lippold et |
| tomato JL1065 | | | al. (2016) ^{a} |
| Pseudomonas syringae pv. | AvrRpt2 | | Examples 1-11; |
| tomato NY 15125 | | | Mazo-Molina et al. |
| | | | (2019)^{c} |
| Pseudomonas syringae pv. | cysteine protease | KGK92417.1 | |
| tomato NYS-T1 | | | |
| Ralstonia solanacearum | hypothetical | WP_080658778.1 | |
| CMR15 | protein | | |
| Sinorhizobium medicae | AvrRpt2 | NZ_AQUS01000051.1 | Eschen-Lippold et |
| WSM1369 | | | al. (2016) ^{a} |
| Sinorhizobium meliloti | cysteine protease | WP_146722358.1 | |
| | avirulence protein | | |
| | AvrRpt2 | | |

Table 10 references include:
^{a}Eschen-Lippold et al., “Bacterial AvrRpt2-like Cysteine Proteases Block Activation of the Arabidopsis Mitogen- Activated Protein Kinases, MPK4 and MPK11,” Plant Physiol 171:2223-8 (2016);
^{b}Dillon et al., “Molecular Evolution of Pseudomonas syringae Type III Secreted Effector Proteins,” Front Plant Sci. 10(418):10.3389/fpls.2019.00418 (2019); and
^{c}Mazo-Molina et al., “The Ptr1 Locus of Solanum lycopersicoides Confers Resistance to Race 1 Strains of Pseudomonas syringae pv. Tomato and to Ralstonia pseudosolanacearum by Recognizing the Type III Effectors AvrRpt2 and RipBN,” Mol. Plant Microbe. Interact. 32:949-60 (2019), each of which is hereby incorporated by reference in its entirety. Each sequence identified in Table 10 is hereby incorporated by reference in its entirety.

### Virus-Induced Gene Silencing (VIGS)

The Ptr1-targeting VIGS sequence was selected using the SGN VIGS Tool (Fernandez-Pozo, et al., “The SGN VIGS Tool: User-friendly Software to Design Virus-Induced Gene Silencing (VIGS) Constructs for Functional Genomics,” Mol. Plant 8:486-88 (2015), which is hereby incorporated by reference in its entirety). The fragment was cloned into pDONR/Zeo (Invitrogen) followed by an LR reaction (Invitrogen) into pQ11 (Liu et al., “Virus-Induced Gene Silencing in Tomato,” Plant J. 31:777-86 (2002), which is hereby incorporated by reference in its entirety). The resulting pQ11:Ptr1 (TRV:Ptr1) construct was transformed into A. tumefaciens GV2260. The control, pQ11:EC1 (TRV:EC1), contains a small DNA fragment from E. coli and was described previously (Rosli et al., “Transcriptomics-Based Screen for Genes Induced by Flagellin and Repressed by Pathogen Effectors Identifies a Cell Wall-Associated Kinase Involved in Plant Immunity,” Genome Biol. 14:R139 (2013), which is hereby incorporated by reference in its entirety). VIGS constructs were prepared for infections in N. benthamiana as described previously (Chakravarthy et al., “Identification of Nicotiana benthamiana Genes Involved in Pathogen-Associated Molecular Pattern-Triggered Immunity,” Mol. Plant-Microbe. Interact. 23:715-26 (2010), which is hereby incorporated by reference in its entirety). Cell death assays were performed five-to-six weeks after agroinfiltration with the VIGS constructs.

### Generation of Synthetic Ptr1

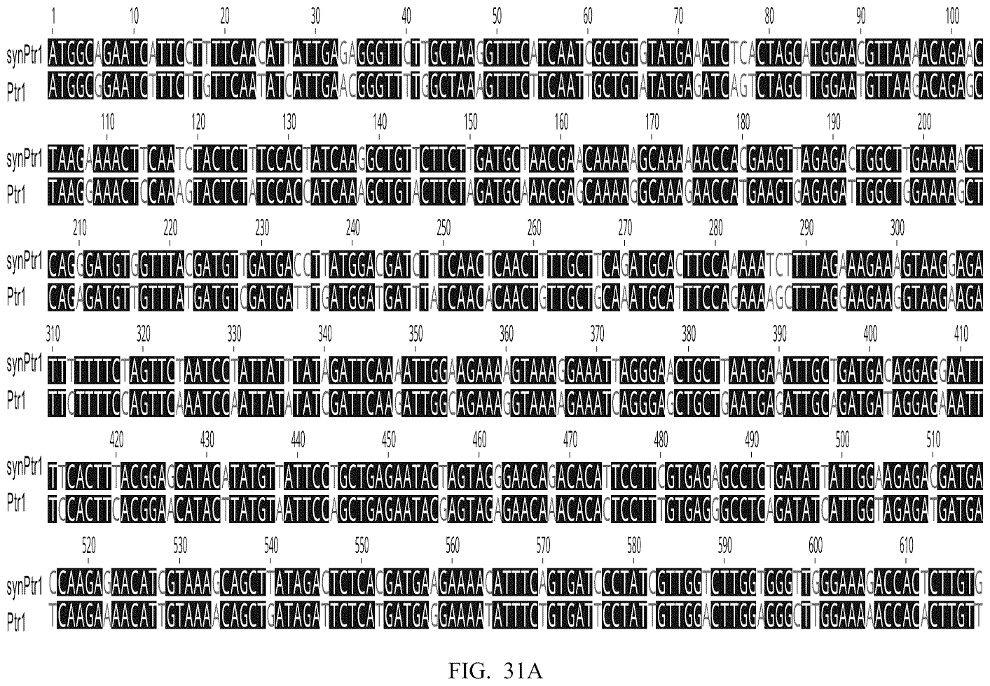

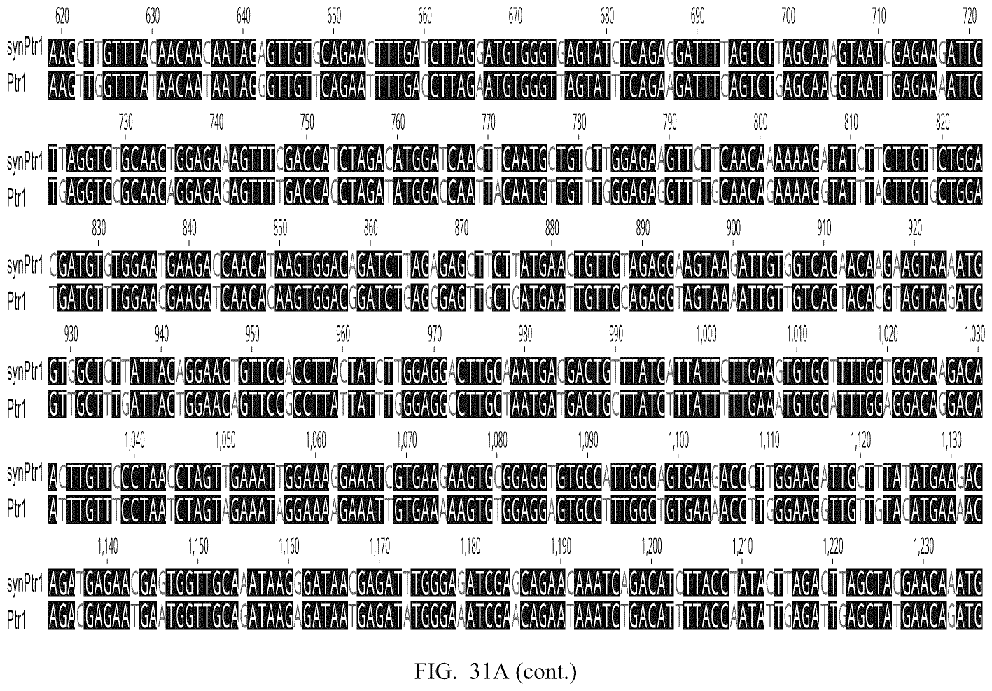

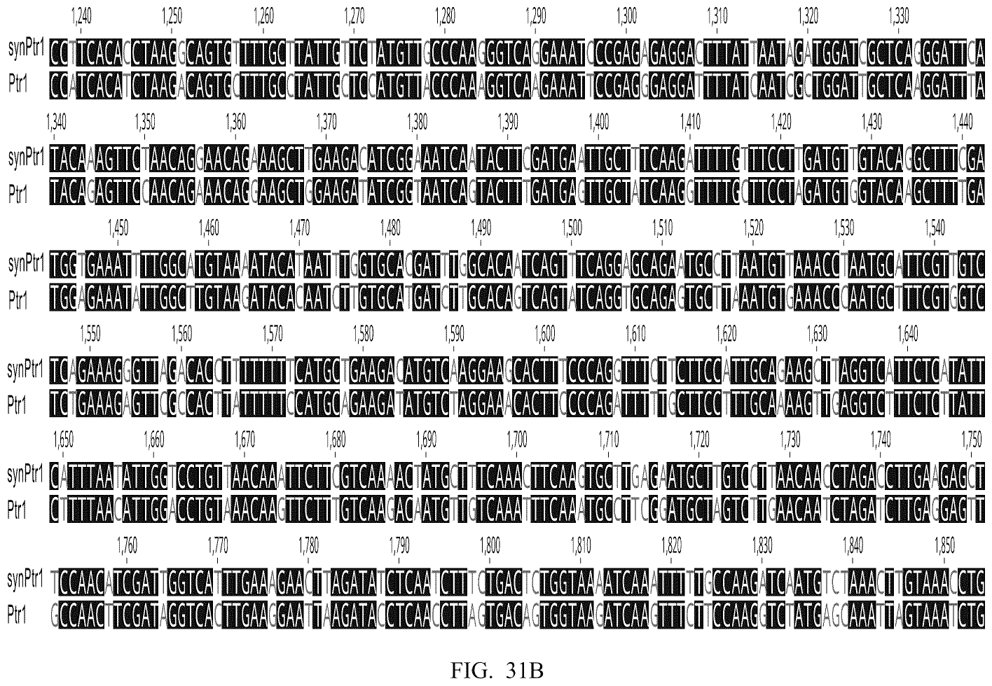

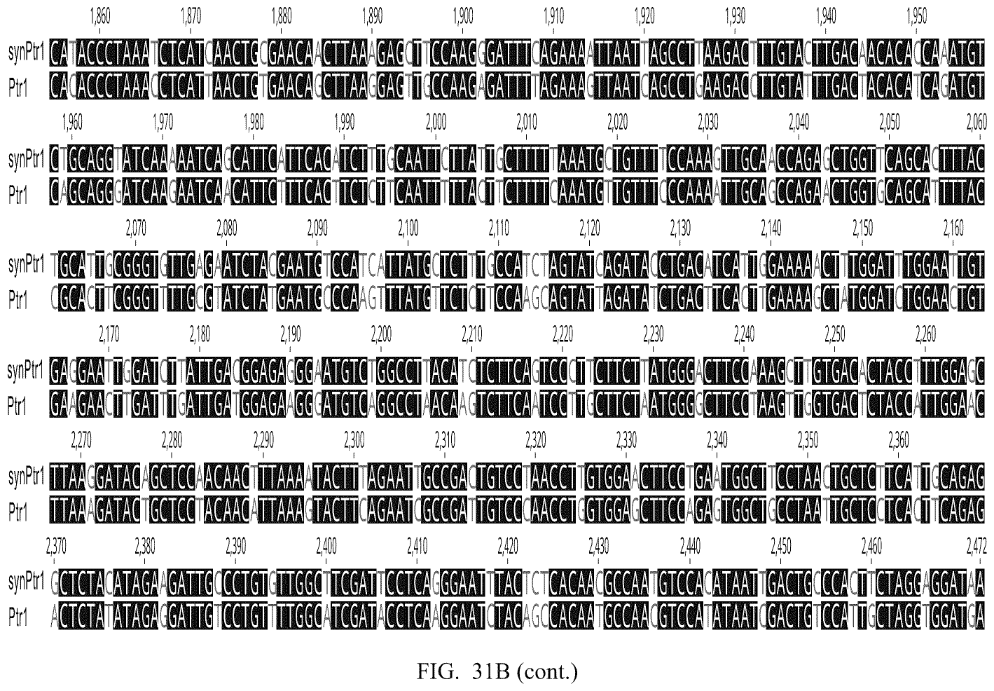

The synthetic Ptr1 (synPtr1) sequence was designed using the Integrated DNA Technologies (IDT) Codon Optimization Tool as previously described (Roberts et al., “Mail Protein Acts Between Host Recognition of Pathogen Effectors and Mitogen-Activated Protein Kinase Signaling,” Mol. Plant-Microbe. Interact. 32:1496-507 (2019), which is hereby incorporated by reference in its entirety) (FIGS. 31A-31B). The stop codon was removed in order to generate a C-terminal HA-tag and KpnI and StuI restriction sites were added to the 5′ and 3′ end, respectively, for cloning into pBTEX. The synPtr1 was synthesized by IDT (Skokie, IL, USA) as a gBlocks fragment and cloned into pBTEX using the In-fusion cloning kit following the manufacturer's instructions (Takara).

### Data Availability

Nucleotide and amino acid sequences have been deposited in GenBank for Ptr1 from S. lycopersicoides (GenBank accession no. MT134103), N. benthamiana, NbPtr1a (MT134102) and potato StPtr1 (MT134101), each of which is hereby incorporated by reference in its entirety.

### Example 13: Identifying Candidates for the Ptr1 Gene

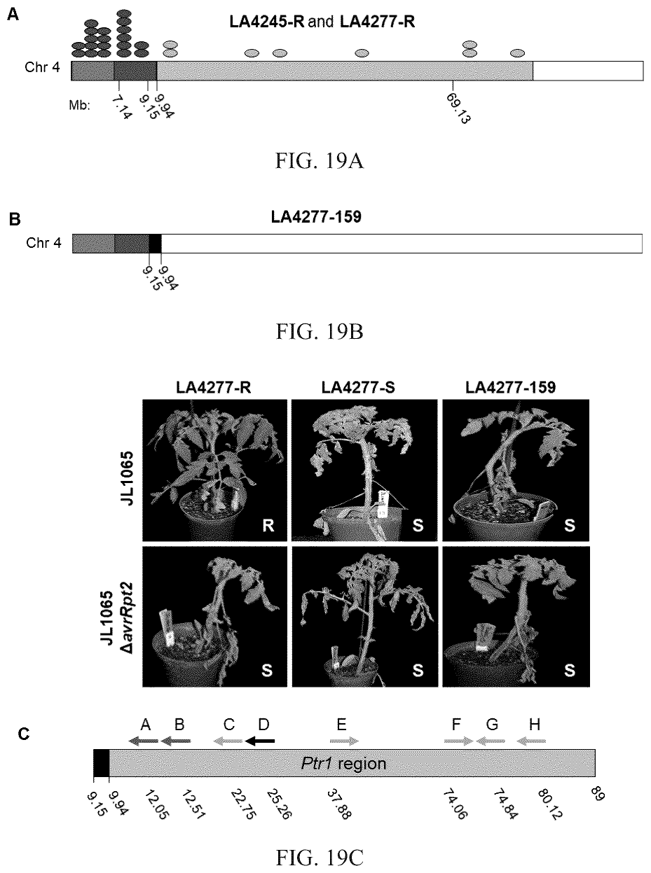

The introgression on chromosome 4 containing the Ptr1 locus is maintained in heterozygous condition and such Ptr1/ptr1 plants are referred to as LA4245-R and LA4277-R. Homozygous Ptr1/Ptr1 plants are referred to as LA4245-Ro and LA4277-Ro and ptr1/ptr1 plants are referred to as LA4245-S and LA4277-S(Examples 1-11; Mazo-Molina et al., “The Ptr1 Locus of Solanum lycopersicoides Confers Resistance to Race 1 Strains of Pseudomonas syringae pv. Tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety). The introgression region shared by LA4245-R and LA4277-R contains 16 annotated NLR-encoding genes in the current assembly of the S. lycopersicoides genome sequence (Powell et al., “A Solanum lycopersicoides Reference Genome Facilitates Biological Discovery in Tomato,” bioRxiv 2020.2004.2016.039636 (2020), which is hereby incorporated by reference in its entirety). An additional ˜50 NLR genes occur only in LA4277-R; the latter genes were not considered as candidates for Ptr1 (FIG. 19A). To identify candidates for the Ptr1 gene, F2 progeny from a selfed LA4277-R plant were screened for recombinants. Plants were genotyped for the presence of the S. lycopersicoides introgression using DNA markers at 7.14, 9.94, and 69.13 megabases (Mb) (FIG. 19A). From 585 F2 plants, one plant, LA4277-159, was identified with a recombination between markers 7.14 and 9.94. An additional marker at 9.15 Mb further delimited the region of the recombination and indicated this plant retained just a small portion of the S. lycopersicoides segment shared by both LA4245-R and LA4277-R (FIG. 19B). This segment contains eight NLR-encoding genes.

To determine if LA4277-159 contained the Ptr1 locus, selfed progeny of the recombinant plant that contain the S. lycopersicoides 0-9.94 Mb segment were identified by using DNA markers. These plants, and controls, were then vacuum infiltrated with Pst JL1065 (which has avrRpt2) and JL1065ΔavrRpt2, which lacks avrRpt2. As expected, LA4277-R, LA4277-S, and the LA4277-159 progeny all developed extensive disease upon inoculation with JL1065ΔavrRpt2 (FIG. 19B). However, when infiltrated with JL1065, LA4277-159 progeny and LA4277-S developed disease whereas LA4277-R plants remained disease-free. Considering the location of the recombination event in LA4277-159 this susceptible phenotype eliminated the eight NLR genes in the LA4277-159 S. lycopersicoides segment as candidates for Ptr1 and indicated Ptr1 is one of the eight NLR genes lying between marker 9.94 and the end of the introgression at 89 Mb (FIG. 19C). These genes were named A, B, C, D, E, F, G, and H based on their sequential location on chromosome 4 (FIG. 19C; Table 11).

TABLE 11
Predicted gene models for the Ptr1 candidates in the S. lycopersicoides
genome sequence. The start and stop of each gene model corresponds to
the genomic coordinates along the chromosome. The number of
nucleotides and amino acids correspond to the gene models. Candidate D
has a nonsense mutation at base pair 391 which terminates its open
reading frame.

| | | | | No. | No. |
|---|---|---|---|---|---|
| | | | | Nucleo- | Amino |
| Candidate | Gene ID | Start | Stop | tides | Acids |
| A | Solyd04g059470 | 12054434 | 12051962 | 2472 | 823 |
| B | Solyd04g059610 | 12505909 | 12503356 | 2553 | 850 |
| C | Solyd04g060430 | 22745706 | 22743765 | 1275 | 424 |
| D | Solyd04g060640 | 25255273 | 25252810 | 2464 | pseudo- |
| | | | | | gene |
| E | Solyd04g061490 | 37884585 | 37886367 | 1782 | 593 |
| F | Solyd04g064510 | 74057098 | 74064935 | 2457 | 818 |
| G | Solyd04g064750 | 74838450 | 74834836 | 3276 | 1091 |
| H | Solyd04g067320 | 80119015 | 80115223 | 3480 | 1159 |

Next, RNA-Seq analysis was used to determine which of the eight Ptr1 candidates is expressed in leaves of a LA4277-Ro (Ptr1/Ptr1) plant (Table 12). Seven-week-old plants were vacuum infiltrated with JL1065 and the abundance of transcripts was determined by 3′ RNA-Seq. Transcripts of only three candidates, A, B and D were detectable by this method (Table 11). Each of these genes was PCR amplified from cDNA derived from a LA4277-Ro plant and sequenced to determine whether they were the same as annotated in the S. lycopersicoides LA2951 genome sequence. In addition, an LA4277 genome sequence was generated and assembled as a comparison. Candidate D was found to be a pseudogene as it contained multiple mutations disrupting the reading frame. The sequence of candidate A was identical between LA2951 and LA4277 whereas one SNP occurred in candidate B which changes an alanine at position 647 in LA2951 to a valine in LA4277 (GCT>GTT).

TABLE 12
Transcripts of three of the Ptr1 candidates are detectable in leaves. 3′ RNA-Seq data
(normalized reads per million, RPMs) of the eight Ptr1 candidates in LA4277-Ro
(Ptr1/Ptr1) plants that were vacuum infiltrated with Pst JL1065 and sampled 5 hr later.

| | | | | | | | Standard |
|---|---|---|---|---|---|---|---|
| Candidate | Gene ID | Rep 1 | Rep 2 | Rep 3 | Rep 4 | Mean | Deviation |
| A | Solyd04g059470 | 5.89 | 4.87 | 3.61 | 2.74 | 4.28 | 1.386 |
| B | Solyd04g059610 | 0.34 | 1.26 | 0.87 | 0 | 0.62 | 0.558 |
| C | Solyd04g060430 | 0 | 0 | 0 | 0 | 0 | 0.000 |
| D | Solyd04g060640 | 1.01 | 0.94 | 1.88 | 1.31 | 1.29 | 0.428 |
| E | Solyd04g061490 | 0 | 0 | 0 | 0 | 0 | 0.000 |
| F | Solyd04g064510 | 0 | 0 | 0 | 0 | 0 | 0.000 |
| G | Solyd04g064750 | 0 | 0 | 0 | 0 | 0 | 0.000 |
| H | Solyd04g067320 | 0 | 0 | 0 | 0 | 0 | 0.000 |

### Example 14: Candidate a Mediates Recognition of AvrRpt2 and RipBN in Nicotiana glutinosa

Candidates A and B were cloned into a binary vector under control of the CaMV 35S promoter and tested by Agrobacterium-mediated transient transformation (“agroinfiltration”). Nicotiana glutinosa was used in these experiments because it was reported previously that AvrRpt2 does not cause cell death in this species whereas it does in N. benthamiana and this would have interfered with the assays (Mudgett et al., “Characterization of the Pseudomonas syringae pv. Tomato AvrRpt2 Protein: Demonstration of Secretion and Processing During Bacterial Pathogenesis,” Mol. Microbiol. 32:927-41 (1999); Day et al., “Molecular Basis for the RIN4 Negative Regulation of RPS2 Disease Resistance,” Plant Cell 17:1292-305 (2005); and Kessens et al., “Determining the GmRIN4 Requirements of the Soybean Disease Resistance Proteins Rpg1b and Rpg1r Using a Nicotiana glutinosa-Based Agroinfiltration System,” PLoS One 9:e108159 (2014), which are hereby incorporated by reference in their entirety).

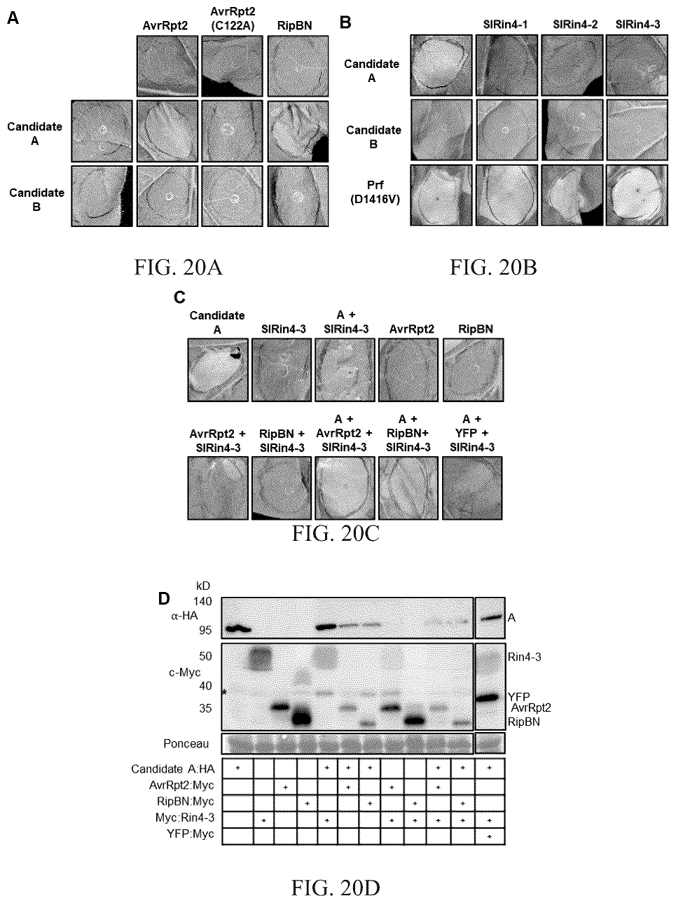

Syringe infiltration into leaves of N. glutinosa of a relatively low titer (OD₆₀₀=0.025) of Agrobacterium carrying constructs of candidate A or B expressed from the CaMV 35S promoter caused no observable host response (FIG. 20A). However, co-expression of candidate A with either AvrRpt2 or RipBN induced cell death two days after infiltration. No cell death was observed when candidate A was co-expressed with the protease-inactive protein AvrRpt2(C122A). Candidate B did not induce cell death when co-expressed with any of the effector proteins (FIG. 20A).

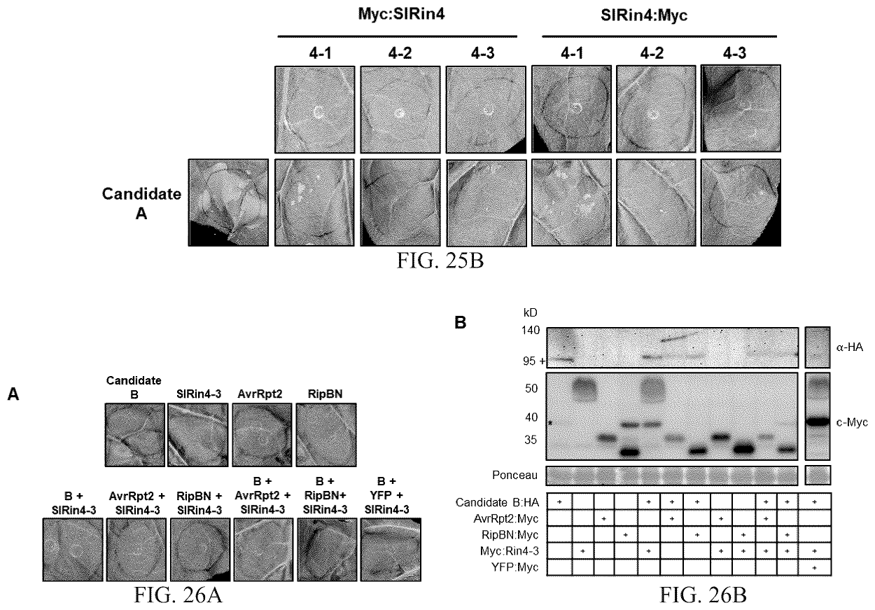

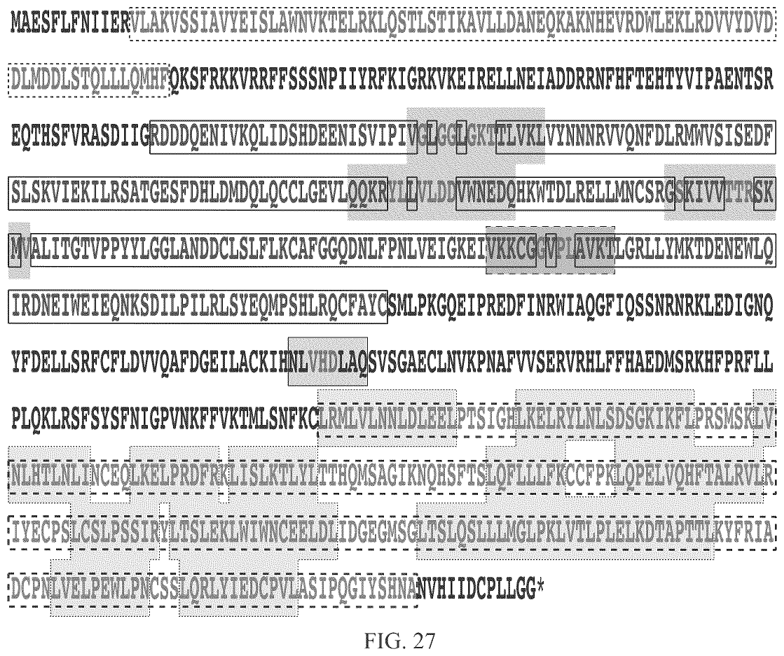

RPS2 can cause cell death on its own when overexpressed in N. benthamiana leaves and this cell death is suppressed by co-expression of AtRIN4 (Day et al., “Molecular Basis for the RIN4 Negative Regulation of RPS2 Disease Resistance,” Plant Cell 17:1292-305 (2005), which is hereby incorporated by reference in its entirety). Therefore Agrobacterium strains carrying the candidate A and B constructs were syringe infiltrated at a four-fold higher titer (OD₆₀₀=0.1). At this titer, candidate A, but not candidate B, induced strong cell death on its own. Tomato has three Rin4 genes that are expressed in leaves (SlRin4-1, SlRin4-2, and SlRin4-3) (FIG. 25A; (Examples 1-11; Mazo-Molina et al., “The Ptr1 Locus of Solanum lycopersicoides Confers Resistance to Race 1 Strains of Pseudomonas syringae pv. tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety). To test whether the tomato Rin4 proteins can suppress cell death caused by candidate A, SlRin4-1, SlRin4-2 and SlRin 4-3 were expressed with candidate A in N. glutinosa leaves. Each of these proteins suppressed candidate A-mediated cell death (FIG. 20B; FIG. 25B). This observation indicated that the tomato Rin4 proteins are functionally equivalent and we therefore chose one, SlRin4-3, as representative of these proteins for some of the subsequent experiments. Candidate B did not cause cell death even at higher Agrobacterium titers nor was it affected by co-expression of SlRin4-3 or either of the effectors (FIG. 20B; FIGS. 26A-26B). A constitutively-active version of Prf was used as a positive control for cell death in these experiments and this response was not suppressed by co-expression with the tomato SlRin4 proteins (FIG. 20B).

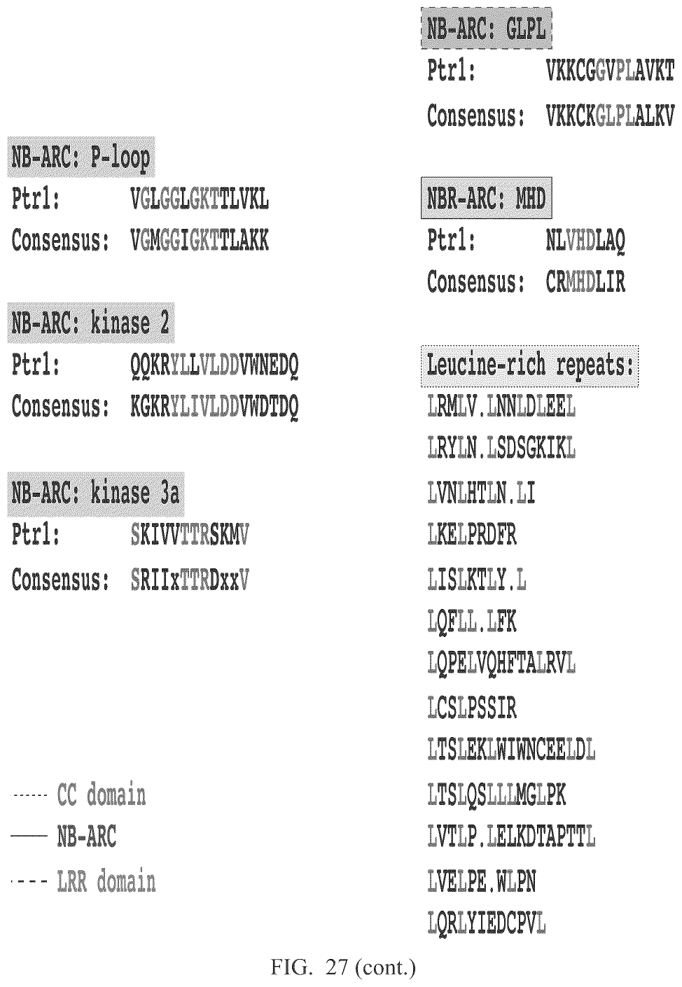

In Arabidopsis, AvrRpt2 cleaves AtRIN4 resulting in the activation of RPS2 (Axtell et al., “Initiation of RPS2-Specified Disease Resistance in Arabidopsis is Coupled to the AvrRpt2-Directed Elimination of RIN4,” Cell 112:369-77 (2003); and Mackey et al., “Arabidopsis RIN4 is a Target of the Type III Virulence Effector AvrRpt2 and Modulates RPS2-Mediated Resistance,” Cell 112:379-89 (2003), which are hereby incorporated by reference in their entirety). To test whether this is the case for candidate A, we co-expressed candidate A with SlRin4-3 with or without the effectors AvrRpt2 and RipBN (FIG. 20C). Co-expression of either one of the effectors with candidate A and SlRin4-3 restored candidate A-induced cell death and this was correlated with a decrease in the abundance of Rin4 protein (FIG. 20D). All proteins were found to be expressed as expected by immunoblotting (FIG. 20D). These experiments demonstrate that candidate A is the Ptr1 gene. The predicted Ptr1 protein is a coiled-coil NLR with 13 leucine-rich repeats and all of the other hallmark motifs of this class of protein (FIG. 27).

### Example 15: Ptr1 is a Pseudogene in Tomato and S. pennellii, but Intact Ptr1 Genes Occur in Other Solanaceous Species

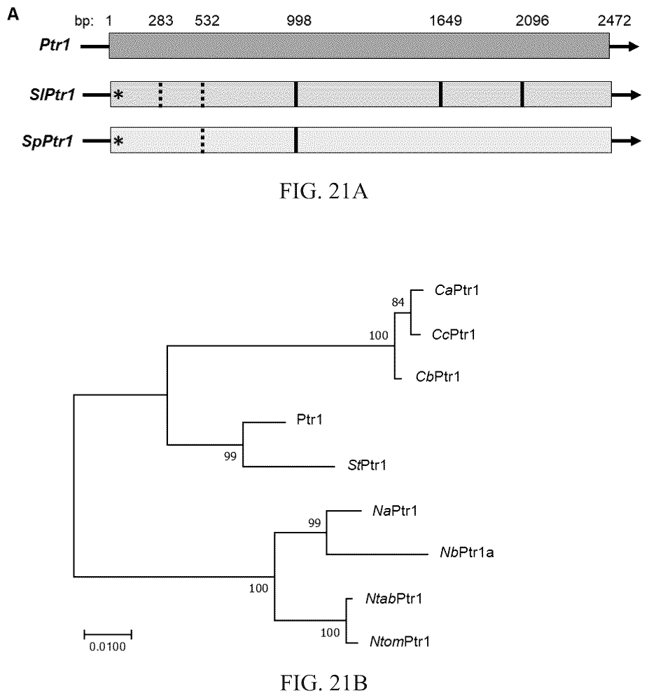

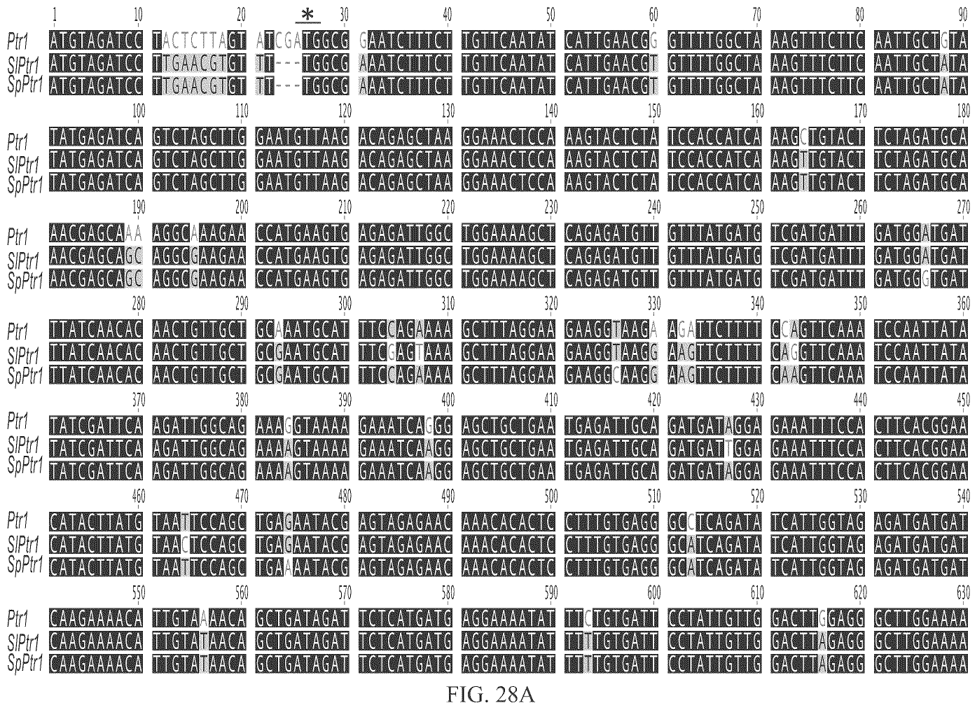

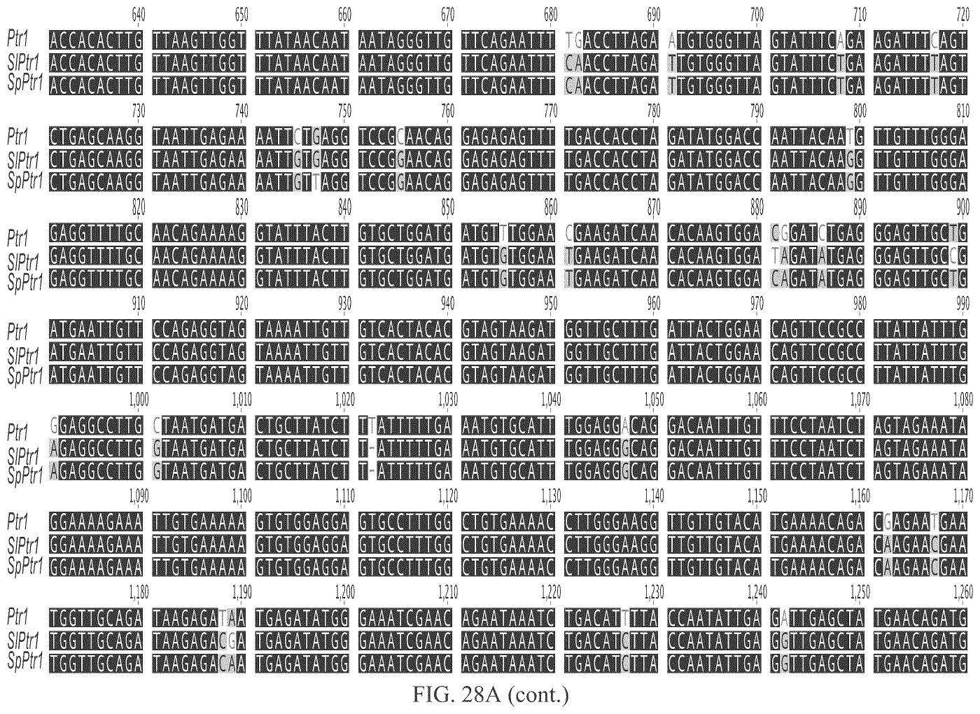

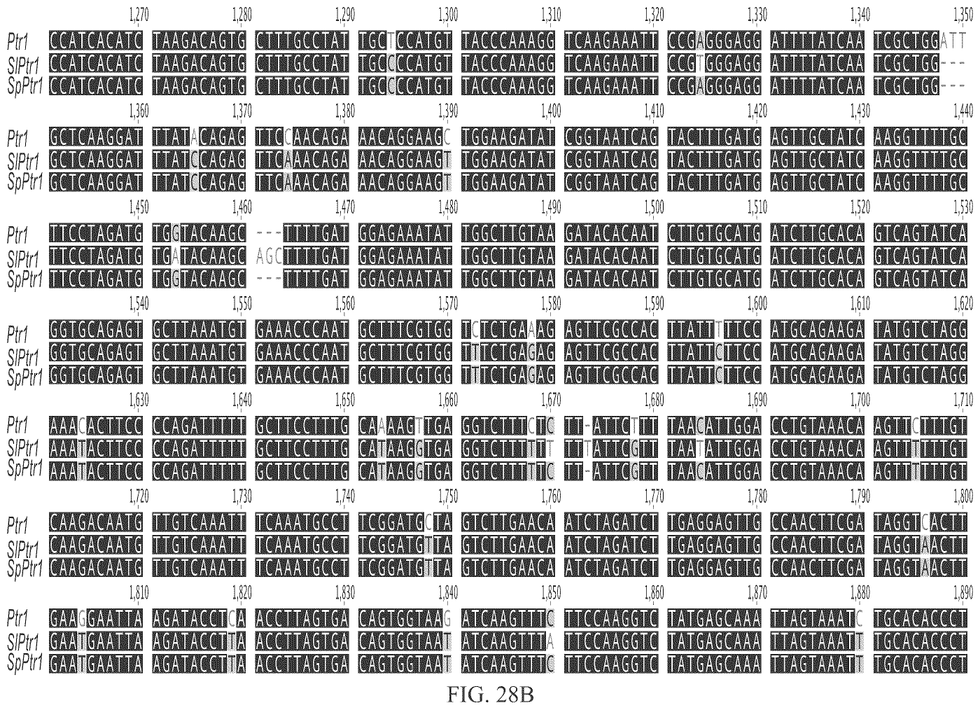

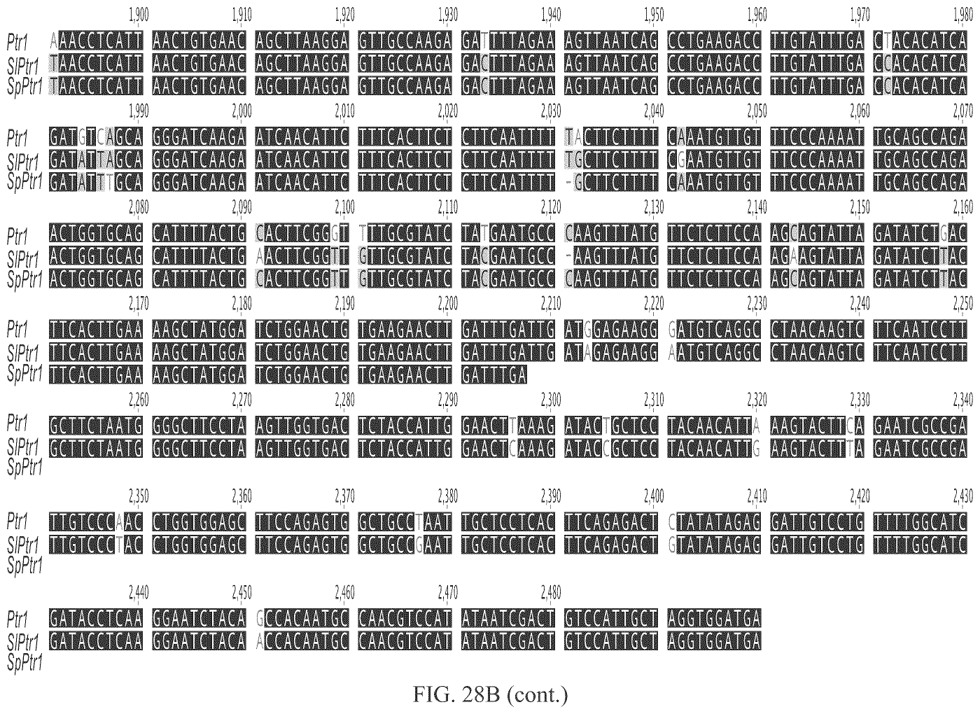

No accessions of tomato or its close wild relatives are known that recognize AvrRpt2. The genome sequences of the tomato reference genome (Heinz 1706) and its wild relative S. pennellii (LA0716; Bolger et al., “The Genome of the Stress-Tolerant Wild Tomato Species Solanum pennellii,” Nat. Genet. 46:1034-8 (2014), which is hereby incorporated by reference in its entirety) were searched, and it was found that, while both have a clear ortholog of Ptr1 (with greater than 95% identical nucleotide sequences) which occurs at a syntenous location compared with S. lycopersicoides, a small deletion abolishes the Ptr1 start codon in both species and multiple other nonsense and frameshift mutations disrupt the reading frames of these Ptr1 genes (FIG. 21A; FIGS. 28A-28B).

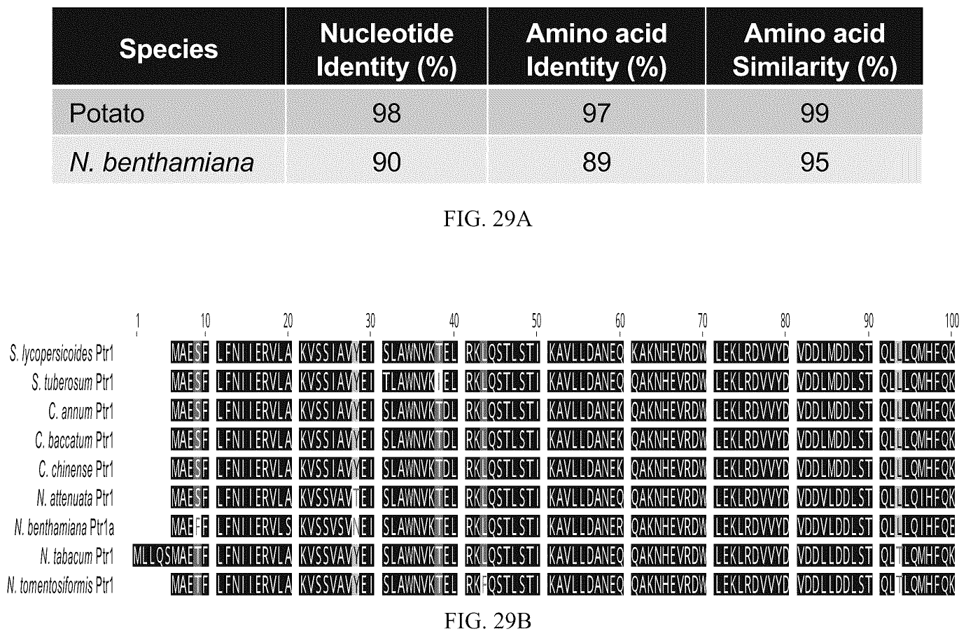

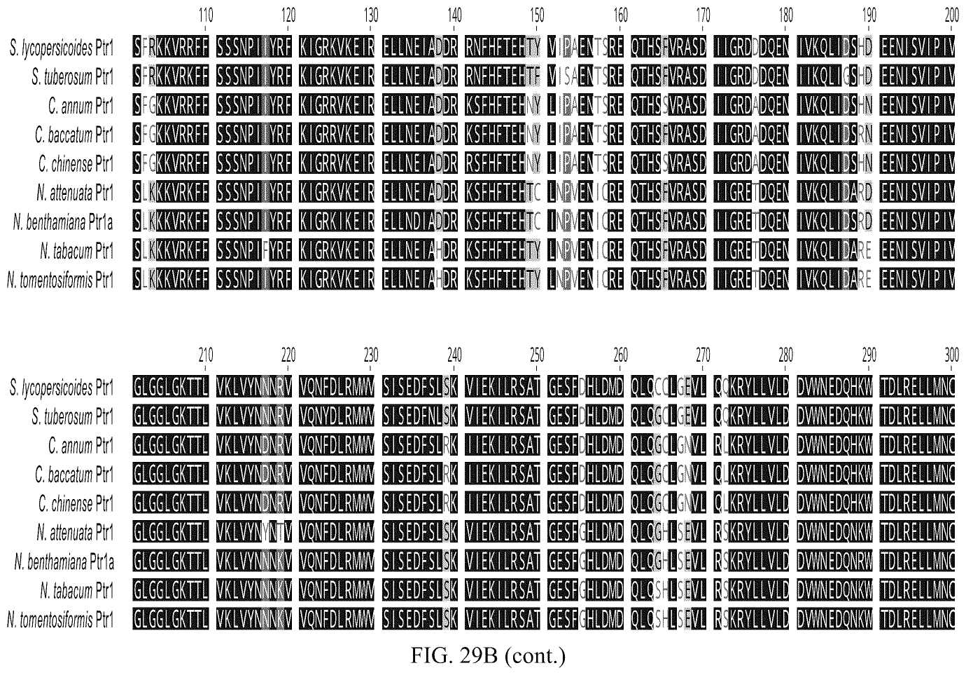

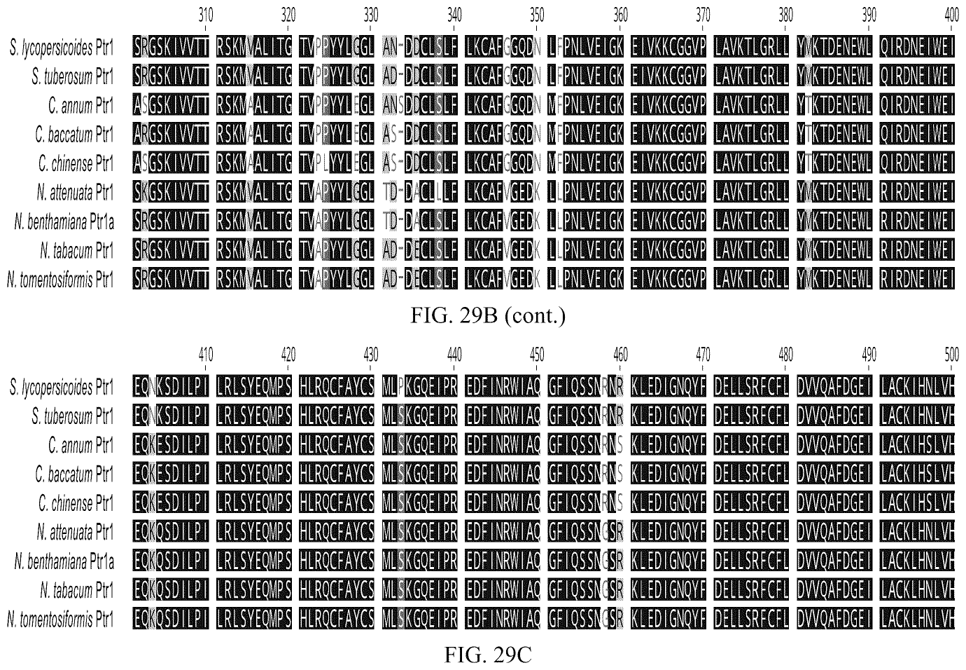

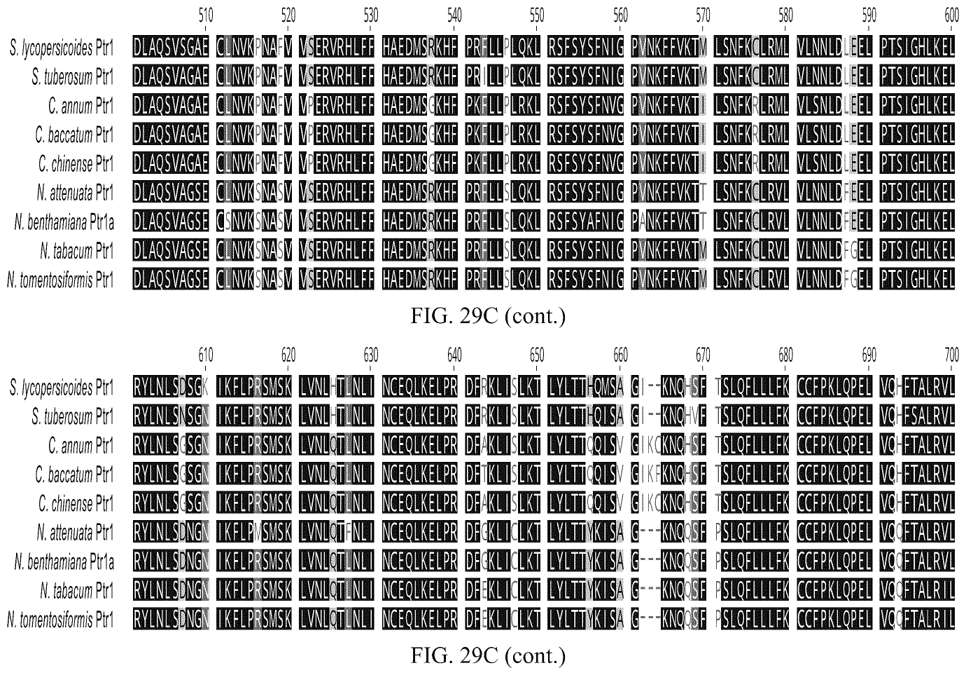

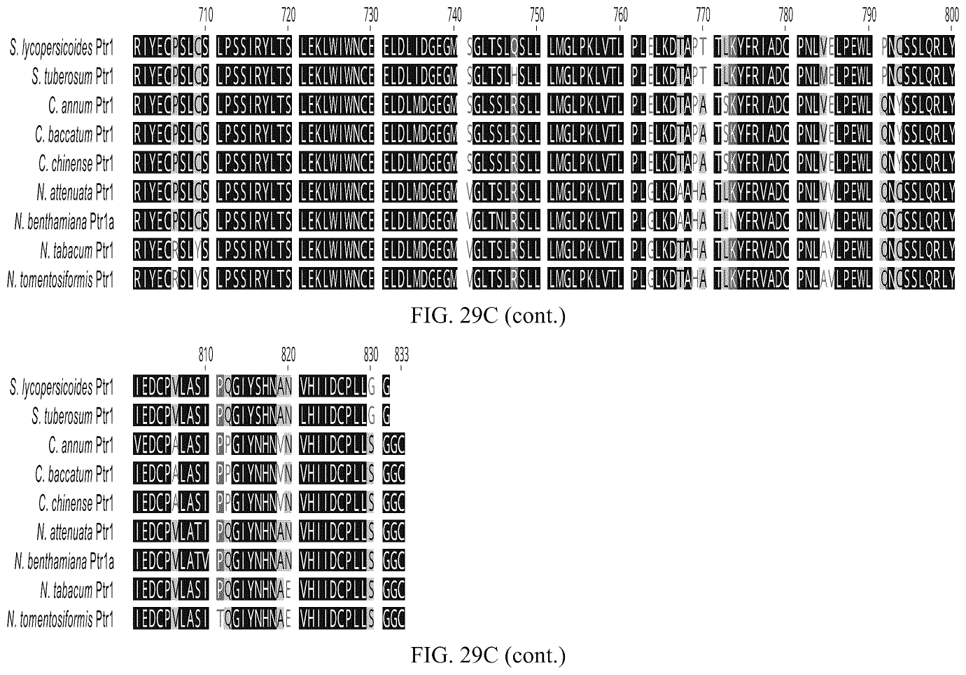

A broader search identified intact Ptr1 orthologs in a variety of other solanaceous plants, including potato, three species of pepper, and four species of tobacco including N. benthamiana (FIG. 21B). In all of these species the predicted amino acid similarity between Ptr1 and that encoded by the orthologs is greater than 95% (FIGS. 29A-29C; Table 9). Ptr1 occurs at syntenous locations in potato and pepper compared with S. lycopersicoides; it was not possible to make this determination in other species based on current genome sequence assemblies.

### Example 16: The Ptr1 Ortholog in N. benthamiana and in Potato Mediates Recognition of AvrRpt2 and RipBN

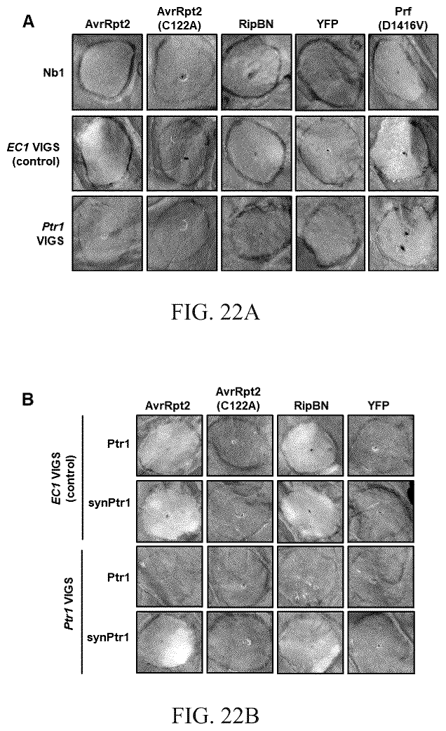

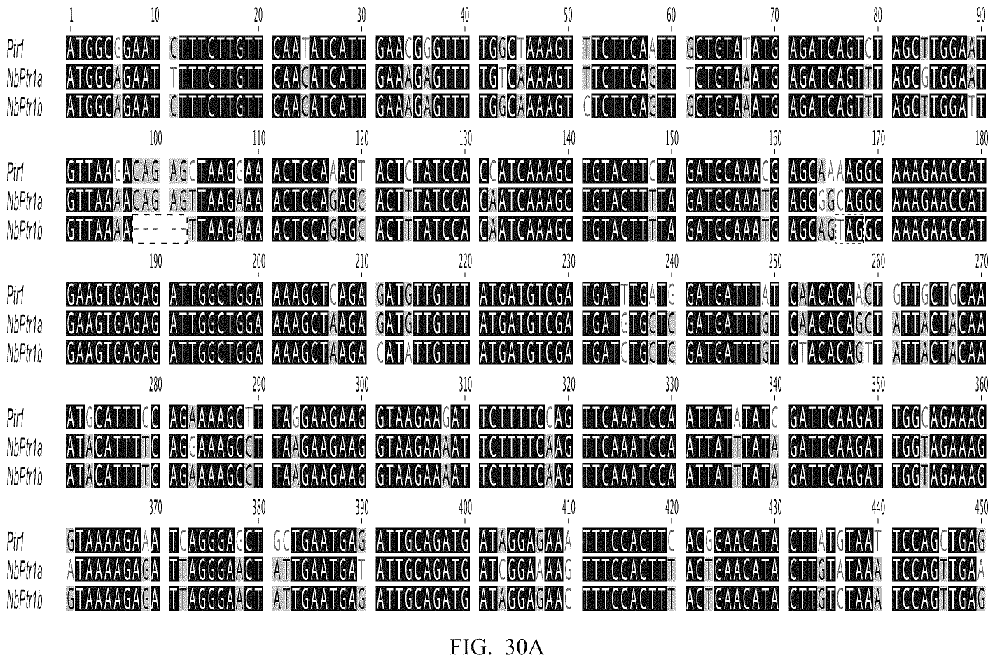

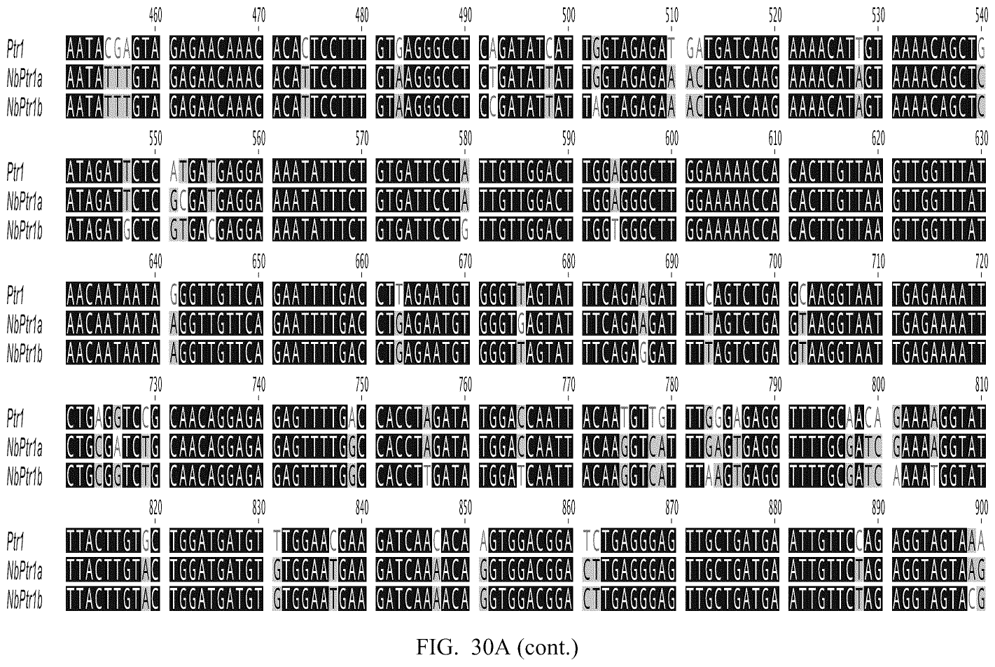

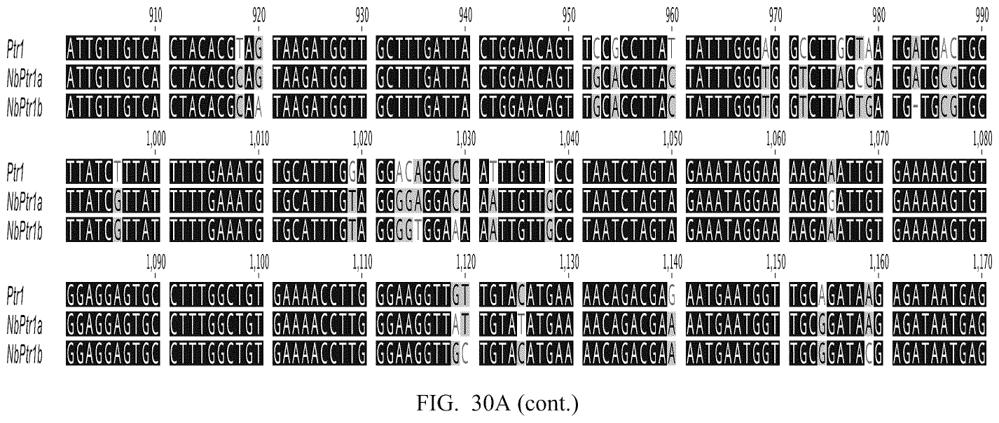

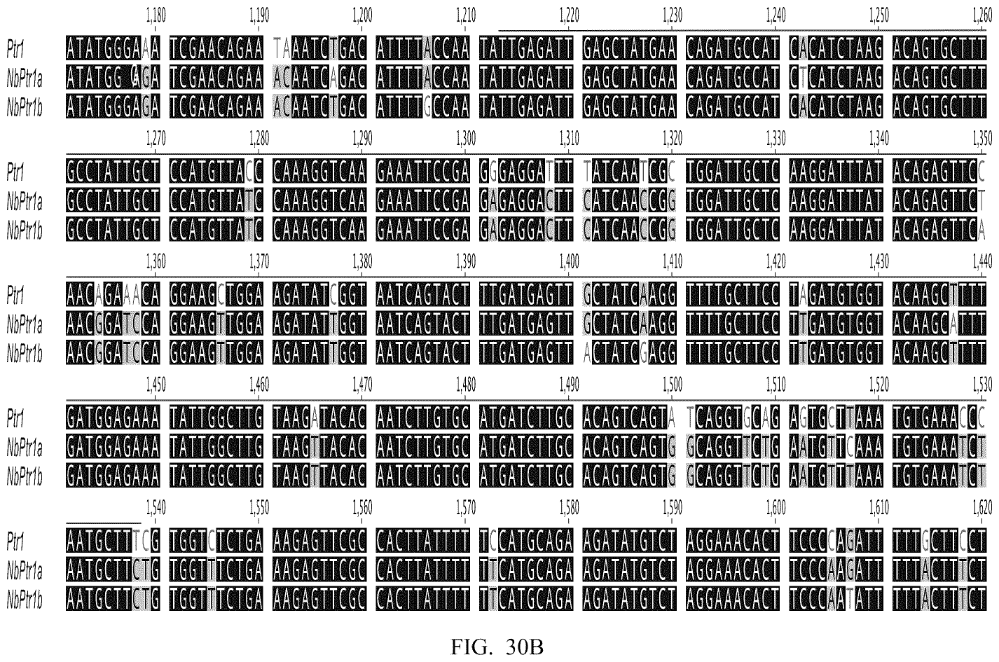

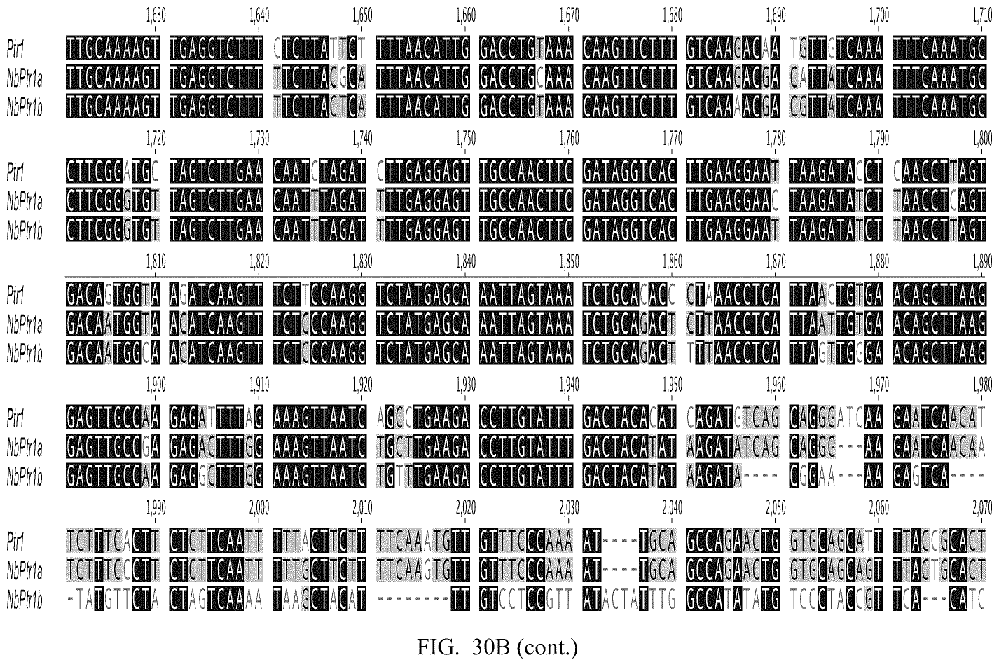

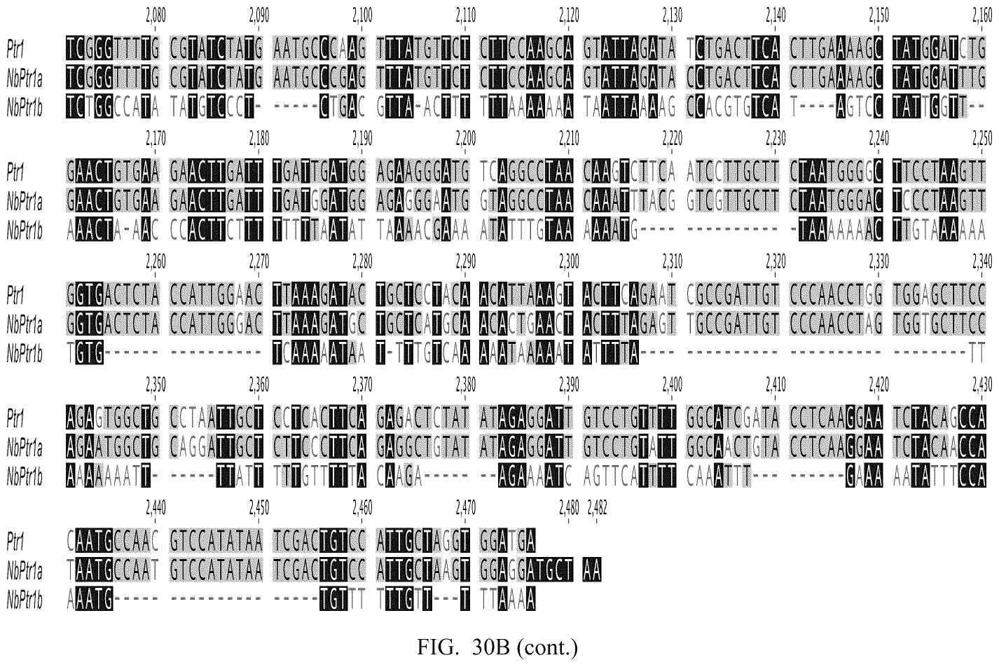

The observation that expression of AvrRpt2 alone in N. benthamiana leaves causes cell death raised the possibility that the Ptr1 ortholog in this species is responsible for this response. As expected for an allotetraploid, N. benthamiana has two Ptr1 orthologs (NbPtr1a and NbPtr1b), although a 5-base pair deletion in NbPtr1b leads to a premature stop codon (FIGS. 30A-30B). To test whether NbPtr1a plays a role in AvrRpt2-mediated cell death in N. benthamiana, a 325-bp fragment of this gene was selected for use in tobacco rattle virus (TRV)-induced gene silencing (VIGS; FIGS. 30A-30B). N. benthamiana (Nb1; Bombarely et al, “A draft Genome Sequence of Nicotiana benthamiana to Enhance Molecular Plant-Microbe Biology Research,” Mol. Plant-Microbe Interact. 25:1523-30 (2012), which is hereby incorporated by reference in its entirety) plants were infected with TRV:Ptr1 or a control, TRV:EC1, containing a small fragment of E. coli DNA (Rosli et al., “Transcriptomics-Based Screen for Genes Induced by Flagellin and Repressed by Pathogen Effectors Identifies a Cell Wall-Associated Kinase Involved in Plant Immunity,” Genome Biol. 14:R139 (2013), which is hereby incorporated by reference in its entirety). Expression of AvrRpt2 or RipBN, but not of the controls AvrRpt2(C122A) or YFP, induced strong cell death in uninfected Nb1 leaves, as well as those infected with TRV:EC1 (FIG. 22A). No cell death was observed in NbPtr1-silenced plants, indicating the Ptr1 ortholog mediates AvrRpt2 recognition in N. benthamiana. The constitutively-active Prf protein induced cell death in all the plants as expected.

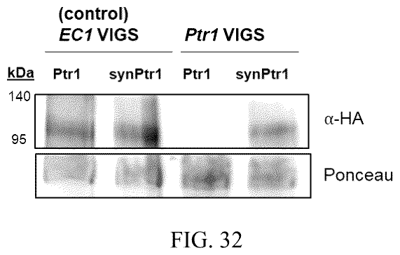

To assure that the loss of AvrRpt2-induced cell death observed in the Ptr1-silenced plants was not due to silencing of an ‘off-target’ gene, a synthetic version of Ptr1 (synPtr1) was developed with a divergent DNA sequence that would make it resistant to silencing, yet encode an identical amino acid sequence (FIGS. 31A-31B). Immunoblotting showed that both Ptr1 and synPtr1 proteins accumulated in the TRV:EC1 control plants, whereas in Ptr1-silenced plants only the protein encoded by synPtr1 accumulated (FIG. 32). Co-expression in Ptr1-silenced plants of synPtr1 with either AvrRpt2 or RipBN induced cell death whereas cell death did not occur in silenced plants co-expressing Ptr1 with the effectors (FIG. 22B). No cell death occurred in any plants in response to AvrRpt2(C122A). These experiments demonstrate that Ptr1 and its function in mediating recognition of proteolytically-active AvrRpt2 is conserved in N. benthamiana.

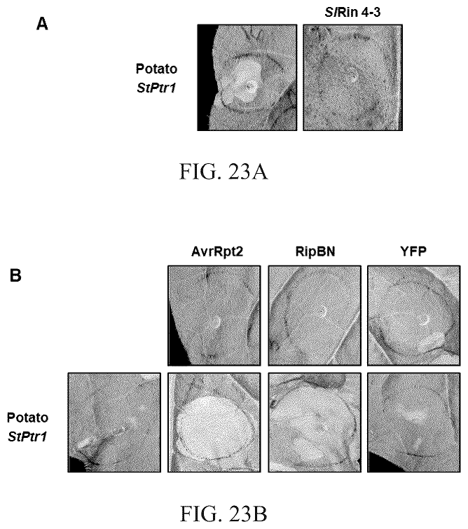

The protein predicted to be encoded by the Ptr1 ortholog in potato (StPtr1) is 97% identical to the Ptr1 protein and it seemed likely that it might also mediate recognition of AvrRpt2 and RipBN (FIGS. 29A-29C). Attempted use of Agrobacterium-infiltration to transiently express AvrRpt2 or RipBN in potato leaves did not cause cell death possibly due to the inefficiency of this method in this species. The StPtr1 ortholog from potato variety Dakota Crisp was cloned and expressed using a high titer of Agrobacterium in N. glutinosa leaves. StPtr1 caused cell death on its own at this titer which was inhibited by co-expression of SlRin4-3 just as observed for Ptr1 (FIG. 23A). As with Ptr1, using a lower titer of Agrobacterium prevented StPtr1-induced cell death and co-expression of AvrRpt2 or RipBN in this condition induced cell death (FIG. 23B). Ptr1 is therefore conserved in diverse solanaceous species, but is a pseudogene in tomato and S. pennellii.

### Example 17: Ptr1 is not Orthologous to Mr5 or RPS2

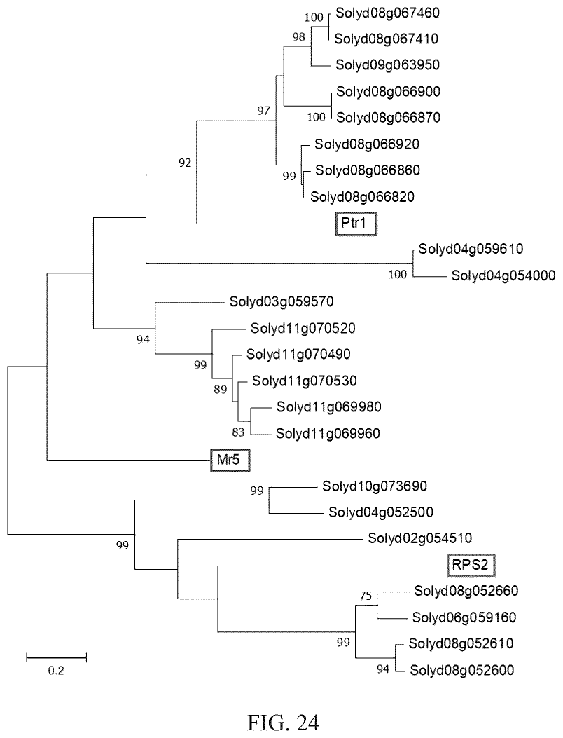

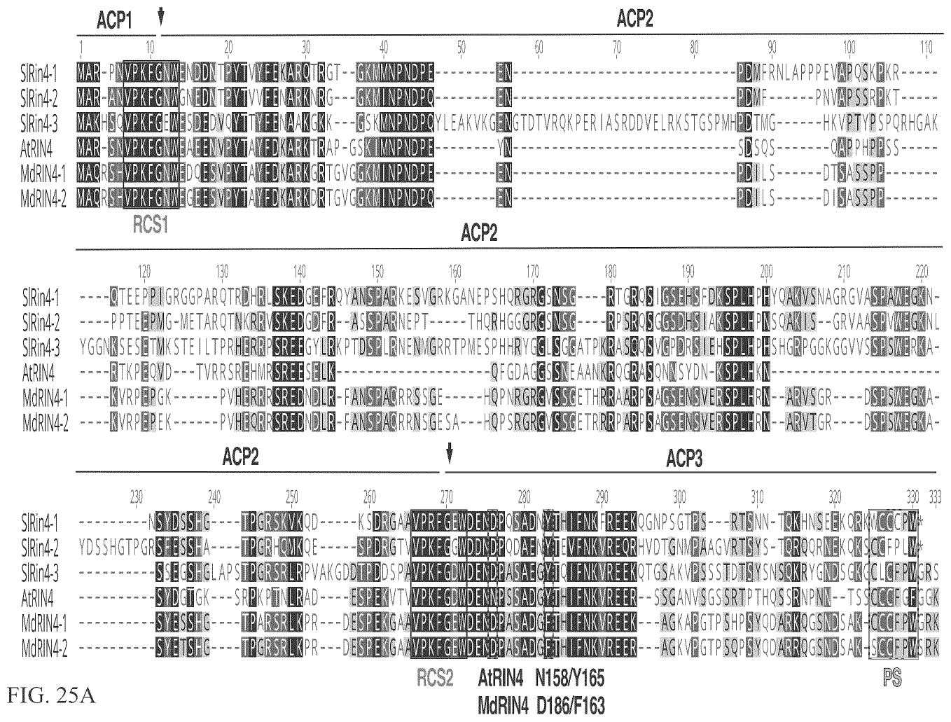

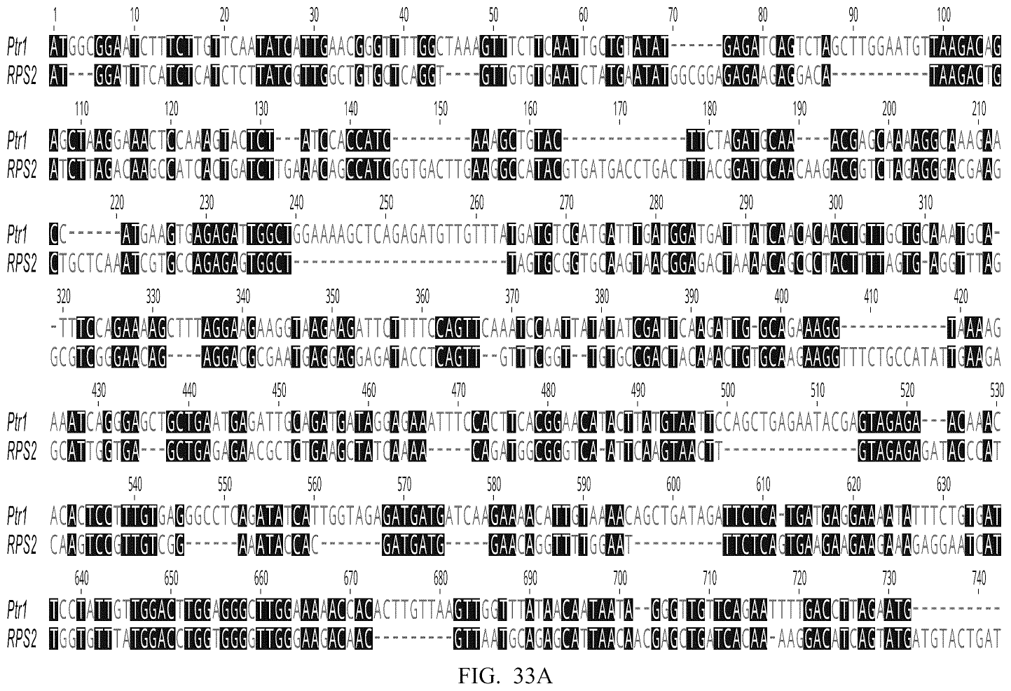

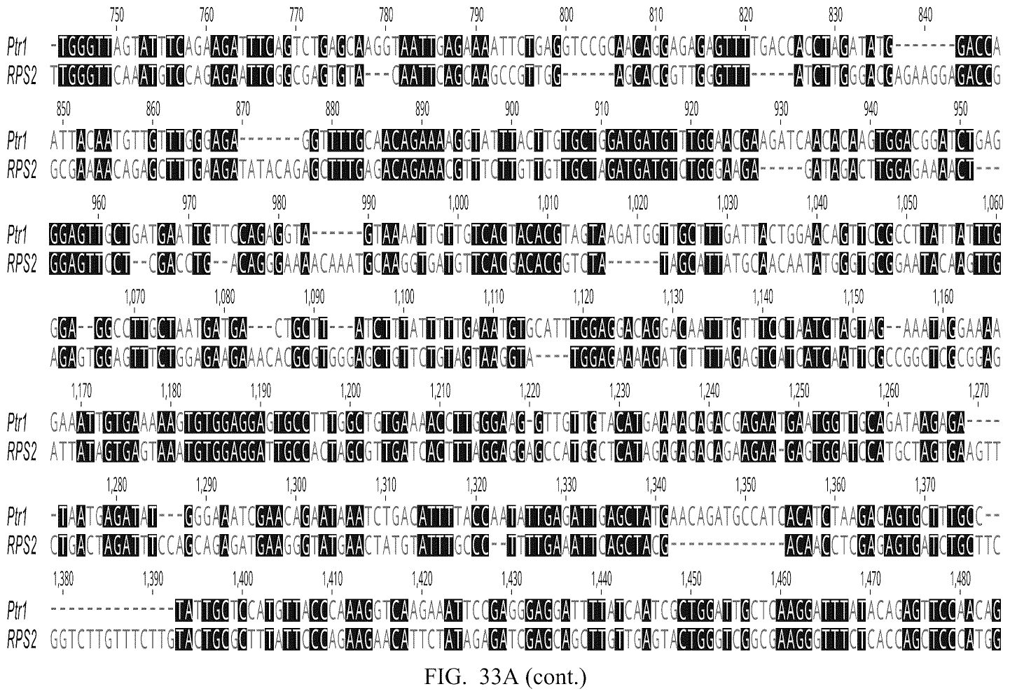

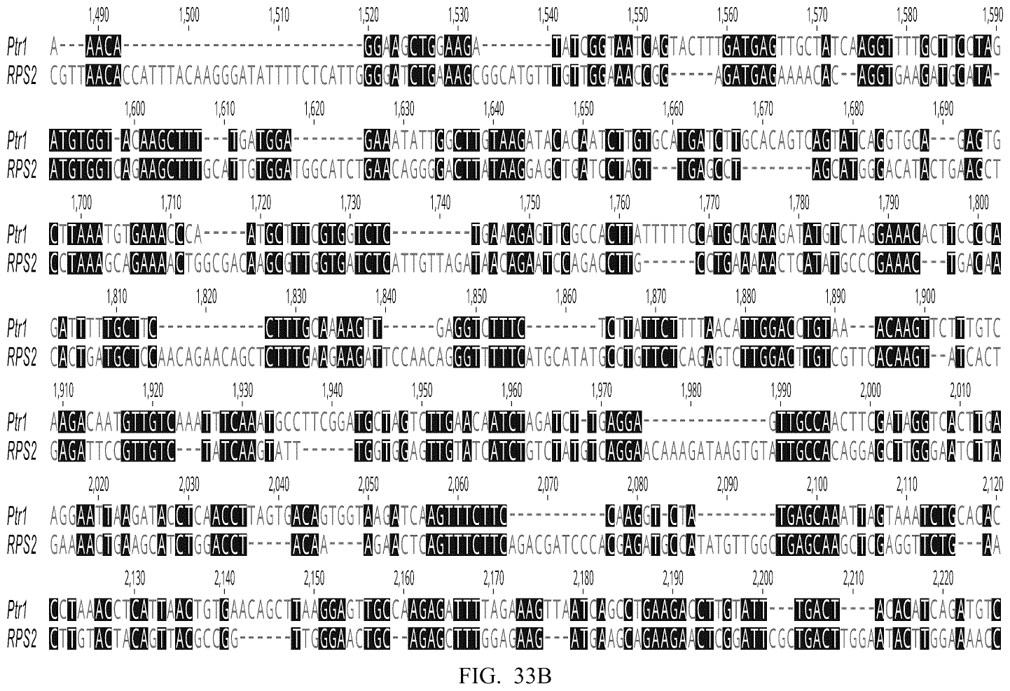

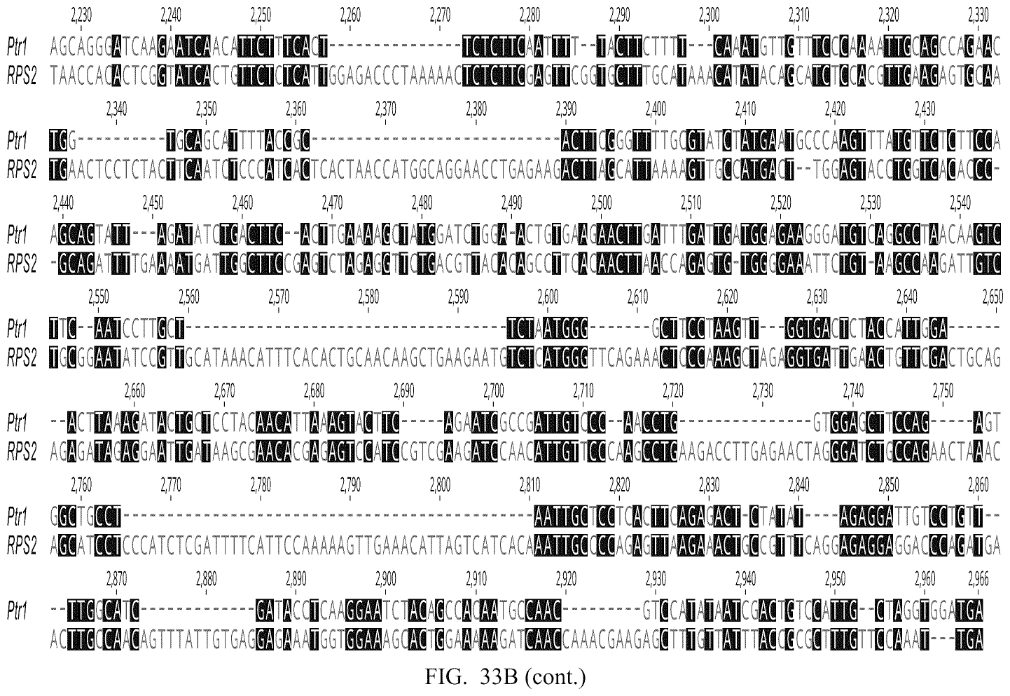

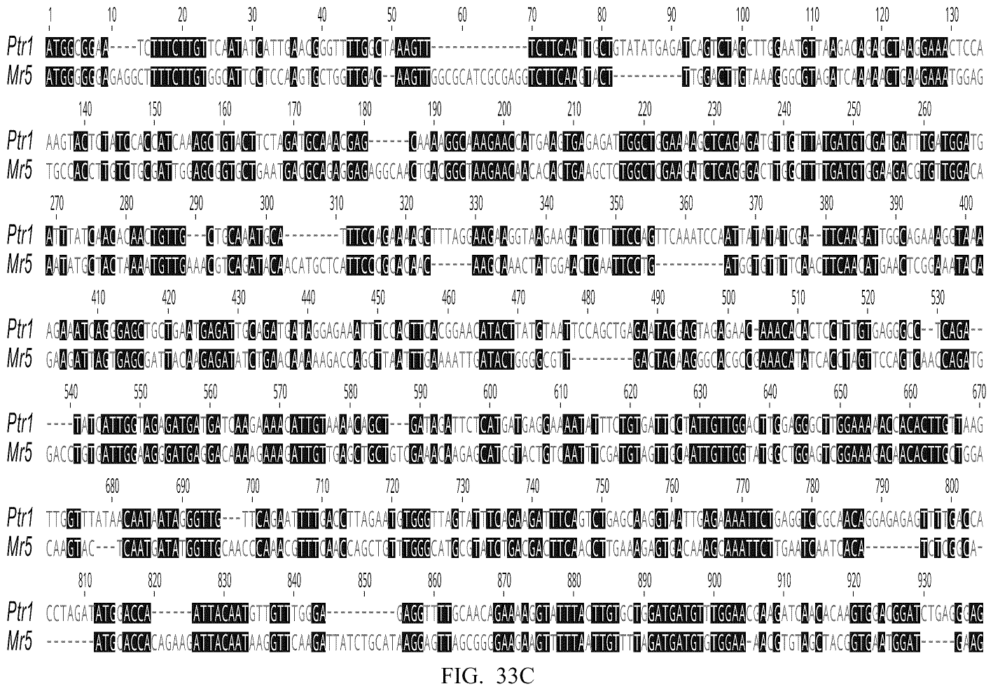

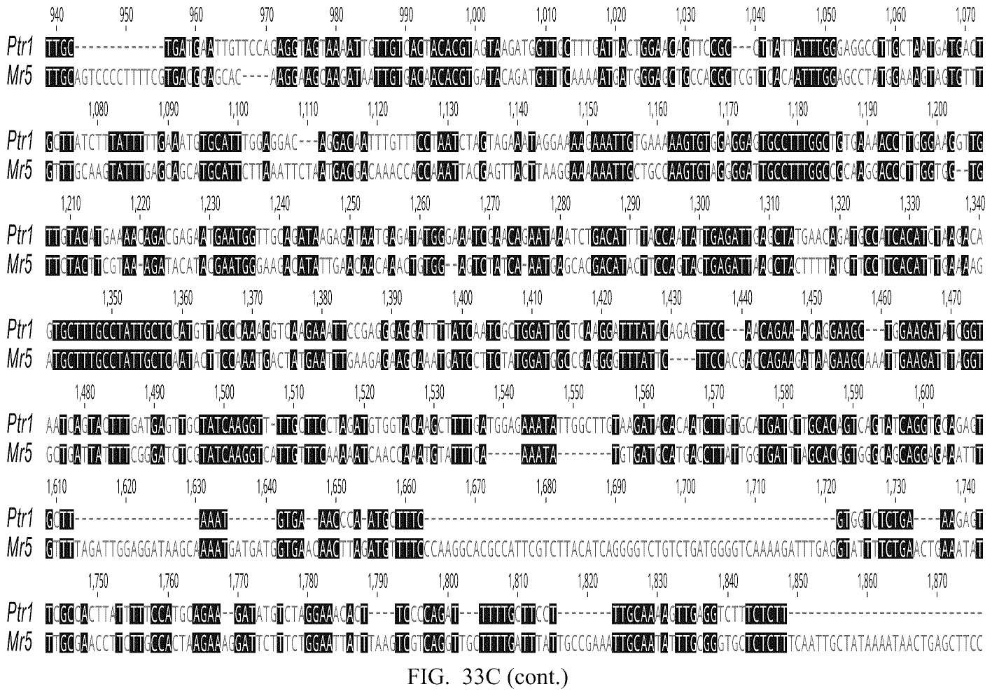

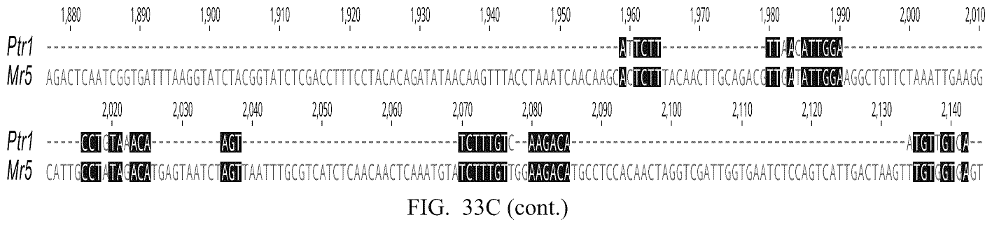

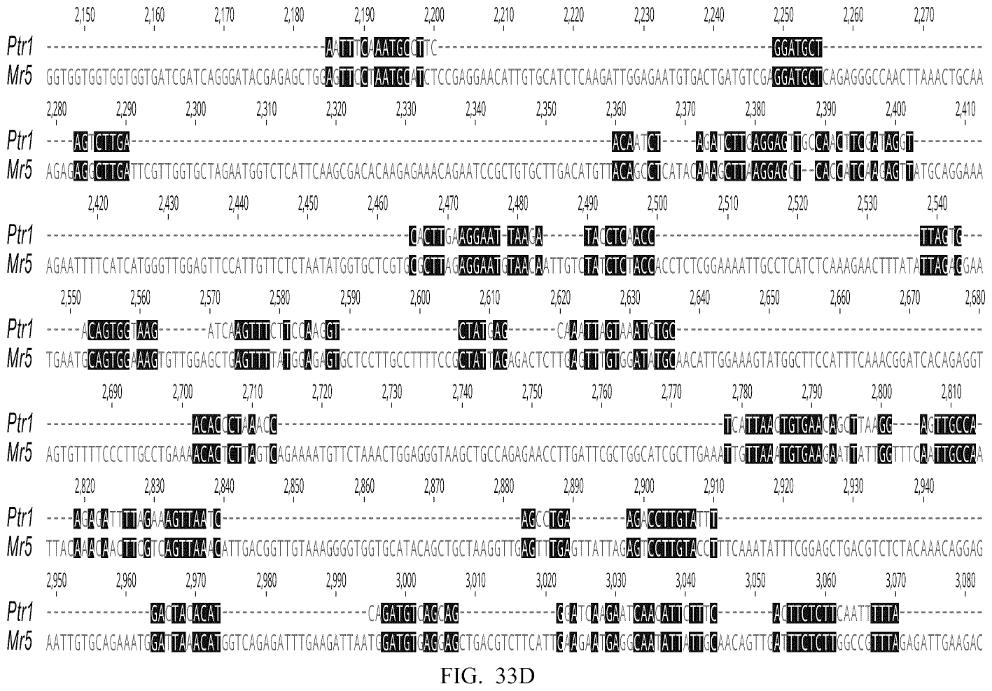

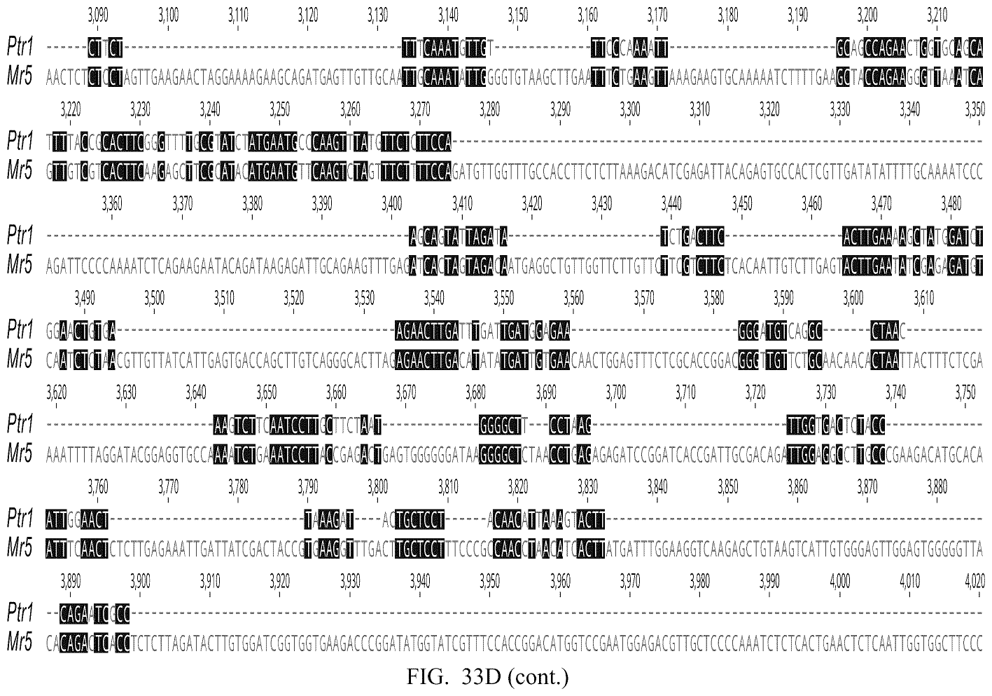

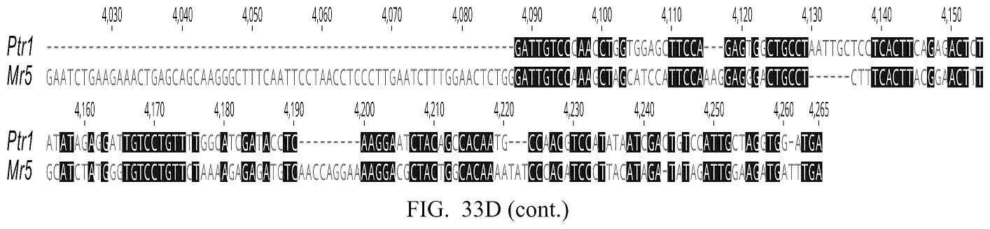

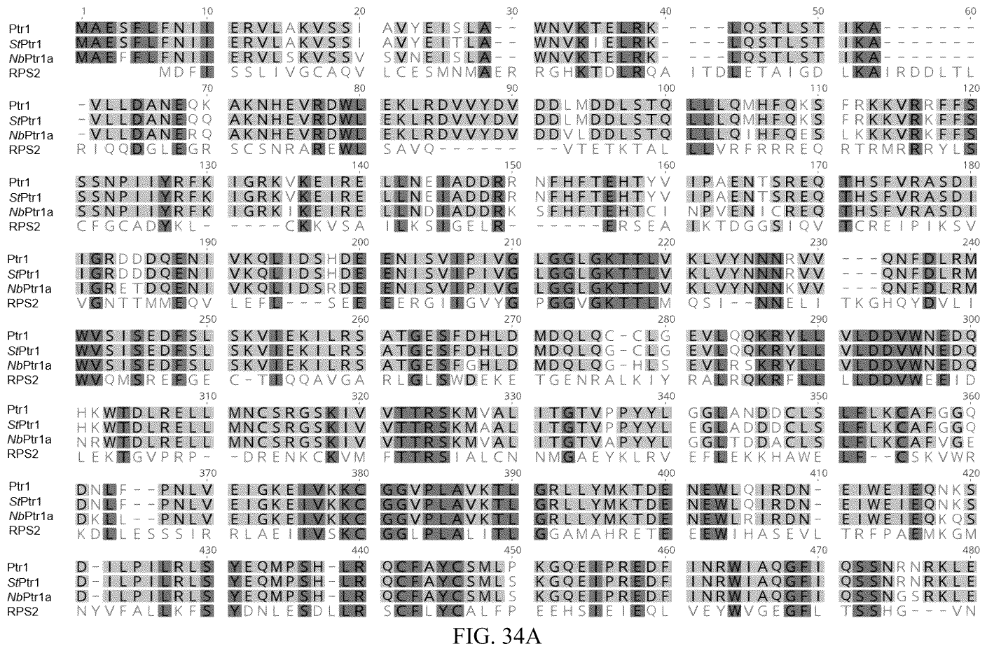

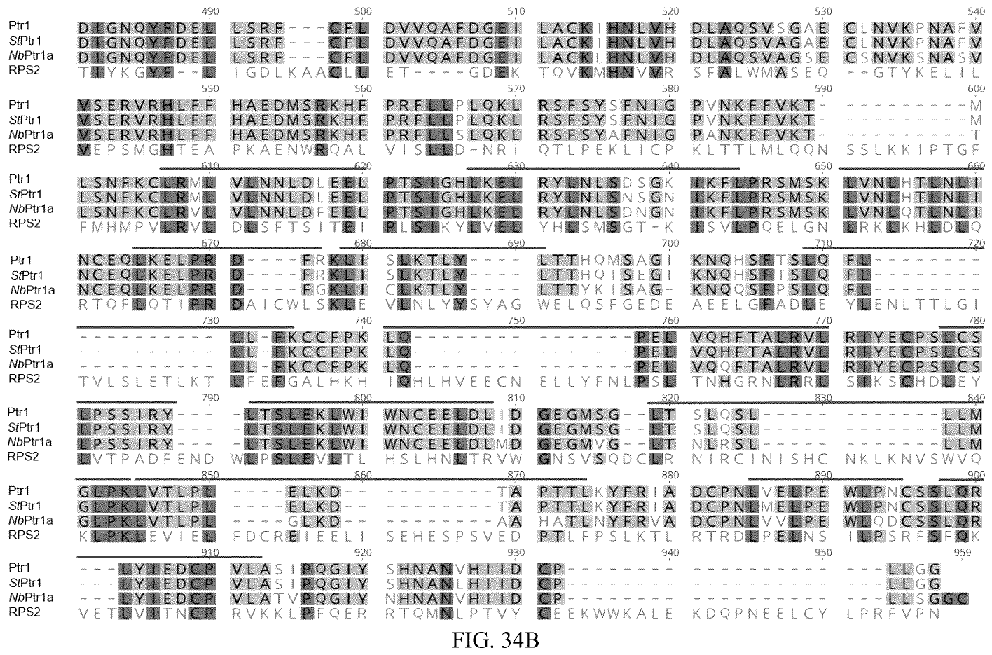

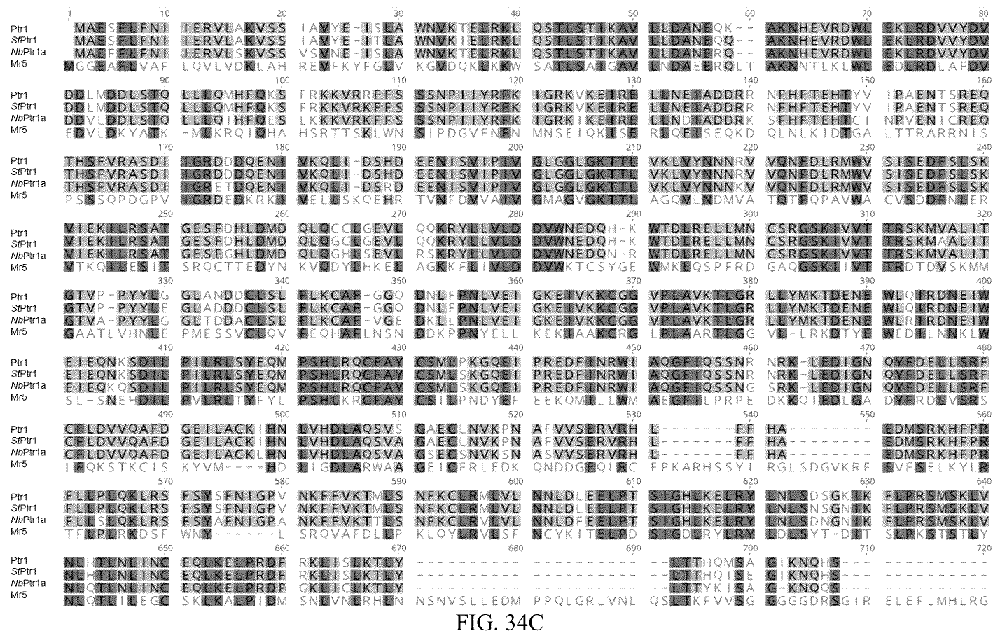

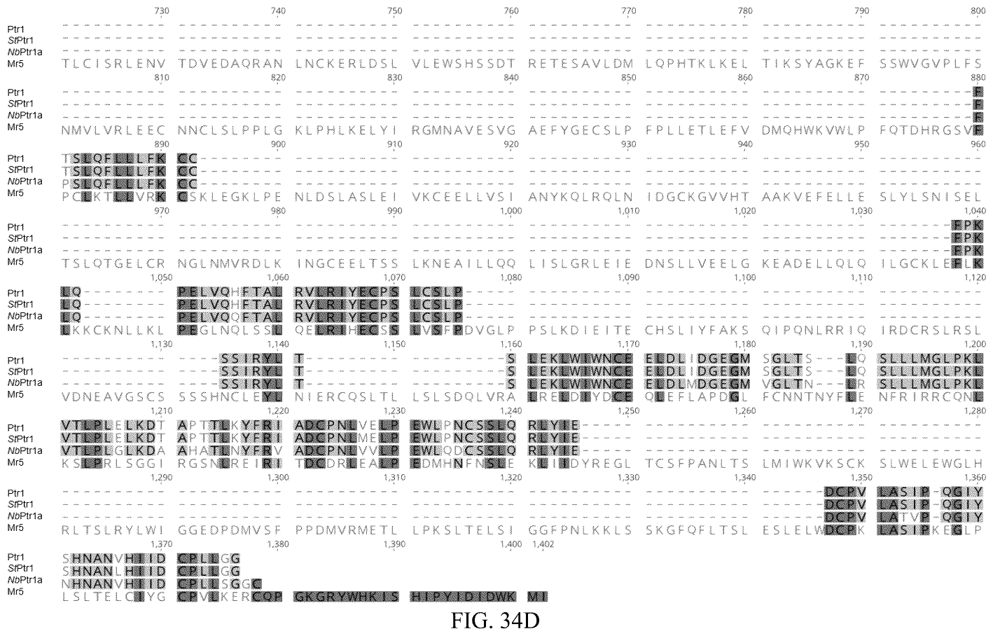

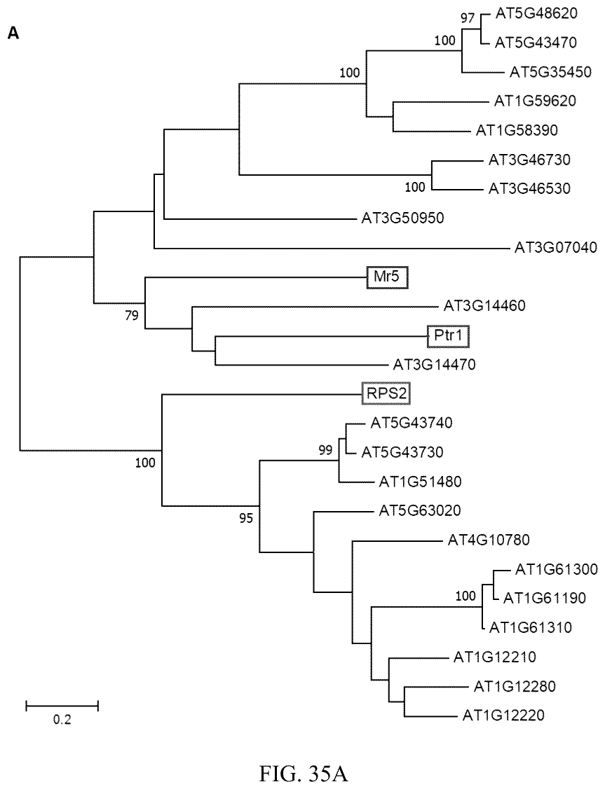

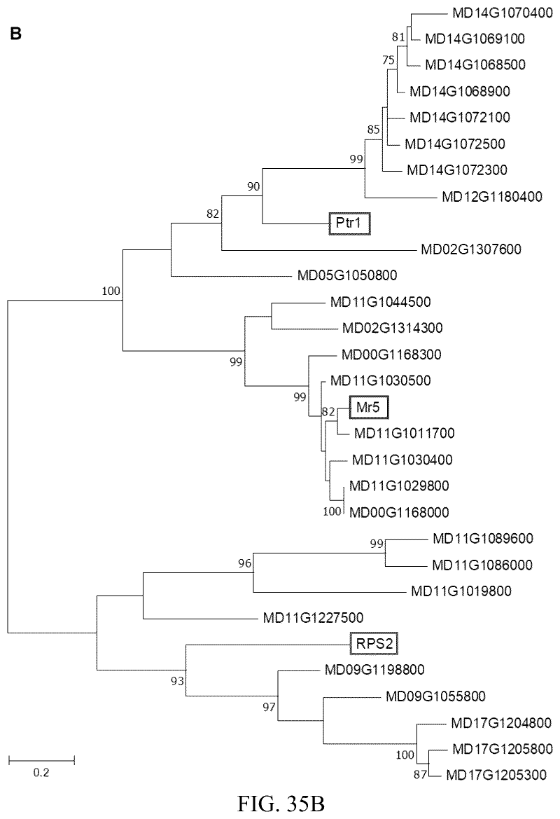

The nucleotide sequence of the Ptr1 gene is 39% identical to RPS2 and 34% identical to Mr5, which mediate recognition of AvrRpt2 in Arabidopsis and apple, respectively (FIGS. 33A-33D). The amino acid sequence identity is similarly low (˜20% identity of the Ptr1 protein with either RPS2 or Mr5) with many of the identical residues being in conserved NLR motifs (FIGS. 34A-34D). To understand the broader relationships of the Ptr1, RPS2, and Mr5 genes the S. lycopersicoides genome sequence was searched to find the most closely related genes for each of them. The NB-ARC domains encoded by these genes were then used in a phylogenetic analysis. This revealed that multiple S. lycopersicoides proteins are more similar to RPS2 and Mr5 than to Ptr1 with each of the proteins belonging to a distinct clade (FIG. 24). A similar analysis looking for the most closely related genes in Arabidopsis and in apple confirmed that multiple genes occur in these two species which are closer to Ptr1 than to either RPS2 or Mr5 (FIGS. 34A-35B).

### Example 18: Discussion of Examples 12-17

A natural outbreak of bacterial speck disease led to the serendipitous discovery of the Ptr1 locus which confers resistance to Pst strains that express the effector AvrRpt2 (Examples 1-11; Mazo-Molina et al., “The Ptr1 Locus of Solanum lycopersicoides Confers Resistance to Race 1 Strains of Pseudomonas syringae pv. tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety). The current annotation of the S. lycopersicoides genome sequence indicated there are 16 NLR-encoding genes in the large introgression segment shared between LA4245-R and LA4277-R and it was hypothesized that one of them is Ptr1. Eight of these NLR-encoding genes are clustered within a ˜2 Mb region while the other eight are dispersed in an almost 80 Mb segment of chromosome 4. As expected when working with a wild relative of tomato, little overall recombination involving the introgressed region was observed. However, a single recombinant among the 585 F2 plants screened eliminated the eight clustered NLR-encoding genes as being candidates for Ptr1. Inspection of the gene models for the remaining eight genes revealed one of them to be a pseudogene and no transcripts were detectable in leaves of five of the remaining genes, leaving just two candidates. Agrobacterium-mediated transient expression assays revealed that one of these two genes is Ptr1 and showed that it mediated recognition of proteolytically-active AvrRpt2 and RipBN. Discussed herein is the use of N. glutinosa, the discovery of Ptr1 homologs in various solanaceous species, and potential use of Ptr1 in breeding programs and the process of convergent evolution which apparently is responsible for the ability of three diverse NLR-encoding genes to mediate recognition of AvrRpt2.

It was reported previously that AvrRpt2 does not induce cell death in N. glutinosa (Kessens et al., “Determining the GmRIN4 Requirements of the Soybean Disease Resistance Proteins Rpg1b and Rpg1r Using a Nicotiana glutinosa-Based Agroinfiltration System,” PLoS One 9:e108159 (2014), which is hereby incorporated by reference in its entirety). Therefore, this species was used for Agrobacterium-mediated transient expression of the Ptr1 candidates because in our conditions AvrRpt2 induces strong cell death in N. benthamiana, the typical species used for agroinfiltration. No genome sequence is currently available for N. glutinosa but it appears to lack a functional Ptr1 gene since these experiments show that the Ptr1 pathway is otherwise intact in this species. N. glutinosa does appear to have one or more functional Rin4 proteins because at a lower titer Ptr1-induced cell death does not occur, whereas co-expression of AvrRpt2 at this low titer (likely leading to cleavage of Rin4) induces cell death. Higher expression of Ptr1 by using higher titers of Agrobacterium causes cell death without AvrRpt2 likely because it disrupts the stoichiometric levels of Ptr1 and Rin4 needed to negatively regulate Ptr1. Indeed, similar to observations with RPS2 and AtRIN4, it was found that co-overexpression of Ptr1 and SlRin4-3 suppressed the ectopic activation of Ptr1.

Arabidopsis RIN4 has two sites (RCS1 and RCS2) each with a consensus sequence which is proteolytically cleaved by AvrRpt2, resulting in three AvrRpt2-cleavage products: ACP1, ACP2, and ACP3 (Coaker et al, “Activation of a Phytopathogenic Bacterial Effector Protein by a Eukaryotic Cyclophilin,” Science 308:548-50 (2005), which is hereby incorporated by reference in its entirety). The C-terminal half of AtRIN4 is necessary and sufficient for the negative regulation of RPS2, but none of the individual cleavage products can alone negatively regulate RPS2. This supports a model that cleavage of RIN4 specifically at RCS2 leads directly to RPS2 activation via loss of suppression by RIN4 (Day et al., “Molecular Basis for the RIN4 Negative Regulation of RPS2 Disease Resistance,” Plant Cell 17:1292-305 (2005); and Kim et al., “The Pseudomonas syringae Effector AvrRpt2 Cleaves its C-Terminally Acylated Target, RIN4, from Arabidopsis Membranes to Block RPM1 Activation,” PNAS 102:6496-501 (2005), which are hereby incorporated by reference in their entirety). Interestingly, different from AtRIN4, apple MdRIN4 does not suppress NLR-dependent autoactivity. Instead, ACP3 released upon cleavage of MdRIN4 is sufficient to activate Mr5 (Prokchorchik et al., “A Host Target of a Bacterial Cysteine Protease Virulence Effector Plays a Key Role in Convergent Evolution of Plant Innate Immune System Receptors,” New Phytol. 225:1327-42 (2019), which is hereby incorporated by reference in its entirety). Investigation of the mechanism by which ACP3 from MdRIN4, and not AtRIN4, activates Mr5 revealed two polymorphic amino acid residues in the N-terminal sequences of ACP3 in AtRIN4 (N158/Y165) and MdRIN4 (D186/F193) (Prokchorchik et al., “A Host Target of a Bacterial Cysteine Protease Virulence Effector Plays a Key Role in Convergent Evolution of Plant Innate Immune System Receptors,” New Phytol. 225:1327-42 (2019), which is hereby incorporated by reference in its entirety). The three tomato Rin4 proteins that are expressed in leaves each have a hybrid of these polymorphisms. That is, instead of having an asparagine (N) as occurs in Arabidopsis, SlRin4-1, 4-2, and 4-3 have an aspartic acid (D) like apple. Moreover, the tomato Rin4 proteins have a tyrosine (Y) like AtRIN4 instead of a phenylalanine (F) as occurs in MdRIN4 (FIG. 25A). Whether or not these differences play a role in activation or suppression of Ptr1 is unknown and their future investigation may shed light on how the SlRin4 proteins regulate Ptr1.

Despite much effort, by many researchers, no source of simply-inherited resistance to race 1 strains of Pst has been discovered among accessions of cultivated tomato or its wild relatives (although some QTLs contributing to race 1 resistance have been reported; (Bao et al., “Identification of a Candidate Gene in Solanum habrochaites for Resistance to a Race 1 Strain of Pseudomonas syringae pv. Tomato,” The Plant Genome 8(3):10.3835/plantgenome2015.02.0006 (2015); Thapa et al., “Identification of QTLs Controlling Resistance to Pseudomonas syringae pv. Tomato Race 1 Strains From the Wild Tomato, Solanum habrochaites LA1777,” Theor. Appl. Genet. 128:681-92 (2015); and Hassan et al., “A Rapid Seedling Resistance Assay Identifies Wild Tomato Lines that are Resistant to Pseudomonas syringae pv. tomato Race 1,” Mol. Plant-Microbe Interact. 30:701-9 (2017), which are hereby incorporated by reference in their entirety). The discovery that the Ptr1 ortholog in both tomato and S. pennellii has multiple mutations including one that disrupts the start codon explains why race 1 resistance involving recognition of AvrRpt2 was never found. This observation suggests that strains of Pst expressing AvrRpt2 were not serious pathogens in the environment where wild relatives of tomato evolved and that there was no selection pressure to retain Ptr1. It will be interesting in the future to determine if all or just some of the accessions of the 12 wild relatives of tomato have nonfunctional versions of Ptr1 and possibly correlate this feature with the regions in which the accessions were originally collected.

The Ptr1 ortholog from potato was able to mediate recognition of AvrRpt2 and RipBN when transiently expressed in N. glutinosa leaves. This ortholog was cloned from the variety Dakota Crisp and has a sequence (i.e., SEQ ID NO: 116) that is 98% similar to a gene annotated in the potato genome sequence as resistance gene analog 1 (RGA1; GenBank No. XP_006340095.1, SEQ ID NO: 131). No function for the RGA1 gene has been reported and we have therefore named it StPtr1. Potato, tomato, and S. lycopersicoides originated in South America and N. benthamiana originated in Australia. The fact that these four diverse species all have highly conserved Ptr1 sequences suggests Ptr1 is ancestral in the Solanaceae and that its function was lost in some clades, such as the tomato clade. The observations described herein also support the hypothesis that bacterial pathogens of potato, N. benthamiana and S. lycopersicoides and likely their progenitors have, over evolutionary time, expressed an AvrRpt2-like protein. This is consistent with the importance of this effector for bacterial virulence and with its presence in many bacterial species and multiple pathovars of P. syringae (FIG. 36).

The observation that expression of AvrRpt2 or RipBN alone causes cell death in N. benthamiana leaves and the discovery of an intact Ptr1 ortholog (NbPtr1a) in this tobacco species raised the possibility that the effector-induced cell death involves Ptr1. Virus-induced gene silencing of the NbPtr1a gene in N. benthamiana and complementation with a synthetic Ptr1 gene that is recalcitrant to silencing confirmed this is the case. N. benthamiana has been used extensively to characterize biochemical aspects of the Pto/Prf complex and to identify and study host proteins that play a role in the Pto/Prf pathway such as Cb110, Cpk6, Epk1, Hsp90, Mail, Nrc2/3, MAPKKKa, MKK2, Sgtl, TFT7 (Ekengren et al., “Two MAPK Cascades, NPR1, and TGA Transcription Factors Play a Role in Pto-Mediated Disease Resistance in Tomato,” Plant J. 36:905-17 (2003); Lu et al., “High throughput Virus-Induced Gene Silencing Implicates Heat Shock Protein 90 in Plant Disease Resistance,” EMBO J. 22:5690-9 (2003); del Pozo et al., “MAPKKKa is a Positive Regulator of Cell Death Associated with both Plant Immunity and Disease,” EMBO J. 23:3072-82 (2004); Mucyn et al., “Regulation of Tomato Prf by Pto-like Protein Kinases,” Mol. Plant-Microbe. Interact. 22:391-401 (2009); Oh et al., “Tomato 14-3-3 Protein 7 Positively Regulates Immunity-Associated Programmed Cell Death by Enhancing Protein Abundance and Signaling Ability of MAPKKKa,” Plant Cell 22:260-72 (2010); Oh et al., “Tomato 14-3-3 Protein TFT7 Interacts with a MAP Kinase Kinase to Regulate Immunity-Associated Programmed Cell Death Mediated by Diverse Disease Resistance Proteins,” J. Biol. Chem. 286:14129-36 (2011); de 1a Torre et al., “The Tomato Calcium Sensor Cbl10 and its Interacting Protein Kinase Cipk6 Define a Signaling Pathway in Plant Immunity,” Plant Cell 25:2748-64 (2013); Kud et al., “SGT1 Interacts with the Prf Resistance Protein and is Required for Prf Accumulation and Prf-Mediated Defense Signaling,” Biochem. Biophys. Res. Comm. 431:501-5 (2013); Ntoukakis et al., “The Tomato Prf Complex is a Molecular Trap for Bacterial Effectors Based on Pto Transphosphorylation,” PLoS Pathog. 9:e1003123 (2013); Saur et al., “The N-Terminal Domain of the Tomato Immune Protein Prf Contains Multiple Homotypic and Pto Kinase Interaction Sites,” J. Biol. Chem. 290:11258-67 (2015); Wu et al., “NLR Network Mediates Immunity to Diverse Plant Pathogens,” PNAS 114:8113-8 (2017); Roberts et al., “Mail Protein Acts Between Host Recognition of Pathogen Effectors and Mitogen-Activated Protein Kinase Signaling,” Mol. Plant-Microbe. Interact. 32:1496-507 (2019); and Wu et al., “Tomato Prf requires NLR Helpers NRC2 and NRC3 to Confer Resistance Against the Bacterial Speck Pathogen Pseudomonas syringae pv. Tomato,” bioRxiv, doi:10.1101/595744 (2019), which are hereby incorporated by reference in their entirety). It will be interesting in the future to determine if all of these proteins play a role in the Ptr1 pathway or if the Pto/Prf and Ptr1 pathways use some divergent host components.

It was considered previously how the Ptr1 locus could play an important role in protection of tomato against bacterial speck disease (Examples 1-11; Mazo-Molina et al., “The Ptr1 Locus of Solanum lycopersicoides Confers Resistance to Race 1 Strains of Pseudomonas syringae pv. Tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety). The identification of the Ptr1 gene will now allow it to be efficiently tracked as it is backcrossed into various breeding lines. Ptr1 and Pto are located on different chromosomes (4 and 5, respectively) which will facilitate the development of tomato varieties containing both genes. Such varieties would confer resistance to all currently known races of Pst. The low rate of recombination between S. lycopersicoides and tomato DNA could interfere with introgression of a small segment of S. lycopersicoides carrying the Ptr1 gene. Methods are available to address low recombination such as using a bridge species as discussed previously (Examples 1-11; Mazo-Molina et al., “The Ptr1 Locus of Solanum lycopersicoides Confers Resistance to Race 1 Strains of Pseudomonas syringae pv. tomato and to Ralstonia pseudosolanacearum by Recognizing the Type III Effectors AvrRpt2 and RipBN,” Mol. Plant Microbe. Interact. 32:949-60 (2019), which is hereby incorporated by reference in its entirety). Finally, if the new method of CRISPR ‘Prime editing’ proves to be feasible in tomato then it might be employed to ‘repair’ the Ptr1 pseudogene present in tomato (Anzalone et al., “Search-and-Replace Genome Editing Without Double-Strand Breaks or Donor DNA,” Nature 576:149-57 (2019), which is hereby incorporated by reference in its entirety). Such an approach would greatly simplify the introgression of the modified gene into advanced breeding lines for use in development of speck-resistant tomato varieties.

By using a phylogenetic analysis, it was found that the most similar genes to RPS2 and Mr5 in S. lycopersicoides fall into distinct clades which are distantly related to Ptr1. The same conclusion came from phylogenetic trees of the most closely related genes to Ptr1 in the apple and Arabidopsis genomes. These observations indicate that Ptr1, which is present in at least three solanaceous species, is not orthologous to either RPS2 or Mr5 and likely arose by convergent evolution to mediate recognition of AvrRpt2. Ptr1, RPS2 and Mr5 represent just the third case where non-orthologous NLR-encoding genes in different plant species have been reported to mediate recognition of the same effector. In soybean, the R genes Rpg1b and Rpg1r recognize AvrB and AvrRpm1, respectively, whereas in Arabidopsis RPM1 recognizes both of these effectors. Although all of these genes encode CC-NLR proteins, which detect alteration of RIN4, the Rpg1 genes are not orthologous to RPM1(Ashfield et al., “Convergent Evolution of Disease Resistance Gene Specificity in two Flowering Plant Families,” Plant Cell 16:309-18 (2004), which is hereby incorporated by reference in its entirety). The other example is the recent discovery of an NLR-encoding gene in barley, Pbr1, whose protein detects AvrPphB-directed cleavage of PBS1 as does the Arabidopsis RPS5 protein; the RPS5 and Pbr1 genes are not orthologous (Carter et al., “Convergent Evolution of Effector Protease Recognition by Arabidopsis and Barley,” Mol. Plant-Microbe. Interact. 32:550-65 (2018), which is hereby incorporated by reference in its entirety). These three examples demonstrate the remarkable plasticity inherent in the modular structure of NLR proteins which facilitates the generation of common resistance specificities from highly divergent genes.

The specific mechanisms by which RPS2 and Mr5 are activated upon cleavage of Rin4 are unknown (Prokchorchik et al., “A Host Target of a Bacterial Cysteine Protease Virulence Effector Plays a Key Role in Convergent Evolution of Plant Innate Immune System Receptors,” New Phytol. 225:1327-42 (2019); and Toruno et al., “Regulated disorder: Posttranslational modifications control the RIN4 plant immune signaling hub,” Mol. Plant Microbe. Interact. 32:56-64 (2019), which are hereby incorporated by reference in their entirety). The availability now of amino acid sequences of three diverse proteins that mediate recognition of AvrRpt2 might provide insights into subdomains of the proteins that are involved in the response to AvrRpt2 (and to Rin4 cleavage products). As expected, there are a number of residues in common between the Ptr1, StPtr1, NbPtr1a, RPS2, and Mr5 proteins in the NB-ARC domain (FIGS. 34A-34D). More interesting perhaps is the presence of some conserved residues in the LRR domain which is typically more divergent among NLR proteins. Whether these conserved residues play a role in mediating the response to AvrRpt2 will be a focus of future work using the Agrobacterium infiltration assay in N. glutinosa.

TABLE 13
Additional Sequences.

| | SEQ | |
|---|---|---|
| Description | ID NO: | Sequence |
| SlRin4-1 | 85 | MARPNVPKFGNWENDDNTPYTVYFEKARQTRGTGKMMNPNDP |
| | | EENPDMFRNLAPPPEVAPQSKPKRQTEEPPIGRGGPARQTRDHRL |
| | | SKEDGEFRQYANSPARKESVGRKGANEPSHQRGRGSNSGRTGR |
| | | QSIGSEHSFDKSPLHPHYQAKVSNAGRGVASPAWEGKNNSYDSS |
| | | HGTPGRSKVKQDKSDRGAAVPRFGEWDENDPQSADNYTHIFNK |
| | | FREEKQGNPSGTPSRTSNNTQKHNSEEKQRKWCCCPW |
| SlRin4-2 | 86 | MARANVPKFGNWGNEDNTPYTVVFENARKNRGGKMINPNDPQ |
| | | ENPDMFPNVAPSSRPKTPPTEEPMGMETARQTNKRRVSKEDGDF |
| | | RASSPARNEPTTHQRHGGGRGSNSGRPSRQSGGSDHSIAKSPLHP |
| | | NSQAKISGRVAASPVWEGKNLYDSSHGTPGRSFESSHATPGRHQ |
| | | MKQESPDRGTVVPKFGGWDDNDPQDAENYTEVFNKVREQRHV |
| | | DTGNMPAAGVRTSYSTQRQQRNEKQK |
| SlRin4-3 | 87 | MAGSHVPKFGNWDGENVPYTAYFENARKSNSKGGKMINPNDP |
| | | EENPEAFAYCGDEDANINISPLVEKHQYHYDHRRNPSVESGQNK |
| | | SIGPTNSNSESFGDSQRKSVSGFSVNQPTRRRRTSDVKKNKNDRG |
| | | NGFVPPSPNRPMKNSRNPSDDLSCSSAASVPKFGAWDEKDPKSG |
| | | EGFTVIFNKVKEEKHIAAAKFPVVQPQSNMSSSNNHKKNAKSKV |
| | | FCCLF |
| AtRin4 | 88 | MARSNVPKFGNWEAEENVPYTAYFDKARKTRAPGSKIMNPNDP |
| | | EYNSDSQSQAPPHPPSSRTKPEQVDTVRRSREHMRSREESELKQF |
| | | GDAGGSSNEAANKRQGRASQNNSYDNKSPLHKNSYDGTGKSRP |
| | | KPTNLRADESPEKVTVVPKFGDWDENNPSSADGYTHIFNKVREE |
| | | RSSGANVSGSSRTPTHQSSRNPNNTSSCCCFGFGGK |
| MdRin4-1 | 89 | MAQRSHVPKFGNWEDQESVPYTAYFDKARKGRTGVGGKMINP |
| | | NDPEENPDILSDTSASSPPKVRPEPGKPVHERRRSREDNDLRFAN |
| | | SPAQRRSSGEHQPNRGRGVSSGETHRRAARPSAGSENSVERSPL |
| | | HRNARVSGRDSPSWEGKASYESSHGTPARSRLKPRDESPEKGAA |
| | | VPKFGEWDENDPASADGFTHIFNKVREEKAGKAPGTPSHPSYQD |
| | | ARKQGSNDSAKCCCFPWGRK |
| MdRin4-2 | 90 | MAQRSHVPKFGNWEGEESVPYTAYFDKARKDRTGVGGKMINP |
| | | NDPQENPDILSDISASSPPKVRPEPEKPVHEQRRSREDNDLRFANS |
| | | PAQRRNSGESAHQPSRGRGVSSGETRRRPARPSAGSENSVERSPL |
| | | HRNARVTGRDSPSWEGKASYETSHGTPGRSRLKPRDESPEKGAA |
| | | VPKFGEWDENDPASADGFTHIFNKVREERAGKVPGTPSQPSYQD |
| | | ARRQGSNDSAKSCCFPWSRK |
| NB-ARC: P- | 91 | VGLGGLGKTTLVKL |
| loop, Ptr1 | | |
| NB-ARC: P- | 92 | VGMGGIGKTTLAKK |
| loop, Consensus | | |
| NB-ARC: kinase | 93 | QQKRYLLVLDDVWNEDQ |
| 2, Ptr1 | | |
| NB-ARC: kinase | 94 | KGKRYLIVLDDVWDTDQ |
| 2, Consensus | | |
| NB-ARC: kinase | 95 | SKIVVTTRSKMV |
| 3a, Ptr1 | | |
| NB-ARC: kinase | 96 | SRIIXTTRDXXV |
| 3a, Consensus | | |
| NB-ARC: | 97 | VKKCGGVPLAVKT |
| GLPL, Ptr1 | | |
| NB-ARC: | 98 | VKKCKGLPLALKV |
| GLPL, | | |
| Consensus | | |
| NB-ARC: MHD, | 99 | NLVHDLAQ |
| Ptr1 | | |
| NB-ARC: MHD, | 100 | CRMHDLIR |
| Consensus | | |
| Leucine-rich | 101 | LRMLVLNNLDLEEL |
| repeat | | |
| Leucine-rich | 102 | LRYLNLSDSGKIKL |
| repeat | | |
| Leucine-rich | 103 | LVNLHTLNLI |
| repeat | | |
| Leucine-rich | 104 | LKELPRDFR |
| repeat | | |
| Leucine-rich | 105 | LISLKTLYL |
| repeat | | |
| Leucine-rich | 106 | LQFLLLFK |
| repeat | | |
| Leucine-rich | 107 | LQPELVQHFTALRVL |
| repeat | | |
| Leucine-rich | 108 | LCSLPSSIR |
| repeat | | |
| Leucine-rich | 109 | LTSLEKLWIWNCEELDL |
| repeat | | |
| Leucine-rich | 110 | LTSLQSLLLMGLPK |
| repeat | | |
| Leucine-rich | 111 | LVTLPLELKDTAPTTL |
| repeat | | |
| Leucine-rich | 112 | LVELPEWLPN |
| repeat | | |
| Leucine-rich | 113 | LQRLYIEDCPVL |
| repeat | | |
| SlPtr1 | 114 | ATGTAGATCCTTGAACGTGTTTTGGCGAAATCTTTCTTGTTCA |
| | | ATATCATTGAACGTGTTTTGGCTAAAGTTTCTTCAATTGCTAT |
| | | ATATGAGATCAGTCTAGCTTGGAATGTTAAGACAGAGCTAAG |
| | | GAAACTCCAAAGTACTCTATCCACCATCAAAGTTGTACTTCTA |
| | | GATGCAAACGAGCAGCAGGCGAAGAACCATGAAGTGAGAGA |
| | | TTGGCTGGAAAAGCTCAGAGATGTTGTTTATGATGTCGATGAT |
| | | TTGATGGATGATTTATCAACACAACTGTTGCTGCGAATGCATT |
| | | TCGAGTAAAGCTTTAGGAAGAAGGTAAGGAAGTTCTTTTCAG |
| | | GTTCAAATCCAATTATATATCGATTCAAGATTGGCAGAAAAG |
| | | TAAAAGAAATCAAGGAGCTGCTGAATGAGATTGCAGATGATT |
| | | GGAGAAATTTCCACTTCACGGAACATACTTATGTAACTCCAG |
| | | CTGAGAATACGAGTAGAGAACAAACACACTCCTTTGTGAGGG |
| | | CATCAGATATCATTGGTAGAGATGATGATCAAGAAAACATTG |
| | | TATAACAGCTGATAGATTCTCATGATGAGGAAAATATTTTTGT |
| | | GATTCCTATTGTTGGACTTAGAGGGCTTGGAAAAACCACACTT |
| | | GTTAAGTTGGTTTATAACAATAATAGGGTTGTTCAGAATTTCA |
| | | ACCTTAGATTGTGGGTTAGTATTTCTGAAGATTTTAGTCTGAG |
| | | CAAGGTAATTGAGAAAATTGTGAGGTCCGGAACAGGAGAGA |
| | | GTTTTGACCACCTAGATATGGACCAATTACAAGGTTGTTTGGG |
| | | AGAGGTTTTGCAACAGAAAAGGTATTTACTTGTGCTGGATGA |
| | | TGTGTGGAATGAAGATCAACACAAGTGGATAGATATGAGGGA |
| | | GTTGCCGATGAATTGTTCCAGAGGTAGTAAAATTGTTGTCACT |
| | | ACACGTAGTAAGATGGTTGCTTTGATTACTGGAACAGTTCCGC |
| | | CTTATTATTTGAGAGGCCTTGGTAATGATGACTGCTTATCTTA |
| | | TTTTTGAAATGTGCATTTGGAGGGCAGGACAATTTGTTTCCTA |
| | | ATCTAGTAGAAATAGGAAAAGAAATTGTGAAAAAGTGTGGA |
| | | GGAGTGCCTTTGGCTGTGAAAACCTTGGGAAGGTTGTTGTAC |
| | | ATGAAAACAGACAAGAACGAATGGTTGCAGATAAGAGACGA |
| | | TGAGATATGGGAAATCGAACAGAATAAATCTGACATCTTACC |
| | | AATATTGAGGTTGAGCTATGAACAGATGCCATCACATCTAAG |
| | | ACAGTGCTTTGCCTATTGCCCCATGTTACCCAAAGGTCAAGAA |
| | | ATTCCGTGGGAGGATTTTATCAATCGCTGGGCTCAAGGATTTA |
| | | TCCAGAGTTCAAACAGAAACAGGAAGTTGGAAGATATCGGTA |
| | | ATCAGTACTTTGATGAGTTGCTATCAAGGTTTTGCTTCCTAGA |
| | | TGTGATACAAGCAGCTTTTGATGGAGAAATATTGGCTTGTAA |
| | | GATACACAATCTTGTGCATGATCTTGCACAGTCAGTATCAGGT |
| | | GCAGAGTGCTTAAATGTGAAACCCAATGCTTTCGTGGTTTCTG |
| | | AGAGAGTTCGCCACTTATTCTTCCATGCAGAAGATATGTCTAG |
| | | GAAATACTTCCCCAGATTTTTGCTTCCTTTGCATAAGGTGAGG |
| | | TCTTTTTTTTTATTCGTTTAATATTGGACCTGTAAACAAGTTTT |
| | | TTGTCAAGACAATGTTGTCAAATTTCAAATGCCTTCGGATGTT |
| | | AGTCTTGAACAATCTAGATCTTGAGGAGTTGCCAACTTCGATA |
| | | GGTAACTTGAATGAATTAAGATACCTTAACCTTAGTGACAGT |
| | | GGTAATATCAAGTTTATTCCAAGGTCTATGAGCAAATTAGTA |
| | | AATTTGCACACCCTTAACCTCATTAACTGTGAACAGCTTAAGG |
| | | AGTTGCCAAGAGACTTTAGAAAGTTAATCAGCCTGAAGACCT |
| | | TGTATTTGACCACACATCAGATATTAGCAGGGATCAAGAATC |
| | | AACATTCTTTCACTTCTCTTCAATTTTTGCTTCTTTTCGAATGT |
| | | TGTTTCCCAAAATTGCAGCCAGAACTGGTGCAGCATTTTACTG |
| | | AACTTCGGTTGTTGCGTATCTACGAATGCCAAGTTTATGTTCT |
| | | CTTCCAAGAAGTATTAGATATCTTACTTCACTTGAAAAGCTAT |
| | | GGATCTGGAACTGTGAAGAACTTGATTTGATTGATAGAGAAG |
| | | GAATGTCAGGCCTAACAAGTCTTCAATCCTTGCTTCTAATGGG |
| | | GCTTCCTAAGTTGGTGACTCTACCATTGGAACTCAAAGATACC |
| | | GCTCCTACAACATTGAAGTACTTTAGAATCGCCGATTGTCCCT |
| | | ACCTGGTGGAGCTTCCAGAGTGGCTGCCGAATTGCTCCTCACT |
| | | TCAGAGACTGTATATAGAGGATTGTCCTGTTTTGGCATCGATA |
| | | CCTCAAGGAATCTACAACCACAATGCCAACGTCCATATAATC |
| | | GACTGTCCATTGCTAGGTGGATGA |
| SpPtr1 | 115 | ATGTAGATCCTTGAACGTGTTTTGGCGAAATCTTTCTTGTTCA |
| | | ATATCATTGAACGTGTTTTGGCTAAAGTTTCTTCAATTGCTAT |
| | | ATATGAGATCAGTCTAGCTTGGAATGTTAAGACAGAGCTAAG |
| | | GAAACTCCAAAGTACTCTATCCACCATCAAAGTTGTACTTCTA |
| | | GATGCAAACGAGCAGCAGGCGAAGAACCATGAAGTGAGAGA |
| | | TTGGCTGGAAAAGCTCAGAGATGTTGTTTATGATGTCGATGAT |
| | | TTGATGGGTGATTTATCAACACAACTGTTGCTGCGAATGCATT |
| | | TCCAGAAAAGCTTTAGGAAGAAGGCAAGGAAGTTCTTTTCAA |
| | | GTTCAAATCCAATTATATATCGATTCAAGATTGGCAGAAAAG |
| | | TAAAAGAAATCAAGGAGCTGCTGAATGAGATTGCAGATGATA |
| | | GGAGAAATTTCCACTTCACGGAACATACTTATGTAATTCCAGC |
| | | TGAAAATACGAGTAGAGAACAAACACACTCCTTTGTGAGGGC |
| | | ATCAGATATCATTGGTAGAGATGATGATCAAGAAAACATTGT |
| | | ATAACAGCTGATAGATTCTCATGATGAGGAAAATATTTTTGTG |
| | | ATTCCTATTGTTGGACTTAGAGGGCTTGGAAAAACCACACTTG |
| | | TTAAGTTGGTTTATAACAATAATAGGGTTGTTCAGAATTTCAA |
| | | CCTTAGATTGTGGGTTAGTATTTCTGAAGATTTTAGTCTGAGC |
| | | AAGGTAATTGAGAAAATTGTTAGGTCCGGAACAGGAGAGAGT |
| | | TTTGACCACCTAGATATGGACCAATTACAAGGTTGTTTGGGA |
| | | GAGGTTTTGCAACAGAAAAGGTATTTACTTGTGCTGGATGAT |
| | | GTGTGGAATGAAGATCAACACAAGTGGACAGATATGAGGGA |
| | | GTTGCTGATGAATTGTTCCAGAGGTAGTAAAATTGTTGTCACT |
| | | ACACGTAGTAAGATGGTTGCTTTGATTACTGGAACAGTTCCGC |
| | | CTTATTATTTGAGAGGCCTTGGTAATGATGACTGCTTATCTTA |
| | | TTTTTGAAATGTGCATTTGGAGGGCAGGACAATTTGTTTCCTA |
| | | ATCTAGTAGAAATAGGAAAAGAAATTGTGAAAAAGTGTGGA |
| | | GGAGTGCCTTTGGCTGTGAAAACCTTGGGAAGGTTGTTGTAC |
| | | ATGAAAACAGACAAGAACGAATGGTTGCAGATAAGAGACAA |
| | | TGAGATATGGGAAATCGAACAGAATAAATCTGACATCTTACC |
| | | AATATTGAGGTTGAGCTATGAACAGATGCCATCACATCTAAG |
| | | ACAGTGCTTTGCCTATTGCCCCATGTTACCCAAAGGTCAAGAA |
| | | ATTCCGAGGGAGGATTTTATCAATCGCTGGGCTCAAGGATTT |
| | | ATCCAGAGTTCAAACAGAAACAGGAAGTTGGAAGATATCGGT |
| | | AATCAGTACTTTGATGAGTTGCTATCAAGGTTTTGCTTCCTAG |
| | | ATGTGGTACAAGCTTTTGATGGAGAAATATTGGCTTGTAAGA |
| | | TACACAATCTTGTGCATGATCTTGCACAGTCAGTATCAGGTGC |
| | | AGAGTGCTTAAATGTGAAACCCAATGCTTTCGTGGTTTCTGAG |
| | | AGAGTTCGCCACTTATTCTTCCATGCAGAAGATATGTCTAGGA |
| | | AATACTTCCCCAGATTTTTGCTTCCTTTGCATAAGGTGAGGTC |
| | | TTTTTCTTATTCGTTTAACATTGGACCTGTAAACAAGTTTTTTG |
| | | TCAAGACAATGTTGTCAAATTTCAAATGCCTTCGGATGTTAGT |
| | | CTTGAACAATCTAGATCTTGAGGAGTTGCCAACTTCGATAGGT |
| | | AACTTGAATGAATTAAGATACCTTAACCTTAGTGACAGTGGT |
| | | AATATCAAGTTTCTTCCAAGGTCTATGAGCAAATTAGTAAATT |
| | | TGCACACCCTTAACCTCATTAACTGTGAACAGCTTAAGGAGTT |
| | | GCCAAGAGACTTTAGAAAGTTAATCAGCCTGAAGACCTTGTA |
| | | TTTGACCACACATCAGATATTTGCAGGGATCAAGAATCAACA |
| | | TTCTTTCACTTCTCTTCAATTTTGCTTCTTTTCAAATGTTGTTTC |
| | | CCAAAATTGCAGCCAGAACTGGTGCAGCATTTTACTGCACTTC |
| | | GGTTGTTGCGTATCTACGAATGCCCAAGTTTATGTTCTCTTCC |
| | | AAGCAGTATTAGATATCTTACTTCACTTGAAAAGCTATGGATC |
| | | TGGAACTGTGAAGAACTTGATTTGA |
| S. tuberosum | 116 | MAESFLFNIIERVLAKVSSIAVYEITLAWNVKIELRKLQSTLSTIK |
| Ptr1 | | AVLLDANEQQAKNHEVRDWLEKLRDVVYDVDDLMDDLSTQLL |
| | | LQMHFQKSFRKKVRKFFSSSNPIIYRFKIGRKVKEIRELLNEIADD |
| | | RRNFHFTEHTFVISAENTSREQTHSFVRASDIIGRDDDQENIIKQLI |
| | | GSHDEENISVIPIVGLGGLGKTTLVKLVYNNNRVVQNYDLRMW |
| | | VSISEDFNLSKVIEKILRSATGESFDHLDMDQLQGCLGEVLQQKR |
| | | YLLVLDDVWNEDQHKWTDLRELLMNCSRGSKIVVTTRSKMVA |
| | | LITGTVPPYYLGGLADDDCLSLFLKCAFGGQDNLFPNLVEIGKEI |
| | | VKKCGGVPLAVKTLGRLLYMKTDENEWLQIRDNEIWEIEQNKS |
| | | DILPILRLSYEQMPSHLRQCFAYCSMLSKGQEIPREDFINRWIAQG |
| | | FIQSSNRNRKLEDIGNQYFDELLSRFCFLDVVQAFDGEILACKIHN |
| | | LVHDLAQSVAGAECLNVKPNAFVVSERVRHLFFHAEDMSRKHF |
| | | PRILLPLQKLRSFSYSFNIGPVNKFFVKTMLSNFKCLRMLVLNNL |
| | | DLEELPTSIGHLKELRYLNLSNSGNIKFLPRSMSKLVNLHTLNLIN |
| | | CEQLKELPRDFRKLISLKTLYLTTHQISAGIKNQHVFTSLQFLLLF |
| | | KCCFPKLQPELVQHFSALRVLRIYECPSLCSLPSSIRYLTSLEKLWI |
| | | WNCEELDLIDGEGMSGLTSLHSLLLMGLPKLVTLPLELKDTAPT |
| | | TLKYFRIADCPNLMELPEWLPNCSSLQRLYIEDCPVLASIPQGIYS |
| | | HNANLHIIDCPLLGG |
| C. annuum Ptr1 | 117 | MAESFLFNIIERVLAKVSSIAVYEISLAWNVKTDLRKLQSTLSTIK |
| | | AVLLDANEKQAKNHEVRDWLEKLRDVVYDVDDLMDDLSTQLL |
| | | LQMHFQKSFGKKVRRFFSSSNPIIYRFKIGRRVKEIRELLNEIADD |
| | | RKSFHFTEHNYLIPAENTSREQTHSSVRASDIIGRDADQENIVKQL |
| | | IDSHNEENISVIPIVGLGGLGKTTLVKLVYNDNRVVQNFDLRMW |
| | | VSISEDFSLRKIIEKILRSATGESFDHLDMDQLQGCLGNVLRLKRY |
| | | LLVLDDVWNEDQHKWTDLRELLMNCASGSKIVVTTRSKMAALI |
| | | TGTVPPYYLEGLANSDDCLSLFLKCAFGGQDNMFPNLVEIGKEI |
| | | VKKCGGVPLAVKTLGRLLYTKTDENEWLQIRDNEIWEIEQKESD |
| | | ILPILRLSYEQMPSHLRQCFAYCSMLSKGQEIPREDFINRWIAQGF |
| | | IQSSNRNSKLEDIGNQYFDELLSRFCFLDVVQAFDGEILACKIHSL |
| | | VHDLAQSVAGAECLNVKPNAFVVPERVRHLFFHAEDMSGKHFP |
| | | KFLLPLRKLRSFSYSFNVGPVNKFFVKTILSNFKRLRMLVLSNLD |
| | | LEELPTSIGHLKELRYLNLSGSGNIKFLPRSMSKLVNLQTLNLINC |
| | | EQLKELPRDFAKLISLKTLYLTTQQISVGIKCKNQHSFTSLQFLLL |
| | | FKCCFPKLQPELVQHFTALRVLRIYECPSLCSLPSSIRYLTSLEKL |
| | | WIWNCEELDLMDGEGMSGLSSLRSLLLMGLPKLVTLPLELKDT |
| | | APATSKYFRIADCPNLVELPEWLQNYSSLQRLYVEDCPALASIPP |
| | | GIYNHNVNVHIIDCPLLSGGC |
| C. baccatum Ptr1 | 118 | MAESFLFNIIERVLAKVSSIAVYEISLAWNVKTDLRKLQSTLSTIK |
| | | AVLLDANEKQAKNHEVRDWLEKLRDVVYDVDDLMDDLSTQLL |
| | | LQMHFQKSFGKKVRRFFSSSNPIIYRFKIGRRVKEIRELLNEIADD |
| | | RKSFHFTEHNYLIPAENTSREQTHSFVRASDIIGRDADQENIVKQL |
| | | IDSRNEENISVIPIVGLGGLGKTTLVKLVYNDNRVVQNFDLRMW |
| | | VSISEDFSLRKIIEKILRSATGESFDHLDMDQLQGCLGNVLQLKR |
| | | YLLVLDDVWNEDQHKWTDLRELLMNCARGSKIVVTTRSKMAA |
| | | LITGTVPPYYLEGLASDDCLSLFLKCAFGGQDNMFPNLVEIGKEI |
| | | VKKCGGVPLAVKTLGRLLYTKTDENEWLQIRDNEIWEIEQKESD |
| | | ILPILRLSYEQMPSHLRQCFAYCSMLSKGQEIPREDFINRWIAQGF |
| | | IQSSNRNSKLEDIGNQYFDELLSRFCFLDVVQAFDGEILACKIHSL |
| | | VHDLAQSVAGAECLNVKPNAFVVPERVRHLFFHAEDMSGKHFP |
| | | KFLLPLRKLRSFSYSFNVGPVNKFFVKTILSNFKRLRMLVLSNLD |
| | | LEELPTSIGHLKELRYLNLSGSGNIKFLPRSMSKLVNLQTLNLINC |
| | | EQLKELPRDFTKLISLKTLYLTTQQISVGIKFKNQHSFTSLQFLLLF |
| | | KCCFPKLQPELVQHFTALRVLRIYECPSLCSLPSSIRYLTSLEKLW |
| | | IWNCEELDLMDGEGMSGLSSLRSLLLMGLPKLVTLPLELKDTAP |
| | | ATSKYFRIADCPNLVELPEWLQNYSSLQRLYIEDCPALASIPPGIY |
| | | NHNVNVHIIDCPLLSGGC |
| C. chinense Ptr1 | 119 | MAESFLFNIIERVLAKVSSIAVYEISLAWNVKTDLRKLQSTLSTIK |
| | | AVLLDANEKQAKNHEVRDWLEKLRDVVYDVDDLMDDLSTQLL |
| | | LQMHFQKSFGKKVRRFFSSSNPIIYRFKIGRRVKEIRELLNEIADD |
| | | RRSFHFTEHNYLIPAENTSREQTHSSVRASDIIGRDADQENIVKQL |
| | | IDSHNEENISVIPIVGLGGLGKTTLVKLVYNDNRVVQNFDLRMW |
| | | VSISEDFSLRKIIEKILRSATGESFDHLDMDQLQGCLGNVLQLKR |
| | | YLLVLDDVWNEDQHKWTDLRELLMNCASGSKIVVTTRSKMAA |
| | | LITGTVPLYYLEGLASDDCLSLFLKCAFGGQDNMFPNLVEIGKEI |
| | | VKKCGGVPLAVKTLGRLLYTKTDENEWLQIRDNEIWEIEQKESD |
| | | ILPILRLSYEQMPSHLRQCFAYCSMLSKGQEIPREDFINRWIAQGF |
| | | IQSSNRNSKLEDIGNQYFDELLSRFCFLDVVQAFDGEILACKIHSL |
| | | VHDLAQSVAGAECLNVKPNAFVVPERVRHLFFHAEDMSGKHFP |
| | | KFLLPLRKLRSFSYSFNVGPVNKFFVKTILSNFKRLRMLVLSNLD |
| | | LEELPTSIGHLKELRYLNLSGSGNIKFLPRSMSKLVNLQTLNLINC |
| | | EQLKELPRDFAKLISLKTLYLTTQQISVGIKCKNQHSFTSLQFLLL |
| | | FKCCFPKLQPELVQHFTALRVLRIYECPSLCSLPSSIRYLTSLEKL |
| | | WIWNCEELDLMDGEGMSGLSSLRSLLLMGLPKLVTLPLELKDT |
| | | APATSKYFRIADCPNLVELPEWLQNYSSLQRLYIEDCPALASIPPG |
| | | IYNHNVNVHIIDCPLLSGGC |
| N. attenuata Ptr1 | 120 | MAESFLFNIIERVLAKVSSVAVTEISLAWNVKTELRKLQSTLSTIK |
| | | AVLLDANEQQAKNHEVRDWLEKLRDVVYDVDDVLDDLSTQLL |
| | | LQIHFQKSLKKKVRKFFSSSNPIIYRFKIGRKVKEIRELLNEIADDR |
| | | KSFHFTEHTCLNPVENICREQTHSFVRASDIIGRETDQENIVKQLI |
| | | DARDEENISVIPIVGLGGLGKTTLVKLVYNYNTVVQNFDLRMW |
| | | VSISEDFSLSKVIEKILRSATGESFGHLDMDQLQGHLSEVLRSKRY |
| | | LLVLDDVWNEDQNKWTDLRELLMNCSKGSKIVVTTRSKMVALI |
| | | TGTVAPYYLGGLTDDACLLLFLKCAFVGEDKLLPNLVEIGKEIV |
| | | KKCGGVPLAVKTLGRLLYMKTDENEWLRIRDNEIWEIEQKQSDI |
| | | LPILRLSYEQMPSHLRQCFAYCSMLSKGQEIPREDFINRWIAQGFI |
| | | QSSNGSRKLEDIGNQYFDELLSRFCFLDVVQAFDGEILACKLHNL |
| | | VHDLAQSVAGSECLNVKSNASVVSERVRHLFFHAEDMSRKHFP |
| | | RFLLSLQKLRSFSYSFNIGPVNKFFVKTTLSNFKCLRVLVLNNLD |
| | | FEELPTSIGHLKELRYLNLSDNGNIKFLPMSMSKLVNLQTFNLIN |
| | | CEQLKELPRDFGKLICLKTLYLTTYKISAGKNQQSFPSLQFLLLFK |
| | | CCFPKLQPELVQQFTALRVLRIYECPSLCSLPSSIRYLTSLEKLWI |
| | | WNCEELDLMDGEGMVGLTSLRSLLLMGLPKLVTLPLGLKDAAH |
| | | ATLKYFRVADCPNLVVLPEWLQNCSSLQRLYIEDCPVLATIPQGI |
| | | YNHNANVHIIDCPLLSGGC |
| N. benthamiana | 121 | MAEFFLFNIIERVLSKVSSVSVNEISLAWNVKTELRKLQSTLSTIK |
| (Nb) Ptr1a | | AVLLDANERQAKNHEVRDWLEKLRDVVYDVDDVLDDLSTQLL |
| | | LQIHFQESLKKKVRKFFSSSNPIIYRFKIGRKIKEIRELLNDIADDR |
| | | KSFHFTEHTCINPVENICREQTHSFVRASDIIGRETDQENIVKQLID |
| | | SRDEENISVIPIVGLGGLGKTTLVKLVYNNNKVVQNFDLRMWVS |
| | | ISEDFSLSKVIEKILRSATGESFGHLDMDQLQGHLSEVLRSKRYLL |
| | | VLDDVWNEDQNRWTDLRELLMNCSRGSKIVVTTRSKMVALITG |
| | | TVAPYYLGGLTDDACLSLFLKCAFVGEDKLLPNLVEIGKEIVKK |
| | | CGGVPLAVKTLGRLLYMKTDENEWLRIRDNEIWEIEQKQSDILPI |
| | | LRLSYEQMPSHLRQCFAYCSMLSKGQEIPREDFINRWIAQGFIQS |
| | | SNGSRKLEDIGNQYFDELLSRFCFLDVVQAFDGEILACKLHNLV |
| | | HDLAQSVAGSECSNVKSNASVVSERVRHLFFHAEDMSRKHFPRF |
| | | LLSLQKLRSFSYAFNIGPANKFFVKTTLSNFKCLRVLVLNNLDFE |
| | | ELPTSIGHLKELRYLNLSDNGNIKFLPRSMSKLVNLQTLNLINCE |
| | | QLKELPRDFGKLICLKTLYLTTYKISAGKNQQSFPSLQFLLLFKCC |
| | | FPKLQPELVQQFTALRVLRIYECPSLCSLPSSIRYLTSLEKLWIWN |
| | | CEELDLMDGEGMVGLTNLRSLLLMGLPKLVTLPLGLKDAAHAT |
| | | LNYFRVADCPNLVVLPEWLQDCSSLQRLYIEDCPVLATVPQGIY |
| | | NHNANVHIIDCPLLSGGC |
| N. tabacum Ptr1 | 122 | MLLQSMAETFLFNIIERVLAKVSSVAVYEISLAWNVKTELRKLQS |
| | | TLSTIKAVLLDANEQQAKNHEVRDWLEKLRDVVYDVDDLLDDL |
| | | STQLTLQMHFQKSLKKKVRKFFSSSNPIFYRFKIGRKVKEIRELLN |
| | | EIAHDRKSFHFTEHTYLNPVENICREQTHSFVRASDIIGRETDQEN |
| | | IVKQLIDAREEENISVIPIVGLGGLGKTTLVKLVVVQNFD |
| | | LRMWVSISEDFSLSKVIEKILRSATGESFGHLDMDQLQSHLSEVL |
| | | RSKRYLLVLDDVWNEDQNKWTDLRELLMNCSRGSKIVVTTRSK |
| | | MVALITGTVAPYYLGGLADDECLSLFLKCAFVGEDKLLPNLVEI |
| | | GKEIVKKCGGVPLAVKTLGRLLYMKTDENEWLRIRDNEIWEIEQ |
| | | KQSDILPILRLSYEQMPSHLRQCFAYCSMLSKGQEIPREDFINRWI |
| | | AQGFIQSSNGSRKLEDIGNQYFDELLSRFCFLDVVQAFDGEILAC |
| | | KLHNLVHDLAQSVAGSECLNVKSNASVVSERVRHLFFHAEDMS |
| | | RKHFPRFLLSLQKLRSFSYSFNIGPVNKFFVKTMLSNFKCLRVLV |
| | | LNNLDFGELPTSIGHLKELRYLNLSDNGNIKFLPRSMSKLVNLQT |
| | | LNLINCEQLKELPRDFEKLICLKTLYLTTYKISAGKNQQSFPSLQF |
| | | LLLFKCCFPKLQPELVQQFTALRILRIYECRSLYSLPSSIRYLTSLE |
| | | KLWIWNCEELDLMDGEGMVGLTSLRSLLLMGLPKLVTLPLGLK |
| | | DTAHATLKYFRVADCPNLAVLPEWLQNCSSLQRLYIEDCPVLAS |
| | | IPQGIYNHNAEVHIIDCPLLSGGC |
| N. tomentosiformis | 123 | MAETFLFNIIERVLAKVSSVAVYEISLAWNVKTELRKFQSTLSTIK |
| Ptr1 | | AVLLDANEQQAKNHEVRDWLEKLRDVVYDVDDLLDDLSTQLT |
| | | LQMHFQKSLKKKVRKFFSSSNPIIYRFKIGRKVKEIRELLNEIAHD |
| | | RKSFHFTEHTYLNPVENICREQTHSFVRASDIIGRETDQENIVKQL |
| | | IDAREEENISVIPIVGLGGLGKTTLVKLVYNNNKVVQNFDLRMW |
| | | VSISEDFSLSKVIEKILRSATGESFGHLDMDQLQSHLSEVLRSKRY |
| | | LLVLDDVWNEDQNKWTDLRELLMNCSRGSKIVVTTRSKMVALI |
| | | TGTVAPYYLGGLADDECLSLFLKCAFVGEDKLLPNLVEIGKEIV |
| | | KKCGGVPLAVKTLGRLLYMKTDENEWLRIRDNEIWEIEQKQSDI |
| | | LPILRLSYEQMPSHLRQCFAYCSMLSKGQEIPREDFINRWIAQGFI |
| | | QSSNGSRKLEDIGNQYFDELLSRFCFLDVVQAFDGEILACKLHNL |
| | | VHDLAQSVAGSECLNVKSNASVVSERVRHLFFHAEDMSRKHFP |
| | | RFLLSLQKLRSFSYSFNIGPVNKFFVKTMLSNFKCLRVLVLNNLD |
| | | FGELPTSIGHLKELRYLNLSDNGNIKFLPRSMSKLVNLQTLNLINC |
| | | EQLKELPRDFEKLICLKTLYLTTYKISAGKNQQSFPSLQFLLLFKC |
| | | CFPKLQPELVQQFTALRILRIYECRSLYSLPSSIRYLTSLEKLWIW |
| | | NCEELDLMDGEGMVGLTSLRSLLLMGLPKLVTLPLGLKDTAHA |
| | | TLKYFRVADCPNLAVLPEWLQNCSSLQRLYIEDCPVLASITQGIY |
| | | NHNAEVHIIDCPLLSGGC |
| NbPtr1a | 124 | ATGGCAGAATTTTTCTTGTTCAACATCATTGAAAGAGTTTTGT |
| | | CAAAAGTTTCTTCAGTTTCTGTAAATGAGATCAGTTTAGCGTG |
| | | GAATGTTAAAACAGAGTTAAGAAAACTCCAGAGCACTTTATC |
| | | CACAATCAAAGCTGTACTTTTAGATGCAAATGAGCGGCAGGC |
| | | AAAGAACCATGAAGTGAGAGATTGGCTGGAAAAGCTAAGAG |
| | | ATGTTGTTTATGATGTCGATGATGTGCTCGATGATTTGTCAAC |
| | | ACAGCTATTACTACAAATACATTTTCAGGAAAGCCTTAAGAA |
| | | GAAGGTAAGAAAATTCTTTTCAAGTTCAAATCCAATTATTTAT |
| | | AGATTCAAGATTGGTAGAAAGATAAAAGAGATTAGGGAACT |
| | | ATTGAATGATATTGCAGATGATCGGAAAAGTTTCCACTTTACT |
| | | GAACATACTTGTATAAATCCAGTTGAAAATATTTGTAGAGAA |
| | | CAAACACATTCCTTTGTAAGGGCCTCTGATATTATTGGTAGAG |
| | | AAACTGATCAAGAAAACATAGTAAAACAGCTCATAGATTCTC |
| | | GCGATGAGGAAAATATTTCTGTGATTCCTATTGTTGGACTTGG |
| | | AGGGCTTGGAAAAACCACACTTGTTAAGTTGGTTTATAACAA |
| | | TAATAAGGTTGTTCAGAATTTTGACCTGAGAATGTGGGTGAG |
| | | TATTTCAGAAGATTTTAGTCTGAGTAAGGTAATTGAGAAAATT |
| | | CTGCGATCTGCAACAGGAGAGAGTTTTGGCCACCTAGATATG |
| | | GACCAATTACAAGGTCATTTGAGTGAGGTTTTGCGATCGAAA |
| | | AGGTATTTACTTGTACTGGATGATGTGTGGAATGAAGATCAA |
| | | AACAGGTGGACGGACTTGAGGGAGTTGCTGATGAATTGTTCT |
| | | AGAGGTAGTAAGATTGTTGTCACTACACGCAGTAAGATGGTT |
| | | GCTTTGATTACTGGAACAGTTGCACCTTACTATTTGGGTGGTC |
| | | TTACCGATGATGCGTGCTTATCGTTATTTTTGAAATGTGCATT |
| | | TGTAGGGGAGGACAAATTGTTGCCTAATCTAGTAGAAATAGG |
| | | AAAAGAGATTGTGAAAAAGTGTGGAGGAGTGCCTTTGGCTGT |
| | | GAAAACCTTGGGAAGGTTATTGTATATGAAAACAGACGAAAA |
| | | TGAATGGTTGCGGATAAGAGATAATGAGATATGGGAGATCGA |
| | | ACAGAAACAATCAGACATTTTACCAATATTGAGATTGAGCTA |
| | | TGAACAGATGCCATCTCATCTAAGACAGTGCTTTGCCTATTGC |
| | | TCCATGTTATCCAAAGGTCAAGAAATTCCGAGAGAGGACTTC |
| | | ATCAACCGGTGGATTGCTCAAGGATTTATACAGAGTTCTAAC |
| | | GGATCCAGGAAGTTGGAAGATATTGGTAATCAGTACTTTGAT |
| | | GAGTTGCTATCAAGGTTTTGCTTCCTTGATGTGGTACAAGCAT |
| | | TTGATGGAGAAATATTGGCTTGTAAGTTACACAATCTTGTGCA |
| | | TGATCTTGCACAGTCAGTGGCAGGTTCTGAATGTTCAAATGTG |
| | | AAATCTAATGCTTCTGTGGTTTCTGAAAGAGTTCGCCACTTAT |
| | | TTTTTCATGCAGAAGATATGTCTAGGAAACACTTCCCAAGATT |
| | | TTTACTTTCTTTGCAAAAGTTGAGGTCTTTTTCTTACGCATTTA |
| | | ACATTGGACCTGCAAACAAGTTCTTTGTCAAGACGACATTATC |
| | | AAATTTCAAATGCCTTCGGGTGTTAGTCTTGAACAATTTAGAT |
| | | TTTGAGGAGTTGCCAACTTCGATAGGTCACTTGAAGGAACTA |
| | | AGATATCTTAACCTCAGTGACAATGGTAACATCAAGTTTCTCC |
| | | CAAGGTCTATGAGCAAATTAGTAAATCTGCAGACTCTTAACC |
| | | TCATTAATTGTGAACAGCTTAAGGAGTTGCCGAGAGACTTTG |
| | | GAAAGTTAATCTGCTTGAAGACCTTGTATTTGACTACATATAA |
| | | GATATCAGCAGGGAAGAATCAACAATCTTTCCCTTCTCTTCAA |
| | | TTTTTGCTTCTTTTCAAGTGTTGTTTCCCAAAATTGCAGCCAGA |
| | | ACTGGTGCAGCAGTTTACTGCACTTCGGGTTTTGCGTATCTAT |
| | | GAATGCCCGAGTTTATGTTCTCTTCCAAGCAGTATTAGATACC |
| | | TGACTTCACTTGAAAAGCTATGGATTTGGAACTGTGAAGAAC |
| | | TTGATTTGATGGATGGAGAGGGAATGGTAGGCCTAACAAATT |
| | | TACGGTCGTTGCTTCTAATGGGACTCCCTAAGTTGGTGACTCT |
| | | ACCATTGGGACTTAAAGATGCTGCTCATGCAACACTGAACTA |
| | | CTTTAGAGTTGCCGATTGTCCCAACCTAGTGGTGCTTCCAGAA |
| | | TGGCTGCAGGATTGCTCTTCCCTTCAGAGGCTGTATATAGAGG |
| | | ATTGTCCTGTATTGGCAACTGTACCTCAAGGAATCTACAACCA |
| | | TAATGCCAATGTCCATATAATCGACTGTCCATTGCTAAGTGGA |
| | | GGATGCTAA |
| NbPtr1b | 125 | ATGGCAGAATCTTTCTTGTTCAACATCATTGAAAGAGTTTTGG |
| | | CAAAAGTCTCTTCAGTTGCTGTAAATGAGATCAGTTTAGCTTG |
| | | GATTGTTAAAATTAAGAAAACTCCAGAGCACTTTATCCACAA |
| | | TCAAAGCTGTACTTTTAGATGCAAATGAGCAGTAGGCAAAGA |
| | | ACCATGAAGTGAGAGATTGGCTGGAAAAGCTAAGACATATTG |
| | | TTTATGATGTCGATGATCTGCTCGATGATTTGTCTACACAGTT |
| | | ATTACTACAAATACATTTTCAGAAAAGCCTTAAGAAGAAGGT |
| | | AAGAAAATTCTTTTCAAGTTCAAATCCAATTATTTATAGATTC |
| | | AAGATTGGTAGAAAGGTAAAAGAGATTAGGGAACTATTGAAT |
| | | GAGATTGCAGATGATAGGAGAACTTTCCACTTTACTGAACAT |
| | | ACTTGTCTAAATCCAGTTGAGAATATTTGTAGAGAACAAACA |
| | | CATTCCTTTGTAAGGGCCTCCGATATTATTAGTAGAGAAACTG |
| | | ATCAAGAAAACATAGTAAAACAGCTCATAGATGCTCGTGACG |
| | | AGGAAAATATTTCTGTGATTCCTGTTGTTGGACTTGGTGGGCT |
| | | TGGAAAAACCACACTTGTTAAGTTGGTTTATAACAATAATAA |
| | | GGTTGTTCAGAATTTTGACCTGAGAATGTGGGTTAGTATTTCA |
| | | GAGGATTTTAGTCTGAGTAAGGTAATTGAGAAAATTCTGCGG |
| | | TCTGCAACAGGAGAGAGTTTTGGCCACCTTGATATGGATCAA |
| | | TTACAAGGTCATTTAAGTGAGGTTTTGCGATCAAAATGGTATT |
| | | TACTTGTACTGGATGATGTGTGGAATGAAGATCAAAACAGGT |
| | | GGACGGACTTGAGGGAGTTGCTGATGAATTGTTCTAGAGGTA |
| | | GTACGATTGTTGTCACTACACGCAATAAGATGGTTGCTTTGAT |
| | | TACTGGAACAGTTGCACCTTACTATTTGGGTGGTCTTACTGAT |
| | | GTGCGTGCTTATCGTTATTTTTGAAATGTGCATTTGTAGGGGT |
| | | GGAAAAATTGTTGCCTAATCTAGTAGAAATAGGAAAAGAAAT |
| | | TGTGAAAAAGTGTGGAGGAGTGCCTTTGGCTGTGAAAACCTT |
| | | GGGAAGGTTGCTGTACATGAAAACAGACGAAAATGAATGGTT |
| | | GCGGATACGAGATAATGAGATATGGGAGATCGAACAGAAAC |
| | | AATCTGACATTTTGCCAATATTGAGATTGAGCTATGAACAGAT |
| | | GCCATCACATCTAAGACAGTGCTTTGCCTATTGCTCCATGTTA |
| | | TCCAAAGGTCAAGAAATTCCGAGAGAGGACTTCATCAACCGG |
| | | TGGATTGCTCAAGGATTTATACAGAGTTCAAACGGATCCAGG |
| | | AAGTTGGAAGATATTGGTAATCAGTACTTTGATGAGTTACTAT |
| | | CGAGGTTTTGCTTCCTTGATGTGGTACAAGCTTTTGATGGAGA |
| | | AATATTGGCTTGTAAGTTACACAATCTTGTGCATGATCTTGCA |
| | | CAGTCAGTGGCAGGTTCTGAATGTTTAAATGTGAAATCTAAT |
| | | GCTTCTGTGGTTTCTGAAAGAGTTCGCCACTTATTTTTTCATGC |
| | | AGAAGATATGTCTAGGAAACACTTCCCAATATTTTTACTTTCT |
| | | TTGCAAAAGTTGAGGTCTTTTTCTTACTCATTTAACATTGGAC |
| | | CTGTAAACAAGTTCTTTGTCAAAACGACGTTATCAAATTTCAA |
| | | ATGCCTTCGGGTGTTAGTCTTGAACAATTTAGATTTTGAGGAG |
| | | TTGCCAACTTCGATAGGTCACTTGAAGGAATTAAGATATCTTA |
| | | ACCTTAGTGACAATGGCAACATCAAGTTTCTCCCAAGGTCTAT |
| | | GAGCAAATTAGTAAATCTGCAGACTTTTAACCTCATTAGTTGG |
| | | GAACAGCTTAAGGAGTTGCCAAGAGGCTTTGGAAAGTTAATC |
| | | TGTTTGAAGACCTTGTATTTGACTACATATAAGATACGGAAA |
| | | AGAGTCATATGTTCTACTAGTCAAAATAAGCTACATTTGTCCT |
| | | CCGTTATACTATTTGGCCATATATGTCCCTACCGTTCACATCT |
| | | CTGGCCATATATGTCCCTCTGACGTTAACTTTTTTAAAAAAAT |
| | | AATTAAAAGCCACGTGTCATAGTCCTATTGGTTAAACTAAAC |
| | | CCACTTCTTTTTTTTAATATTAAAACGAAAATATTTGTAAAAA |
| | | ATGTAAAAAAACTTGTAAAAAATGTGTCAAAAATAATTTTGT |
| | | CAAAAATAAAAATATTTTATTAAAAAAATTTTATTTTTGTTTT |
| | | TACAAGAAGAAAATCAGTTCATTTTCAAATTTGAAAAATATTT |
| | | CCAAAATGTGTTTTTTGTTTTTAAAA |
| SynPtr1 | 126 | ATGGCAGAATCATTCCTTTTCAACATTATTGAGAGGGTTCTTG |
| | | CTAAGGTTTCATCAATCGCTGTGTATGAAATCTCACTAGCATG |
| | | GAACGTTAAAACAGAACTAAGAAAACTTCAATCTACTCTTTC |
| | | CACTATCAAGGCTGTTCTTCTTGATGCTAACGAACAAAAAGC |
| | | AAAAAACCACGAAGTTAGAGACTGGCTTGAAAAACTCAGGG |
| | | ATGTGGTTTACGATGTTGATGACCTTATGGACGATCTTTCAAC |
| | | TCAACTTTTGCTTCAGATGCACTTCCAAAAATCTTTTAGAAAG |
| | | AAAGTAAGGAGATTTTTTTCTAGTTCTAATCCTATTATTTATA |
| | | GATTCAAAATTGGAAGAAAAGTAAAGGAAATTAGGGAACTG |
| | | CTTAATGAAATTGCTGATGACAGGAGGAATTTTCACTTTACGG |
| | | AGCATACATATGTTATTCCTGCTGAGAATACTAGTAGGGAAC |
| | | AGACACATTCCTTCGTGAGAGCCTCTGATATTATTGGAAGAG |
| | | ACGATGACCAAGAGAACATCGTAAAGCAGCTTATAGACTCTC |
| | | ACGATGAAGAAAACATTTCAGTGATCCCTATCGTTGGTCTTGG |
| | | TGGGTTGGGAAAGACCACTCTTGTGAAGCTTGTTTACAACAA |
| | | CAATAGAGTTGTGCAGAACTTTGATCTTAGGATGTGGGTGAG |
| | | TATCTCAGAGGATTTTAGTCTTAGCAAAGTAATCGAGAAGAT |
| | | TCTTAGGTCTGCAACTGGAGAAAGTTTCGACCATCTAGACAT |
| | | GGATCAACTTCAATGCTGTCTTGGAGAAGTTCTTCAACAAAA |
| | | AAGATATCTTCTTGTTCTGGACGATGTGTGGAATGAAGACCA |
| | | ACATAAGTGGACAGATCTTAGAGAGCTTCTTATGAACTGTTCT |
| | | AGAGGAAGTAAGATTGTGGTCACAACAAGAAGTAAAATGGT |
| | | GGCTCTTATTACAGGAACTGTTCCACCTTACTATCTTGGAGGA |
| | | CTTGCAAATGACGACTGTTTATCATTATTCTTGAAGTGTGCTT |
| | | TTGGTGGACAAGACAACTTGTTCCCTAACCTAGTTGAAATTGG |
| | | AAAGGAAATCGTGAAGAAGTGCGGAGGTGTGCCATTGGCAGT |
| | | GAAGACCCTTGGAAGATTGCTTTATATGAAGACAGATGAGAA |
| | | CGAGTGGTTGCAAATAAGGGATAACGAGATTTGGGAGATCGA |
| | | GCAGAACAAATCAGACATCTTACCTATACTTAGACTTAGCTA |
| | | CGAACAAATGCCTTCACACCTAAGGCAGTGTTTTGCTTATTGT |
| | | TCTATGTTGCCCAAGGGTCAGGAAATCCCGAGAGAGGACTTT |
| | | ATTAATAGATGGATCGCTCAGGGATTCATACAAAGTTCTAAC |
| | | AGGAACAGAAAGCTTGAAGACATCGGAAATCAATACTTCGAT |
| | | GAATTGCTTTCAAGATTTTGTTTCCTTGATGTTGTACAGGCTTT |
| | | CGATGGTGAAATTTTGGCATGTAAAATACATAATTTGGTGCA |
| | | CGATTTGGCACAATCAGTTTCAGGAGCAGAATGCCTTAATGTT |
| | | AAACCTAATGCATTCGTTGTCTCAGAAAGGGTTAGACACCTTT |
| | | TTTTTCATGCTGAAGACATGTCAAGGAAGCACTTTCCCAGGTT |
| | | TCTTCTTCCATTGCAGAAGCTTAGGTCATTCTCATATTCATTTA |
| | | ATATTGGTCCTGTTAACAAATTCTTCGTCAAAACTATGCTTTC |
| | | AAACTTCAAGTGCTTGAGAATGCTTGTCCTTAACAACCTAGAC |
| | | CTTGAAGAGCTTCCAACATCGATTGGTCATTTGAAAGAACTTA |
| | | GATATCTCAATCTTTCTGACTCTGGTAAAATCAAATTTTTGCC |
| | | AAGATCAATGTCTAAACTTGTAAACCTGCATACCCTAAATCTC |
| | | ATCAACTGCGAACAACTTAAAGAGCTTCCAAGGGATTTCAGA |
| | | AAATTAATTAGCCTTAAGACTTTGTACTTGACAACACACCAA |
| | | ATGTCTGCAGGTATCAAAAATCAGCATTCATTCACATCTTTGC |
| | | AATTCTTATTGCTTTTTAAATGCTGTTTTCCAAAGTTGCAACC |
| | | AGAGCTGGTTCAGCACTTTACTGCATTGCGGGTGTTGAGAATC |
| | | TACGAATGTCCATCATTATGCTCTTTGCCATCTAGTATCAGAT |
| | | ACCTGACATCATTGGAAAAACTTTGGATTTGGAATTGTGAGG |
| | | AATTGGATCTTATTGACGGAGAGGGAATGTCTGGCCTTACAT |
| | | CTCTTCAGTCCCTTCTTCTTATGGGACTTCCAAAGCTTGTGAC |
| | | ACTACCTTTGGAGCTTAAGGATACAGCTCCAACAACTTTAAA |
| | | ATACTTTAGAATTGCCGACTGTCCTAACCTTGTGGAACTTCCT |
| | | GAATGGCTTCCTAACTGCTCTTCATTGCAGAGGCTCTACATAG |
| | | AAGATTGCCCTGTGTTGGCTTCGATTCCTCAGGGAATTTACTC |
| | | TCACAACGCCAATGTCCACATAATTGACTGCCCACTTCTAGGA |
| | | GGATAA |
| RPS2 | 127 | ATGGATTTCATCTCATCTCTTATCGTTGGCTGTGCTCAGGTGTT |
| | | GTGTGAATCTATGAATATGGCGGAGAGAAGAGGACATAAGA |
| | | CTGATCTTAGACAAGCCATCACTGATCTTGAAACAGCCATCG |
| | | GTGACTTGAAGGCCATACGTGATGACCTGACTTTACGGATCC |
| | | AACAAGACGGTCTAGAGGGACGAAGCTGCTCAAATCGTGCCA |
| | | GAGAGTGGCTTAGTGCGGTGCAAGTAACGGAGACTAAAACA |
| | | GCCCTACTTTTAGTGAGGTTTAGGCGTCGGGAACAGAGGACG |
| | | CGAATGAGGAGGAGATACCTCAGTTGTTTCGGTTGTGCCGAC |
| | | TACAAACTGTGCAAGAAGGTTTCTGCCATATTGAAGAGCATT |
| | | GGTGAGCTGAGAGAACGCTCTGAAGCTATCAAAACAGATGGC |
| | | GGGTCAATTCAAGTAACTTGTAGAGAGATACCCATCAAGTCC |
| | | GTTGTCGGAAATACCACGATGATGGAACAGGTTTTGGAATTT |
| | | CTCAGTGAAGAAGAAGAAAGAGGAATCATTGGTGTTTATGGA |
| | | CCTGGTGGGGTTGGGAAGACAACGTTAATGCAGAGCATTAAC |
| | | AACGAGCTGATCACAAAAGGACATCAGTATGATGTACTGATT |
| | | TGGGTTCAAATGTCCAGAGAATTCGGCGAGTGTACAATTCAG |
| | | CAAGCCGTTGGAGCACGGTTGGGTTTATCTTGGGACGAGAAG |
| | | GAGACCGGCGAAAACAGAGCTTTGAAGATATACAGAGCTTTG |
| | | AGACAGAAACGTTTCTTGTTGTTGCTAGATGATGTCTGGGAA |
| | | GAGATAGACTTGGAGAAAACTGGAGTTCCTCGACCTGACAGG |
| | | GAAAACAAATGCAAGGTGATGTTCACGACACGGTCTATAGCA |
| | | TTATGCAACAATATGGGTGCGGAATACAAGTTGAGAGTGGAG |
| | | TTTCTGGAGAAGAAACACGCGTGGGAGCTGTTCTGTAGTAAG |
| | | GTATGGAGAAAAGATCTTTTAGAGTCATCATCAATTCGCCGG |
| | | CTCGCGGAGATTATAGTGAGTAAATGTGGAGGATTGCCACTA |
| | | GCGTTGATCACTTTAGGAGGAGCCATGGCTCATAGAGAGACA |
| | | GAAGAAGAGTGGATCCATGCTAGTGAAGTTCTGACTAGATTT |
| | | CCAGCAGAGATGAAGGGTATGAACTATGTATTTGCCCTTTTG |
| | | AAATTCAGCTACGACAACCTCGAGAGTGATCTGCTTCGGTCTT |
| | | GTTTCTTGTACTGCGCTTTATTCCCAGAAGAACATTCTATAGA |
| | | GATCGAGCAGCTTGTTGAGTACTGGGTCGGCGAAGGGTTTCT |
| | | CACCAGCTCCCATGGCGTTAACACCATTTACAAGGGATATTTT |
| | | CTCATTGGGGATCTGAAAGCGGCATGTTTGTTGGAAACCGGA |
| | | GATGAGAAAACACAGGTGAAGATGCATAATGTGGTCAGAAG |
| | | CTTTGCATTGTGGATGGCATCTGAACAGGGGACTTATAAGGA |
| | | GCTGATCCTAGTTGAGCCTAGCATGGGACATACTGAAGCTCC |
| | | TAAAGCAGAAAACTGGCGACAAGCGTTGGTGATCTCATTGTT |
| | | AGATAACAGAATCCAGACCTTGCCTGAAAAACTCATATGCCC |
| | | GAAACTGACAACACTGATGCTCCAACAGAACAGCTCTTTGAA |
| | | GAAGATTCCAACAGGGTTTTTCATGCATATGCCTGTTCTCAGA |
| | | GTCTTGGACTTGTCGTTCACAAGTATCACTGAGATTCCGTTGT |
| | | CTATCAAGTATTTGGTGGAGTTGTATCATCTGTCTATGTCAGG |
| | | AACAAAGATAAGTGTATTGCCACAGGAGCTTGGGAATCTTAG |
| | | AAAACTGAAGCATCTGGACCTACAAAGAACTCAGTTTCTTCA |
| | | GACGATCCCACGAGATGCCATATGTTGGCTGAGCAAGCTCGA |
| | | GGTTCTGAACTTGTACTACAGTTACGCCGGTTGGGAACTGCA |
| | | GAGCTTTGGAGAAGATGAAGCAGAAGAACTCGGATTCGCTGA |
| | | CTTGGAATACTTGGAAAACCTAACCACACTCGGTATCACTGTT |
| | | CTCTCATTGGAGACCCTAAAAACTCTCTTCGAGTTCGGTGCTT |
| | | TGCATAAACATATACAGCATCTCCACGTTGAAGAGTGCAATG |
| | | AACTCCTCTACTTCAATCTCCCATCACTCACTAACCATGGCAG |
| | | GAACCTGAGAAGACTTAGCATTAAAAGTTGCCATGACTTGGA |
| | | GTACCTGGTCACACCCGCAGATTTTGAAAATGATTGGCTTCCG |
| | | AGTCTAGAGGTTCTGACGTTACACAGCCTTCACAACTTAACCA |
| | | GAGTGTGGGGAAATTCTGTAAGCCAAGATTGTCTGCGGAATA |
| | | TCCGTTGCATAAACATTTCACACTGCAACAAGCTGAAGAATG |
| | | TCTCATGGGTTCAGAAACTCCCAAAGCTAGAGGTGATTGAAC |
| | | TGTTCGACTGCAGAGAGATAGAGGAATTGATAAGCGAACACG |
| | | AGAGTCCATCCGTCGAAGATCCAACATTGTTCCCAAGCCTGA |
| | | AGACCTTGAGAACTAGGGATCTGCCAGAACTAAACAGCATCC |
| | | TCCCATCTCGATTTTCATTCCAAAAAGTTGAAACATTAGTCAT |
| | | CACAAATTGCCCCAGAGTTAAGAAACTGCCGTTTCAGGAGAG |
| | | GAGGACCCAGATGAACTTGCCAACAGTTTATTGTGAGGAGAA |
| | | ATGGTGGAAAGCACTGGAAAAAGATCAACCAAACGAAGAGC |
| | | TTTGTTATTTACCGCGCTTTGTTCCAAATTGA |
| Mr5 | 128 | ATGGGGGGAGAGGCTTTTCTTGTGGCATTCCTCCAAGTGCTGG |
| | | TTGACAAGTTGGCGCATCGCGAGGTCTTCAAGTACTTTGGACT |
| | | TGTAAAGGGCGTAGATCAAAAACTGAAGAAATGGAGTGCCA |
| | | CCTTGTCTGCGATTGGAGCGGTGCTGAATGACGCAGAGGAGA |
| | | GGCAACTGACGGCTAAGAACAACACACTGAAGCTCTGGCTCG |
| | | AAGATCTCAGGGACTTGGCTTTTGATGTGGAAGACGTGTTGG |
| | | ACAAATATGCTACTAAAATGTTGAAACGTCAGATACAACATG |
| | | CTCATTCCCGCACAACAAGCAAACTATGGAACTCAATTCCTG |
| | | ATGGTGTTTTCAACTTCAACATGAACTCGGAAATACAGAAGA |
| | | TTAGTGAGCGATTACAAGAGATATCTGAACAAAAAGACCAGC |
| | | TTAATTTGAAAATTGATACTGGGGCGTTGACTACAAGGGCAC |
| | | GCCGAAACATATCACCTAGTTCCAGTCAACCAGATGGACCTG |
| | | TGATTGGAAGGGATGAGGACAAAAGAAAGATTGTTGAGCTGC |
| | | TGTCGAAACAAGAGCATCGTACTGTCAATTTCGATGTAGTTGC |
| | | AATTGTTGGTATGGCTGGAGTCGGAAAGACAACACTTGCTGG |
| | | ACAAGTACTCAATGATATGGTTGCAACCCAAACGTTTCAACC |
| | | AGCTGTTTGGGCATGCGTATCTGACGACTTCAACCTTGAAAG |
| | | AGTGACAAAGCAAATTCTTGAATCAATCACATCTCGGCAATG |
| | | CACCACAGAAGATTACAATAAGGTTCAAGATTATCTGCATAA |
| | | GGAGTTAGCGGGGAAGAAGTTTTTAATTGTTTTAGATGATGT |
| | | GTGGAAAACGTGTAGCTACGGTGAATGGATGAAGTTGCAGTC |
| | | CCCTTTTCGTGACGGAGCACAAGGAAGCAAGATAATTGTGAC |
| | | AACACGTGATACAGATGTTTCAAAAATGATGGGAGCTGCCAC |
| | | GCTCGTTCACAATTTGGAGCCTATGGAAAGTAGTGTTTGTTTG |
| | | CAAGTATTTGAGCAGCATGCATTCTTAAATTCTAATGACGACA |
| | | AACCACCAAATTACGAGTTACTTAAGGAAAAAATTGCTGCCA |
| | | AGTGTAGGGGATTGCCTTTGGCCGCAAGGACCCTTGGTGGTG |
| | | TTCTACTTCGTAAAGATACATACGAATGGGAAGACATATTGA |
| | | ACAACAAACTGTGGAGTCTATCAAATGAGCACGACATACTTC |
| | | CAGTACTGAGATTAACCTACTTTTATCTTCCTTCACATTTGAA |
| | | AAGATGCTTTGCCTATTGCTCAATACTTCCAAATGACTATGAA |
| | | TTTGAAGAGAAGCAAATGATCCTTCTATGGATGGCCGAGGGG |
| | | TTTATTCTTCCACGACCAGAAGATAAGAAGCAAATTGAAGAT |
| | | TTAGGTGCTGATTATTTTCGGGATCTCGTATCAAGGTCATTGT |
| | | TTCAAAAATCAACCAAATGTATTTCAAAATATGTGATGCATG |
| | | ACCTTATTGGTGATTTAGCACGGTGGGCAGCAGGAGAAATTT |
| | | GTTTTAGATTGGAGGATAAGCAAAATGATGATGGTGAACAAC |
| | | TTAGATGTTTTCCCAAGGCACGCCATTCGTCTTACATCAGGGG |
| | | TCTGTCTGATGGGGTCAAAAGATTTGAGGTATTTTCTGAACTG |
| | | AAATATTTGCGAACCTTCTTGCCACTAAGAAAGGATTCTTTCT |
| | | GGAATTATTTAAGTCGTCAGGTTGCTTTTGATTTATTGCCGAA |
| | | ATTGCAATATTTGCGGGTGCTCTCTTTCAATTGCTATAAAATA |
| | | ACTGAGCTTCCAGACTCAATCGGTGATTTAAGGTATCTACGGT |
| | | ATCTCGACCTTTCCTACACAGATATAACAAGTTTACCTAAATC |
| | | AACAAGCACTCTTTACAACTTGCAGACGTTGATATTGGAAGG |
| | | CTGTTCTAAATTGAAGGCATTGCCTATAGACATGAGTAATCTA |
| | | GTTAATTTGCGTCATCTCAACAACTCAAATGTATCTTTGTTGG |
| | | AAGACATGCCTCCACAACTAGGTCGATTGGTGAATCTCCAGT |
| | | CATTGACTAAGTTTGTGGTGAGTGGTGGTGGTGGTGGTGATC |
| | | GATCAGGGATACGAGAGCTGGAGTTCCTAATGCATCTCCGAG |
| | | GAACATTGTGCATCTCAAGATTGGAGAATGTGACTGATGTCG |
| | | AGGATGCTCAGAGGGCCAACTTAAACTGCAAAGAGAGGCTTG |
| | | ATTCGTTGGTGCTAGAATGGTCTCATTCAAGCGACACAAGAG |
| | | AAACAGAATCCGCTGTGCTTGACATGTTACAGCCTCATACAA |
| | | AGCTTAAGGAGCTCACCATCAAGAGTTATGCAGGAAAAGAAT |
| | | TTTCATCATGGGTTGGAGTTCCATTGTTCTCTAATATGGTGCT |
| | | CGTGCGCTTAGAGGAATGTAACAATTGTCTATCTCTACCACCT |
| | | CTCGGAAAATTGCCTCATCTCAAAGAACTTTATATTAGAGGA |
| | | ATGAATGCAGTGGAAAGTGTTGGAGCTGAGTTTTATGGAGAG |
| | | TGCTCCTTGCCTTTTCCGCTATTAGAGACTCTTGAGTTTGTGG |
| | | ATATGCAACATTGGAAAGTATGGCTTCCATTTCAAACGGATC |
| | | ACAGAGGTAGTGTTTTCCCTTGCCTGAAAACACTCTTAGTCAG |
| | | AAAATGTTCTAAACTGGAGGGTAAGCTGCCAGAGAACCTTGA |
| | | TTCGCTGGCATCGCTTGAAATTGTTAAATGTGAAGAATTATTG |
| | | GTTTCAATTGCCAATTACAAACAACTTCGTCAGTTAAACATTG |
| | | ACGGTTGTAAAGGGGTGGTGCATACAGCTGCTAAGGTTGAGT |
| | | TTGAGTTATTAGAGTCCTTGTACCTTTCAAATATTTCGGAGCT |
| | | GACGTCTCTACAAACAGGAGAATTGTGCAGAAATGGATTAAA |
| | | CATGGTCAGAGATTTGAAGATTAATGGATGTGAGGAGCTGAC |
| | | GTCTTCATTGAAGAATGAGGCAATATTATTGCAACAGTTGATT |
| | | TCTCTTGGCCGTTTAGAGATTGAAGACAACTCTCTCCTAGTTG |
| | | AAGAACTAGGAAAAGAAGCAGATGAGTTGTTGCAATTGCAA |
| | | ATATTGGGGTGTAAGCTTGAATTTCTGAAGTTAAAGAAGTGC |
| | | AAAAATCTTTTGAAGCTACCAGAAGGGTTAAATCAGTTGTCG |
| | | TCACTTCAAGAGCTTCGCATACATGAATGTTCAAGTCTAGTTT |
| | | CTTTTCCAGATGTTGGTTTGCCACCTTCTCTTAAAGACATCGA |
| | | GATTACAGAGTGCCACTCGTTGATATATTTTGCAAAATCCCAG |
| | | ATTCCCCAAAATCTCAGAAGAATACAGATAAGAGATTGCAGA |
| | | AGTTTGAGATCACTAGTAGACAATGAGGCTGTTGGTTCTTGTT |
| | | CTTCGTCTTCTCACAATTGTCTTGAGTACTTGAATATCGAGAG |
| | | ATGTCAATCTCTAACGTTGTTATCATTGAGTGACCAGCTTGTC |
| | | AGGGCACTTAGAGAACTTGACATATATGATTGTGAACAACTG |
| | | GAGTTTCTCGCACCGGACGGGTTGTTCTGCAACAACACTAATT |
| | | ACTTTCTCGAAAATTTTAGGATACGGAGGTGCCAAAATCTGA |
| | | AATCCTTACCGAGACTGAGTGGGGGGATAAGGGGCTCTAACC |
| | | TGAGAGAGATCCGGATCACCGATTGCGACAGATTGGAGGCCT |
| | | TGCCCGAAGACATGCACAATTTCAACTCTCTTGAGAAATTGAT |
| | | TATCGACTACCGTGAAGGTTTGACTTGCTCCTTTCCCGCCAAC |
| | | CTAACATCACTTATGATTTGGAAGGTCAAGAGCTGTAAGTCA |
| | | TTGTGGGAGTTGGAGTGGGGGTTACACAGACTCACCTCTCTTA |
| | | GATACTTGTGGATCGGTGGTGAAGACCCGGATATGGTATCGT |
| | | TTCCACCGGACATGGTCCGAATGGAGACGTTGCTCCCCAAAT |
| | | CTCTCACTGAACTCTCAATTGGTGGCTTCCCGAATCTGAAGAA |
| | | ACTGAGCAGCAAGGGCTTTCAATTCCTAACCTCCCTTGAATCT |
| | | TTGGAACTCTGGGATTGTCCAAAGCTAGCATCCATTCCAAAG |
| | | GAGGGACTGCCTCTTTCACTTACGGAACTTTGCATCTATGGGT |
| | | GTCCTGTTCTAAAAGAGAGATGTCAACCAGGAAAAGGACGCT |
| | | ACTGGCACAAAATATCCCACATCCCTTACATAGATATAGATT |
| | | GGAAGATGATTTGA |
| RPS2 | 129 | MDFISSLIVGCAQVLCESMNMAERRGHKTDLRQAITDLETAIGD |
| | | LKAIRDDLTLRIQQDGLEGRSCSNRAREWLSAVQVTETKTALLL |
| | | VRFRRREQRTRMRRRYLSCFGCADYKLCKKVSAILKSIGELRER |
| | | SEAIKTDGGSIQVTCREIPIKSVVGNTTMMEQVLEFLSEEEERGII |
| | | GVYGPGGVGKTTLMQSINNELITKGHQYDVLIWVQMSREFGEC |
| | | TIQQAVGARLGLSWDEKETGENRALKIYRALRQKRFLLLLDDV |
| | | WEEIDLEKTGVPRPDRENKCKVMFTTRSIALCNNMGAEYKLRV |
| | | EFLEKKHAWELFCSKVWRKDLLESSSIRRLAEIIVSKCGGLPLALI |
| | | TLGGAMAHRETEEEWIHASEVLTRFPAEMKGMNYVFALLKFSY |
| | | DNLESDLLRSCFLYCALFPEEHSIEIEQLVEYWVGEGFLTSSHGV |
| | | NTIYKGYFLIGDLKAACLLETGDEKTQVKMHNVVRSFALWMAS |
| | | EQGTYKELILVEPSMGHTEAPKAENWRQALVISLLDNRIQTLPEK |
| | | LICPKLTTLMLQQNSSLKKIPTGFFMHMPVLRVLDLSFTSITEIPLS |
| | | IKYLVELYHLSMSGTKISVLPQELGNLRKLKHLDLQRTQFLQTIP |
| | | RDAICWLSKLEVLNLYYSYAGWELQSFGEDEAEELGFADLEYLE |
| | | NLTTLGITVLSLETLKTLFEFGALHKHIQHLHVEECNELLYFNLPS |
| | | LTNHGRNLRRLSIKSCHDLEYLVTPADFENDWLPSLEVLTLHSLH |
| | | NLTRVWGNSVSQDCLRNIRCINISHCNKLKNVSWVQKLPKLEVI |
| | | ELFDCREIEELISEHESPSVEDPTLFPSLKTLRTRDLPELNSILPSRF |
| | | SFQKVETLVITNCPRVKKLPFQERRTQMNLPTVYCEEKWWKAL |
| | | EKDQPNEELCYLPRFVPN |
| Mr5 | 130 | MGGEAFLVAFLQVLVDKLAHREVFKYFGLVKGVDQKLKKWSA |
| | | TLSAIGAVLNDAEERQLTAKNNTLKLWLEDLRDLAFDVEDVLD |
| | | KYATKMLKRQIQHAHSRTTSKLWNSIPDGVFNFNMNSEIQKISE |
| | | RLQEISEQKDQLNLKIDTGALTTRARRNISPSSSQPDGPVIGRDED |
| | | KRKIVELLSKQEHRTVNFDVVAIVGMAGVGKTTLAGQVLNDMV |
| | | ATQTFQPAVWACVSDDFNLERVTKQILESITSRQCTTEDYNKVQ |
| | | DYLHKELAGKKFLIVLDDVWKTCSYGEWMKLQSPFRDGAQGS |
| | | KIIVTTRDTDVSKMMGAATLVHNLEPMESSVCLQVFEQHAFLNS |
| | | NDDKPPNYELLKEKIAAKCRGLPLAARTLGGVLLRKDTYEWEDI |
| | | LNNKLWSLSNEHDILPVLRLTYFYLPSHLKRCFAYCSILPNDYEF |
| | | EEKQMILLWMAEGFILPRPEDKKQIEDLGADYFRDLVSRSLFQKS |
| | | TKCISKYVMHDLIGDLARWAAGEICFRLEDKQNDDGEQLRCFPK |
| | | ARHSSYIRGLSDGVKRFEVFSELKYLRTFLPLRKDSFWNYLSRQV |
| | | AFDLLPKLQYLRVLSFNCYKITELPDSIGDLRYLRYLDLSYTDITS |
| | | LPKSTSTLYNLQTLILEGCSKLKALPIDMSNLVNLRHLNNSNVSL |
| | | LEDMPPQLGRLVNLQSLTKFVVSGGGGGDRSGIRELEFLMHLRG |
| | | TLCISRLENVTDVEDAQRANLNCKERLDSLVLEWSHSSDTRETE |
| | | SAVLDMLQPHTKLKELTIKSYAGKEFSSWVGVPLFSNMVLVRLE |
| | | ECNNCLSLPPLGKLPHLKELYIRGMNAVESVGAEFYGECSLPFPL |
| | | LETLEFVDMQHWKVWLPFQTDHRGSVFPCLKTLLVRKCSKLEG |
| | | KLPENLDSLASLEIVKCEELLVSIANYKQLRQLNIDGCKGVVHTA |
| | | AKVEFELLESLYLSNISELTSLQTGELCRNGLNMVRDLKINGCEE |
| | | LTSSLKNEAILLQQLISLGRLEIEDNSLLVEELGKEADELLQLQIL |
| | | GCKLEFLKLKKCKNLLKLPEGLNQLSSLQELRIHECSSLVSFPDV |
| | | GLPPSLKDIEITECHSLIYFAKSQIPQNLRRIQIRDCRSLRSLVDNE |
| | | AVGSCSSSSHNCLEYLNIERCQSLTLLSLSDQLVRALRELDIYDCE |
| | | QLEFLAPDGLFCNNTNYFLENFRIRRCQNLKSLPRLSGGIRGSNL |
| | | REIRITDCDRLEALPEDMIHNFNSLEKLIIDYREGLTCSFPANLTSL |
| | | MIWKVKSCKSLWELEWGLHRLTSLRYLWIGGEDPDMVSFPPDM |
| | | VRMETLLPKSLTELSIGGFPNLKKLSSKGFQFLTSLESLELWDCP |
| | | KLASIPKEGLPLSLTELCIYGCPVLKERCQPGKGRYWHKISHIPYI |
| | | DIDWKMI |
| StPtr1 (GenBank | 131 | MAESFLFNIIERVLAKVSSIAVYEITLAWNVKIELRKLQSILSTIK |
| Accession No. | | AVLLDANEQQAKNHEVRDWLEKLRDVVYDVDDLMDDLSTQLL |
| XP_006340095.1) | | LQMHFQKSFRKKVRKFFSSSNPIIYRFKIGRKVKEIRELLNEIADD |
| | | RRNFHFTEHTYVIPAENTSREQTHSFVRASDIIGRDDDQENIVKQ |
| | | LIDSHDEENISVIPIVGLGGLGKTTLVKLVYNNNRVVQNFDLRM |
| | | WVSISEDFSLSKVIEKILRSATGESFDHLDMDQLQGCLGEVLQQK |
| | | RYLLVLDDVWNEDQHKWTDLRELLMNCSRGSKIVVTTRSKMA |
| | | ALITGTVPPYYLEGLADDDCLSLFLKCAFGGQDNLFPNLVEIGKE |
| | | IVKKCGGVPLAVKTLGRLLYMKTDENEWLQIRDNEIWEIEQNKS |
| | | DILPILRLSYEQMPSHLRQCFAYCSMLSKGQBPREDFINRWIAQG |
| | | FIQSSNRNRKLEDIGNQYFDELLSRFCFLDVVQAFDGEILACKIHN |
| | | LVHDLAQSVAGAECLNVKPNAFVVSERVRHLFFHAEDMSRKHF |
| | | PRFLLPLQKLRSFSYSFNIGPVNKFFVKTMLSNFKCLRMLVLNNL |
| | | DLEELPTSIGHLKELRYLNLSNSGNIKFLPRSMSKLVNLHTLNLIN |
| | | CRQLKELPRDERKLISLKTLYLTTHQISEGIKNQHSFTSLQFLLLF |
| | | KCCFPKLQPELVQHFTALRVLRIYECPSLCSLPSSIRYLTSLEKLW |
| | | IWNCEELDLIDGEGMSGLTSLQSLLIMGLPKLVTLPLELKDTAPT |
| | | TLKYFRIADCPNLMELPEWLPNCSSLQRLYIEDCPVLASIPQGIYS |
| | | HNANLHIIDCPLLGG |
| StPtr1 (coding | 132 | ATGGCGGAATCTTTCTTGTTCAATATCATTGAACGAGTTTTGG |
| sequence from | | CTAAAGTTTCTTCAATTGCTGTATATGAGATCACTCTAGCTTG |
| GenBank | | GAATGTTAAGATAGAGCTAAGGAAACTCCAAAGTACTCTATC |
| Accession No. | | CACCATCAAAGCTGTACTTCTAGATGCAAACGAGCAACAGGC |
| XM_006340033.2) | | AAAGAACCATGAAGTGAGAGATTGGCTGGAAAAGCTCAGAG |
| | | ATGTTGTTTATGATGTCGATGATTTGATGGATGATTTATCAAC |
| | | ACAACTGTTGCTGCAAATGCATTTCCAGAAAAGCTTTAGGAA |
| | | GAAGGTAAGAAAATTCTTTTCAAGTTCAAATCCAATTATATAT |
| | | CGATTCAAGATTGGCAGAAAGGTAAAAGAAATCAGGGAGCT |
| | | ACTGAATGAGATTGCAGATGATAGGAGAAATTTCCACTTCAC |
| | | GGAACATACGTATGTAATTCCAGCTGAGAATACGAGTAGAGA |
| | | ACAAACACACTCCTTTGTGAGGGCATCAGATATCATTGGTAG |
| | | AGATGATGATCAAGAAAACATTGTAAAACAGCTGATAGATTC |
| | | TCATGATGAGGAAAATATTTCTGTGATTCCTATTGTTGGACTT |
| | | GGAGGGCTTGGAAAAACCACACTTGTTAAGTTGGTTTATAAC |
| | | AATAATAGGGTTGTTCAGAATTTTGACCTTAGAATGTGGGTTA |
| | | GTATTTCAGAAGATTTTAGTCTGAGCAAGGTAATTGAGAAAA |
| | | TTCTGAGGTCTGCAACGGGAGAGAGTTTTGACCACCTAGATA |
| | | TGGACCAATTACAAGGTTGTTTGGGAGAGGTTTTGCAACAGA |
| | | AAAGGTATTTACTTGTGCTGGATGATGTGTGGAATGAAGATC |
| | | AACACAAGTGGACAGATCTGAGGGAGTTGCTGATGAATTGTT |
| | | CCAGAGGTAGTAAAATTGTTGTCACTACACGTAGTAAGATGG |
| | | CTGCTTTGATTACTGGAACAGTTCCGCCTTATTATTTGGAAGG |
| | | CCTTGCTGATGATGACTGCTTATCTTTATTTTTGAAATGTGCAT |
| | | TTGGAGGGCAGGACAATTTGTTTCCTAATCTAGTAGAAATAG |
| | | GAAAAGAAATTGTGAAAAAGTGTGGAGGAGTGCCTTTGGCTG |
| | | TGAAAACCTTGGGAAGGTTATTGTACATGAAAACAGACGAGA |
| | | ATGAATGGTTGCAAATAAGAGATAATGAGATATGGGAAATCG |
| | | AACAGAATAAATCTGACATTTTACCAATATTGAGATTGAGCT |
| | | ATGAACAGATGCCATCACATCTAAGACAGTGCTTTGCCTATTG |
| | | CTCCATGTTATCCAAAGGTCAAGAAATTCCGAGGGAGGATTT |
| | | TATCAATCGGTGGATTGCTCAAGGATTTATACAGAGTTCAAA |
| | | CAGAAACAGGAAGCTGGAAGATATAGGTAATCAGTACTTTGA |
| | | TGAGTTGCTATCAAGGTTTTGCTTCCTAGATGTGGTACAAGCT |
| | | TTTGATGGAGAAATATTGGCTTGTAAGATACACAATCTTGTGC |
| | | ATGATCTTGCACAGTCAGTAGCAGGTGCAGAATGCTTAAATG |
| | | TGAAACCCAATGCTTTCGTGGTTTCTGAAAGAGTTCGCCACTT |
| | | ATTCTTCCATGCAGAAGATATGTCTAGGAAACACTTCCCCAG |
| | | ATTTTTGCTTCCTTTGCAAAAGTTGAGGTCTTTCTCTTATTCTT |
| | | TTAACATTGGACCTGTAAACAAGTTCTTTGTCAAGACAATGTT |
| | | GTCAAATTTCAAATGCCTTCGGATGTTAGTCTTGAACAATCTA |
| | | GATCTTGAGGAGTTGCCAACTTCGATAGGTCACTTGAAGGAA |
| | | TTAAGATACCTTAACCTTAGCAACAGTGGTAATATCAAGTTTC |
| | | TTCCAAGGTCTATGAGCAAATTAGTAAATCTGCACACCCTAA |
| | | ACCTCATTAACTGTGAACAGCTTAAGGAGTTGCCAAGAGACT |
| | | TTAGAAAGTTAATCAGCCTAAAGACCTTGTATTTGACTACACA |
| | | TCAGATATCAGAAGGGATCAAGAATCAACATTCTTTCACTTCT |
| | | CTTCAATTTTTGCTTCTTTTCAAATGTTGTTTCCCAAAATTGCA |
| | | GCCAGAACTGGTGCAGCATTTTACTGCACTTCGGGTTTTGCGT |
| | | ATCTATGAATGCCCAAGTTTATGTTCTCTTCCAAGCAGTATTA |
| | | GATATCTGACTTCACTTGAAAAGCTATGGATCTGGAACTGTG |
| | | AAGAACTTGATTTGATAGATGGAGAAGGAATGTCAGGCCTAA |
| | | CAAGTCTTCAATCCTTGCTTCTAATGGGGCTTCCTAAGTTGGT |
| | | GACTCTACCATTGGAACTTAAAGATACTGCTCCTACAACATTA |
| | | AAGTACTTCAGAATCGCCGATTGTCCCAACCTGATGGAGCTTC |
| | | CAGAGTGGCTGCCCAATTGCTCCTCACTTCAGAGACTGTATAT |
| | | AGAGGACTGTCCTGTTTTGGCATCGATACCTCAAGGAATCTAC |
| | | AGCCACAATGCCAATCTCCATATAATCGACTGTCCATTGCTAG |
| | | GTGGATGA |

Although certain embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1.-30. (canceled)

31. A method of identifying a candidate plant suitable for breeding that displays enhanced disease resistance, said method comprising:
providing a candidate plant;
analyzing the candidate plant for the presence, in its genome, of a Ptr1 polynucleotide;
identifying, based on said analyzing, a candidate plant suitable for breeding that includes in its genome, a Ptr1 polynucleotide; and
breeding the identified plant with at least one other plant.

32. The method according to claim 31, wherein said analyzing comprises:
isolating genomic DNA from the plant, germplasm, pollen, or seed of the plant;
analyzing genomic DNA from the plant, germplasm, pollen, or seed of the plant for the presence of the Ptr1 polynucleotide; and
detecting the Ptr1 polynucleotide.

33. The method according to claim 31, wherein said breeding comprises crossing, making hybrids, backcrossing, self-crossing, double haploid breeding, and/or combinations thereof.

34. The method according to claim 31, wherein the Ptr1 polynucleotide encodes the amino acid sequence of SEQ ID NO:19.

35. The method according to claim 31, wherein the Ptr1 polynucleotide comprises the nucleotide sequence of SEQ ID NO:18.

36. The method according to claim 31, wherein said providing comprises providing a transgenic plant transformed with a nucleic acid molecule comprising a Ptr1 polynucleotide.

37. The method according to claim 31, wherein said providing comprises:
transforming a plant or plant cell with a nucleic acid construct comprising:
a nucleic acid molecule comprising a Ptr1 polynucleotide;
a 5′ heterologous DNA promoter sequence; and
a 3′ terminator sequence, wherein the nucleic acid molecule, the DNA promoter sequence, and the terminator sequence are operatively coupled to permit transcription of the nucleic acid molecule, and
growing the transgenic plant or a plant grown from the transgenic plant cell under conditions effective to express the nucleic acid molecule in said transgenic plant or said plant grown from the transgenic plant cell.

38.-45. (canceled)
